SEQUENCE LISTING (711 sequences)

SEQ ID NO: 1 - protein, 12 aa, synthetic construct
  REGION 1..12: note = Description of Artificial Sequence: Synthetic peptide
LLADTTHHRPWT

SEQ ID NO: 2 - protein, 12 aa, synthetic construct
  REGION 1..12: note = Description of Artificial Sequence: Synthetic peptide
VIGESTHHRPWS

SEQ ID NO: 3 - protein, 12 aa, synthetic construct
  REGION 1..12: note = Description of Artificial Sequence: Synthetic peptide
LIADSTHHSPWT

SEQ ID NO: 4 - protein, 12 aa, synthetic construct
  REGION 1..12: note = Description of Artificial Sequence: Synthetic peptide
ILAESTHHKPWT

SEQ ID NO: 5 - protein, 12 aa, synthetic construct
  REGION 1..12: note = Description of Artificial Sequence: Synthetic peptide
ILAETTHHRPWS

SEQ ID NO: 6 - protein, 12 aa, synthetic construct
  REGION 1..12: note = Description of Artificial Sequence: Synthetic peptide
IIGESSHHKPFT

SEQ ID NO: 7 - protein, 12 aa, synthetic construct
  REGION 1..12: note = Description of Artificial Sequence: Synthetic peptide
GLGDTTHHRPWG

SEQ ID NO: 8 - protein, 12 aa, synthetic construct
  REGION 1..12: note = Description of Artificial Sequence: Synthetic peptide
VLGDTTHHKPWT

SEQ ID NO: 9 - protein, 12 aa, synthetic construct
  REGION 1..12: note = Description of Artificial Sequence: Synthetic peptide
IVADSTHHRPWT

SEQ ID NO: 10 - protein, 11 aa, synthetic construct
  REGION 1..11: note = Description of Artificial Sequence: Synthetic peptide
STADTSHHRPS

SEQ ID NO: 11 - protein, 12 aa, synthetic construct
  REGION 1..12: note = Description of Artificial Sequence: Synthetic peptide
TSGGESTHHRPS

SEQ ID NO: 12 - protein, 12 aa, synthetic construct
  REGION 1..12: note = Description of Artificial Sequence: Synthetic peptide
TSGGESSHHKPS

SEQ ID NO: 13 - protein, 12 aa, synthetic construct
  REGION 1..12: note = Description of Artificial Sequence: Synthetic peptide
TGSGDSSHHRPS

SEQ ID NO: 14 - protein, 13 aa, synthetic construct
  REGION 1..13: note = Description of Artificial Sequence: Synthetic peptide
GSSGESTHHKPST

SEQ ID NO: 15 - protein, 12 aa, synthetic construct
  REGION 1..12: note = Description of Artificial Sequence: Synthetic peptide
VGADSTHHRPVT

SEQ ID NO: 16 - protein, 12 aa, synthetic construct
  REGION 1..12: note = Description of Artificial Sequence: Synthetic peptide
GAADTTHHRPVT

SEQ ID NO: 17 - protein, 12 aa, synthetic construct
  REGION 1..12: note = Description of Artificial Sequence: Synthetic peptide
AGADTTHHRPVT

SEQ ID NO: 18 - protein, 12 aa, synthetic construct
  REGION 1..12: note = Description of Artificial Sequence: Synthetic peptide
GGADTTHHRPAT

SEQ ID NO: 19 - protein, 12 aa, synthetic construct
  REGION 1..12: note = Description of Artificial Sequence: Synthetic peptide
GGADTTHHRPGT

SEQ ID NO: 20 - protein, 24 aa, synthetic construct
  REGION 1..24: note = Description of Artificial Sequence: Synthetic peptide
LLADTTHHRPWTVIGESTHHRPWS

SEQ ID NO: 21 - protein, 36 aa, synthetic construct
  REGION 1..36: note = Description of Artificial Sequence: Synthetic polypeptide
LLADTTHHRPWTVIGESTHHRPWSIIGESSHHKPFT

SEQ ID NO: 22 - protein, 60 aa, synthetic construct
  REGION 1..60: note = Description of Artificial Sequence: Synthetic polypeptide
LLADTTHHRPWTVIGESTHHRPWSIIGESSHHKPFTGLGDTTHHRPWGILAESTHHKPWT

SEQ ID NO: 23 - protein, 24 aa, synthetic construct
  REGION 1..24: note = Description of Artificial Sequence: Synthetic peptide
LLADTTHHRPWTILAESTHHKPWT

SEQ ID NO: 24 - protein, 60 aa, synthetic construct
  REGION 1..60: note = Description of Artificial Sequence: Synthetic polypeptide
LLADTTHHRPWTILAESTHHKPWTLLADTTHHRPWTILAESTHHKPWTLLADTTHHRPWT

SEQ ID NO: 25 - protein, 24 aa, synthetic construct
  REGION 1..24: note = Description of Artificial Sequence: Synthetic peptide
LLADTTHHRPWTGLGDTTHHRPWG

SEQ ID NO: 26 - protein, 36 aa, synthetic construct
  REGION 1..36: note = Description of Artificial Sequence: Synthetic polypeptide
LLADTTHHRPWTGLGDTTHHRPWGLLADTTHHRPWT

SEQ ID NO: 27 - protein, 60 aa, synthetic construct
  REGION 1..60: note = Description of Artificial Sequence: Synthetic polypeptide
LLADTTHHRPWTGLGDTTHHRPWGLLADTTHHRPWTGLGDTTHHRPWGLLADTTHHRPWT

SEQ ID NO: 28 - protein, 84 aa, synthetic construct
  REGION 1..84: note = Description of Artificial Sequence: Synthetic polypeptide
LLADTTHHRPWTGLGDTTHHRPWGLLADTTHHRPWTGLGDTTHHRPWGLLADTTHHRPWTGLGDTTHHRPWGLLADTTHHRPWT

SEQ ID NO: 29 - protein, 60 aa, synthetic construct
  REGION 1..60: note = Description of Artificial Sequence: Synthetic polypeptide
STADTSHHRPSTSGGESTHHRPSTSGGESSHHKPSTGSGDSSHHRPSGSSGESTHHKPST

SEQ ID NO: 30 - protein, 60 aa, synthetic construct
  REGION 1..60: note = Description of Artificial Sequence: Synthetic polypeptide
VGADSTHHRPVTGAADTTHHRPVTAGADTTHHRPVTGGADTTHHRPATGGADTTHHRPGT

SEQ ID NO: 31 - protein, 108 aa, synthetic construct
  REGION 1..108: note = Description of Artificial Sequence: Synthetic polypeptide
STADTSHHRPSLLADTTHHRPWTTSGGESTHHRPSVGADSTHHRPVTTSGGESSHHKPSGAADTTHHRPVTTGSGDSSHHRPSGSSGESTHHKPSTGGADTTHHRPAT

SEQ ID NO: 32 - protein, 114 aa, Homo sapiens
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 33 - protein, 37 aa, synthetic construct
  REGION 1..37: note = Description of Artificial Sequence: Synthetic polypeptide
TGGSGEGGTGASTGGSAGTGGSGGTTSGEAGGSSGAG

SEQ ID NO: 34 - protein, 5 aa, synthetic construct
  REGION 1..5: note = Description of Artificial Sequence: Synthetic peptide
GAGTG

SEQ ID NO: 35 - skipped

SEQ ID NO: 36 - protein, 12 aa, synthetic construct
  REGION 1..12: note = Description of Artificial Sequence: Synthetic peptide
AAADTTHHRPWT

SEQ ID NO: 37 - protein, 60 aa, synthetic construct
  REGION 1..60: note = Description of Artificial Sequence: Synthetic polypeptide
AAADTTHHRPWTAAADTTHHRPWTAAADTTHHRPWTAAADTTHHRPWTAAADTTHHRPWT

SEQ ID NO: 38 - protein, 60 aa, synthetic construct
  REGION 1..60: note = Description of Artificial Sequence: Synthetic polypeptide
LLADAAHHRPWTLLADAAHHRPWTLLADAAHHRPWTLLADAAHHRPWTLLADAAHHRPWT

SEQ ID NO: 39 - protein, 60 aa, synthetic construct
  REGION 1..60: note = Description of Artificial Sequence: Synthetic polypeptide
LLADTTAARPWTLLADTTAARPWTLLADTTAARPWTLLADTTAARPWTLLADTTAARPWT

SEQ ID NO: 40 - protein, 24 aa, synthetic construct
  REGION 1..24: note = Description of Artificial Sequence: Synthetic peptide
LLADTTHHRPWTLLADTTHHRPWT

SEQ ID NO: 41 - protein, 36 aa, synthetic construct
  REGION 1..36: note = Description of Artificial Sequence: Synthetic polypeptide
LLADTTHHRPWTLLADTTHHRPWTLLADTTHHRPWT

SEQ ID NO: 42 - protein, 60 aa, synthetic construct
  REGION 1..60: note = Description of Artificial Sequence: Synthetic polypeptide
LLADTTHHRPWTLLADTTHHRPWTLLADTTHHRPWTLLADTTHHRPWTLLADTTHHRPWT

SEQ ID NO: 43 - protein, 120 aa, synthetic construct
  REGION 1..120: note = Description of Artificial Sequence: Synthetic polypeptide
STSGSTVIGESTHHRPWSLIADSTHHSPWTILAESTHHKPWTILAETTHHRPWSIIGESSHHKPFTGLGDTTHHRPWGVLGDTTHHKPWTIVADSTHHRPWTGQVLPTTTPSSPSTTSGS

SEQ ID NO: 44 - protein, 12 aa, synthetic construct
  REGION 1..12: note = Description of Artificial Sequence: Synthetic peptide
GQVLPTTTPSSP

SEQ ID NO: 45 - protein, 6 aa, synthetic construct
  REGION 1..6: note = Description of Artificial Sequence: Synthetic peptide
GSGATG

SEQ ID NO: 46 - protein, 53 aa, Homo sapiens
NSDSECPLSHDGYCLHDGVCMYIEALDKYACNCVVGYIGERCQYRDLKWWELR

SEQ ID NO: 47 - protein, 66 aa, Homo sapiens
EEAEIPREVIERLARSQIHSIRDLQRLLEIDSVGSEDSLDTSLRAHGVHATKHVPEKRPLPIRRKR

SEQ ID NO: 48 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 49 - protein, 140 aa, Homo sapiens
FNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 50 - protein, 146 aa, Homo sapiens
PALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 51 - protein, 180 aa, Homo sapiens
EENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 52 - protein, 137 aa, Homo sapiens
ENSTSPLSADPPVAAAVVSHFNDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 53 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 54 - protein, 112 aa, Homo sapiens [sequence omitted]

SEQ ID NO: 55 - protein, 139 aa, Homo sapiens
AVRPLRRRQPKKSNELPQANRLPGIFDDVHGSHGRQVCRRHELYVSFQDLGWLDWVIAPQGYSAYYCEGECSFPLDSCMNATNHAILQSLVHLMMPDAVPKACCAPTKLSATSVLYYDSSNNVILRKHRNMVVKACGCH

SEQ ID NO: 56 - protein, 139 aa, Homo sapiens
AVRPLRRRQPKKSNELPQANRLPGIFDDVRGSHGRQVCRRHELYVSFQDLGWLDWVIAPQGYSAYYCEGECSFPLDSCMNATNHAILQSLVHLMKPNAVPKACCAPTKLSATSVLYYDSSNNVILRKHRNMVVKACGCH

SEQ ID NO: 57 - protein, 110 aa, Homo sapiens
QWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 58 - protein, 116 aa, Homo sapiens
SPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 59 - protein, 138 aa, Homo sapiens
AANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 60 - protein, 139 aa, Homo sapiens
SASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 61 - protein, 139 aa, Homo sapiens
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 62 - protein, 110 aa, Homo sapiens
SAGAGSHCQKTSLRVNFEDIGWDSWIIAPKEYEAYECKGGCFFPLADDVTPTKHAIVQTLVHLKFPTKVGKACCVPTKLSPISVLYKDDMGVPTLKYHYEGMSVAECGCR

SEQ ID NO: 63 - protein, 108 aa, Homo sapiens
NAKGNYCKRTPLYIDFKEIGWDSWIIAPPGYEAYECRGVCNYPLAEHLTPTKHAIIQALVHLKNSQKASKACCVPTKLEPISILYLDKGVVTYKFKYEGMAVSECGCR

SEQ ID NO: 64 - protein, 109 aa, Homo sapiens
NLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 65 - protein, 129 aa, Homo sapiens
TALAGTRTAQGSGGGAGRGHGRRGRSRCSRKPLHVDFKELGWDDWIIAPLDYEAYHCEGLCDFPLRSHLEPTNHAIIQTLLNSMAPDAAPASCCVPARLSPISILYIDAANNVVYKQYEDMVVEACGCR

SEQ ID NO: 66 - protein, 120 aa, Homo sapiens
TAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYKQYEDMVVESCGCR

SEQ ID NO: 67 - protein, 125 aa, Homo sapiens
QADGISAEVTASSSKHSGPENNQCSLHPFQISFRQLGWDHWIIAPPFYTPNYCKGTCLRVLRDGLNSPNHAIIQNLINQLVDQSVPRPSCVPYKYVPISVLMIEANGSILYKEYEGMIAESCTCR

SEQ ID NO: 68 - protein, 447 aa, Homo sapiens
IPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAVVQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQG

SEQ ID NO: 69 - protein, 119 aa, Homo sapiens
DAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 70 - protein, 114 aa, Homo sapiens
AAIPVPKLSCKNLCHRHQLFINFRDLGWHKWIIAPKGFMANYCHGECPFSLTISLNSSNYAFMQALMHAVDPEIPQAVCIPTKLSPISMLYQDNNDNVILRHYEDMVVDECGCG

SEQ ID NO: 71 - protein, 120 aa, Homo sapiens
APLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 72 - skipped

SEQ ID NO: 73 - protein, 109 aa, Homo sapiens
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 74 - protein, 114 aa, Homo sapiens
ARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 75 - protein, 206 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 76 - protein, 290 aa, Homo sapiens
FSLMSLLESLDPDWTPDQYDYSYEDYNQEENTSSTLTHAENPDWYYTEDQADPCQPNPCEHGGDCLVHGSTFTCSCLAPFSGNKCQKVQNTCKDNPCGRGQCLITQSPPYYRCVCKHPYTGPSCSQVVPVCRPNPCQNGATCSRHKRRSKFTCACPDQFKGKFCEIGSDDCYVGDGYSYRGKMNRTVNQHACLYWNSHLLLQENYNMFMEDAETHGIGEHNFCRNPDADEKPWCFIKVTNDKVKWEYCDVSACSAQDVAYPEESPTEPSTKLPGFDSCGKTEIAERKIKR

SEQ ID NO: 77 - protein, 400 aa, Homo sapiens
AEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL

SEQ ID NO: 78 - skipped

SEQ ID NO: 79 - protein, 163 aa, Homo sapiens
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 80 - protein, 177 aa, Homo sapiens
GPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 81 - protein, 243 aa, Homo sapiens
TNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 82 - protein, 343 aa, Homo sapiens
ANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 83 - protein, 335 aa, Homo sapiens
SWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 84 - protein, 333 aa, Homo sapiens
SWWYIGALGARVICDNIPGLVSRQRQLCQRYPDIMRSVGEGAREWIRECQHQFRHHRWNCTTLDRDHTVFGRVMLRSSREAAFVYAISSAGVVHAITRACSQGELSVCSCDPYTRGRHHDQRGDFDWGGCSDNIHYGVRFAKAFVDAKEKRLKDARALMNLHNNRCGRTAVRRFLKLECKCHGVSGSCTLRTCWRALSDFRRTGDYLRRRYDGAVQVMATQDGANFTAARQGYRRATRTDLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 85 - protein, 334 aa, Homo sapiens
GYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 86 - protein, 334 aa, Homo sapiens
SYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 87 - protein, 329 aa, Homo sapiens
SNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 88 - protein, 319 aa, Homo sapiens
IIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 89 - protein, 342 aa, Homo sapiens
QLLTDANSWWSLALNPVQRPEMFIIGAQPVCSQLPGLSPGQRKLCQLYQEHMAYIGEGAKTGIKECQHQFRQRRWNCSTADNASVFGRVMQIGSRETAFTHAVSAAGVVNAISRACREGELSTCGCSRTARPKDLPRDWLWGGCGDNVEYGYRFAKEFVDAREREKNFAKGSEEQGRVLMNLQNNEAGRRAVYKMADVACKCHGVSGSCSLKTCWLQLAEFRKVGDRLKEKYDSAAAMRVTRKGRLELVNSRFTQPTPEDLVYVDPSPDYCLRNESTGSLGTQGRLCNKTSEGMDGCELMCCGRGYNQFKSVQVERCHCKFHWCCFVRCKKCTEIVDQYICK

SEQ ID NO: 90 - protein, 341 aa, Homo sapiens
LWWAVGSPLVMDPTSICRKARRLAGRQAELCQAEPEVVAELARGARLGVRECQFQFRFRRWNCSSHSKAFGRILQQDIRETAFVFAITAAGASHAVTQACSMGELLQCGCQAPRGRAPPRPSGLPGTPGPPGPAGSPEGSAAWEWGGCGDDVDFGDEKSRLFMDARHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 91 - protein, 318 aa, Homo sapiens
LGASIICNKIPGLAPRQRAICQSRPDAIIVIGEGSQMGLDECQFQFRNGRWNCSALGERTVFGKELKVGSREAAFTYAIIAAGVAHAITAACTQGNLSDCGCDKEKQGQYHRDEGWKWGGCSADIRYGIGFAKVFVDAREIKQNARTLMNLHNNEAGRKILEENMKLECKCHGVSGSCTTKTCWTTLPQFRELGYVLKDKYNEAVHVEPVRASRNKRPTFLKIKKPLSYRKPMDTDLVYIEKSPNYCEEDPVTGSVGTQGRACNKTAPQASGCDLMCCGRGYNTHQYARVWQCNCKFHWCCYVKCNTCSERTEMYTCK

SEQ ID NO: 92 - protein, 325 aa, Homo sapiens
ALSSVVALGANIICNKIPGLAPRQRAICQSRPDAIIVIGEGAQMGINECQYQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMQLECKCHGVSGSCTTKTCWTTLPKFREVGHLLKEKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 93 - protein, 327 aa, Homo sapiens
VNNFLITGPKAYLTYTTSVALGAQSGIEECKFQFAWERWNCPENALQLSTHNRLRSATRETSFIHAISSAGVMYIITKNCSMGDFENCGCDGSNNGKTGGHGWIWGGCSDNVEFGERISKLFVDSLEKGKDARALMNLHNNRAGRLAVRATMKRTCKCHGISGSCSIQTCWLQLAEFREMGDYLKAKYDQALKIEMDKRQLRAGNSAEGHWVPAEAFLPSAEAELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGKGSA

SEQ ID NO: 94 - protein, 329 aa, Homo sapiens
WSVNNFLMTGPKAYLIYSSSVAAGAQSGIEECKYQFAWDRWNCPERALQLSSHGGLRSANRETAFVHAISSAGVMYTLTRNCSLGDFDNCGCDDSRNGQLGGQGWLWGGCSDNVGFGEAISKQFVDALETGQDARAAMNLHNNEAGRKAVKGTMKRTCKCHGVSGSCTTQTCWLQLPEFREVGAHLKEKYHAALKVDLLQGAGNSAAGRGAIADTFRSISTRELVHLEDSPDYCLENKTLGLLGTEGRECLRRGRALGRWERRSCRRLCGDCGLAVEERRAETVSSCNCKFHWCCAVRCEQCRRRVTKYFCSRAERPRGGAAHKPGRKP

SEQ ID NO: 95 - protein, 336 aa, Homo sapiens
YFGLTGSEPLTILPLTLEPEAAAQAHYKACDRLKLERKQRRMCRRDPGVAETLVEAVSMSALECQFQFRFERWNCTLEGRYRASLLKRGFKETAFLYAISSAGLTHALAKACSAGRMERCTCDEAPDLENREAWQWGGCGDNLKYSSKFVKEFLGRRSSKDLRARVDFHNNLVGVKVIKAGVETTCKCHGVSGSCTVRTCWRQLAPFHEVGKHLKHKYETALKVGSTTNEAAGEAGAISPPRGRASGAGGSDPLPRTPELVHLDDSPSFCLAGRFSPGTAGRRCHREKNCESICCGRGHNTQSRVVTRPCQCQVRWCCYVECRQCTQREEVYTCKG

SEQ ID NO: 96 - protein, 335 aa, Homo sapiens
SYFGLTGREVLTPFPGLGTAAAPAQGGAHLKQCDLLKLSRRQKQLCRREPGLAETLRDAAHLGLLECQFQFRHERWNCSLEGRMGLLKRGFKETAFLYAVSSAALTHTLARACSAGRMERCTCDDSPGLESRQAWQWGVCGDNLKYSTKFLSNFLGSKRGNKDLRARADAHNTHVGIKAVKSGLRTTCKCHGVSGSCAVRTCWKQLSPFRETGQVLKLRYDSAVKVSSATNEALGRLELWAPARQGSLTKGLAPRSGDLVYMEDSPSFCRPSKYSPGTAGRVCSREASCSSLCCGRGYDTQSRLVAFSCHCQVQWCCYVECQQCVQEELVYTCKH

SEQ ID NO: 97 - protein, 382 aa, Homo sapiens
MPRSAPNDILDLRLPPEPVLNANTVCLTLPGLSRRQMEVCVRHPDVAASAIQGIQIAIHECQHQFRDQRWNCSSLETRNKIPYESPIFSRGFRESAFAYAIAAAGVVHAVSNACALGKLKACGCDASRRGDEEAFRRKLHRLQLDALQRGKGLSHGVPEHPALPTASPGLQDSWEWGGCSPDMGFGERFSKDFLDSREPHRDIHARMRLHNNRVGRQAVMENMRRKCKCHGTSGSCQLKTCWQVTPEFRTVGALLRSRFHRATLIRPHNRNGGQLEPGPAGAPSPAPGAPGPRRRASPADLVYFEKSPDFCEREPRLDSAGTVGRLCNKSSAGSDGCGSMCCGRGHNILRQTRSERCHCRFHWCCFVVCEECRITEWVSVCK

SEQ ID NO: 98 - protein, 361 aa, Homo sapiens
NEILGLKLPGEPPLTANTVCLTLSGLSKRQLGLCLRNPDVTASALQGLHIAVHECQHQLRDQRWNCSALEGGGRLPHHSAILKRGFRESAFSFSMLAAGVMHAVATACSLGKLVSCGCGWKGSGEQDRLRAKLLQLQALSRGKSFPHSLPSPGPGSSPSPGPQDTWEWGGCNHDMDFGEKFSRDFLDSREAPRDIQARMRIHNNRVGRQVVTENLKRKCKCHGTSGSCQFKTCWRAAPEFRAVGAALRERLGRAIFIDTHNRNSGAFQPRLRPRRLSGELVYFEKSPDFCERDPTMGSPGTRGRACNKTSRLLDGCGSLCCGRGHNVLRQTRVERCHCRFHWCCYVLCDECKVTEWVNVCK

SEQ ID NO: 99 - protein, 330 aa, Homo sapiens
IKWLALSKTPSALALNQTQHCKQLEGLVSAQVQLCRSNLELMHTVVHAAREVMKACRRAFADMRWNCSSIELAPNYLLDLERGTRESAFVYALSAAAISHAIARACTSGDLPGCSCGPVPGEPPGPGNRWGGCADNLSYGLLMGAKFSDAPMKVKKTGSQANKLMRLHNSEVGRQALRASLEMKCKCHGVSGSCSIRTCWKGLQELQDVAADLKTRYLSATKVVHRPMGTRKHLVPKDLDIRPVKDSELVYLQSSPDFCMKNEKVGSHGTQDRQCNKTSNGSDSCDLMCCGRGYNPYTDRVVERCHCKYHWCCYVTCRRCERTVERYVCK

SEQ ID NO: 100 - protein, 336 aa, Homo sapiens
NWMWLGIASFGVPEKLGCANLPLNSRQKELCKRKPYLLPSIREGARLGIQECGSQFRHERWNCMITAAATTAPMGASPLFGYELSSGTKETAFIYAVMAAGLVHSVTRSCSAGNMTECSCDTTLQNGGSASEGWHWGGCSDDVQYGMWFSRKFLDFPIGNTTGKENKVLLAMNLHNNEAGRQAVAKLMSVDCRCHGVSGSCAVKTCWKTMSSFEKIGHLLKDKYENSIQISDKTKRKMRRREKDQRKIPIHKDDLLYVNKSPNYCVEDKKLGIPGTQGRECNRTSEGADGCNLLCCGRGYNTHVVRHVERCECKFIWCCYVRCRRCESMTDVHTCK

SEQ ID NO: 101 - protein, 65 aa, Homo sapiens
SHLVKCAEKEKTFCVNGGECFMVKDLSNPSRYLCKCPNEFTGDRCQNYVMASFYKHLGIEFMEAE

SEQ ID NO: 102-151 - skipped (50 entries)

SEQ ID NO: 152 - protein, 50 aa, Homo sapiens
VVSHFNDCPDSHTQFCFHGTCRFLVQEDKPACVCHSGYVGARCEHADLLA

SEQ ID NO: 153-167 - skipped (15 entries)

SEQ ID NO: 168 - protein, 1286 aa, Homo sapiens
IPVPQSKPLERHVEKSMNLHLLARSNVSVQDELNASGTIKESGVLVHEGDRGRQENTQDGHKGEGNGSKWAEVGGKSFSTYSTLANEEGNIEGWNGDTGKAETYGHDGIHGKEENITANGIQGQVSIIDNAGATNRSNTNGNTDKNTQNGDVGDAGHNEDVAVVQEDGPQVAGSNNSTDNEDEIIENSCRNEGNTSEITPQINSKRNGTKEAEVTPGTGEDAGLDNSDGSPSGNGADEDEDEGSGDDEDEEAGNGKDSSNNSKGQEGQDHGKEDDHDSSIGQNSDSKEYYDPEGKEDPHNEVDGDKTSKSEENSAGIPEDNGSQRIEDTQKLNHRESKRVENRITKESETHAVGKSQDKGIEIKGPSSGNRNITKEVGKGNEGKEDKGQHGMILGKGNVKTQGEVVNIEGPGQKSEPGNKVGHSNTGSDSNSDGYDSYDFDDKSMQGDDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSNSSDSSDSSDSSNSSESSDSSDSSDSDSSDSSDSSNSNSSDSDSSNSSDSSDSSNSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSSDSSDSSDGSDSDSSNRSDSSNSSDSSDSSDSSNSSDSSDSSDSNESSNSSDSSDSSNSSDSDSSDSSNSSDSSDSSNSSDSSESSNSSDNSNSSDSSNSSDSSDSSDSSNSSDSSNSSDSSNSSDSSDSNSSDSSDSSNSSDSSDSSDSSDSSDSSDSSNSSDSSDSSDSSDSSNSSDSSNSSDSSNSSDSSDSSDSSDSSDSSDSSDSSDSSNSSDSSDSSDSSDSSDSSDSSDSSDSSESSDSSDSSNSSDSSDSSDSSDSSDSSDSSDSSDSSDSSNSSDSSDSSDSSDSSDSSNSSDSSDSSESSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD

SEQ ID NO: 169-175 - skipped (7 entries)

SEQ ID NO: 176 - protein, 247 aa, Homo sapiens
IYGGFKSTAGKHPWQASLQSSLPLTISMPQGHFCGGALIHPCWVLTAAHCTDIKTRHLKVVLGDQDLKKEEFHEQSFRVEKIFKYSHYNERDEIPHNDIALLKLKPVDGHCALESKYVKTVCLPDGSFPSGSECHISGWGVTETGKGSRQLLDAKVKLIANTLCNSRQLYDHMIDDSMICAGNLQKPGQDTCQGDSGGPLTCEKDGTYYVYGIVSWGLECGKRPGVYTQVTKFLNWIKATIKSESGF

SEQ ID NO: 177-267 - skipped (91 entries)

SEQ ID NO: 268 - protein, 839 aa, Homo sapiens
DDPNSSDESNGNDDANSESDNNSSSRGDASYNSDESKDNGNGSDSKGAEDDDSDSTSDTNNSDSNGNGNNGNDDNDKSDSGKGKSDSSDSDSSDSSNSSDSSDSSDSDSSDSNSSSDSDSSDSDSSDSSDSDSSDSSNSSDSSDSSDSSDSSDSSDSSDSKSDSSKSESDSSDSDSKSDSSDSNSSDSSDNSDSSDSSNSSNSSDSSDSSDSSDSSSSSDSSNSSDSSDSSDSSNSSESSDSSDSSDSDSSDSSDSSNSNSSDSDSSNSSDSSDSSNSSDSSDSSDSSNSSDSSDSSDSSNSSDSSDSSDSSDSSDSSNSSDSNDSSNSSDSSDSSNSSDSSNSSDSSDSSDSSDSDSSNSSDSSNSSDSSDSSNSSDSSDSSDSSDGSDSDSSNRSDSSNSSDSSDSSDSSNSSDSSDSSDSNESSNSSDSSDSSNSSDSDSSDSSNSSDSSDSSNSSDSSESSNSSDNSNSSDSSNSSDSSDSSDSSNSSDSSNSSDSSNSSDSSDSNSSDSSDSSNSSDSSDSSDSSDSSDSSDSSNSSDSSDSSDSSDSSNSSDSSNSSDSSNSSDSSDSSDSSDSSDSSDSSDSSDSSNSSDSSDSSDSSDSSDSSDSSDSSDSSESSDSSDSSNSSDSSDSSDSSDSSDSSDSSDSSDSSDSSNSSDSSDSSDSSDSSDSSNSSDSSDSSESSDSSDSSDSSDSSDSSDSSDSSDSSDSSNSSDSSDSSDSSDSSDSSDSSDSSDSSDSSDSSDSSDSSDSSDSSDSSDSSDSNESSDSSDSSDSSDSSNSSDSSDSSDSSDSTSDSNDESDSQSKSGNGNNNGSDSDSDSEGSDSNHSTSDD

SEQ ID NO: 269-400 - skipped (132 entries)

SEQ ID NO: 401 - protein, 48 aa, synthetic construct
  REGION 1..48: note = Description of Artificial Sequence: Synthetic polypeptide
LLADTTHHRPWTVIGESTHHRPWSIIGESSHHKPFTGLGDTTHHRPWG

SEQ ID NO: 402 - protein, 48 aa, synthetic construct
  REGION 1..48: note = Description of Artificial Sequence: Synthetic polypeptide
VIGESTHHRPWSIIGESSHHKPFTGLGDTTHHRPWGILAESTHHKPWT

SEQ ID NO: 403 - protein, 1416 aa, synthetic construct
  REGION 1..1416: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1..1416: note = See specification as filed for detailed description of substitutions and preferred embodiments
LLADTTHHRPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 404 - protein, 1416 aa, synthetic construct
  REGION 1..1416: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1..1416: note = See specification as filed for detailed description of substitutions and preferred embodiments
VIGESTHHRPWSXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 405 - protein, 1416 aa, synthetic construct
  REGION 1..1416: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1..1416: note = See specification as filed for detailed description of substitutions and preferred embodiments
ILAESTHHKPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 406 - protein, 1416 aa, synthetic construct
  REGION 1..1416: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1..1416: note = See specification as filed for detailed description of substitutions and preferred embodiments
IIGESSHHKPFTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 407 - protein, 1416 aa, synthetic construct
  REGION 1..1416: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1..1416: note = See specification as filed for detailed description of substitutions and preferred embodiments
GLGDTTHHRPWGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 408 - protein, 60 aa, synthetic construct
  REGION 1..60: note = Description of Artificial Sequence: Synthetic polypeptide
  MOD_RES 13..60: note = This region may encompass one or more of the following sequences: "VIGESTHHRPWS" or "ILAESTHHKPWT" or "IIGESSHHKPFT" or "GLGDTTHHRPWG"
  REGION 1..60: note = See specification as filed for detailed description of substitutions and preferred embodiments
LLADTTHHRPWTXXXXXXXXXXXXXXXXXXXXXXXXXXXXXXXXXXXXXXXXXXXXXXXX

SEQ ID NO: 409 - protein, 60 aa, synthetic construct
  REGION 1..60: note = Description of Artificial Sequence: Synthetic polypeptide
  MOD_RES 13..60: note = This region may encompass one or more of the following sequences: "LLADTTHHRPWT" or "ILAESTHHKPWT" or "IIGESSHHKPFT" or "GLGDTTHHRPWG"
  REGION 1..60: note = See specification as filed for detailed description of substitutions and preferred embodiments
VIGESTHHRPWSXXXXXXXXXXXXXXXXXXXXXXXXXXXXXXXXXXXXXXXXXXXXXXXX

SEQ ID NO: 410 - protein, 60 aa, synthetic construct
  REGION 1..60: note = Description of Artificial Sequence: Synthetic polypeptide
  MOD_RES 13..60: note = This region may encompass one or more of the following sequences: "LLADTTHHRPWT" or "VIGESTHHRPWS" or "IIGESSHHKPFT" or "GLGDTTHHRPWG"
  REGION 1..60: note = See specification as filed for detailed description of substitutions and preferred embodiments
ILAESTHHKPWTXXXXXXXXXXXXXXXXXXXXXXXXXXXXXXXXXXXXXXXXXXXXXXXX

SEQ ID NO: 411 - protein, 60 aa, synthetic construct
  REGION 1..60: note = Description of Artificial Sequence: Synthetic polypeptide
  MOD_RES 13..60: note = This region may encompass one or more of the following sequences: "LLADTTHHRPWT" or "ILAESTHHKPWT" or "VIGESTHHRPWS" or "GLGDTTHHRPWG"
  REGION 1..60: note = See specification as filed for detailed description of substitutions and preferred embodiments
IIGESSHHKPFTXXXXXXXXXXXXXXXXXXXXXXXXXXXXXXXXXXXXXXXXXXXXXXXX

SEQ ID NO: 412 - protein, 60 aa, synthetic construct
  REGION 1..60: note = Description of Artificial Sequence: Synthetic polypeptide
  MOD_RES 13..60: note = This region may encompass one or more of the following sequences: "LLADTTHHRPWT" or "ILAESTHHKPWT" or "IIGESSHHKPFT" or "VIGESTHHRPWS"
  REGION 1..60: note = See specification as filed for detailed description of substitutions and preferred embodiments
GLGDTTHHRPWGXXXXXXXXXXXXXXXXXXXXXXXXXXXXXXXXXXXXXXXXXXXXXXXX

SEQ ID NO: 413 - protein, 60 aa, synthetic construct; identical to SEQ ID NO: 408
  REGION 1..60: note = Description of Artificial Sequence: Synthetic polypeptide
  MOD_RES 13..60: note = This region may encompass two or more of the following sequences: "VIGESTHHRPWS" or "ILAESTHHKPWT" or "IIGESSHHKPFT" or "GLGDTTHHRPWG"
  REGION 1..60: note = See specification as filed for detailed description of substitutions and preferred embodiments

SEQ ID NO: 414 - protein, 60 aa, synthetic construct; identical to SEQ ID NO: 409
  REGION 1..60: note = Description of Artificial Sequence: Synthetic polypeptide
  MOD_RES 13..60: note = This region may encompass two or more of the following sequences: "LLADTTHHRPWT" or "ILAESTHHKPWT" or "IIGESSHHKPFT" or "GLGDTTHHRPWG"
  REGION 1..60: note = See specification as filed for detailed description of substitutions and preferred embodiments

SEQ ID NO: 415 - protein, 60 aa, synthetic construct; identical to SEQ ID NO: 410
  REGION 1..60: note = Description of Artificial Sequence: Synthetic polypeptide
  MOD_RES 13..60: note = This region may encompass two or more of the following sequences: "LLADTTHHRPWT" or "VIGESTHHRPWS" or "IIGESSHHKPFT" or "GLGDTTHHRPWG"
  REGION 1..60: note = See specification as filed for detailed description of substitutions and preferred embodiments

SEQ ID NO: 416 - protein, 60 aa, synthetic construct; identical to SEQ ID NO: 411
  REGION 1..60: note = Description of Artificial Sequence: Synthetic polypeptide
  MOD_RES 13..60: note = This region may encompass two or more of the following sequences: "LLADTTHHRPWT" or "ILAESTHHKPWT" or "VIGESTHHRPWS" or "GLGDTTHHRPWG"
  REGION 1..60: note = See specification as filed for detailed description of substitutions and preferred embodiments

SEQ ID NO: 417 - protein, 60 aa, synthetic construct; identical to SEQ ID NO: 412
  REGION 1..60: note = Description of Artificial Sequence: Synthetic polypeptide
  MOD_RES 13..60: note = This region may encompass two or more of the following sequences: "LLADTTHHRPWT" or "ILAESTHHKPWT" or "IIGESSHHKPFT" or "VIGESTHHRPWS"
  REGION 1..60: note = See specification as filed for detailed description of substitutions and preferred embodiments

SEQ ID NO: 418 - protein, 60 aa, synthetic construct; identical to SEQ ID NO: 408
  REGION 1..60: note = Description of Artificial Sequence: Synthetic polypeptide
  MOD_RES 13..60: note = This region may encompass three or more of the following sequences: "VIGESTHHRPWS" or "ILAESTHHKPWT" or "IIGESSHHKPFT" or "GLGDTTHHRPWG"
  REGION 1..60: note = See specification as filed for detailed description of substitutions and preferred embodiments

SEQ ID NO: 419 - protein, 60 aa, synthetic construct; identical to SEQ ID NO: 409
  REGION 1..60: note = Description of Artificial Sequence: Synthetic polypeptide
  MOD_RES 13..60: note = This region may encompass three or more of the following sequences: "LLADTTHHRPWT" or "ILAESTHHKPWT" or "IIGESSHHKPFT" or "GLGDTTHHRPWG"
  REGION 1..60: note = See specification as filed for detailed description of substitutions and preferred embodiments

SEQ ID NO: 420 - protein, 60 aa, synthetic construct; identical to SEQ ID NO: 410
  REGION 1..60: note = Description of Artificial Sequence: Synthetic polypeptide
  MOD_RES 13..60: note = This region may encompass three or more of the following sequences: "LLADTTHHRPWT" or "VIGESTHHRPWS" or "IIGESSHHKPFT" or "GLGDTTHHRPWG"
  REGION 1..60: note = See specification as filed for detailed description of substitutions and preferred embodiments

SEQ ID NO: 421 - protein, 60 aa, synthetic construct; identical to SEQ ID NO: 411
  REGION 1..60: note = Description of Artificial Sequence: Synthetic polypeptide
  MOD_RES 13..60: note = This region may encompass three or more of the following sequences: "LLADTTHHRPWT" or "ILAESTHHKPWT" or "VIGESTHHRPWS" or "GLGDTTHHRPWG"
  REGION 1..60: note = See specification as filed for detailed description of substitutions and preferred embodiments

SEQ ID NO: 422 - protein, 60 aa, synthetic construct; identical to SEQ ID NO: 412
  REGION 1..60: note = Description of Artificial Sequence: Synthetic polypeptide
  MOD_RES 13..60: note = This region may encompass three or more of the following sequences: "LLADTTHHRPWT" or "ILAESTHHKPWT" or "IIGESSHHKPFT" or "VIGESTHHRPWS"
  REGION 1..60: note = See specification as filed for detailed description of substitutions and preferred embodiments

SEQ ID NO: 423-500 - skipped (78 entries)

SEQ ID NO: 501 - protein, 152 aa, synthetic construct
  REGION 1..152: note = Description of Artificial Sequence: Synthetic polypeptide
ASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 502 - protein, 217 aa, synthetic construct
  REGION 1..217: note = Description of Artificial Sequence: Synthetic polypeptide
MPIGSLLADTTHHRPWTVIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 503 - protein, 212 aa, synthetic construct
  REGION 1..212: note = Description of Artificial Sequence: Synthetic polypeptide
LLADTTHHRPWTVIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 504 - protein, 200 aa, synthetic construct
  REGION 1..200: note = Description of Artificial Sequence: Synthetic polypeptide
VIGESTHHRPWSIIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 505 - protein, 188 aa, synthetic construct
  REGION 1..188: note = Description of Artificial Sequence: Synthetic polypeptide
IIGESSHHKPFTGLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 506 - protein, 176 aa, synthetic construct
  REGION 1..176: note = Description of Artificial Sequence: Synthetic polypeptide
GLGDTTHHRPWGILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 507 - protein, 164 aa, synthetic construct
  REGION 1..164: note = Description of Artificial Sequence: Synthetic polypeptide
ILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 508 - protein, 126 aa, synthetic construct
  REGION 1..126: note = Description of Artificial Sequence: Synthetic polypeptide
  MOD_RES 1: note = V, L, I, G, S, T or A
  MOD_RES 2: note = I, L, V, Q, T, S, G or A
  MOD_RES 3: note = G, A, V or S
  MOD_RES 4: note = E, D, L or G
  MOD_RES 5: note = S, T, P T, E or D
  MOD_RES 6: note = T or S
  MOD_RES 7: note = H, T or S
  MOD_RES 8: note = H or T
  MOD_RES 9: note = R, S, K, P or H
  MOD_RES 10: note = P, S, R or K
  MOD_RES 11: note = W, F, S, P, V, A or G
  MOD_RES 12: note = absent or is S, T G, or A
  REGION 12..13: note = NON_CONS - Residues at these positions can be separated by a linker of unknown length
XXXXXXXXXXXXQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 509 - protein, 164 aa, synthetic construct
  REGION 1..164: note = Description of Artificial Sequence: Synthetic polypeptide
  MOD_RES 1: note = V, L, I, G, S, T or A
  MOD_RES 2: note = I, L, V, Q, T, S, G or A
  MOD_RES 3: note = G, A, V or S
  MOD_RES 4: note = E, D, L or G
  MOD_RES 5: note = S, T, P T, E or D
  MOD_RES 6: note = T or S
  MOD_RES 7: note = H, T or S
  MOD_RES 8: note = H or T
  MOD_RES 9: note = R, S, K, P or H
  MOD_RES 10: note = P, S, R or K
  MOD_RES 11: note = W, F, S, P, V, A or G
  MOD_RES 12: note = absent or is S, T G, or A
XXXXXXXXXXXXASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 510 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
LLADTTHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 511 - protein, 164 aa, synthetic construct
  REGION 1..164: note = Description of Artificial Sequence: Synthetic polypeptide
LLADTTHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 512 - protein, 164 aa, synthetic construct
  REGION 1..164: note = Description of Artificial Sequence: Synthetic polypeptide
VIGESTHHRPWSASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 513 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
LIADSTHHSPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 514 - protein, 164 aa, synthetic construct
  REGION 1..164: note = Description of Artificial Sequence: Synthetic polypeptide
LIADSTHHSPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 515 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
ILAESTHHKPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 516 - protein, 164 aa, synthetic construct; identical to SEQ ID NO: 507
  REGION 1..164: note = Description of Artificial Sequence: Synthetic polypeptide

SEQ ID NO: 517 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
ILAETTHHRPWSQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 518 - protein, 164 aa, synthetic construct
  REGION 1..164: note = Description of Artificial Sequence: Synthetic polypeptide
ILAETTHHRPWSASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 519 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
IIGESSHHKPFTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 520 - protein, 164 aa, synthetic construct
  REGION 1..164: note = Description of Artificial Sequence: Synthetic polypeptide
IIGESSHHKPFTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 521 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
GLGDTTHHRPWGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 522 - protein, 164 aa, synthetic construct
  REGION 1..164: note = Description of Artificial Sequence: Synthetic polypeptide
GLGDTTHHRPWGASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 523 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
VLGDTTHHKPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 524 - protein, 164 aa, synthetic construct
  REGION 1..164: note = Description of Artificial Sequence: Synthetic polypeptide
VLGDTTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 525 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
IVADSTHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 526 - protein, 164 aa, synthetic construct
  REGION 1..164: note = Description of Artificial Sequence: Synthetic polypeptide
IVADSTHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 527 - protein, 125 aa, synthetic construct
  REGION 1..125: note = Description of Artificial Sequence: Synthetic polypeptide
  REGION 11..12: note = NON_CONS - Residues at these positions can be separated by a linker of unknown length
STADTSHHRPSQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 528 - protein, 163 aa, synthetic construct
  REGION 1..163: note = Description of Artificial Sequence: Synthetic polypeptide
STADTSHHRPSASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 529 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
TSGGESTHHRPSQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 530 - protein, 164 aa, synthetic construct
  REGION 1..164: note = Description of Artificial Sequence: Synthetic polypeptide
TSGGESTHHRPSASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 531 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
TSGGESSHHKPSQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 532 - protein, 164 aa, synthetic construct
  REGION 1..164: note = Description of Artificial Sequence: Synthetic polypeptide
TSGGESSHHKPSASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 533 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
TGSGDSSHHRPSQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 534 - protein, 164 aa, synthetic construct
  REGION 1..164: note = Description of Artificial Sequence: Synthetic polypeptide
TGSGDSSHHRPSASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 535 - protein, 127 aa, synthetic construct
  REGION 1..127: note = Description of Artificial Sequence: Synthetic polypeptide
  REGION 13..14: note = NON_CONS - Residues at these positions can be separated by a linker of unknown length
GSSGESTHHKPSTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 536 - protein, 165 aa, synthetic construct
  REGION 1..165: note = Description of Artificial Sequence: Synthetic polypeptide
GSSGESTHHKPSTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 537 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
VGADSTHHRPVTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 538 - protein, 164 aa, synthetic construct
  REGION 1..164: note = Description of Artificial Sequence: Synthetic polypeptide
VGADSTHHRPVTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 539 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
GAADTTHHRPVTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 540 - protein, 164 aa, synthetic construct
  REGION 1..164: note = Description of Artificial Sequence: Synthetic polypeptide
GAADTTHHRPVTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 541 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
AGADTTHHRPVTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 542 - protein, 164 aa, synthetic construct
  REGION 1..164: note = Description of Artificial Sequence: Synthetic polypeptide
AGADTTHHRPVTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 543 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
GGADTTHHRPATQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 544 - protein, 164 aa, synthetic construct
  REGION 1..164: note = Description of Artificial Sequence: Synthetic polypeptide
GGADTTHHRPATASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 545 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
GGADTTHHRPGTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 546 - protein, 164 aa, synthetic construct
  REGION 1..164: note = Description of Artificial Sequence: Synthetic polypeptide
GGADTTHHRPGTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 547 - protein, 138 aa, synthetic construct
  REGION 1..138: note = Description of Artificial Sequence: Synthetic polypeptide
  REGION 24..25: note = NON_CONS - Residues at these positions can be separated by a linker of unknown length
LLADTTHHRPWTVIGESTHHRPWSQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 548 - protein, 176 aa, synthetic construct
  REGION 1..176: note = Description of Artificial Sequence: Synthetic polypeptide
LLADTTHHRPWTVIGESTHHRPWSASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 549 - protein, 150 aa, synthetic construct
  REGION 1..150: note = Description of Artificial Sequence: Synthetic polypeptide
  REGION 36..37: note = NON_CONS - Residues at these positions can be separated by a linker of unknown length
LLADTTHHRPWTVIGESTHHRPWSIIGESSHHKPFTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 550 - protein, 188 aa, synthetic construct
  REGION 1..188: note = Description of Artificial Sequence: Synthetic polypeptide
LLADTTHHRPWTVIGESTHHRPWSIIGESSHHKPFTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 551 - protein, 174 aa, synthetic construct
  REGION 1..174: note = Description of Artificial Sequence: Synthetic polypeptide
  REGION 60..61: note = NON_CONS - Residues at these positions can be separated by a linker of unknown length
LLADTTHHRPWTVIGESTHHRPWSIIGESSHHKPFTGLGDTTHHRPWGILAESTHHKPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 552 - protein, 212 aa, synthetic construct; identical to SEQ ID NO: 503
  REGION 1..212: note = Description of Artificial Sequence: Synthetic polypeptide

SEQ ID NO: 553 - protein, 138 aa, synthetic construct
  REGION 1..138: note = Description of Artificial Sequence: Synthetic polypeptide
  REGION 24..25: note = NON_CONS - Residues at these positions can be separated by a linker of unknown length
LLADTTHHRPWTILAESTHHKPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 554 - protein, 176 aa, synthetic construct
  REGION 1..176: note = Description of Artificial Sequence: Synthetic polypeptide
LLADTTHHRPWTILAESTHHKPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 555 - protein, 174 aa, synthetic construct
  REGION 1..174: note = Description of Artificial Sequence: Synthetic polypeptide
  REGION 60..61: note = NON_CONS - Residues at these positions can be separated by a linker of unknown length
LLADTTHHRPWTILAESTHHKPWTLLADTTHHRPWTILAESTHHKPWTLLADTTHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 556 - protein, 212 aa, synthetic construct
  REGION 1..212: note = Description of Artificial Sequence: Synthetic polypeptide
LLADTTHHRPWTILAESTHHKPWTLLADTTHHRPWTILAESTHHKPWTLLADTTHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 557 - protein, 138 aa, synthetic construct
  REGION 1..138: note = Description of Artificial Sequence: Synthetic polypeptide
  REGION 24..25: note = NON_CONS - Residues at these positions can be separated by a linker of unknown length
LLADTTHHRPWTGLGDTTHHRPWGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 558 - protein, 176 aa, synthetic construct
  REGION 1..176: note = Description of Artificial Sequence: Synthetic polypeptide
LLADTTHHRPWTGLGDTTHHRPWGASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 559 - protein, 150 aa, synthetic construct
  REGION 1..150: note = Description of Artificial Sequence: Synthetic polypeptide
  REGION 36..37: note = NON_CONS - Residues at these positions can be separated by a linker of unknown length
LLADTTHHRPWTGLGDTTHHRPWGLLADTTHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 560 - protein, 188 aa, synthetic construct
  REGION 1..188: note = Description of Artificial Sequence: Synthetic polypeptide
LLADTTHHRPWTGLGDTTHHRPWGLLADTTHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 561 - protein, 174 aa, synthetic construct
  REGION 1..174: note = Description of Artificial Sequence: Synthetic polypeptide
  REGION 60..61: note = NON_CONS - Residues at these positions can be separated by a linker of unknown length
LLADTTHHRPWTGLGDTTHHRPWGLLADTTHHRPWTGLGDTTHHRPWGLLADTTHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 562 - protein, 212 aa, synthetic construct
  REGION 1..212: note = Description of Artificial Sequence: Synthetic polypeptide
LLADTTHHRPWTGLGDTTHHRPWGLLADTTHHRPWTGLGDTTHHRPWGLLADTTHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 563 - protein, 198 aa, synthetic construct
  REGION 1..198: note = Description of Artificial Sequence: Synthetic polypeptide
  REGION 84..85: note = NON_CONS - Residues at these positions can be separated by a linker of unknown length
LLADTTHHRPWTGLGDTTHHRPWGLLADTTHHRPWTGLGDTTHHRPWGLLADTTHHRPWTGLGDTTHHRPWGLLADTTHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 564 - protein, 236 aa, synthetic construct
  REGION 1..236: note = Description of Artificial Sequence: Synthetic polypeptide
LLADTTHHRPWTGLGDTTHHRPWGLLADTTHHRPWTGLGDTTHHRPWGLLADTTHHRPWTGLGDTTHHRPWGLLADTTHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 565 - protein, 174 aa, synthetic construct
  REGION 1..174: note = Description of Artificial Sequence: Synthetic polypeptide
  REGION 60..61: note = NON_CONS - Residues at these positions can be separated by a linker of unknown length
STADTSHHRPSTSGGESTHHRPSTSGGESSHHKPSTGSGDSSHHRPSGSSGESTHHKPSTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 566 - protein, 212 aa, synthetic construct
  REGION 1..212: note = Description of Artificial Sequence: Synthetic polypeptide
STADTSHHRPSTSGGESTHHRPSTSGGESSHHKPSTGSGDSSHHRPSGSSGESTHHKPSTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 567 - protein, 174 aa, synthetic construct
  REGION 1..174: note = Description of Artificial Sequence: Synthetic polypeptide
  REGION 60..61: note = NON_CONS - Residues at these positions can be separated by a linker of unknown length
VGADSTHHRPVTGAADTTHHRPVTAGADTTHHRPVTGGADTTHHRPATGGADTTHHRPGTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 568 - protein, 212 aa, synthetic construct
  REGION 1..212: note = Description of Artificial Sequence: Synthetic polypeptide
VGADSTHHRPVTGAADTTHHRPVTAGADTTHHRPVTGGADTTHHRPATGGADTTHHRPGTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 569 - protein, 222 aa, synthetic construct
  REGION 1..222: note = Description of Artificial Sequence: Synthetic polypeptide
  REGION 108..109: note = NON_CONS - Residues at these positions can be separated by a linker of unknown length
STADTSHHRPSLLADTTHHRPWTTSGGESTHHRPSVGADSTHHRPVTTSGGESSHHKPSGAADTTHHRPVTTGSGDSSHHRPSGSSGESTHHKPSTGGADTTHHRPATQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 570 - protein, 260 aa, synthetic construct
  REGION 1..260: note = Description of Artificial Sequence: Synthetic polypeptide
STADTSHHRPSLLADTTHHRPWTTSGGESTHHRPSVGADSTHHRPVTTSGGESSHHKPSGAADTTHHRPVTTGSGDSSHHRPSGSSGESTHHKPSTGGADTTHHRPATASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 571 - protein, 228 aa, synthetic construct
  REGION 1..228: note = Description of Artificial Sequence: Synthetic polypeptide
  REGION 114..115: note = NON_CONS - Residues at these positions can be separated by a linker of unknown length
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 572 - protein, 266 aa, synthetic construct
  REGION 1..266: note = Description of Artificial Sequence: Synthetic polypeptide
QAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCRASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 573 - protein, 151 aa, synthetic construct
  REGION 1..151: note = Description of Artificial Sequence: Synthetic polypeptide
  REGION 37..38: note = NON_CONS - Residues at these positions can be separated by a linker of unknown length
TGGSGEGGTGASTGGSAGTGGSGGTTSGEAGGSSGAGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 574 - protein, 189 aa, synthetic construct
  REGION 1..189: note = Description of Artificial Sequence: Synthetic polypeptide
TGGSGEGGTGASTGGSAGTGGSGGTTSGEAGGSSGAGASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 575 - protein, 119 aa, synthetic construct
  REGION 1..119: note = Description of Artificial Sequence: Synthetic polypeptide
  REGION 5..6: note = NON_CONS - Residues at these positions can be separated by a linker of unknown length
GAGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 576 - protein, 157 aa, synthetic construct
  REGION 1..157: note = Description of Artificial Sequence: Synthetic polypeptide
GAGTGASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 577 - protein, 126 aa, synthetic construct; identical to SEQ ID NO: 508
  REGION 1..126: note = Description of Artificial Sequence: Synthetic polypeptide
  MOD_RES 1: note = V, L, I, G, S, T or A
  MOD_RES 2: note = I, L, V, Q, T, S, G or A
  MOD_RES 3: note = G, A, V or S
  MOD_RES 4: note = E, D, L or G
  MOD_RES 5: note = S, T, P T, E or D
  MOD_RES 6: note = T or S
  MOD_RES 7: note = H, T or S
  MOD_RES 8: note = H or T
  MOD_RES 9: note = R, S, K, P or H
  MOD_RES 10: note = P, S, R or K
  MOD_RES 11: note = W, F, S, P, V, A or G
  MOD_RES 12: note = absent or is S, T G, or A
  REGION 12..13: note = NON_CONS - Residues at these positions can be separated by a linker of unknown length

SEQ ID NO: 578 - protein, 164 aa, synthetic construct; identical to SEQ ID NO: 509
  REGION 1..164: note = Description of Artificial Sequence: Synthetic polypeptide
  MOD_RES 1: note = V, L, I, G, S, T or A
  MOD_RES 2: note = I, L, V, Q, T, S, G or A
  MOD_RES 3: note = G, A, V or S
  MOD_RES 4: note = E, D, L or G
  MOD_RES 5: note = S, T, P T, E or D
  MOD_RES 6: note = T or S
  MOD_RES 7: note = H, T or S
  MOD_RES 8: note = H or T
  MOD_RES 9: note = R, S, K, P or H
  MOD_RES 10: note = P, S, R or K
  MOD_RES 11: note = W, F, S, P, V, A or G
  MOD_RES 12: note = absent or is S, T G, or A

SEQ ID NO: 579 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
AAADTTHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 580 - protein, 164 aa, synthetic construct
  REGION 1..164: note = Description of Artificial Sequence: Synthetic polypeptide
AAADTTHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 581 - protein, 174 aa, synthetic construct
  REGION 1..174: note = Description of Artificial Sequence: Synthetic polypeptide
  REGION 60..61: note = NON_CONS - Residues at these positions can be separated by a linker of unknown length
AAADTTHHRPWTAAADTTHHRPWTAAADTTHHRPWTAAADTTHHRPWTAAADTTHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 582 - protein, 212 aa, synthetic construct
  REGION 1..212: note = Description of Artificial Sequence: Synthetic polypeptide
AAADTTHHRPWTAAADTTHHRPWTAAADTTHHRPWTAAADTTHHRPWTAAADTTHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 583 - protein, 174 aa, synthetic construct
  REGION 1..174: note = Description of Artificial Sequence: Synthetic polypeptide
  REGION 60..61: note = NON_CONS - Residues at these positions can be separated by a linker of unknown length
LLADAAHHRPWTLLADAAHHRPWTLLADAAHHRPWTLLADAAHHRPWTLLADAAHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 584 - protein, 212 aa, synthetic construct
  REGION 1..212: note = Description of Artificial Sequence: Synthetic polypeptide
LLADAAHHRPWTLLADAAHHRPWTLLADAAHHRPWTLLADAAHHRPWTLLADAAHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 585 - protein, 174 aa, synthetic construct
  REGION 1..174: note = Description of Artificial Sequence: Synthetic polypeptide
  REGION 60..61: note = NON_CONS - Residues at these positions can be separated by a linker of unknown length
LLADTTAARPWTLLADTTAARPWTLLADTTAARPWTLLADTTAARPWTLLADTTAARPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 586 - protein, 212 aa, synthetic construct
  REGION 1..212: note = Description of Artificial Sequence: Synthetic polypeptide
LLADTTAARPWTLLADTTAARPWTLLADTTAARPWTLLADTTAARPWTLLADTTAARPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 587 - protein, 138 aa, synthetic construct
  REGION 1..138: note = Description of Artificial Sequence: Synthetic polypeptide
  REGION 24..25: note = NON_CONS - Residues at these positions can be separated by a linker of unknown length
LLADTTHHRPWTLLADTTHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 588 - protein, 176 aa, synthetic construct
  REGION 1..176: note = Description of Artificial Sequence: Synthetic polypeptide
LLADTTHHRPWTLLADTTHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 589 - protein, 150 aa, synthetic construct
  REGION 1..150: note = Description of Artificial Sequence: Synthetic polypeptide
  REGION 36..37: note = NON_CONS - Residues at these positions can be separated by a linker of unknown length
LLADTTHHRPWTLLADTTHHRPWTLLADTTHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 590 - protein, 188 aa, synthetic construct
  REGION 1..188: note = Description of Artificial Sequence: Synthetic polypeptide
LLADTTHHRPWTLLADTTHHRPWTLLADTTHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 591 - protein, 174 aa, synthetic construct
  REGION 1..174: note = Description of Artificial Sequence: Synthetic polypeptide
  REGION 60..61: note = NON_CONS - Residues at these positions can be separated by a linker of unknown length
LLADTTHHRPWTLLADTTHHRPWTLLADTTHHRPWTLLADTTHHRPWTLLADTTHHRPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 592 - protein, 212 aa, synthetic construct
  REGION 1..212: note = Description of Artificial Sequence: Synthetic polypeptide
LLADTTHHRPWTLLADTTHHRPWTLLADTTHHRPWTLLADTTHHRPWTLLADTTHHRPWTASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 593 - protein, 234 aa, synthetic construct
  REGION 1..234: note = Description of Artificial Sequence: Synthetic polypeptide
  REGION 120..121: note = NON_CONS - Residues at these positions can be separated by a linker of unknown length
STSGSTVIGESTHHRPWSLIADSTHHSPWTILAESTHHKPWTILAETTHHRPWSIIGESSHHKPFTGLGDTTHHRPWGVLGDTTHHKPWTIVADSTHHRPWTGQVLPTTTPSSPSTTSGSQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 594 - protein, 272 aa, synthetic construct
  REGION 1..272: note = Description of Artificial Sequence: Synthetic polypeptide
STSGSTVIGESTHHRPWSLIADSTHHSPWTILAESTHHKPWTILAETTHHRPWSIIGESSHHKPFTGLGDTTHHRPWGVLGDTTHHKPWTIVADSTHHRPWTGQVLPTTTPSSPSTTSGSASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 595 - protein, 162 aa, synthetic construct
  REGION 1..162: note = Description of Artificial Sequence: Synthetic polypeptide
  REGION 48..49: note = NON_CONS - Residues at these positions can be separated by a linker of unknown length
LLADTTHHRPWTVIGESTHHRPWSIIGESSHHKPFTGLGDTTHHRPWGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 596 - protein, 200 aa, synthetic construct
  REGION 1..200: note = Description of Artificial Sequence: Synthetic polypeptide
LLADTTHHRPWTVIGESTHHRPWSIIGESSHHKPFTGLGDTTHHRPWGASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 597 - protein, 162 aa, synthetic construct
  REGION 1..162: note = Description of Artificial Sequence: Synthetic polypeptide
  REGION 48..49: note = NON_CONS - Residues at these positions can be separated by a linker of unknown length
VIGESTHHRPWSIIGESSHHKPFTGLGDTTHHRPWGILAESTHHKPWTQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 598 - protein, 200 aa, synthetic construct; identical to SEQ ID NO: 504
  REGION 1..200: note = Description of Artificial Sequence: Synthetic polypeptide

SEQ ID NO: 599 - protein, 1530 aa, synthetic construct
  REGION 1..1530: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1416..1417: note = NON_CONS - Residues at these positions can be separated by a linker of unknown length
  REGION 1..1530: note = See specification as filed for detailed description of substitutions and preferred embodiments
LLADTTHHRPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 600 - protein, 1568 aa, synthetic construct
  REGION 1..1568: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1..1568: note = See specification as filed for detailed description of substitutions and preferred embodiments
LLADTTHHRPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 601 - protein, 1530 aa, synthetic construct
  REGION 1..1530: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1416..1417: note = NON_CONS - Residues at these positions can be separated by a linker of unknown length
  REGION 1..1530: note = See specification as filed for detailed description of substitutions and preferred embodiments
VIGESTHHRPWSXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 602 - protein, 1568 aa, synthetic construct
  REGION 1..1568: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1..1568: note = See specification as filed for detailed description of substitutions and preferred embodiments
VIGESTHHRPWSXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 603 - protein, 1530 aa, synthetic construct
  REGION 1..1530: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1416..1417: note = NON_CONS - Residues at these positions can be separated by a linker of unknown length
  REGION 1..1530: note = See specification as filed for detailed description of substitutions and preferred embodiments
ILAESTHHKPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 604 - protein, 1568 aa, synthetic construct
  REGION 1..1568: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1..1568: note = See specification as filed for detailed description of substitutions and preferred embodiments
ILAESTHHKPWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 605 - protein, 1530 aa, synthetic construct
  REGION 1..1530: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1416..1417: note = NON_CONS - Residues at these positions can be separated by a linker of unknown length
  REGION 1..1530: note = See specification as filed for detailed description of substitutions and preferred embodiments
IIGESSHHKPFTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 606 - protein, 1568 aa, synthetic construct
  REGION 1..1568: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1..1568: note = See specification as filed for detailed description of substitutions and preferred embodiments
IIGESSHHKPFTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 607 - protein, 1530 aa, synthetic construct
  REGION 1..1530: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1416..1417: note = NON_CONS - Residues at these positions can be separated by a linker of unknown length
  REGION 1..1530: note = See specification as filed for detailed description of substitutions and preferred embodiments
GLGDTTHHRPWGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 608 - protein, 1568 aa, synthetic construct
  REGION 1..1568: note = Description of Artificial Sequence: Synthetic polypeptide
  MOD_RES 13..1416: note = This region may encompass one or more of the following sequences: "LLADTTHHRPWT" or "VIGESTHHRPWS" or "LIADSTHHSPWT" or "ILAESTHHKPWT" or "ILAETTHHRPWS" or "IIGESSHHKPFT" or "GLGDTTHHRPWG" or "VLGDTTHHKPWT" or "IVADSTHHRPWT" or
  MOD_RES 13..1416: note = CONT. FROM ABOVE: "STADTSHHRPS" or "TSGGESTHHRPS" or "TSGGESSHHKPS" or "TGSGDSSHHRPS" or "GSSGESTHHKPST" or "VGADSTHHRPVT" or "GAADTTHHRPVT" or "AGADTTHHRPVT" or "GGADTTHHRPAT" or "GGADTTHHRPGT" or "LLADTTHHRPWTVIGESTHHRPWS"
  MOD_RES 13..1416: note = CONT. FROM ABOVE: or "LLADTTHHRPWTVIGESTHHRPWSIIGESSHHKPFT" or "LLADTTHHRPWTVIG ESTHHRPWSIIGESSHHKPFTGLGDTTHHRPWGILAESTHHKPWT" or "LLADTTHHRPWTILAESTHHKPWT" or "LLADTTHHRPWTILAESTHHKPWTLLAD TTHHRPWTILAESTHHKPWTLLADTTHHRPWT" or "LLADTTHHRPWTGLGDTTHHRPWG"
  MOD_RES 13..1416: note = CONT. FROM ABOVE: or "LLADTTHHRPWTGLGDTTHHRPWGLLADTTHHRPWT" or "LLADTTHHRPWTGLG DTTHHRPWGLLADTTHHRPWTGLGDTTHHRPWGLLADTTHHRPWT" or "LLADTTH HRPWTGLGDTTHHRPWGLLADTTHHRPWTGLGDTTHHRPWGLLADTTHHRP WTGLGDTTHHRPWGLLADTTHHRPWT" or "STADTSHHRPSTSGGESTHHRPSTSGGESS
  MOD_RES 13..1416: note = CONT. FROM ABOVE: HHKPSTGSGDSSHHRPSGSSGESTHHKPST" or "VGADSTHHR PVTGAADTTHHRPVTAGADTTHHRPVTGGADTTHHRPATGGADTTHHRPGT" or "STADTS HHRPSLLADTTHHRPWTTSGGESTHHRPSVGADSTHHRPVTTSGGESSHH KPSGAADTTHHRP VTTGSGDSSHHRPSGSSGESTHHKPSTGGADTTHHRPAT" or "XXXXXXXXXXXX" or
  MOD_RES 13..1416: note = CONT. FROM ABOVE: "AAADTTHHRPWT" or "AAADTTHHRPWTAAADTTHHRPWTAA ADTTHHRPWTAAADTTHHRPWTAAADTTHHRPWT" or "LLADAAHHRPWTLLADAAHHRPW TLLADAAHHRPWTLLADAAHHRPWTLLADAAHHRPWT" or "LLADTTAARPWTLLADTTAA RPWTLLADTTAARPWTLLADTTAARPWTLLADTTAARPWT" or "LLADTTHHRPWTLLADT
  MOD_RES 13..1416: note = CONT. FROM ABOVE: THHRPWT" or "LLADTTHHRPWTLLADTTHHRPWTLLADTTHH RPWT" or "LLADTTHHRPWTLLADTTHHRPWTLLADTTHHRPWTLLADTTHHRPWTLLADT THHRPWT" or "STSGSTVIGESTHHRPWSLIADSTHHSPWTILAESTHHKPWTILAETTHH RPWSIIGESSHHKPFTGLGDTTHHRPWGVLGDTTHHKPWTIVADSTHHRPWTGQVLPT TTPSS
  MOD_RES 13..1416: note = CONT. FROM ABOVE: PSTTSGS" or "LLADTTHHRPWTVIGESTHHRPWSIIGESSHH KPFTGLGDTTHHRPWG" or "VIGESTHHRPWSIIGESSHHKPFTGLGDTTHHRPWGILAE STHHKPWT" and wherein X's respectively = V, L, I, G, S, T or A; I, L, V, Q, T, S, G or A; G, A, V or S; E, D, L or G; S, T, P
  MOD_RES 13..1416: note = CONT. FROM ABOVE: T, E or D; T or S; H, T or S; H or T; R, S, K, P or H; P, S, R or K; W, F, S, P, V, A or G; and absent, S, T G, or A
  REGION 1..1568: note = See specification as filed for detailed description of substitutions and preferred embodiments
GLGDTTHHRPWGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 609 - protein, 174 aa, synthetic construct
  REGION 1..174: note = Description of Artificial Sequence: Synthetic polypeptide
  MOD_RES 13..60: note = This region may encompass one or more of the following sequences: "VIGESTHHRPWS" or "ILAESTHHKPWT" or "IIGESSHHKPFT" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments
LLADTTHHRPWTXXXXXXXXXXXXXXXXXXXXXXXXXXXXXXXXXXXXXXXXXXXXXXXXQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 610 - protein, 212 aa, synthetic construct
  REGION 1..212: note = Description of Artificial Sequence: Synthetic polypeptide
  MOD_RES 13..60: note = This region may encompass one or more of the following sequences: "VIGESTHHRPWS" or "ILAESTHHKPWT" or "IIGESSHHKPFT" or "GLGDTTHHRPWG"
  REGION 1..212: note = See specification as filed for detailed description of substitutions and preferred embodiments
LLADTTHHRPWTXXXXXXXXXXXXXXXXXXXXXXXXXXXXXXXXXXXXXXXXXXXXXXXXASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 611 - protein, 174 aa, synthetic construct
  REGION 1..174: note = Description of Artificial Sequence: Synthetic polypeptide
  MOD_RES 13..60: note = This region may encompass one or more of the following sequences: "LLADTTHHRPWT" or "ILAESTHHKPWT" or "IIGESSHHKPFT" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments
VIGESTHHRPWSXXXXXXXXXXXXXXXXXXXXXXXXXXXXXXXXXXXXXXXXXXXXXXXXQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 612 - protein, 212 aa, synthetic construct
  REGION 1..212: note = Description of Artificial Sequence: Synthetic polypeptide
  MOD_RES 13..60: note = This region may encompass one or more of the following sequences: "LLADTTHHRPWT" or "ILAESTHHKPWT" or "IIGESSHHKPFT" or "GLGDTTHHRPWG"
  REGION 1..212: note = See specification as filed for detailed description of substitutions and preferred embodiments
VIGESTHHRPWSXXXXXXXXXXXXXXXXXXXXXXXXXXXXXXXXXXXXXXXXXXXXXXXXASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 613 - protein, 174 aa, synthetic construct
  REGION 1..174: note = Description of Artificial Sequence: Synthetic polypeptide
  MOD_RES 13..60: note = This region may encompass one or more of the following sequences: "LLADTTHHRPWT" or "VIGESTHHRPWS" or "IIGESSHHKPFT" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments
ILAESTHHKPWTXXXXXXXXXXXXXXXXXXXXXXXXXXXXXXXXXXXXXXXXXXXXXXXXQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 614 - protein, 212 aa, synthetic construct
  REGION 1..212: note = Description of Artificial Sequence: Synthetic polypeptide
  MOD_RES 13..60: note = This region may encompass one or more of the following sequences: "LLADTTHHRPWT" or "VIGESTHHRPWS" or "IIGESSHHKPFT" or "GLGDTTHHRPWG"
  REGION 1..212: note = See specification as filed for detailed description of substitutions and preferred embodiments
ILAESTHHKPWTXXXXXXXXXXXXXXXXXXXXXXXXXXXXXXXXXXXXXXXXXXXXXXXXASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 615 - protein, 174 aa, synthetic construct
  REGION 1..174: note = Description of Artificial Sequence: Synthetic polypeptide
  MOD_RES 13..60: note = This region may encompass one or more of the following sequences: "LLADTTHHRPWT" or "ILAESTHHKPWT" or "VIGESTHHRPWS" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments
IIGESSHHKPFTXXXXXXXXXXXXXXXXXXXXXXXXXXXXXXXXXXXXXXXXXXXXXXXXQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 616 - protein, 212 aa, synthetic construct
  REGION 1..212: note = Description of Artificial Sequence: Synthetic polypeptide
  MOD_RES 13..60: note = This region may encompass one or more of the following sequences: "LLADTTHHRPWT" or "ILAESTHHKPWT" or "VIGESTHHRPWS" or "GLGDTTHHRPWG"
  REGION 1..212: note = See specification as filed for detailed description of substitutions and preferred embodiments
IIGESSHHKPFTXXXXXXXXXXXXXXXXXXXXXXXXXXXXXXXXXXXXXXXXXXXXXXXXASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 617 - protein, 174 aa, synthetic construct
  REGION 1..174: note = Description of Artificial Sequence: Synthetic polypeptide
  MOD_RES 13..60: note = This region may encompass one or more of the following sequences: "LLADTTHHRPWT" or "ILAESTHHKPWT" or "IIGESSHHKPFT" or "VIGESTHHRPWS"
  REGION 60..61: note = NON_CONS - Residues at these positions can be separated by a linker of unknown length
  REGION 1..174: note = See specification as filed for detailed description of substitutions and preferred embodiments
GLGDTTHHRPWGXXXXXXXXXXXXXXXXXXXXXXXXXXXXXXXXXXXXXXXXXXXXXXXXQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 618 - protein, 212 aa, synthetic construct
  REGION 1..212: note = Description of Artificial Sequence: Synthetic polypeptide
  MOD_RES 13..60: note = This region may encompass one or more of the following sequences: "LLADTTHHRPWT" or "ILAESTHHKPWT" or "IIGESSHHKPFT" or "VIGESTHHRPWS"
  REGION 1..212: note = See specification as filed for detailed description of substitutions and preferred embodiments
GLGDTTHHRPWGXXXXXXXXXXXXXXXXXXXXXXXXXXXXXXXXXXXXXXXXXXXXXXXXASGAGGSEGGGSEGGTSGATGAGTSTSGGGASTGGGTGQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 619 - protein, 174 aa, synthetic construct; identical to SEQ ID NO: 609
  REGION 1..174: note = Description of Artificial Sequence: Synthetic polypeptide
  MOD_RES 13..60: note = This region may encompass two or more of the following sequences: "VIGESTHHRPWS" or "ILAESTHHKPWT" or "IIGESSHHKPFT" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments

SEQ ID NO: 620 - protein, 212 aa, synthetic construct; identical to SEQ ID NO: 610
  REGION 1..212: note = Description of Artificial Sequence: Synthetic polypeptide
  MOD_RES 13..60: note = This region may encompass two or more of the following sequences: "VIGESTHHRPWS" or "ILAESTHHKPWT" or "IIGESSHHKPFT" or "GLGDTTHHRPWG"
  REGION 1..212: note = See specification as filed for detailed description of substitutions and preferred embodiments

SEQ ID NO: 621 - protein, 174 aa, synthetic construct; identical to SEQ ID NO: 611
  REGION 1..174: note = Description of Artificial Sequence: Synthetic polypeptide
  MOD_RES 13..60: note = This region may encompass two or more of the following sequences: "LLADTTHHRPWT" or "ILAESTHHKPWT" or "IIGESSHHKPFT" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments

SEQ ID NO: 622 - protein, 212 aa, synthetic construct; identical to SEQ ID NO: 612
  REGION 1..212: note = Description of Artificial Sequence: Synthetic polypeptide
  MOD_RES 13..60: note = This region may encompass two or more of the following sequences: "LLADTTHHRPWT" or "ILAESTHHKPWT" or "IIGESSHHKPFT" or "GLGDTTHHRPWG"
  REGION 1..212: note = See specification as filed for detailed description of substitutions and preferred embodiments

SEQ ID NO: 623 - protein, 174 aa, synthetic construct; identical to SEQ ID NO: 613
  REGION 1..174: note = Description of Artificial Sequence: Synthetic polypeptide
  MOD_RES 13..60: note = This region may encompass two or more of the following sequences: "LLADTTHHRPWT" or "VIGESTHHRPWS" or "IIGESSHHKPFT" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments

SEQ ID NO: 624 - protein, 212 aa, synthetic construct; identical to SEQ ID NO: 614
  REGION 1..212: note = Description of Artificial Sequence: Synthetic polypeptide
  MOD_RES 13..60: note = This region may encompass two or more of the following sequences: "LLADTTHHRPWT" or "VIGESTHHRPWS" or "IIGESSHHKPFT" or "GLGDTTHHRPWG"
  REGION 1..212: note = See specification as filed for detailed description of substitutions and preferred embodiments

SEQ ID NO: 625 - protein, 174 aa, synthetic construct; identical to SEQ ID NO: 615
  REGION 1..174: note = Description of Artificial Sequence: Synthetic polypeptide
  MOD_RES 13..60: note = This region may encompass two or more of the following sequences: "LLADTTHHRPWT" or "ILAESTHHKPWT" or "VIGESTHHRPWS" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments

SEQ ID NO: 626 - protein, 212 aa, synthetic construct; identical to SEQ ID NO: 616
  REGION 1..212: note = Description of Artificial Sequence: Synthetic polypeptide
  MOD_RES 13..60: note = This region may encompass two or more of the following sequences: "LLADTTHHRPWT" or "ILAESTHHKPWT" or "VIGESTHHRPWS" or "GLGDTTHHRPWG"
  REGION 1..212: note = See specification as filed for detailed description of substitutions and preferred embodiments

SEQ ID NO: 627 - protein, 174 aa, synthetic construct; identical to SEQ ID NO: 617
  REGION 1..174: note = Description of Artificial Sequence: Synthetic polypeptide
  MOD_RES 13..60: note = This region may encompass two or more of the following sequences: "LLADTTHHRPWT" or "ILAESTHHKPWT" or "IIGESSHHKPFT" or "VIGESTHHRPWS"
  REGION 60..61: note = NON_CONS - Residues at these positions can be separated by a linker of unknown length
  REGION 1..174: note = See specification as filed for detailed description of substitutions and preferred embodiments

SEQ ID NO: 628 - protein, 212 aa, synthetic construct; identical to SEQ ID NO: 618
  REGION 1..212: note = Description of Artificial Sequence: Synthetic polypeptide
  MOD_RES 13..60: note = This region may encompass two or more of the following sequences: "LLADTTHHRPWT" or "ILAESTHHKPWT" or "IIGESSHHKPFT" or "VIGESTHHRPWS"
  REGION 1..212: note = See specification as filed for detailed description of substitutions and preferred embodiments

SEQ ID NO: 629 - protein, 174 aa, synthetic construct; identical to SEQ ID NO: 609
  REGION 1..174: note = Description of Artificial Sequence: Synthetic polypeptide
  MOD_RES 13..60: note = This region may encompass three or more of the following sequences: "VIGESTHHRPWS" or "ILAESTHHKPWT" or "IIGESSHHKPFT" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments

SEQ ID NO: 630 - protein, 212 aa, synthetic construct; identical to SEQ ID NO: 610
  REGION 1..212: note = Description of Artificial Sequence: Synthetic polypeptide
  MOD_RES 13..60: note = This region may encompass three or more of the following sequences: "VIGESTHHRPWS" or "ILAESTHHKPWT" or "IIGESSHHKPFT" or "GLGDTTHHRPWG"
  REGION 1..212: note = See specification as filed for detailed description of substitutions and preferred embodiments

SEQ ID NO: 631 - protein, 174 aa, synthetic construct; identical to SEQ ID NO: 611
  REGION 1..174: note = Description of Artificial Sequence: Synthetic polypeptide
  MOD_RES 13..60: note = This region may encompass three or more of the following sequences: "LLADTTHHRPWT" or "ILAESTHHKPWT" or "IIGESSHHKPFT" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments

SEQ ID NO: 632 - protein, 212 aa, synthetic construct; identical to SEQ ID NO: 612
  REGION 1..212: note = Description of Artificial Sequence: Synthetic polypeptide
  MOD_RES 13..60: note = This region may encompass three or more of the following sequences: "LLADTTHHRPWT" or "ILAESTHHKPWT" or "IIGESSHHKPFT" or "GLGDTTHHRPWG"
  REGION 1..212: note = See specification as filed for detailed description of substitutions and preferred embodiments

SEQ ID NO: 633 - protein, 174 aa, synthetic construct; identical to SEQ ID NO: 613
  REGION 1..174: note = Description of Artificial Sequence: Synthetic polypeptide
  MOD_RES 13..60: note = This region may encompass three or more of the following sequences: "LLADTTHHRPWT" or "VIGESTHHRPWS" or "IIGESSHHKPFT" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments

SEQ ID NO: 634 - protein, 212 aa, synthetic construct; identical to SEQ ID NO: 614
  REGION 1..212: note = Description of Artificial Sequence: Synthetic polypeptide
  MOD_RES 13..60: note = This region may encompass three or more of the following sequences: "LLADTTHHRPWT" or "VIGESTHHRPWS" or "IIGESSHHKPFT" or "GLGDTTHHRPWG"
  REGION 1..212: note = See specification as filed for detailed description of substitutions and preferred embodiments

SEQ ID NO: 635 - protein, 174 aa, synthetic construct; identical to SEQ ID NO: 615
  REGION 1..174: note = Description of Artificial Sequence: Synthetic polypeptide
  MOD_RES 13..60: note = This region may encompass three or more of the following sequences: "LLADTTHHRPWT" or "ILAESTHHKPWT" or "VIGESTHHRPWS" or "GLGDTTHHRPWG"
  REGION 60..61: note = NON_CONS - Residues at these positions can be separated by a linker of unknown length
  REGION 1..174: note = See specification as filed for detailed description of substitutions and preferred embodiments

SEQ ID NO: 636 - protein, 212 aa, synthetic construct; identical to SEQ ID NO: 616
  REGION 1..212: note = Description of Artificial Sequence: Synthetic polypeptide
  MOD_RES 13..60: note = This region may encompass three or more of the following sequences: "LLADTTHHRPWT" or "ILAESTHHKPWT" or "VIGESTHHRPWS" or "GLGDTTHHRPWG"
  REGION 1..212: note = See specification as filed for detailed description of substitutions and preferred embodiments

SEQ ID NO: 637 - protein, 174 aa, synthetic construct; identical to SEQ ID NO: 617
  REGION 1..174: note = Description of Artificial Sequence: Synthetic polypeptide
  MOD_RES 13..60: note = This region may encompass three or more of the following sequences: "LLADTTHHRPWT" or "ILAESTHHKPWT" or "IIGESSHHKPFT" or "VIGESTHHRPWS"
  REGION 60..61: note = NON_CONS - Residues at these positions can be separated by a linker of unknown length
  REGION 1..174: note = See specification as filed for detailed description of substitutions and preferred embodiments

SEQ ID NO: 638 - protein, 212 aa, synthetic construct; identical to SEQ ID NO: 618
  REGION 1..212: note = Description of Artificial Sequence: Synthetic polypeptide
  MOD_RES 13..60: note = This region may encompass three or more of the following sequences: "LLADTTHHRPWT" or "ILAESTHHKPWT" or "IIGESSHHKPFT" or "VIGESTHHRPWS"
  REGION 1..212: note = See specification as filed for detailed description of substitutions and preferred embodiments

SEQ ID NO: 639 - protein, 126 aa, synthetic construct
  REGION 1..126: note = Description of Artificial Sequence: Synthetic polypeptide
  REGION 12..13: note = NON_CONS - Residues at these positions can be separated by a linker of unknown length
VIGESTHHRPWSQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 640-700 - skipped (61 entries)

SEQ ID NO: 701 - protein, 38 aa, synthetic construct
  REGION 1..38: note = Description of Artificial Sequence: Synthetic polypeptide
ASGAGGSEGGGSEGGTSGATGAGTSTSGGGASTGGGTG

SEQ ID NO: 702 - protein, 4 aa, synthetic construct
  REGION 1..4: note = Description of Artificial Sequence: Synthetic peptide
GSEG

SEQ ID NO: 703 - protein, 4 aa, synthetic construct
  REGION 1..4: note = Description of Artificial Sequence: Synthetic peptide
SEGG

SEQ ID NO: 704 - protein, 5 aa, synthetic construct
  REGION 1..5: note = Description of Artificial Sequence: Synthetic peptide
MPIGS

SEQ ID NO: 705 - protein, 4 aa, synthetic construct
  REGION 1..4: note = Description of Artificial Sequence: Synthetic peptide
GSGS

SEQ ID NO: 706 - protein, 6 aa, synthetic construct
  REGION 1..6: note = Description of Artificial Sequence: Synthetic peptide
GSGSGS

SEQ ID NO: 707 - protein, 4 aa, synthetic construct
  REGION 1..4: note = Description of Artificial Sequence: Synthetic peptide
SGSG

SEQ ID NO: 708 - protein, 6 aa, synthetic construct
  REGION 1..6: note = Description of Artificial Sequence: Synthetic peptide
SGSGSG

SEQ ID NO: 709 - protein, 4 aa, synthetic construct
  REGION 1..4: note = Description of Artificial Sequence: Synthetic peptide
GSSG

SEQ ID NO: 710 - protein, 5 aa, synthetic construct
  REGION 1..5: note = Description of Artificial Sequence: Synthetic peptide
GGGGS

SEQ ID NO: 711 - DNA, 17 bp, synthetic construct
  misc_feature 1..17: note = Description of Artificial Sequence: Synthetic oligonucleotide
  source 1..17: mol_type = other DNA
aaataaaatacgaaatg

(19) United States
(12) Patent Application Publication
(10) Pub. No.: US 2025/0179136 A1
(43) Pub. Date: Jun. 5, 2025

(54) COMPOSITIONS AND METHODS FOR TARGETED THERAPEUTIC DELIVERY TO BONE

(71) Applicant: THERADAPTIVE, INC., Frederick, MD (US)

(72) Inventors: Luis ALVAREZ, Lexington, MA (US); David STEWART, Monrovia, MD (US); Hyeon PARK, Clarksburg, MD (US); Todd HEIL, Mt. Airy, MD (US)

(21) Appl. No.: 19/059,012

(22) Filed: Feb. 20, 2025

Related U.S. Application Data
(62) Division of application No. 18/046,810, filed on Oct. 14, 2022, now Pat. No. 12,264,187.
Application No. 18/046,810 is a continuation of application No. PCT/US21/27230, filed on Apr. 14, 2021.
(60) Provisional application No. 63/010,639, filed on Apr. 15, 2020.

(51) Int. Cl. C07K 14/51; A61K 38/18; A61P 19/00; C07K 14/475; C12N 15/62; C12N 15/63

(52) U.S. Cl. CPC C07K 14/51; A61K 38/18; A61P 19/00; C07K 14/475; C12N 15/62; C12N 15/63

(57) ABSTRACT

Provided herein are polypeptides comprising a therapeutic targeted for delivery to an organ or tissue, and uses thereof.

### PRIORITY CLAIM

This application is a divisional of U.S. patent application Ser. No. 18/046,810, filed Oct. 14, 2022, which is a continuation of International Application Serial No. PCT/US2021/027230, filed Apr. 14, 2021, which claims the benefit of U.S. Provisional Patent Application Ser. No. 63/010,639, filed Apr. 15, 2020, all of which are incorporated herein by reference in their entirety.

### GOVERNMENT RIGHTS

This invention was made with government support under W81XWH-18-C-0182 and W81XWH-15-C-0028 awarded by the Department of Defense. The government has certain rights in the invention.

### SEQUENCE LISTING

The instant application contains a Sequence Listing which has been submitted electronically in XML format and is hereby incorporated by reference in its entirety. Said XML copy, created on Feb. 20, 2025, is named 50222-705.401.xml and is 660,102 bytes in size.

### BACKGROUND

In the U.S., an average of six million people break a bone annually. Of these bone defects, the majority heal without problems. However, 5-10% will heal poorly or not at all (American Academy of Orthopaedic Surgeons). Large defects, also known as critical-sized bone defects, may not heal spontaneously and can lead to non-unions. A non-union occurs when there is no indication of healing for at least three months and no expectation of further healing (American Academy of Orthopaedic Surgeons). Currently, bone grafting is regarded as the “gold standard” for treating large or segmental bone defects. However, there are limitations with this treatment such as donor site pain, risk of rejection, limited donor supply and risk of transmission of infectious disease.

The repair of critical and complex bone and cartilage defects is also limited by poor tissue regrowth on implanted orthopedic substrates. Reasons for poor tissue regrowth include the loss of implanted progenitor cells within 48 hours post-implantation and the scarcity of progenitor cells. Another contributing factor is that current orthopedic substrates fail to facilitate sufficient tissue regeneration.

### SUMMARY

The present disclosure is based on the discovery that the use of a composition comprising a mammalian growth factor (e.g., bone morphogenetic protein 2 (BMP-2)) or a functional portion thereof, and a targeting polypeptide (e.g., a polypeptide that binds to bone or a carrier material) can vastly improve bone healing and accelerate tissue regrowth. In non-limiting embodiments, the targeting polypeptide binds to a carrier material, such as calcium phosphate (e.g., β-tricalcium phosphate or β-TCP), hydroxyapatite, or other materials suitable for use in an implantable device. Thus, provided herein are chimeric polypeptides comprising one or more targeting polypeptides and a mammalian growth factor, compositions comprising any of these chimeric polypeptides (and optionally, a carrier material), and methods of promoting bone or cartilage formulation, methods of replacing and/or repairing bone or cartilage, and methods of treating a bone fracture or bone loss that include administration of any of these compositions. The compositions and methods provided herein can increase and sustain the number of progenitor cells at sites of bone and/or cartilage injury through stem cell capture. The compositions and methods provided herein can also be applied to soft tissue repair or localized delivery of a therapeutic.

Provided herein is a polypeptide composition comprising: a targeting polypeptide comprising a sequence at least 80% identical to SEQ ID NO: 22 (LLADTTHHRPWT VIGESTHHRPWS IIGESSHHKPFT GLGDTTHHRPWG ILAESTHHKPWT), and a therapeutic polypeptide comprising a sequence at least 80% identical to a sequence selected from SEQ ID NOS: 32, 46-71, 73-77, 79-101, 152, 168, 176, 268. In some embodiments, the therapeutic polypeptide comprises a sequence at least 90% identical to a sequence selected from SEQ ID NOS: 32, 46-71, 73-77, 79-101, 152, 168, 176, 268. In some embodiments, the therapeutic polypeptide comprises a sequence selected from SEQ ID NOS: 32, 46-71, 73-77, 79-101, 152, 168, 176, 268. In some embodiments, the therapeutic polypeptide comprises a sequence at least 80%, 85%, 90%, or 95% identical to SEQ ID NO: 32. In some embodiments, the therapeutic polypeptide comprises SEQ ID NO: 32. In some embodiments, the targeting polypeptide comprises a sequence at least 90% or 95% identical to SEQ ID NO: 22. In some embodiments, the targeting polypeptide comprises SEQ ID NO: 22. In some embodiments, the targeting polypeptide comprises a sequence at least 90% or 95% identical to SEQ ID NO: 22. In some embodiments, the targeting polypeptide comprises SEQ ID NO: 22. In some embodiments, the targeting polypeptide comprises a sequence at least 90% or 95% identical to SEQ ID NO: 22. In some embodiments, the targeting polypeptide comprises SEQ ID NO: 22. In some embodiments, the targeting polypeptide comprises a sequence at least 90% or 95% identical to SEQ ID NO: 22. In some embodiments, the targeting polypeptide comprises SEQ ID NO: 22. In some embodiments, the targeting polypeptide comprises a sequence at least 90% or 95% identical to SEQ ID NO: 22. In some embodiments, the targeting polypeptide comprises SEQ ID NO: 22.

Also provided herein is a polypeptide composition comprising a sequence at least about 80% identical to SEQ ID NO: 551

((X)QAKHKQRKRLKSSCKRHPLYVDFSDVGWNDWIVAPPGYHAFYCHG
ECPFPLADHLNSTNHAIVQTLVNSVNSKIPKACCVPTELSAISMLYLDE
NEKVVLKNYQDMVVEGCGCR),

wherein the polypeptide composition comprises X, and X comprises SEQ ID NO: 22 (LLADTTHHRPWTVIGESTHHRPWSIIGESSHHKPFTGLGDTTHHRPWGILAESTHHKPWT). In some embodiments, the composition comprises a sequence at least about 90% or 95% identical to SEQ ID NO: 551.

Also provided herein is a polypeptide composition comprising a sequence at least about 80% identical to SEQ ID NO: 639

((X)QAKHKQRKRLKSSCKRHPLYVDFSDVGWNDWIVAPPGYHAFYCHG
ECPFPLADHLNSTNHAIVQTLVNSVNSKIPKACCVPTELSAISMLYLDE
NEKVVLKNYQDMVVEGCGCR),

wherein the polypeptide composition comprises X, and X comprises SEQ ID NO: 2 (VIGESTHHRPWS). In some embodiments, the composition comprises a sequence at least about 90% or 95% identical to SEQ ID NO: 639.

Also provided herein is a polypeptide composition comprising a sequence at least about 80% identical to SEQ ID NO: 519

((X)QAKHKQRKRLKSSCKRHPLYVDFSDVGWNDWIVAPPGYHAFYCHG
ECPFPLADHLNSTNHAIVQTLVNSVNSKIPKACCVPTELSAISMLYLDE
NEKVVLKNYQDMVVEGCGCR),

wherein the polypeptide composition comprises X, and X comprises SEQ ID NO: 6 (IIGESSHHKPFT). In some embodiments, the composition comprises a sequence at least about 90% or 95% identical to SEQ ID NO: 519.

Also provided herein is a polypeptide composition comprising a sequence at least about 80% identical to SEQ ID NO: 521

((X)QAKHKQRKRLKSSCKRHPLYVDFSDVGWNDWIVAPPGYHAFYCHG
ECPFPLADHLNSTNHAIVQTLVNSVNSKIPKACCVPTELSAISMLYLDE
NEKVVLKNYQDMVVEGCGCR),

wherein the polypeptide composition comprises X, and X comprises SEQ ID NO: 7 (GLGDTTHHRPWG). In some embodiments, the composition comprises a sequence at least about 90% or 95% identical to SEQ ID NO: 521.

Also provided herein is a polypeptide composition comprising a sequence at least about 80% identical to SEQ ID NO: 515

((X)QAKHKQRKRLKSSCKRHPLYVDFSDVGWNDWIVAPPGYHAFYCHG
ECPFPLADHLNSTNHAIVQTLVNSVNSKIPKACCVPTELSAISMLYLDE
NEKVVLKNYQDMVVEGCGCR),

wherein the polypeptide composition comprises X, and X comprises SEQ ID NO: 4 (ILAESTHHKPWT). In some embodiments, the composition comprises a sequence at least about 90% or 95% identical to SEQ ID NO: 515.

Also provided herein is a polypeptide composition comprising a sequence at least about 80% identical to SEQ ID NO: 501. In some embodiments, the sequence is at least about 90% or 95% identical to SEQ ID NO: 501. In some embodiments, the sequence comprises SEQ ID NO: 501.

Also provided herein is a polypeptide composition comprising a sequence at least about 80% identical to SEQ ID NO: 507. In some embodiments, the sequence is at least about 90% or 95% identical to SEQ ID NO: 507. In some embodiments, the sequence comprises SEQ ID NO: 507.

Also provided herein is a polypeptide composition comprising a sequence at least about 80% identical to SEQ ID NO: 506. In some embodiments, the sequence is at least about 90% or 95% identical to SEQ ID NO: 506. In some embodiments, the sequence comprises SEQ ID NO: 506.

Also provided herein is a polypeptide composition comprising a sequence at least about 80% identical to SEQ ID NO: 505. In some embodiments, the sequence is at least about 90% or 95% identical to SEQ ID NO: 505. In some embodiments, the sequence comprises SEQ ID NO: 505.

Also provided herein is a polypeptide composition comprising a sequence at least about 80% identical to SEQ ID NO: 504. In some embodiments, the sequence is at least about 90% or 95% identical to SEQ ID NO: 504. In some embodiments, the sequence comprises SEQ ID NO: 504.

Also provided herein is a polypeptide composition comprising a sequence at least about 80% identical to SEQ ID NO: 503. In some embodiments, the sequence is at least about 90% or 95% identical to SEQ ID NO: 503. In some embodiments, the sequence comprises SEQ ID NO: 503.

Also provided herein is a polypeptide composition comprising a sequence at least about 80% identical to SEQ ID NO: 502. In some embodiments, the sequence is at least about 90% or 95% identical to SEQ ID NO: 502. In some embodiments, the sequence comprises SEQ ID NO: 502.

Also provided herein is a polypeptide composition comprising: a targeting polypeptide comprising a sequence at least 80%, identical to SEQ ID NO: 22

(LLADTTHHRPWTVIGESTHHRPWSIIGESSHHKPFTGLGDTTHHRPWG
ILAESTHHKPWT),

and a therapeutic polypeptide comprising a bone morphogenetic protein (BMP). In some embodiments, the BMP is BMP-2.

Also provided herein is a nucleic acid encoding a polypeptide composition described herein. Also provided is a vector comprising the nucleic acid.

Also provided herein is a device comprises a polypeptide composition described herein, and a carrier material. In some embodiments, the polypeptide composition is bound to the carrier material. In some embodiments, the carrier material comprises calcium phosphate. In some embodiments, the carrier material comprises hydroxyapatite. In some embodiments, the carrier material comprises alpha tricalcium phosphate, fluorapatite, bone (e.g., demineralized bone), glasses (bioglasses) such as silicates, vanadates, and related ceramic minerals, or chelated divalent metal ions, or any combination thereof.

Also provided herein is a method of treating a subject in need thereof, the method comprising delivering to the subject a polypeptide composition described herein, or a device described herein. In some embodiments, the polypeptide composition or the device is delivered to treat a bone defect in the subject. In some embodiments, at least about 0.5 mg of polypeptide composition per cc of defect volume is delivered to the subject. In some embodiments, about 0.5 mg to about 10 mg of polypeptide composition per cc of defect volume is delivered to the subject.

Also provided herein is a method of delivering a therapeutic agent to an organ or tissue of a subject, the method comprising delivering to the organ or tissue a carrier material and the therapeutic agent, wherein the therapeutic agent is bound to the carrier material via a targeting polypeptide comprising: (a) a sequence at least 80% identical to SEQ ID NO: 22, (b) a sequence at least 80% identical to SEQ ID NO: 402, (c) a sequence at least 80% identical to SEQ ID NO: 401, (d) a sequence at least 80% identical to SEQ ID NO: 413 ((X1)(X2)), wherein X1 comprises SEQ ID NO: 1, and X2 comprises SEQ ID NO: 2 and SEQ ID NO: 6, (e) a sequence at least 80% identical to SEQ ID NO: 21, (f) a sequence at least 80% identical to SEQ ID NO: 414 ((X1)(X2)), wherein X1 comprises SEQ ID NO: 2, and X2 comprises SEQ ID NO: 6 and SEQ ID NO: 7, (g) a sequence at least 80% identical to SEQ ID NO: 416 ((X1)(X2)), wherein X1 comprises SEQ ID NO: 6, and X2 comprises SEQ ID NO: 7 and SEQ ID NO: 4, (h) a sequence at least 80% identical to SEQ ID NO: 408 ((X1)(X2)), wherein X1 comprises SEQ ID NO: 1, and X2 comprises SEQ ID NO: 2, (i) a sequence at least 80% identical to SEQ ID NO: 20, (j) a sequence at least 80% identical to SEQ ID NO: 409 ((X1)(X2)), wherein X1 comprises SEQ ID NO: 2, and X2 comprises SEQ ID NO: 6, (k) a sequence at least 80% identical to SEQ ID NO: 411 ((X1)(X2)), wherein X1 comprises SEQ ID NO: 6, and X2 comprises SEQ ID NO: 7, (l) a sequence at least 80% identical to SEQ ID NO: 412 ((X1)(X2)), wherein X1 comprises SEQ ID NO: 7, and X2 comprises SEQ ID NO: 4, (m) a sequence at least 80% identical to SEQ ID NO: 2 (VIGESTHHRPWS), (n) a sequence at least 80% identical to SEQ ID NO: 4 (ILAESTHHKPWT), (o) a sequence at least 80% identical to SEQ ID NO: 6 (IIGESSHHKPFT), (p) a sequence at least 80% identical to SEQ ID NO: 7 (GLGDTTHHRPWG), or (q) any combination of two or more of (a) to (p). In some embodiments, the carrier material comprises calcium phosphate. In some embodiments, the targeting polypeptide is connected to the therapeutic agent. In some embodiments, the therapeutic agent comprises a growth factor. In some embodiments, the therapeutic agent comprises BMP. In some embodiments, the BMP comprises BMP-2. In some embodiments, the therapeutic agent comprises a sequence at least about 80% identical to SEQ ID NO: 32. In some embodiments, the therapeutic agent comprises a sequence at least about 80% identical to any one of SEQ ID NOS: 46-71, 73-77, 79-101, 152, 168, 176, 268. In some embodiments, the therapeutic agent is delivered to treat a bone defect in the subject. In some embodiments, at least about 0.5 mg of the therapeutic agent is delivered per cc of defect volume. In some embodiments, about 0.5 mg to about 10 mg of the therapeutic agent is delivered per cc of defect volume.

Also provided herein are chimeric polypeptides comprising: (i) a targeting polypeptide comprising one or more of the following: an amino acid sequence at least about 70%, 75%, 80%, 85%, 90%, 95%, or 100% identical to VIGESTHHRPWS (SEQ ID NO: 2), an amino acid sequence at least about 70%, 75%, 80%, 85%, 90%, 95%, or 100% identical to LIADSTHHSPWT (SEQ ID NO: 3), ILAESTHHKPWT (SEQ ID NO: 4), an amino acid sequence at least about 70%, 75%, 80%, 85%, 90%, 95%, or 100% identical to ILAETTHHRPWS (SEQ ID NO: 5), an amino acid sequence at least about 70%, 75%, 80%, 85%, 90%, 95%, or 100% identical to IIGESSHHKPFT (SEQ ID NO: 6), an amino acid sequence at least about 70%, 75%, 80%, 85%, 90%, 95%, or 100% identical to GLGDTTHHRPWG (SEQ ID NO: 7), an amino acid sequence at least about 70%, 75%, 80%, 85%, 90%, 95%, or 100% identical to VLGDTTHHKPWT (SEQ ID NO: 8), an amino acid sequence at least about 70%, 75%, 80%, 85%, 90%, 95%, or 100% identical to IVADSTHHRPWT (SEQ ID NO: 9), an amino acid sequence at least about 70%, 75%, 80%, 85%, 90%, 95%, or 100% identical to STADTSHHRPS (SEQ ID NO: 10), an amino acid sequence at least about 70%, 75%, 80%, 85%, 90%, 95%, or 100% identical to TSGGESTHHRPS (SEQ ID NO: 11), an amino acid sequence at least about 70%, 75%, 80%, 85%, 90%, 95%, or 100% identical to TSGGESSHHKPS (SEQ ID NO: 12), an amino acid sequence at least about 70%, 75%, 80%, 85%, 90%, 95%, or 100% identical to TGSGDSSHHRPS (SEQ ID NO: 13), an amino acid sequence at least about 70%, 75%, 80%, 85%, 90%, 95%, or 100% identical to GSSGESTHHKPST (SEQ ID NO: 14), an amino acid sequence at least about 70%, 75%, 80%, 85%, 90%, 95%, or 100% identical to VGADSTHHRPVT (SEQ ID NO: 15), an amino acid sequence at least about 70%, 75%, 80%, 85%, 90%, 95%, or 100% identical to GAADTTHHRPVT (SEQ ID NO: 16), an amino acid sequence at least about 70%, 75%, 80%, 85%, 90%, 95%, or 100% identical to AGADTTHHRPVT (SEQ ID NO: 17), an amino acid sequence at least about 70%, 75%, 80%, 85%, 90%, 95%, or 100% identical to GGADTTHHRPAT (SEQ ID NO: 18), and an amino acid sequence at least about 70%, 75%, 80%, 85%, 90%, 95%, or 100% identical to GGADTTHHRPGT (SEQ ID NO: 19); and (ii) a mammalian growth factor.

In some embodiments, the targeting polypeptide comprises the sequence at least about 70%, 75%, 80%, 85%, 90%, 95%, or 100% identical to SEQ ID NO: 2. In some embodiments, the targeting polypeptide comprises the sequence at least about 90% identical to SEQ ID NO: 2. In some embodiments, the targeting polypeptide comprises SEQ ID NO: 2.

In some embodiments, the targeting polypeptide comprises the sequence at least about 70%, 75%, 80%, 85%, 90%, 95%, or 100% identical to SEQ ID NO: 4. In some embodiments, the targeting polypeptide comprises the sequence at least about 90% identical to SEQ ID NO: 4. In some embodiments, the targeting polypeptide comprises SEQ ID NO: 4.

In some embodiments, the targeting polypeptide comprises the sequence at least about 70%, 75%, 80%, 85%, 90%, 95%, or 100% identical to SEQ ID NO: 6. In some embodiments, the targeting polypeptide comprises the sequence at least about 90% identical to SEQ ID NO: 6. In some embodiments, the targeting polypeptide comprises SEQ ID NO: 6.

In some embodiments, the targeting polypeptide comprises the sequence at least about 70%, 75%, 80%, 85%, 90%, 95%, or 100% identical to SEQ ID NO: 7. In some embodiments, the targeting polypeptide comprises the sequence at least about 90% identical to SEQ ID NO: 7. In some embodiments, the targeting polypeptide comprises SEQ ID NO: 7.

In some embodiments, the targeting polypeptide comprises two or more targeting polypeptides. In some embodiments, two or more targeting polypeptides is no more than about 50, 45, 40, 35, 30, 25, 20, 15, or 10 targeting polypeptides. In some embodiments, two or more targeting polypeptides is about 2, 3, 4, 5, 6, 7, 8, 9, 10, 11, 12, 13, 14, 15, 20, 25, or 30 targeting polypeptides. In some embodiments, two or more targeting polypeptides is about 2 to about 10 targeting polypeptides. In some embodiments, two or more targeting polypeptides is about 5 targeting polypeptides.

In some embodiments, each neighboring pair of the two or more targeting polypeptides directly abut each other. In some embodiments, at least two of the two or more targeting polypeptides directly abut each other.

In some embodiments, each neighboring pair of the two or more targeting polypeptides are separated by a linker sequence. In some embodiments, at least two of the two or more targeting polypeptides are separated by a linker sequence.

In some embodiments, the targeting polypeptide comprises at least two different polypeptides.

In some embodiments, the targeting polypeptide includes two or more copies of the same polypeptide.

In some embodiments, the targeting polypeptide further comprises a sequence at least about 70%, 75%, 80%, 85%, 90%, 95%, or 100% identical to SEQ ID NO: 1. In some embodiments, the targeting polypeptide comprises the sequence at least about 90% identical to SEQ ID NO: 1. In some embodiments, the targeting polypeptide comprises SEQ ID NO: 1.

In some embodiments, the targeting polypeptide comprises: (i) a sequence at least about 70%, 75%, 80%, 85%, 90%, 95%, or 100% identical to SEQ ID NO: 1, (ii) a sequence at least about 70%, 75%, 80%, 85%, 90%, 95%, or 100% identical to SEQ ID NO: 2, (iii) a sequence at least about 70%, 75%, 80%, 85%, 90%, 95%, or 100% identical to SEQ ID NO: 4, (iv) a sequence at least about 70%, 75%, 80%, 85%, 90%, 95%, or 100% identical to SEQ ID NO: 6, (v) a sequence at least about 70%, 75%, 80%, 85%, 90%, 95%, or 100% identical to SEQ ID NO: 7, or (vi) any combination of (i) to (v). In some embodiments, the targeting polypeptide comprises (i), (ii), (iii), (iv), and (v). In some embodiments, the targeting polypeptide comprises SEQ ID NO: 2, SEQ ID NO: 4, SEQ ID NO: 6, and SEQ ID NO: 7. In some embodiments, the targeting polypeptide comprises SEQ ID NO: 1, SEQ ID NO: 2, SEQ ID NO: 4, SEQ ID NO: 6, and SEQ ID NO: 7.

In some embodiments, the targeting polypeptide comprises LLADTTHHRPWT (SEQ ID NO: 1) and/or GQVLPTTTPSSP (SEQ ID NO: 44).

In some embodiments, the targeting polypeptide comprises LLADTTHHRPWT (SEQ ID NO: 1) and/or VIGESTHHRPWS (SEQ ID NO: 2).

In some embodiments, the targeting polypeptide comprises a sequence at least about 70%, 75%, 80%, 85%, 90%, 95%, or 100% identical to LLADTTHHRPWTVIGESTHHRPWS (SEQ ID NO: 20). In some embodiments, the targeting polypeptide comprises a sequence at least about 90% identical to SEQ ID NO: 20. In some embodiments, the targeting polypeptide comprises SEQ ID NO: 20.

In some embodiments, the targeting polypeptide comprises a sequence at least about 70%, 75%, 80%, 85%, 90%, 95%, or 100% identical to LLADTTHHRPWT (SEQ ID NO: 1), a sequence at least about 70%, 75%, 80%, 85%, 90%, 95%, or 100% identical to VIGESTHHRPWS (SEQ ID NO: 2), and a sequence at least about 70%, 75%, 80%, 85%, 90%, 95%, or 100% identical to IIGESSHHKPFT (SEQ ID NO: 6). In some embodiments, the targeting polypeptide comprises a sequence at least 90% identical to SEQ ID NO: 1, a sequence at least 90% identical to SEQ ID NO: 2, and a sequence at least 90% identical to SEQ ID NO: 6. In some embodiments, the targeting polypeptide comprises SEQ ID NOS: 1, 2, and 6.

In some embodiments, the targeting polypeptide comprises a sequence at least about 70%, 75%, 80%, 85%, 90%, 95%, or 100% identical to VIGESTHHRPWS (SEQ ID NO: 2), and a sequence at least about 70%, 75%, 80%, 85%, 90%, 95%, or 100% identical to IIGESSHHKPFT (SEQ ID NO: 6). In some embodiments, the targeting polypeptide comprises a sequence at least 90% identical to SEQ ID NO: 2, and a sequence at least 90% identical to SEQ ID NO: 6. In some embodiments, the targeting polypeptide comprises SEQ ID NOS: 2, and 6.

In some embodiments, the targeting polypeptide comprises a sequence at least about 70%, 75%, 80%, 85%, 90%, 95%, or 100% identical to LLADTTHHRPWTVIGESTHHRPWSIIGESSHHKPFT (SEQ ID NO: 21). In some embodiments, the targeting polypeptide comprises a sequence at least about 90% identical to SEQ ID NO: 21. In some embodiments, the targeting polypeptide comprises SEQ ID NO: 21.

In some embodiments, the targeting polypeptide comprises a sequence at least about 70%, 75%, 80%, 85%, 90%, 95%, or 100% identical to LLADTTHHRPWTVIGESTHHRPWSIIGESSHHKPFTGLGDTTHHRPWG (SEQ ID NO: 401). In some embodiments, the targeting polypeptide comprises a sequence at least about 90% identical to SEQ ID NO: 401. In some embodiments, the targeting polypeptide comprises SEQ ID NO: 401.

In some embodiments, the targeting polypeptide comprises a sequence at least about 70%, 75%, 80%, 85%, 90%, 95%, or 100% identical to VIGESTHHRPWSIIGESSHHKPFTGLGDTTHHRPWGILAESTHHKPWT (SEQ ID NO: 402). In some embodiments, the targeting polypeptide comprises a sequence at least about 90% identical to SEQ ID NO: 402. In some embodiments, the targeting polypeptide comprises SEQ ID NO: 402.

In some embodiments, the targeting polypeptide comprises LLADTTHHRPWT (SEQ ID NO: 1), VIGESTHHRPWS (SEQ ID NO: 2), IIGESSHHKPFT (SEQ ID NO: 6), GLGDTTHHRPWG (SEQ ID NO: 7), and ILAESTHHKPWT (SEQ ID NO: 4).

In some embodiments, the targeting polypeptide comprises a sequence at least about 70%, 75%, 80%, 85%, 90%, 95%, or 100% identical to LLADTTHHRPWTVIGESTHHRPWSIIGESSHHKPFTGLGDTTHHRPWGILAESTHHKPWT (SEQ ID NO: 22). In some embodiments, the targeting polypeptide comprises a sequence at least about 80% identical to SEQ ID NO: 22. In some embodiments, the targeting polypeptide comprises a sequence at least about 90% identical to SEQ ID NO: 22. In some embodiments, the targeting polypeptide comprises SEQ ID NO: 22.

In some embodiments, the targeting polypeptide comprises a sequence at least about 70%, 75%, 80%, 85%, 90%, 95%, or 100% identical to LLADTTHHRPWT (SEQ ID NO: 1) and a sequence at least about 70%, 75%, 80%, 85%, 90%, 95%, or 100% identical to ILAESTHHKPWT (SEQ ID NO: 4). In some embodiments, the targeting polypeptide comprises a sequence at least 90% identical to SEQ ID NO: 1 and a sequence at least 90% identical to SEQ ID NO: 4. In some embodiments, the targeting polypeptide comprises SEQ ID NO: 1 and SEQ ID NO: 4.

In some embodiments, the targeting polypeptide comprises a sequence at least about 70%, 75%, 80%, 85%, 90%, 95%, or 100% identical to LLADTTHHRPWTILAESTHHKPWT (SEQ ID NO: 23).

In some embodiments, the targeting polypeptide comprises a sequence at least about 70%, 75%, 80%, 85%, 90%, 95%, or 100% identical to

(SEQ ID NO: 24)
LLADTTHHRPWTILAESTHHKPWTLLADTTHHRPWTILAESTHHKPWTL
LADTTHHRPWT.

In some embodiments, the targeting polypeptide comprises a sequence at least about 70%, 75%, 80%, 85%, 90%, 95%, or 100% identical to LLADTTHHRPWT (SEQ ID NO: 1) and a sequence at least about 70%, 75%, 80%, 85%, 90%, 95%, or 100% identical to GLGDTTHHRPWG (SEQ ID NO: 7). In some embodiments, the targeting polypeptide comprises a sequence at least about 90% identical to SEQ ID NO: 1 and a sequence at least about 70%, 75%, 80%, 85%, 90%, 95%, or 100% identical to SEQ ID NO: 7. In some embodiments, the targeting polypeptide comprises SEQ ID NO: 1 and SEQ ID NO: 7.

In some embodiments, the targeting polypeptide comprises a sequence at least about 70%, 75%, 80%, 85%, 90%, 95%, or 100% identical to LLADTTHHRPWTGLGDTTHHRPWG (SEQ ID NO: 25).

In some embodiments, the targeting polypeptide comprises a sequence at least about 70%, 75%, 80%, 85%, 90%, 95%, or 100% identical to

(SEQ ID NO: 26)
LLADTTHHRPWTGLGDTTHHRPWGLLADTTHHRPWT.

In some embodiments, the targeting polypeptide comprises a sequence at least about 70%, 75%, 80%, 85%, 90%, 95%, or 100% identical to

(SEQ ID NO: 27)
LLADTTHHRPWTGLGDTTHHRPWGLLADTTHHRPWTGLGDTTHHRPWGL
LADTTHHRPWT.

In some embodiments, the targeting polypeptide comprises a sequence at least about 70%, 75%, 80%, 85%, 90%, 95%, or 100% identical

(SEQ ID NO: 28)
LLADTTHHRPWTGLGDTTHHRPWGLLADTTHHRPWTGLGDTTHHRPWGL
LADTTHHRPWTGLGDTTHHRPWGLLADTTHHRPWT.

In some embodiments, the targeting polypeptide comprises a sequence at least about 70%, 75%, 80%, 85%, 90%, 95%, or 100% identical to STADTSHHRPS (SEQ ID NO: 10), a sequence at least about 70%, 75%, 80%, 85%, 90%, 95%, or 100% identical to TSGGESTHHRPS (SEQ ID NO: 11), a sequence at least about 70%, 75%, 80%, 85%, 90%, 95%, or 100% identical to TSGGESSHHKPS (SEQ ID NO: 12), a sequence at least about 70%, 75%, 80%, 85%, 90%, 95%, or 100% identical to TGSGDSSHHRPS (SEQ ID NO: 13), and a sequence at least about 70%, 75%, 80%, 85%, 90%, 95%, or 100% identical to GSSGESTHHKPST (SEQ ID NO: 14).

In some embodiments, the targeting polypeptide comprises a sequence at least about 70%, 75%, 80%, 85%, 90%, 95%, or 100% identical to

(SEQ ID NO: 29)
STADTSHHRPSTSGGESTHHRPSTSGGESSHHKPSTGSGDSSHHRPSGS
SGESTHHKPST.

In some embodiments, the targeting polypeptide comprises a sequence at least about 70%, 75%, 80%, 85%, 90%, 95%, or 100% identical to VGADSTHHRPVT (SEQ ID NO: 15), a sequence at least about 70%, 75%, 80%, 85%, 90%, 95%, or 100% identical to GAADTTHHRPVT (SEQ ID NO: 16), a sequence at least about 70%, 75%, 80%, 85%, 90%, 95%, or 100% identical to AGADTTHHRPVT (SEQ ID NO: 17), a sequence at least about 70%, 75%, 80%, 85%, 90%, 95%, or 100% identical to GGADTTHHRPAT (SEQ ID NO: 18) and a sequence at least about 70%, 75%, 80%, 85%, 90%, 95%, or 100% identical to GGADTTHHRPGT (SEQ ID NO: 19).

In some embodiments, the targeting polypeptide comprises a sequence at least about 70%, 75%, 80%, 85%, 90%, 95%, or 100% identical to

(SEQ ID NO: 30)
VGADSTHHRPVTGAADTTHHRPVTAGADTTHHRPVTGGADTTHHRPATG
GADTTHHRPGT.

In some embodiments, the targeting polypeptide comprises a sequence at least about 70%, 75%, 80%, 85%, 90%, 95%, or 100% identical to STADTSHHRPS (SEQ ID NO: 10), a sequence at least about 70%, 75%, 80%, 85%, 90%, 95%, or 100% identical to LLADTTHHRPWT (SEQ ID NO: 1), a sequence at least about 70%, 75%, 80%, 85%, 90%, 95%, or 100% identical to TSGGESTHHRPS (SEQ ID NO: 11), a sequence at least about 70%, 75%, 80%, 85%, 90%, 95%, or 100% identical to VGADSTHHRPVT (SEQ ID NO: 15), a sequence at least about 70%, 75%, 80%, 85%, 90%, 95%, or 100% identical to TSGGESSHHKPS (SEQ ID NO: 12), a sequence at least about 70%, 75%, 80%, 85%, 90%, 95%, or 100% identical to GAADTTHHRPVT (SEQ ID NO: 16), a sequence at least about 70%, 75%, 80%, 85%, 90%, 95%, or 100% identical to TGSGDSSHHRPS (SEQ ID NO: 13), a sequence at least about 70%, 75%, 80%, 85%, 90%, 95%, or 100% identical to GSSGESTHHKPST (SEQ ID NO: 14), and a sequence at least about 70%, 75%, 80%, 85%, 90%, 95%, or 100% identical to GGADTTHHRPAT (SEQ ID NO: 18).

In some embodiments, the targeting polypeptide comprises a sequence at least about 70%, 75%, 80%, 85%, 90%, 95%, or 100% identical to

(SEQ ID NO: 31)
STADTSHHRPSLLADTTHHRPWTTSGGESTHHRPSVGADSTHHRPVTTS
GGESSHHKPSGAADTTHHRPVTTGSGDSSHHRPSGSSGESTHHKPSTGG
ADTTHHRPAT.

In some embodiments, the targeting polypeptide comprises an amino acid sequence of Formula I: A₀B₀C₀D₀E₀F₀G₀H₀I₀J₀K₀L₀ (Formula I) (SEQ ID NO: 35), wherein: A₀ is V, L, I, G, S, T or A; B₀ is I, L, V, Q, T, S, G or A; C₀ is G, A, V or S; Do is E, D, L or G; E₀ is S, T, P T, E or D; F₀ is T or S; G₀ is H, T or S; H₀ is H or T; I₀ is R, S, K, P or H; J₀ is P, S, R or K; K₀ is W, F, S, P, V, A or G; and L₀ is absent or is S, T G, (or A). In some embodiments, Formula I does not comprise LLADTTHHRPWT (SEQ ID NO: 1).

In some embodiments, the targeting polypeptide comprises two or more amino acid sequences having Formula I. In some embodiments, at least two of the two or more amino acid sequences are different. In some embodiments, at least two or the two or more amino acid sequences are the same. In some embodiments, two or more is about 2, 3, 4, 5, 6, 7, 8, 9, or 10.

In some embodiments, at least two of the two or more amino acid sequences having Formula I directly abut each other. In some embodiments, each of the two or more amino acid sequences having Formula I directly abut each other.

In some embodiments, at least two of the two or more amino acid sequences of Formula I are separated by a linker sequence. In some embodiments, each of the two or more amino acid sequences having Formula I are separated by a linker sequence.

In some embodiments of any of the chimeric polypeptides described herein, the mammalian growth factor comprises one or more of the following: epidermal growth factor (EGF), platelet derived growth factor (PDGF), insulin like growth factor (IGF-1), fibroblast growth factor (FGF), fibroblast growth factor 2 (FGF2), fibroblast growth factor 18 (FGF18), transforming growth factor alpha (TGF-α), transforming growth factor beta (TGF-β), transforming growth factor beta 1 (TGF-β1), transforming growth factor beta 3 (TGF-β3), osteogenic protein 1 (OP-1), osteogenic protein 2 (OP-2), osteogenic protein 3 (OP-3), bone morphogenetic protein 2 (BMP-2), bone morphogenetic protein 3 (BMP-3), bone morphogenetic protein 4 (BMP-4), bone morphogenetic protein 5 (BMP-5), bone morphogenetic protein 6 (BMP-6), bone morphogenetic protein 7 (BMP-7), bone morphogenetic protein (BMP-9), bone morphogenetic protein 10 (BMP-10), bone morphogenetic protein 11 (BMP-11), bone morphogenetic protein 12 (BMP-12) bone morphogenetic protein 13 (BMP-13), bone morphogenetic protein 15 (BMP-15), dentin phosphoprotein (DPP), vegetal related growth factor (Vgr), growth differentiation factor 1 (GDF-1), growth differentiation factor 3 (GDF-3), growth differentiation factor 5 (GDF-5), growth differentiation factor 6 (GDF-6), growth differentiation factor 7 (GDF-7), growth differentiation factor 8 (GDF8), growth differentiation factor 11 (GDF11), growth differentiation factor 15 (GDF15), vascular endothelial growth factor (VEGF), hyaluronic acid binding protein (HABP), collagen binding protein (CBP), fibroblast growth factor 18 (FGF-18), keratinocyte growth factor (KGF), tumor necrosis factor alpha (TNFα), tumor necrosis factor (TNF)-related apoptosis inducing ligand (TRAIL), wnt family member 1 (WNT1), wnt family member 2 (WNT2), wnt family member 2B (WNT2B), wnt family member 3 (WNT3), wnt family member 3A (WNT3A), wnt family member 4 (WNT4), wnt family member 5A (WNT5A), wnt family member 5B (WNT5B), wnt family member 6 (WNT6), wnt family member 7A (WNT7A), wnt family member 7B (WNT7B), wnt family member 8A (WNT8A), wnt family member 8B (WNT8B), wnt family member 9A (WNT9A), wnt family member 9B (WNT9B), wnt family member 10A (WNT10A), wnt family member 10B (WNT10B), wnt family member 11 (WNT11), neuregulin 1 (NRG1), or wnt family member 16 (WNT16).

In some embodiments of any of the chimeric polypeptides described herein, the mammalian growth factor comprises a functional portion of one or more of the following: epidermal growth factor (EGF), platelet derived growth factor (PDGF), insulin like growth factor (IGF-1), fibroblast growth factor (FGF), fibroblast growth factor 2 (FGF2), fibroblast growth factor 18 (FGF18), transforming growth factor alpha (TGF-α), transforming growth factor beta (TGF-β), transforming growth factor beta 1 (TGF-β1), transforming growth factor beta 3 (TGF-β3), osteogenic protein 1 (OP-1), osteogenic protein 2 (OP-2), osteogenic protein 3 (OP-3), bone morphogenetic protein 2 (BMP-2), bone morphogenetic protein 3 (BMP-3), bone morphogenetic protein 4 (BMP-4), bone morphogenetic protein 5 (BMP-5), bone morphogenetic protein 6 (BMP-6), bone morphogenetic protein 7 (BMP-7), bone morphogenetic protein (BMP-9), bone morphogenetic protein 10 (BMP-10), bone morphogenetic protein 11 (BMP-11), bone morphogenetic protein 12 (BMP-12) bone morphogenetic protein 13 (BMP-13), bone morphogenetic protein 15 (BMP-15), dentin phosphoprotein (DPP), vegetal related growth factor (Vgr), growth differentiation factor 1 (GDF-1), growth differentiation factor 3 (GDF-3), growth differentiation factor 5 (GDF-5), growth differentiation factor 6 (GDF-6), growth differentiation factor 7 (GDF-7), growth differentiation factor 8 (GDF8), growth differentiation factor 11 (GDF11), growth differentiation factor 15 (GDF15), vascular endothelial growth factor (VEGF), hyaluronic acid binding protein (HABP), collagen binding protein (CBP), fibroblast growth factor 18 (FGF-18), keratinocyte growth factor (KGF), tumor necrosis factor alpha (TNFα), tumor necrosis factor (TNF)-related apoptosis inducing ligand (TRAIL), wnt family member 1 (WNT1), wnt family member 2 (WNT2), wnt family member 2B (WNT2B), wnt family member 3 (WNT3), wnt family member 3A (WNT3A), wnt family member 4 (WNT4), wnt family member 5A (WNT5A), wnt family member 5B (WNT5B), wnt family member 6 (WNT6), wnt family member 7A (WNT7A), wnt family member 7B (WNT7B), wnt family member 8A (WNT8A), wnt family member 8B (WNT8B), wnt family member 9A (WNT9A), wnt family member 9B (WNT9B), wnt family member 10A (WNT10A), wnt family member 10B (WNT10B), wnt family member 11 (WNT11), neuregulin 1 (NRG1), or wnt family member 16 (WNT16).

In some embodiments, the mammalian growth factor comprises BMP-2. In some embodiments, the mammalian growth factor comprises a functional portion of BMP-2. In some embodiments, the mammalian growth factor comprises the mature BMP-2 peptide without signal sequence. In some embodiments, the functional portion of BMP-2 comprises a sequence at least about 70%, 75%, 80%, 85%, 90%, 95%, or 100% identical to QAKHKQRKRLKSSCKRHPLYVDFSDVGWNDWIVAPPGYHAFYCHGECPFPLADHLNSTNH AIVQTLVNSVNSKIPKACCVPTELSAISMLYLDENEKVVLKNYQDMVVEGCGCR (SEQ ID NO: 32). In some embodiments, the mammalian growth factor comprises a sequence at least about 70%, 75%, 80%, 85%, 90%, 95%, or 100% identical to a sequence of Table B. In some embodiments, the mammalian growth factor comprises a sequence at least about 90% identical to SEQ ID NO: 32. In some embodiments, the mammalian growth factor comprises SEQ ID NO: 32.

In some embodiments, the chimeric polypeptide comprises (a) the targeting polypeptide comprising: (i) a sequence at least about 70%, 75%, 80%, 85%, 90%, 95%, or 100% identical to SEQ ID NO: 1, (ii) a sequence at least about 70%, 75%, 80%, 85%, 90%, 95%, or 100% identical to SEQ ID NO: 2, (iii) a sequence at least about 70%, 75%, 80%, 85%, 90%, 95%, or 100% identical to SEQ ID NO: 4, (iv) a sequence at least about 70%, 75%, 80%, 85%, 90%, 95%, or 100% identical to SEQ ID NO: 6, (v) a sequence at least about 70%, 75%, 80%, 85%, 90%, 95%, or 100% identical to SEQ ID NO: 7, or (vi) any combination of (i) to (v); and (b) the mammalian growth factor comprising a functional portion of BMP-2. In some embodiments, the functional portion of BMP-2 comprises SEQ ID NO: 32. In some embodiments, the chimeric polypeptide comprises the targeting polypeptide comprising SEQ ID NO: 2, SEQ ID NO: 4, SEQ ID NO: 6, and SEQ ID NO: 7; and the mammalian growth factor comprising SEQ ID NO: 32. In some embodiments, the targeting polypeptide and mammalian growth factor are connected via a linker polypeptide.

In some embodiments, the chimeric polypeptide comprises the targeting polypeptide comprising a sequence at least about 70%, 75%, 80%, 85%, 90%, 95%, or 100% identical to SEQ ID NO: 402; and the mammalian growth factor comprising a sequence at least about 70%, 75%, 80%, 85%, 90%, 95%, or 100% identical to SEQ ID NO: 32. In some embodiments, the chimeric polypeptide comprises the targeting polypeptide comprising a sequence at least about 90% identical to SEQ ID NO: 402, and the mammalian growth factor comprising a sequence at least about 90% identical to SEQ ID NO: 32. In some embodiments, the targeting polypeptide and mammalian growth factor are connected via a linker polypeptide.

In some embodiments, the chimeric polypeptide comprises the targeting polypeptide comprising a sequence at least about 70%, 75%, 80%, 85%, 90%, 95%, or 100% identical to SEQ ID NO: 22; and the mammalian growth factor comprising a sequence at least about 70%, 75%, 80%, 85%, 90%, 95%, or 100% identical to SEQ ID NO: 32. In some embodiments, the chimeric polypeptide comprises the targeting polypeptide comprising a sequence at least about 90% identical to SEQ ID NO: 22, and the mammalian growth factor comprising a sequence at least about 90% identical to SEQ ID NO: 32. In some embodiments, the targeting polypeptide and mammalian growth factor are connected via a linker polypeptide.

In some embodiments of any of the chimeric polypeptides described herein, the chimeric polypeptide further comprises a linker sequence. In some embodiments, the linker sequence connects the targeting polypeptide and the mammalian growth factor.

In some embodiments, the linker sequence comprises a sequence at least about 70%, 75%, 80%, 85%, 90%, 95%, or 100% identical to

(SEQ ID NO: 33)
TGGSGEGGTGASTGGSAGTGGSGGTTSGEAGGSSGAG.

In some embodiments, the linker sequence comprises at least about 70%, 75%, 80%, 85%, 90%, 95%, or 100% identical to GAGTG (SEQ ID NO: 34).

In some embodiments, the chimeric polypeptide comprises a flexible linker comprising a sequence of about 5 to about 50 amino acids, where at least about five of the amino acids have no regular secondary structure. In some embodiments, regular secondary structure comprises any helical structure like an alpha helix or 3₁₀ helix or π helix, a beta turn, omega loop, and/or a beta sheet. In some embodiments, the flexible linker comprises at least about 50%, 55%, 60%, 65%, 70%, 75%, 80%, 85% or 90% glycine, alanine, serine, glycine and alanine, glycine and serine, alanine and serine, or glycine, alanine, and serine. In some embodiments, the linker comprises 1, 2, 3, 4, or 5 GSEG (SEQ ID NO: 702). In some embodiments, the linker comprises 1, 2, 3, 4, or 5 SEGG (SEQ ID NO: 703).

In some embodiments, the chimeric polypeptide comprises a linker sequence at least about 70%, 75%, 80%, 85%, 90%, 95%, or 100% identical to ASGAGGSEGGGSEGGTSGATGAGTSTSGGGASTGGGTG (SEQ ID NO: 701). In some embodiments, the linker comprises a sequence at least about 90% identical to SEQ ID NO: 701. In some embodiments, the linker comprises SEQ ID NO: 701.

In some embodiments, the chimeric polypeptide comprises a sequence at least about 70%, 75%, 80%, 85%, 90%, 95%, or 100% identical to MPIGSLLADTTHHRPWTVIGESTHHRPWSIIGESSHHKPFTGLGDTTHHRPWGILAESTHHKP WTASGAGGSEGGGSEGGTSGATGAGTSTSGGGASTGGGTGQAKHKQRKRLKSSCKRHPLY VDFSDVGWNDWIVAPPGYHAFYCHGECPFPLADHLNSTNHAIVQTLVNSVNSKIPKACCVPT ELSAISMLYLDENEKVVLKNYQDMVVEGCGCR (SEQ ID NO: 502). In some embodiments, the chimeric polypeptide comprises a sequence at least about 90% identical to SEQ ID NO: 502. In some embodiments, the chimeric polypeptide comprises a sequence at least about 91% identical to SEQ ID NO: 502. In some embodiments, the chimeric polypeptide comprises a sequence at least about 92% identical to SEQ ID NO: 502. In some embodiments, the chimeric polypeptide comprises a sequence at least about 93% identical to SEQ ID NO: 502. In some embodiments, the chimeric polypeptide comprises a sequence at least about 94% identical to SEQ ID NO: 502. In some embodiments, the chimeric polypeptide comprises a sequence at least about 95% identical to SEQ ID NO: 502. In some embodiments, the chimeric polypeptide comprises a sequence at least about 96% identical to SEQ ID NO: 502. In some embodiments, the chimeric polypeptide comprises a sequence at least about 97% identical to SEQ ID NO: 502. In some embodiments, the chimeric polypeptide comprises a sequence at least about 98% identical to SEQ ID NO: 502. In some embodiments, the chimeric polypeptide comprises a sequence at least about 99% identical to SEQ ID NO: 502. In some embodiments, the chimeric polypeptide comprises SEQ ID NO: 502.

In some embodiments of any of the chimeric polypeptides described herein, the chimeric polypeptide binds to a carrier material. In some embodiments, the targeting polypeptide binds to the carrier material. In some embodiments, the carrier material comprises calcium phosphate. In some embodiments, the carrier material comprises β-TCP. In some embodiments, the carrier material comprises hydroxyapatite. In some embodiments, the targeting polypeptide binds to the carrier material with a K_{D} of about 1 picomolar to about 100 micromolar. In some embodiments, the targeting polypeptide binds to β-TCP with a K_{D} of about 1 picomolar to about 100 micromolar. In some embodiments, the K_{D} is measured using any method known in the art and/or described herein.

Also provided herein are compositions comprising any of one of the chimeric polypeptides described herein.

In some embodiments, the composition comprises a carrier material. In some embodiments, the carrier material comprises calcium phosphate. In some embodiments, the carrier material comprises β-TCP. In some embodiments, the carrier material comprises hydroxyapatite. In some embodiments, the carrier material is formulated as a powder, a putty, or a paste. In some embodiments, the carrier material comprises a scaffold, a fiber, a mesh, or a sponge.

In some embodiments of any of the compositions described herein, the composition is a pharmaceutical composition.

Also provided herein are kits comprising any of the polypeptides, carrier materials and/or compositions described herein.

Also provided herein are targeting polypeptides of any of the chimeric polypeptides described herein.

Also provided herein are methods of promoting bone or cartilage formation in a subject in need thereof that include: administering to the subject a therapeutically effective amount of any of the polypeptides and/or compositions described herein.

Also provided herein are methods of replacing and/or repairing bone or cartilage in a subject in need thereof that include: administering to the subject a therapeutically effective amount of any of the polypeptides and/or compositions described herein.

Also provided herein are methods of treating a bone fracture or bone loss in a subject in need thereof, that include: administering to the subject a therapeutically effective amount of any of the polypeptides and/or compositions described herein.

In some embodiments of any of the methods described herein, the subject has a bone fracture. In some embodiments of any of the methods described herein, the subject has a bone defect. In some embodiments of any of the methods described herein, the subject has a cartilage tear or cartilage defect. In some embodiments of any of the methods described herein, the subject has cartilage loss.

The term “subject” as used herein refers to any mammal. A subject therefore refers to, for example, mice, rats, dogs, cats, horses, cows, pigs, guinea pigs, rats, humans, monkeys, and the like. When the subject is a human, the subject may be referred to herein as a patient. In some embodiments, the subject or “subject in need of treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon), a human, or a rodent (e.g., a mouse, a guinea pig, a hamster, or a rat). In some embodiments, the subject or “subject in need of treatment” may be a non-human mammal, especially mammals that are conventionally used as models for demonstrating therapeutic efficacy in humans (e.g., murine, lapine, porcine, canine, or primate animals) may be employed.

In some embodiments, the term “therapeutically effective amount” refers to an amount of a polypeptide or composition effective to “treat” a disease, condition or disorder in a subject. In some cases, therapeutically effective amount of the polypeptide or composition reduces the severity of symptoms of the disease, condition or disorder. In some instances, the disease, condition or disorder comprises a defect in an organ or tissue.

“Affinity” refers to the strength of the sum total of non-covalent interactions between a β-TCP binding sequence (or a chimeric polypeptide or polypeptide comprising a β-TCP binding sequence) and its binding partner (e.g., β-TCP). Affinity can be measured by common methods known in the art, including those described herein. Affinity can be determined, for example, using surface plasmon resonance (SPR) technology (e.g., BIACORE®) or biolayer interferometry (e.g., FORTEBIO®). Additional methods for determining affinity are known in the art.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A are representative in vivo radiographs (lateromedial orientation) of animals from experimental Group A.

FIG. 1B are representative in vivo radiographs (lateromedial orientation) of animals from experimental Group B.

FIG. 1C are representative in vivo radiographs (lateromedial orientation) of animals from experimental Group C.

FIG. 2 is a representative graph of the mean radiographic score (+SEM, n=9-10) of animals from each Group at four weeks- and eight weeks-post-surgery (for each Group A-C, the bar on the left is 4 weeks and the bar on the right is 8 weeks). *P<0.01 vs Group C1 #P<0.01 vs Group V; † P<0.01 vs Group C at 4 weeks.

FIG. 3A are representative radiographs, sagittal, and transverse micro-CT images at 4-weeks post-operation from animals in Group A, B, or C.

FIG. 3B are representative radiographs, sagittal, and transverse micro-CT images at 4-weeks post-operation from animals in Group A, B, or C.

FIG. 4 is a schematic representation of the varied callus formation in animals from Group A, B, or C at 4 weeks post-surgery. The red-shaded area is an approximation of the mineralized callus volume.

FIG. 5A is a representative graph of micro-CT analyses showing total callus volume in bone specimens obtained at 8 weeks post-surgery from animals in Group A (n=9), B (n=7), or C (n=8). The data are represented as Mean±SEM; *P<0.01 vs B, #P<0.0.1 vs C.

FIG. 5B is a representative graph of micro-CT analyses showing bone mineral volume in bone specimens obtained 8 weeks post-surgery from animals in Group A (n=9), B (n=7), or C (n=8). The data are represented as Mean±SEM; *P<0.01 vs B, #P<0.0.1 vs C.

FIG. 6A is a representative graph of micro-CT analyses of the diaphyseal region volume of bone specimens obtained 4 weeks post-surgery in animals from Group A (n=10), B (n=8), or C (n=7). Bone mineral volume is expressed as the percentage of bone mineral volume to total volume. The data are represented as Mean±SEM; *P<0.05 vs B, #P<0.0.5 vs C. A1 vs B1; p=0.059.

FIG. 6B is a representative graph of micro-CT analyses of the cortical bone volume of bone specimens obtained 4 weeks post-surgery in animals from Group A (n=10), B (n=8), or C (n=7). Bone mineral volume is expressed as the percentage of bone mineral volume to total volume. The data are represented as Mean±SEM; *P<0.05 vs B, #P<0.0.5 vs C.

FIG. 7A is a representative graph of micro-CT analyses of total callus volume in bone specimens obtained 8 weeks post-surgery from animals in Group A (n=9), B (n=7), or C (n=8). The data are represented as Mean±SEM; *P<0.01 vs B, #P<0.0.1 vs C.

FIG. 7B is a representative graph of micro-CT analyses of bone mineral volume in bone specimens obtained 8 weeks post-surgery from animals in Group A (n=9), B (n=7), or C (n=8). The data are represented as Mean±SEM; *P<0.01 vs B, #P<0.0.1 vs C.

FIG. 8 is a representative graph of ultimate load to failure (ULF) at 4- and 8-weeks post-surgery for animals in Group A, B, or C (for each Group A-C, the bar on the left is 4 weeks and the bar on the right is 8 weeks). Data are represented as the mean+/−S.E.M. *P<0.05 vs 4-week groups; #P<0.01 vs 8-week groups.

FIG. 9A are representative histological images of Von Kossa- and H&E-stained bone specimens obtained at 4 weeks post-surgery from animals in Group A, B, or C.

FIG. 9B are representative histological images of TRAP+ osteoclast-stained bone specimens obtained at 4 weeks post-surgery from animals in Groups A, B, or C (left, middle, and right panels, respectively). White arrows indicate osteoclasts, and yellow arrows indicate osteocytes.

FIG. 10A are representative Von Kossa- and H&E-stained histological images of bone specimens obtained at 8 weeks post-surgery from animals in Group A, B, or C.

FIG. 10B are representative TRAP+ osteoclast-stained histological images of bone specimens obtained at 8 weeks post-surgery from animals in Group A, B, or C (left, middle, and right panels, respectively). White arrows indicate osteoclasts, and yellow arrows indicate osteocytes.

FIG. 11 is a representative immunoblot of MCF-7 cell lysate probed with an anti-P-Akt antibody, wherein the MCF-7 cells were incubated with β-TCBP-HRG.

FIG. 12 is a representative immunoblot of MCF-7 lysate probed with an anti-P-Akt antibody, wherein the MCF-7 cells were incubated with β-TCBP-HRG (free) or β-TCBP-HRG (β-TCP-bound).

FIG. 13 outlines the procedure for generating a tibial defect in goats as described in Example 6.

FIG. 14A are Mean Radial BV and Mean Moment Angle plots that illustrate the pattern and extent of new bone mineralization in the defect site for each treatment group.

FIG. 14B are Total Bone Volume plots in the 2.5 cm central region and 5 cm full defect region. Variation of total new bone volume among goats within treatment group was measured. At medium dose and high doses, tBMP-2 demonstrated comparable bone formation that was superior to the low dose and substrate only groups. For each Group A-D, the bar on the left corresponds to the 2.5 cm central region, and the bar on the right corresponds to the 5 cm region.

FIG. 14C is a Dose-Response model fit curve using non-linear least squares regression built based on bone volume data in 2.5 cm central region (red dots). The solid blue central line is the fit of the dose-response model. The three orange points from bottom to top identify the model-estimated doses at which 50%, 75%, and 90% of maximum effect is achieved on the log scale. The orange lines are 75% confidence intervals for such doses. The dose-response analysis showed that there is a statistically significant effect of the dose of tBMP-2 on the total bone volume generated in the central 2.5 cm region (p=0.0003). The modeling curve illustrates that the initial response increases rapidly with dose across the range from “No dose” to “low dose” (=2.14 mg/cc dose), and then more slowly as the plateau is attained. Above 2.14 mg/cc dose, there appears to be little additional benefit of increased tBMP-2 dose to get higher bone volume (dose-response curve becomes plateau line). A dose of 2 mg/cc of defect volume is sufficient to achieve complete response.

FIG. 14D is a ranking of postmortem radiographs (AP and ML views) of the 22 tibia defects. Higher rank number indicates greater bone formation in the tibial defect (1=complete bone healing to 22=no healing). Control and Low dose groups resulted in almost no new bone growth in defect site. Robust bone formation with some bone bridging was seen in the high and medium doses of tBMP-2 groups (9 of 15 goats). High and Medium groups led to a significant large new bone formation in the defect site compared to the other groups (“A” and “B”).

FIG. 15 outlines the procedure for generating and treating a tibial defect in sheep as described in Example 7.

FIG. 16A are representative radiographs at each month of the study described in Example 7, in the treatment group receiving 2 mg tBMP-2 per cc defect volume.

FIG. 16B is a graph of the mean±standard deviation bone volumes as measured from CT images for the study described in Example 7. For each week of treatment, the bars from left to right (dark to light shading) represent: 2 mg/cc tBMP2, 5 mg/cc tBMP2, and autograft control.

FIG. 16C are representative radiographs (AP and ML views) of the tibia defects in all conditions. 2 mg/cc dose groups resulted in significant bone growth, as did the 5 mg/cc and control conditions. Results were generally consistent across the multiple sheep tested.

FIG. 16D is a representative H&E histology image from the 2 mg/cc group at 8 weeks showing normal bone formation adjacent to the cut ends of the original cortex. Arrows indicate cortex (blue, top right arrow), connective (black, bottom right arrow) and cartilaginous tissue (grey, bottom left arrow), and new bone growth (green, top left arrow).

FIG. 16E is a tetrachrome histology image from the 2 mg/cc group. At 8 weeks, residual polymer fibers are visible. Some are surrounded by new bone growth and some are surrounded by connective tissue with multinucleated giant cells associated with the biological breakdown of the fibers.

### DETAILED DESCRIPTION

Provided herein are polypeptides for targeted delivery of a therapeutic agent to a region of a subject comprising a defect. In some embodiments, the region comprises bone and the therapeutic agent is delivered to treat a bone defect. In some embodiments, the polypeptide is a targeting polypeptide comprising one or more of the polypeptides of Table A.

In some embodiments, the targeting polypeptide comprises Formula I: A₀B₀C₀D₀E₀F₀G₀H₀I₀J₀K₀L₀ (Formula I) (SEQ ID NO: 35), where: A₀ is V, L, I, G, S, T or A; B₀ is I, L, V, Q, T, S, G or A; C₀ is G, A, V or S; Do is E, D, L or G; E₀ is S, T, P T, E or D; F₀ is T or S; G₀ is H, T or S; H₀ is H or T; I₀ is R, S, K, P or H; J₀ is P, S, R or K; K₀ is W, F, S, P, V, A or G; and L₀ is absent or is S, T G, (or A). In some embodiments, Formula I does not include LLADTTHHRPWT (SEQ ID NO: 1).

Also provided herein are chimeric polypeptides comprising the targeting polypeptide and a therapeutic agent. In some embodiments, the therapeutic agent comprises one or more of the polypeptides of Table B. In some embodiments, any of the targeting and/or chimeric polypeptides described herein can bind to one or more substrates comprising β TCP (e.g., a Mastergraft strip, Vitoss Foam Pack, chronOS Strip, Vitoss Micromorsels, LifeInk500, Hyperelastic Bone, bioactive glass, β TCP powder, β TCP spray dried powder, hydroxyapatite powder, hydroxyapatite-coated bone screw, β TCP granules, hydroxyapatite granules, and ReBOSSIS). In some embodiments, any of the targeting and/or chimeric polypeptides described herein can bind to silicon nitride or a substrate comprising silicon nitride.

Also provided herein are compositions that comprise any of the targeting and/or chimeric polypeptides described herein. In some embodiments, the compositions further comprise a substrate including β TCP (e.g., a Mastergraft strip, Vitoss Foam Pack, chronOS Strip, Vitoss Micromorsels, LifeInk500, Hyperelastic Bone, bioactive glass, β TCP powder, β TCP spray dried powder, hydroxyapatite powder, hydroxyapatite-coated bone screw, β TCP granules, hydroxyapatite granules, and ReBOSSIS). In some embodiments, the compositions further comprise a substrate comprising silicon nitride.

Also provided herein are kits that include any of these compositions. Also provided are methods of promoting bone or cartilage formation in a subject in need thereof, methods of replacing and/or repairing bone or cartilage in a subject in need thereof, and methods of treating a bone fraction or bone loss in a subject in need thereof that include administering to the subject any of the compositions described herein.

Non-limiting aspects of these polypeptides, compositions, kits, and methods are described below, and can be used in any combination without limitation. Additional aspects of these polypeptides, compositions, kits, and methods are known in the art.

### Chimeric Polypeptides

In one aspect, provided herein are chimeric polypeptides comprising a targeting polypeptide (e.g., a polypeptide capable of binding to a carrier material) and a therapeutic agent. As used herein, the term “chimeric polypeptide” is interchangeable with a “fusion protein”, “polypeptide composition” and the like. In some examples, the therapeutic agent comprises one or more of the peptides of Table B, or a functional portion thereof. In some examples, the chimeric polypeptide comprises a targeting polypeptide comprising a sequence at least about 70%, 75%, 80%, 85%, 90%, 95%, or 100% identical to one or more of SEQ ID NOS: 1-43 or 401-422. In some embodiments, the targeting polypeptide comprises Formula I. In some embodiments, the targeting polypeptide comprises two or more targeting polypeptides. In some embodiments, two or more targeting polypeptides is no more than about 50, 45, 40, 35, 30, 25, 20, 15, or 10 targeting polypeptides. In some embodiments, two or more targeting polypeptides is about 2, 3, 4, 5, 6, 7, 8, 9, 10, 11, 12, 13, 14, 15, 20, 25, or 30 targeting polypeptides. In some embodiments, two or more targeting polypeptides is about 2 to about 10 targeting polypeptides. In some embodiments, two or more targeting polypeptides is about 5 targeting polypeptides.

In some examples, the targeting polypeptide of the chimeric polypeptide comprise Formula I: A₀B₀C₀D₀E₀F₀G₀H₀I₀J₀K₀L₀ (Formula I) (SEQ ID NO: 35), wherein: A₀ is V, L, I, G, S, T, or A; B₀ is I, L, V, Q, T, S, G, or A; C₀ is G, A, V, or S; D₀ is E, D, L, or G; E₀ is S, T, P T, E, or D; F₀ is T or S; G₀ is H, T, or S; H₀ is H or T; I₀ is R, S, K, P, or H; J₀ is P, S, R, or K; K₀ is W, F, S, P, V, A, or G; and L₀ is absent or is S, T, G, or A.

Non-limiting examples of targeting polypeptides that may be present in any of the chimeric polypeptides described herein are listed in Table A below.

TABLE A
Exemplary targeting polypeptide sequences

| | SEQ ID |
|---|---|
| Sequence | NO: |
| LLADTTHHRPWT | 1 |
| VIGESTHHRPWS | 2 |
| LIADSTHHSPWT | 3 |
| ILAESTHHKPWT | 4 |
| ILAETTHHRPWS | 5 |
| IIGESSHHKPFT | 6 |
| GLGDTTHHRPWG | 7 |
| VLGDTTHHKPWT | 8 |
| IVADSTHHRPWT | 9 |
| STADTSHHRPS | 10 |
| TSGGESTHHRPS | 11 |
| TSGGESSHHKPS | 12 |
| TGSGDSSHHRPS | 13 |
| GSSGESTHHKPST | 14 |
| VGADSTHHRPVT | 15 |
| GAADTTHHRPVT | 16 |
| AGADTTHHRPVT | 17 |
| GGADTTHHRPAT | 18 |
| GGADTTHHRPGT | 19 |
| LLADTTHHRPWTVIGESTHHRPWS | 20 |
| LLADTTHHRPWTVIGESTHHRPWSIIGESSHHKPFT | 21 |
| LLADTTHHRPWTVIGESTHHRPWSIIGESSHHKPFTGLGDTTHHRPWGILAESTHHKP | 22 |
| WT | |
| LLADTTHHRPWTILAESTHHKPWT | 23 |
| LLADTTHHRPWTILAESTHHKPWTLLADTTHHRPWT | 24 |
| ILAESTHHKPWTLLADTTHHRPWT | |
| LLADTTHHRPWTGLGDTTHHRPWG | 25 |
| LLADTTHHRPWTGLGDTTHHRPWGLLADTTHHRPWT | 26 |
| LLADTTHHRPWTGLGDTTHHRPWGLLADTTHHRPWT | 27 |
| GLGDTTHHRPWGLLADTTHHRPWT | |
| LLADTTHHRPWTGLGDTTHHRPWGLLADTTHHRPWT | 28 |
| GLGDTTHHRPWGLLADTTHHRPWTGLGDTTHHRPWGLLADTTHHRPWT | |
| STADTSHHRPSTSGGESTHHRPSTSGGESSHHKPST | 29 |
| GSGDSSHHRPSGSSGESTHHKPST | |
| VGADSTHHRPVTGAADTTHHRPVTAGADTTHHRPVT | 30 |
| GGADTTHHRPATGGADTTHHRPGT | |
| STADTSHHRPSLLADTTHHRPWTTSGGESTHHRPSVGADSTHHRPVT | 31 |
| TSGGESSHHKPSGAADTTHHRPVTTGSGDSSHHRPSGSSGESTHHKPS | |
| TGGADTTHHRPAT | |
| AAADTTHHRPWT | 36 |
| AAADTTHHRPWTAAADTTHHRPWTAAADTTHHRPWTAAAD | 37 |
| TTHHRPWTAAADTTHHRPWT | |
| LLADAAHHRPWTLLADAAHHRPWTLLADAAHHRPWT | 38 |
| LLADAAHHRPWTLLADAAHHRPWT | |
| LLADTTAARPWTLLADTTAARPWTLLADTTAARPWT | 39 |
| LLADTTAARPWTLLADTTAARPWT | |
| LLADTTHHRPWTLLADTTHHRPWT | 40 |
| LLADTTHHRPWTLLADTTHHRPWTLLADTTHHRPWT | 41 |
| LLADTTHHRPWTLLADTTHHRPWTLLADTTHHRPWT | 42 |
| LLADTTHHRPWTLLADTTHHRPWT | |
| STSGSTVIGESTHHRPWSLIADSTHHSPWTILAESTHHKPWT | 43 |
| ILAETTHHRPWSIIGESSHHKPFTGLGDTTHHRPWGVLGDTTHHKPWT | |
| IVADSTHHRPWTGQVLPTTTPSSPSTTSGS | |
| LLADTTHHRPWTVIGESTHHRPWSIIGESSHHKPFTGLGDTTHHRPWG | 401 |
| VIGESTHHRPWSIIGESSHHKPFTGLGDTTHHRPWGILAESTHHKPWT | 402 |
| (X1)(X2), wherein X1 comprises SEQ ID NO: 1 and X2 comprises one or more of SEQ ID | 403 |
| NOS: 1-31,35-43, or 401-402. | |
| (X1)(X2), wherein X1 comprises SEQ ID NO: 2 and X2 comprises one or more of SEQ ID | 404 |
| NOS: 1-31,35-43, or 401-402. | |
| (X1)(X2), wherein X1 comprises SEQ ID NO: 4 and X2 comprises one or more of SEQ ID | 405 |
| NOS: 1-31,35-43, or 401-402. | |
| (X1)(X2), wherein X1 comprises SEQ ID NO: 6 and X2 comprises one or more of SEQ ID | 406 |
| NOS: 1-31,35-43, or 401-402. | |
| (X1)(X2), wherein X1 comprises SEQ ID NO: 7 and X2 comprises one or more of SEQ ID | 407 |
| NOS: 1-31,35-43, or 401-402. | |
| (X1)(X2), wherein X1 comprises SEQ ID NO: 1 and X2 comprises one or more of SEQ ID | 408 |
| NOS: 2, 4, 6, or 7. | |
| (X1)(X2), wherein X1 comprises SEQ ID NO: 2 and X2 comprises one or more of SEQ ID | 409 |
| NOS: 1,4, 6, or 7. | |
| (X1)(X2), wherein X1 comprises SEQ ID NO: 4 and X2 comprises one or more of SEQ ID | 410 |
| NOS: 1,2, 6, or 7. | |
| (X1)(X2), wherein X1 comprises SEQ ID NO: 6 and X2 comprises one or more of SEQ ID | 411 |
| NOS: 1,4, 2, or 7. | |
| (X1)(X2), wherein X1 comprises SEQ ID NO: 7 and X2 comprises one or more of SEQ ID | 412 |
| NOS: 1,4, 6, or 2. | |
| (X1)(X2), wherein X1 comprises SEQ ID NO: 1 and X2 comprises two or more of SEQ ID | 413 |
| NOS: 2, 4, 6, or 7. | |
| (X1)(X2), wherein X1 comprises SEQ ID NO: 2 and X2 comprises two or more of SEQ ID | 414 |
| NOS: 1,4, 6, or 7. | |
| (X1)(X2), wherein X1 comprises SEQ ID NO: 4 and X2 comprises two or more of SEQ ID | 415 |
| NOS: 1,2, 6, or 7. | |
| (X1)(X2), wherein X1 comprises SEQ ID NO: 6 and X2 comprises two or more of SEQ ID | 416 |
| NOS: 1,4, 2, or 7. | |
| (X1)(X2), wherein X1 comprises SEQ ID NO: 7 and X2 comprises two or more of SEQ ID | 417 |
| NOS: 1,4, 6, or 2. | |
| (X1)(X2), wherein X1 comprises SEQ ID NO: 1 and X2 comprises three or more of | 418 |
| SEQ ID NOS: 2, 4, 6, or 7. | |
| (X1)(X2), wherein X1 comprises SEQ ID NO: 2 and X2 comprises three or more of | 419 |
| SEQ ID NOS: 1,4, 6, or 7. | |
| (X1)(X2), wherein X1 comprises SEQ ID NO: 4 and X2 comprises three or more of | 420 |
| SEQ ID NOS: 1,2, 6, or 7. | |
| (X1)(X2), wherein X1 comprises SEQ ID NO: 6 and X2 comprises three or more of | 421 |
| SEQ ID NOS: 1,4, 2, or 7. | |
| (X1)(X2), wherein X1 comprises SEQ ID NO: 7 and X2 comprises three or more of | 422 |
| SEQ ID NOS: 1,4, 6, or 2. | |

TABLE C
Exemplary Chimeric Polypeptides

| | SEQ ID |
|---|---|
| Sequence | NO |
| ASGAGGSEGGGSEGGTSGATGAGTSTSGGGASTGGGTGQAKHKQRKRLKSSCKRHPL | 501 |
| YVDFSDVGWNDWIVAPPGYHAFYCHGECPFPLADHLNSTNHAIVQTLVNSVNSKIPK | |
| ACCVPTELSAISMLYLDENEKVVLKNYQDMVVEGCGCR | |
| MPIGSLLADTTHHRPWTVIGESTHHRPWSIIGESSHHKPFTGLGDTTHHRPWGILAEST | 502 |
| HHKPWTASGAGGSEGGGSEGGTSGATGAGTSTSGGGASTGGGTGQAKHKQRKRLKS | |
| SCKRHPLYVDFSDVGWNDWIVAPPGYHAFYCHGECPFPLADHLNSTNHAIVQTLVNS | |
| VNSKIPKACCVPTELSAISMLYLDENEKVVLKNYQDMVVEGCGCR | |
| LLADTTHHRPWTVIGESTHHRPWSIIGESSHHKPFTGLGDTTHHRPWGILAESTHHKP | 503 |
| WTASGAGGSEGGGSEGGTSGATGAGTSTSGGGASTGGGTGQAKHKQRKRLKSSCKR | |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| VIGESTHHRPWSIIGESSHHKPFTGLGDTTHHRPWGILAESTHHKPWTASGAGGSEGG | 504 |
| GSEGGTSGATGAGTSTSGGGASTGGGTGQAKHKQRKRLKSSCKRHPLYVDFSDVGW | |
| NDWIVAPPGYHAFYCHGECPFPLADHLNSTNHAIVQTLVNSVNSKIPKACCVPTELSAI | |
| SMLYLDENEKVVLKNYQDMVVEGCGCR | |
| IIGESSHHKPFTGLGDTTHHRPWGILAESTHHKPWTASGAGGSEGGGSEGGTSGATGA | 505 |
| GTSTSGGGASTGGGTGQAKHKQRKRLKSSCKRHPLYVDFSDVGWNDWIVAPPGYHA | |
| FYCHGECPFPLADHLNSTNHAIVQTLVNSVNSKIPKACCVPTELSAISMLYLDENEKVV | |
| LKNYQDMVVEGCGCR | |
| GLGDTTHHRPWGILAESTHHKPWTASGAGGSEGGGSEGGTSGATGAGTSTSGGGAST | 506 |
| GGGTGQAKHKQRKRLKSSCKRHPLYVDFSDVGWNDWIVAPPGYHAFYCHGECPFPL | |
| ADHLNSTNHAIVQTLVNSVNSKIPKACCVPTELSAISMLYLDENEKVVLKNYQDMVV | |
| EGCGCR | |
| ILAESTHHKPWTASGAGGSEGGGSEGGTSGATGAGTSTSGGGASTGGGTGQAKHKQR | 507 |
| KRLKSSCKRHPLYVDFSDVGWNDWIVAPPGYHAFYCHGECPFPLADHLNSTNHAIVQ | |
| TLVNSVNSKIPKACCVPTELSAISMLYLDENEKVVLKNYQDMVVEGCGCR | |
| (X)QAKHKQRKRLKSSCKRHPLYVDFSDVGWNDWIVAPPGYHAFYCHGECPFPLADH | 508 |
| LNSTNHAIVQTLVNSVNSKIPKACCVPTELSAISMLYLDENEKVVLKNYQDMVVEGC | |
| GCR | |
| Wherein X comprises Formula 1 and optionally a linker (e.g., | |
| X may comprise Formula I + linker) | |
| (X)ASGAGGSEGGGSEGGTSGATGAGTSTSGGGASTGGGTGQAKHKQRKRLKSSCKR | 509 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises Formula I | |
| (X)QAKHKQRKRLKSSCKRHPLYVDFSDVGWNDWIVAPPGYHAFYCHGECPFPLADH | 510 |
| LNSTNHAIVQTLVNSVNSKIPKACCVPTELSAISMLYLDENEKVVLKNYQDMVVEGC | |
| GCR | |
| Wherein X comprises SEQ ID NO: 1, and optionally a linker | |
| (X)ASGAGGSEGGGSEGGTSGATGAGTSTSGGGASTGGGTGQAKHKQRKRLKSSCKR | 511 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 1 | |
| (X)QAKHKQRKRLKSSCKRHPLYVDFSDVGWNDWIVAPPGYHAFYCHGECPFPLADH | 639 |
| LNSTNHAIVQTLVNSVNSKIPKACCVPTELSAISMLYLDENEKVVLKNYQDMVVEGC | |
| GCR | |
| Wherein X comprises SEQ ID NO: 2, and optionally a linker | |
| (X)ASGAGGSEGGGSEGGTSGATGAGTSTSGGGASTGGGTGQAKHKQRKRLKSSCKR | 512 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 2 | |
| (X)QAKHKQRKRLKSSCKRHPLYVDFSDVGWNDWIVAPPGYHAFYCHGECPFPLADH | 513 |
| LNSTNHAIVQTLVNSVNSKIPKACCVPTELSAISMLYLDENEKVVLKNYQDMVVEGC | |
| GCR | |
| Wherein X comprises SEQ ID NO: 3, and optionally a linker | |
| (X)ASGAGGSEGGGSEGGTSGATGAGTSTSGGGASTGGGTGQAKHKQRKRLKSSCKR | 514 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 3 | |
| (X)QAKHKQRKRLKSSCKRHPLYVDFSDVGWNDWIVAPPGYHAFYCHGECPFPLADH | 515 |
| LNSTNHAIVQTLVNSVNSKIPKACCVPTELSAISMLYLDENEKVVLKNYQDMVVEGC | |
| GCR | |
| Wherein X comprises SEQ ID NO: 4, and optionally a linker | |
| (X)ASGAGGSEGGGSEGGTSGATGAGTSTSGGGASTGGGTGQAKHKQRKRLKSSCKR | 516 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 4 | |
| (X)QAKHKQRKRLKSSCKRHPLYVDFSDVGWNDWIVAPPGYHAFYCHGECPFPLADH | 517 |
| LNSTNHAIVQTLVNSVNSKIPKACCVPTELSAISMLYLDENEKVVLKNYQDMVVEGC | |
| GCR | |
| Wherein X comprises SEQ ID NO: 5, and optionally a linker | |
| (X)ASGAGGSEGGGSEGGTSGATGAGTSTSGGGASTGGGTGQAKHKQRKRLKSSCKR | 518 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 5 | |
| (X)QAKHKQRKRLKSSCKRHPLYVDFSDVGWNDWIVAPPGYHAFYCHGECPFPLADH | 519 |
| LNSTNHAIVQTLVNSVNSKIPKACCVPTELSAISMLYLDENEKVVLKNYQDMVVEGC | |
| GCR | |
| Wherein X comprises SEQ ID NO: 6, and optionally a linker | |
| (X)ASGAGGSEGGGSEGGTSGATGAGTSTSGGGASTGGGTGQAKHKQRKRLKSSCKR | 520 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 6 | |
| (X)QAKHKQRKRLKSSCKRHPLYVDFSDVGWNDWIVAPPGYHAFYCHGECPFPLADH | 521 |
| LNSTNHAIVQTLVNSVNSKIPKACCVPTELSAISMLYLDENEKVVLKNYQDMVVEGC | |
| GCR | |
| Wherein X comprises SEQ ID NO: 7, and optionally a linker | |
| (X)ASGAGGSEGGGSEGGTSGATGAGTSTSGGGASTGGGTGQAKHKQRKRLKSSCKR | 522 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 7 | |
| (X)QAKHKQRKRLKSSCKRHPLYVDFSDVGWNDWIVAPPGYHAFYCHGECPFPLADH | 523 |
| LNSTNHAIVQTLVNSVNSKIPKACCVPTELSAISMLYLDENEKVVLKNYQDMVVEGC | |
| GCR | |
| Wherein X comprises SEQ ID NO: 8, and optionally a linker | |
| (X)ASGAGGSEGGGSEGGTSGATGAGTSTSGGGASTGGGTGQAKHKQRKRLKSSCKR | 524 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 8 | |
| (X)QAKHKQRKRLKSSCKRHPLYVDFSDVGWNDWIVAPPGYHAFYCHGECPFPLADH | 525 |
| LNSTNHAIVQTLVNSVNSKIPKACCVPTELSAISMLYLDENEKVVLKNYQDMVVEGC | |
| GCR | |
| Wherein X comprises SEQ ID NO: 9, and optionally a linker | |
| (X)ASGAGGSEGGGSEGGTSGATGAGTSTSGGGASTGGGTGQAKHKQRKRLKSSCKR | 526 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 9 | |
| (X)QAKHKQRKRLKSSCKRHPLYVDFSDVGWNDWIVAPPGYHAFYCHGECPFPLADH | 527 |
| LNSTNHAIVQTLVNSVNSKIPKACCVPTELSAISMLYLDENEKVVLKNYQDMVVEGC | |
| GCR | |
| Wherein X comprises SEQ ID NO: 10, and optionally a linker | |
| (X)ASGAGGSEGGGSEGGTSGATGAGTSTSGGGASTGGGTGQAKHKQRKRLKSSCKR | 528 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 10 | |
| (X)QAKHKQRKRLKSSCKRHPLYVDFSDVGWNDWIVAPPGYHAFYCHGECPFPLADH | 529 |
| LNSTNHAIVQTLVNSVNSKIPKACCVPTELSAISMLYLDENEKVVLKNYQDMVVEGC | |
| GCR | |
| Wherein X comprises SEQ ID NO: 11, and optionally a linker | |
| (X)ASGAGGSEGGGSEGGTSGATGAGTSTSGGGASTGGGTGQAKHKQRKRLKSSCKR | 530 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 11 | |
| (X)QAKHKQRKRLKSSCKRHPLYVDFSDVGWNDWIVAPPGYHAFYCHGECPFPLADH | 531 |
| LNSTNHAIVQTLVNSVNSKIPKACCVPTELSAISMLYLDENEKVVLKNYQDMVVEGC | |
| GCR | |
| Wherein X comprises SEQ ID NO: 12, and optionally a linker | |
| (X)ASGAGGSEGGGSEGGTSGATGAGTSTSGGGASTGGGTGQAKHKQRKRLKSSCKR | 532 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 12 | |
| (X)QAKHKQRKRLKSSCKRHPLYVDFSDVGWNDWIVAPPGYHAFYCHGECPFPLADH | 533 |
| LNSTNHAIVQTLVNSVNSKIPKACCVPTELSAISMLYLDENEKVVLKNYQDMVVEGC | |
| GCR | |
| Wherein X comprises SEQ ID NO: 13, and optionally a linker | |
| (X)ASGAGGSEGGGSEGGTSGATGAGTSTSGGGASTGGGTGQAKHKQRKRLKSSCKR | 534 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 13 | |
| (X)QAKHKQRKRLKSSCKRHPLYVDFSDVGWNDWIVAPPGYHAFYCHGECPFPLADH | 535 |
| LNSTNHAIVQTLVNSVNSKIPKACCVPTELSAISMLYLDENEKVVLKNYQDMVVEGC | |
| GCR | |
| Wherein X comprises SEQ ID NO: 14, and optionally a linker | |
| (X)ASGAGGSEGGGSEGGTSGATGAGTSTSGGGASTGGGTGQAKHKQRKRLKSSCKR | 536 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 14 | |
| (X)QAKHKQRKRLKSSCKRHPLYVDFSDVGWNDWIVAPPGYHAFYCHGECPFPLADH | 537 |
| LNSTNHAIVQTLVNSVNSKIPKACCVPTELSAISMLYLDENEKVVLKNYQDMVVEGC | |
| GCR | |
| Wherein X comprises SEQ ID NO: 15, and optionally a linker | |
| (X)ASGAGGSEGGGSEGGTSGATGAGTSTSGGGASTGGGTGQAKHKQRKRLKSSCKR | 538 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 15 | |
| (X)QAKHKQRKRLKSSCKRHPLYVDFSDVGWNDWIVAPPGYHAFYCHGECPFPLADH | 539 |
| LNSTNHAIVQTLVNSVNSKIPKACCVPTELSAISMLYLDENEKVVLKNYQDMVVEGC | |
| GCR | |
| Wherein X comprises SEQ ID NO: 16, and optionally a linker | |
| (X)ASGAGGSEGGGSEGGTSGATGAGTSTSGGGASTGGGTGQAKHKQRKRLKSSCKR | 540 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 16 | |
| (X)QAKHKQRKRLKSSCKRHPLYVDFSDVGWNDWIVAPPGYHAFYCHGECPFPLADH | 541 |
| LNSTNHAIVQTLVNSVNSKIPKACCVPTELSAISMLYLDENEKVVLKNYQDMVVEGC | |
| GCR | |
| Wherein X comprises SEQ ID NO: 17, and optionally a linker | |
| (X)ASGAGGSEGGGSEGGTSGATGAGTSTSGGGASTGGGTGQAKHKQRKRLKSSCKR | 542 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 17 | |
| (X)QAKHKQRKRLKSSCKRHPLYVDFSDVGWNDWIVAPPGYHAFYCHGECPFPLADH | 543 |
| LNSTNHAIVQTLVNSVNSKIPKACCVPTELSAISMLYLDENEKVVLKNYQDMVVEGC | |
| GCR | |
| Wherein X comprises SEQ ID NO: 18, and optionally a linker | |
| (X)ASGAGGSEGGGSEGGTSGATGAGTSTSGGGASTGGGTGQAKHKQRKRLKSSCKR | 544 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 18 | |
| (X)QAKHKQRKRLKSSCKRHPLYVDFSDVGWNDWIVAPPGYHAFYCHGECPFPLADH | 545 |
| LNSTNHAIVQTLVNSVNSKIPKACCVPTELSAISMLYLDENEKVVLKNYQDMVVEGC | |
| GCR | |
| Wherein X comprises SEQ ID NO: 19, and optionally a linker | |
| (X)ASGAGGSEGGGSEGGTSGATGAGTSTSGGGASTGGGTGQAKHKQRKRLKSSCKR | 546 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 19 | |
| (X)QAKHKQRKRLKSSCKRHPLYVDFSDVGWNDWIVAPPGYHAFYCHGECPFPLADH | 547 |
| LNSTNHAIVQTLVNSVNSKIPKACCVPTELSAISMLYLDENEKVVLKNYQDMVVEGC | |
| GCR | |
| Wherein X comprises SEQ ID NO: 20, and optionally a linker | |
| (X)ASGAGGSEGGGSEGGTSGATGAGTSTSGGGASTGGGTGQAKHKQRKRLKSSCKR | 548 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 20 | |
| (X)QAKHKQRKRLKSSCKRHPLYVDFSDVGWNDWIVAPPGYHAFYCHGECPFPLADH | 549 |
| LNSTNHAIVQTLVNSVNSKIPKACCVPTELSAISMLYLDENEKVVLKNYQDMVVEGC | |
| GCR | |
| Wherein X comprises SEQ ID NO: 21, and optionally a linker | |
| (X)ASGAGGSEGGGSEGGTSGATGAGTSTSGGGASTGGGTGQAKHKQRKRLKSSCKR | 550 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 21 | |
| (X)QAKHKQRKRLKSSCKRHPLYVDFSDVGWNDWIVAPPGYHAFYCHGECPFPLADH | 551 |
| LNSTNHAIVQTLVNSVNSKIPKACCVPTELSAISMLYLDENEKVVLKNYQDMVVEGC | |
| GCR | |
| Wherein X comprises SEQ ID NO: 22, and optionally a linker | |
| (X)ASGAGGSEGGGSEGGTSGATGAGTSTSGGGASTGGGTGQAKHKQRKRLKSSCKR | 552 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 22 | |
| (X)QAKHKQRKRLKSSCKRHPLYVDFSDVGWNDWIVAPPGYHAFYCHGECPFPLADH | 553 |
| LNSTNHAIVQTLVNSVNSKIPKACCVPTELSAISMLYLDENEKVVLKNYQDMVVEGC | |
| GCR | |
| Wherein X comprises SEQ ID NO: 23, and optionally a linker | |
| (X)ASGAGGSEGGGSEGGTSGATGAGTSTSGGGASTGGGTGQAKHKQRKRLKSSCKR | 554 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 23 | |
| (X)QAKHKQRKRLKSSCKRHPLYVDFSDVGWNDWIVAPPGYHAFYCHGECPFPLADH | 555 |
| LNSTNHAIVQTLVNSVNSKIPKACCVPTELSAISMLYLDENEKVVLKNYQDMVVEGC | |
| GCR | |
| Wherein X comprises SEQ ID NO: 24, and optionally a linker | |
| (X)ASGAGGSEGGGSEGGTSGATGAGTSTSGGGASTGGGTGQAKHKQRKRLKSSCKR | 556 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 24 | |
| (X)QAKHKQRKRLKSSCKRHPLYVDFSDVGWNDWIVAPPGYHAFYCHGECPFPLADH | 557 |
| LNSTNHAIVQTLVNSVNSKIPKACCVPTELSAISMLYLDENEKVVLKNYQDMVVEGC | |
| GCR | |
| Wherein X comprises SEQ ID NO: 25, and optionally a linker | |
| (X)ASGAGGSEGGGSEGGTSGATGAGTSTSGGGASTGGGTGQAKHKQRKRLKSSCKR | 558 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 25 | |
| (X)QAKHKQRKRLKSSCKRHPLYVDFSDVGWNDWIVAPPGYHAFYCHGECPFPLADH | 559 |
| LNSTNHAIVQTLVNSVNSKIPKACCVPTELSAISMLYLDENEKVVLKNYQDMVVEGC | |
| GCR | |
| Wherein X comprises SEQ ID NO: 26, and optionally a linker | |
| (X)ASGAGGSEGGGSEGGTSGATGAGTSTSGGGASTGGGTGQAKHKQRKRLKSSCKR | 560 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 26 | |
| (X)QAKHKQRKRLKSSCKRHPLYVDFSDVGWNDWIVAPPGYHAFYCHGECPFPLADH | 561 |
| LNSTNHAIVQTLVNSVNSKIPKACCVPTELSAISMLYLDENEKVVLKNYQDMVVEGC | |
| GCR | |
| Wherein X comprises SEQ ID NO: 27, and optionally a linker | |
| (X)ASGAGGSEGGGSEGGTSGATGAGTSTSGGGASTGGGTGQAKHKQRKRLKSSCKR | 562 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 27 | |
| (X)QAKHKQRKRLKSSCKRHPLYVDFSDVGWNDWIVAPPGYHAFYCHGECPFPLADH | 563 |
| LNSTNHAIVQTLVNSVNSKIPKACCVPTELSAISMLYLDENEKVVLKNYQDMVVEGC | |
| GCR | |
| Wherein X comprises SEQ ID NO: 28, and optionally a linker | |
| (X)ASGAGGSEGGGSEGGTSGATGAGTSTSGGGASTGGGTGQAKHKQRKRLKSSCKR | 564 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 28 | |
| (X)QAKHKQRKRLKSSCKRHPLYVDFSDVGWNDWIVAPPGYHAFYCHGECPFPLADH | 565 |
| LNSTNHAIVQTLVNSVNSKIPKACCVPTELSAISMLYLDENEKVVLKNYQDMVVEGC | |
| GCR | |
| Wherein X comprises SEQ ID NO: 29, and optionally a linker | |
| (X)ASGAGGSEGGGSEGGTSGATGAGTSTSGGGASTGGGTGQAKHKQRKRLKSSCKR | 566 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 29 | |
| (X)QAKHKQRKRLKSSCKRHPLYVDFSDVGWNDWIVAPPGYHAFYCHGECPFPLADH | 567 |
| LNSTNHAIVQTLVNSVNSKIPKACCVPTELSAISMLYLDENEKVVLKNYQDMVVEGC | |
| GCR | |
| Wherein X comprises SEQ ID NO: 30, and optionally a linker | |
| (X)ASGAGGSEGGGSEGGTSGATGAGTSTSGGGASTGGGTGQAKHKQRKRLKSSCKR | 568 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 30 | |
| (X)QAKHKQRKRLKSSCKRHPLYVDFSDVGWNDWIVAPPGYHAFYCHGECPFPLADH | 569 |
| LNSTNHAIVQTLVNSVNSKIPKACCVPTELSAISMLYLDENEKVVLKNYQDMVVEGC | |
| GCR | |
| Wherein X comprises SEQ ID NO: 31, and optionally a linker | |
| (X)ASGAGGSEGGGSEGGTSGATGAGTSTSGGGASTGGGTGQAKHKQRKRLKSSCKR | 570 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 31 | |
| (X)QAKHKQRKRLKSSCKRHPLYVDFSDVGWNDWIVAPPGYHAFYCHGECPFPLADH | 571 |
| LNSTNHAIVQTLVNSVNSKIPKACCVPTELSAISMLYLDENEKVVLKNYQDMVVEGC | |
| GCR | |
| Wherein X comprises SEQ ID NO: 32, and optionally a linker | |
| (X)ASGAGGSEGGGSEGGTSGATGAGTSTSGGGASTGGGTGQAKHKQRKRLKSSCKR | 572 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 32 | |
| (X)QAKHKQRKRLKSSCKRHPLYVDFSDVGWNDWIVAPPGYHAFYCHGECPFPLADH | 573 |
| LNSTNHAIVQTLVNSVNSKIPKACCVPTELSAISMLYLDENEKVVLKNYQDMVVEGC | |
| GCR | |
| Wherein X comprises SEQ ID NO: 33, and optionally a linker | |
| (X)ASGAGGSEGGGSEGGTSGATGAGTSTSGGGASTGGGTGQAKHKQRKRLKSSCKR | 574 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 33 | |
| (X)QAKHKQRKRLKSSCKRHPLYVDFSDVGWNDWIVAPPGYHAFYCHGECPFPLADH | 575 |
| LNSTNHAIVQTLVNSVNSKIPKACCVPTELSAISMLYLDENEKVVLKNYQDMVVEGC | |
| GCR | |
| Wherein X comprises SEQ ID NO: 34, and optionally a linker | |
| (X)ASGAGGSEGGGSEGGTSGATGAGTSTSGGGASTGGGTGQAKHKQRKRLKSSCKR | 576 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 34 | |
| (X)QAKHKQRKRLKSSCKRHPLYVDFSDVGWNDWIVAPPGYHAFYCHGECPFPLADH | 577 |
| LNSTNHAIVQTLVNSVNSKIPKACCVPTELSAISMLYLDENEKVVLKNYQDMVVEGC | |
| GCR | |
| Wherein X comprises SEQ ID NO: 35, and optionally a linker | |
| (X)ASGAGGSEGGGSEGGTSGATGAGTSTSGGGASTGGGTGQAKHKQRKRLKSSCKR | 578 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 35 | |
| (X)QAKHKQRKRLKSSCKRHPLYVDFSDVGWNDWIVAPPGYHAFYCHGECPFPLADH | 579 |
| LNSTNHAIVQTLVNSVNSKIPKACCVPTELSAISMLYLDENEKVVLKNYQDMVVEGC | |
| GCR | |
| Wherein X comprises SEQ ID NO: 36, and optionally a linker | |
| (X)ASGAGGSEGGGSEGGTSGATGAGTSTSGGGASTGGGTGQAKHKQRKRLKSSCKR | 580 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 36 | |
| (X)QAKHKQRKRLKSSCKRHPLYVDFSDVGWNDWIVAPPGYHAFYCHGECPFPLADH | 581 |
| LNSTNHAIVQTLVNSVNSKIPKACCVPTELSAISMLYLDENEKVVLKNYQDMVVEGC | |
| GCR | |
| Wherein X comprises SEQ ID NO: 37, and optionally a linker | |
| (X)ASGAGGSEGGGSEGGTSGATGAGTSTSGGGASTGGGTGQAKHKQRKRLKSSCKR | 582 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 37 | |
| (X)QAKHKQRKRLKSSCKRHPLYVDFSDVGWNDWIVAPPGYHAFYCHGECPFPLADH | 583 |
| LNSTNHAIVQTLVNSVNSKIPKACCVPTELSAISMLYLDENEKVVLKNYQDMVVEGC | |
| GCR | |
| Wherein X comprises SEQ ID NO: 38, and optionally a linker | |
| (X)ASGAGGSEGGGSEGGTSGATGAGTSTSGGGASTGGGTGQAKHKQRKRLKSSCKR | 584 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 38 | |
| (X)QAKHKQRKRLKSSCKRHPLYVDFSDVGWNDWIVAPPGYHAFYCHGECPFPLADH | 585 |
| LNSTNHAIVQTLVNSVNSKIPKACCVPTELSAISMLYLDENEKVVLKNYQDMVVEGC | |
| GCR | |
| Wherein X comprises SEQ ID NO: 39, and optionally a linker | |
| (X)ASGAGGSEGGGSEGGTSGATGAGTSTSGGGASTGGGTGQAKHKQRKRLKSSCKR | 586 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 39 | |
| (X)QAKHKQRKRLKSSCKRHPLYVDFSDVGWNDWIVAPPGYHAFYCHGECPFPLADH | 587 |
| LNSTNHAIVQTLVNSVNSKIPKACCVPTELSAISMLYLDENEKVVLKNYQDMVVEGC | |
| GCR | |
| Wherein X comprises SEQ ID NO: 40, and optionally a linker | |
| (X)ASGAGGSEGGGSEGGTSGATGAGTSTSGGGASTGGGTGQAKHKQRKRLKSSCKR | 588 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 40 | |
| (X)QAKHKQRKRLKSSCKRHPLYVDFSDVGWNDWIVAPPGYHAFYCHGECPFPLADH | 589 |
| LNSTNHAIVQTLVNSVNSKIPKACCVPTELSAISMLYLDENEKVVLKNYQDMVVEGC | |
| GCR | |
| Wherein X comprises SEQ ID NO: 41, and optionally a linker | |
| (X)ASGAGGSEGGGSEGGTSGATGAGTSTSGGGASTGGGTGQAKHKQRKRLKSSCKR | 590 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 41 | |
| (X)QAKHKQRKRLKSSCKRHPLYVDFSDVGWNDWIVAPPGYHAFYCHGECPFPLADH | 591 |
| LNSTNHAIVQTLVNSVNSKIPKACCVPTELSAISMLYLDENEKVVLKNYQDMVVEGC | |
| GCR | |
| Wherein X comprises SEQ ID NO: 42, and optionally a linker | |
| (X)ASGAGGSEGGGSEGGTSGATGAGTSTSGGGASTGGGTGQAKHKQRKRLKSSCKR | 592 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 42 | |
| (X)QAKHKQRKRLKSSCKRHPLYVDFSDVGWNDWIVAPPGYHAFYCHGECPFPLADH | 593 |
| LNSTNHAIVQTLVNSVNSKIPKACCVPTELSAISMLYLDENEKVVLKNYQDMVVEGC | |
| GCR | |
| Wherein X comprises SEQ ID NO: 43, and optionally a linker | |
| (X)ASGAGGSEGGGSEGGTSGATGAGTSTSGGGASTGGGTGQAKHKQRKRLKSSCKR | 594 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 43 | |
| (X)QAKHKQRKRLKSSCKRHPLYVDFSDVGWNDWIVAPPGYHAFYCHGECPFPLADH | 595 |
| LNSTNHAIVQTLVNSVNSKIPKACCVPTELSAISMLYLDENEKVVLKNYQDMVVEGC | |
| GCR | |
| Wherein X comprises SEQ ID NO: 401, and optionally a linker | |
| (X)ASGAGGSEGGGSEGGTSGATGAGTSTSGGGASTGGGTGQAKHKQRKRLKSSCKR | 596 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 401 | |
| (X)QAKHKQRKRLKSSCKRHPLYVDFSDVGWNDWIVAPPGYHAFYCHGECPFPLADH | 597 |
| LNSTNHAIVQTLVNSVNSKIPKACCVPTELSAISMLYLDENEKVVLKNYQDMVVEGC | |
| GCR | |
| Wherein X comprises SEQ ID NO: 402, and optionally a linker | |
| (X)ASGAGGSEGGGSEGGTSGATGAGTSTSGGGASTGGGTGQAKHKQRKRLKSSCKR | 598 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 402 | |
| (X)QAKHKQRKRLKSSCKRHPLYVDFSDVGWNDWIVAPPGYHAFYCHGECPFPLADH | 599 |
| LNSTNHAIVQTLVNSVNSKIPKACCVPTELSAISMLYLDENEKVVLKNYQDMVVEGC | |
| GCR | |
| Wherein X comprises SEQ ID NO: 403, and optionally a linker | |
| (X)ASGAGGSEGGGSEGGTSGATGAGTSTSGGGASTGGGTGQAKHKQRKRLKSSCKR | 600 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 403 | |
| (X)QAKHKQRKRLKSSCKRHPLYVDFSDVGWNDWIVAPPGYHAFYCHGECPFPLADH | 601 |
| LNSTNHAIVQTLVNSVNSKIPKACCVPTELSAISMLYLDENEKVVLKNYQDMVVEGC | |
| GCR | |
| Wherein X comprises SEQ ID NO: 404, and optionally a linker | |
| (X)ASGAGGSEGGGSEGGTSGATGAGTSTSGGGASTGGGTGQAKHKQRKRLKSSCKR | 602 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 404 | |
| (X)QAKHKQRKRLKSSCKRHPLYVDFSDVGWNDWIVAPPGYHAFYCHGECPFPLADH | 603 |
| LNSTNHAIVQTLVNSVNSKIPKACCVPTELSAISMLYLDENEKVVLKNYQDMVVEGC | |
| GCR | |
| Wherein X comprises SEQ ID NO: 405, and optionally a linker | |
| (X)ASGAGGSEGGGSEGGTSGATGAGTSTSGGGASTGGGTGQAKHKQRKRLKSSCKR | 604 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 405 | |
| (X)QAKHKQRKRLKSSCKRHPLYVDFSDVGWNDWIVAPPGYHAFYCHGECPFPLADH | 605 |
| LNSTNHAIVQTLVNSVNSKIPKACCVPTELSAISMLYLDENEKVVLKNYQDMVVEGC | |
| GCR | |
| Wherein X comprises SEQ ID NO: 406, and optionally a linker | |
| (X)ASGAGGSEGGGSEGGTSGATGAGTSTSGGGASTGGGTGQAKHKQRKRLKSSCKR | 606 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 406 | |
| (X)QAKHKQRKRLKSSCKRHPLYVDFSDVGWNDWIVAPPGYHAFYCHGECPFPLADH | 607 |
| LNSTNHAIVQTLVNSVNSKIPKACCVPTELSAISMLYLDENEKVVLKNYQDMVVEGC | |
| GCR | |
| Wherein X comprises SEQ ID NO: 407, and optionally a linker | |
| (X)ASGAGGSEGGGSEGGTSGATGAGTSTSGGGASTGGGTGQAKHKQRKRLKSSCKR | 608 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 407 | |
| (X)QAKHKQRKRLKSSCKRHPLYVDFSDVGWNDWIVAPPGYHAFYCHGECPFPLADH | 609 |
| LNSTNHAIVQTLVNSVNSKIPKACCVPTELSAISMLYLDENEKVVLKNYQDMVVEGC | |
| GCR | |
| Wherein X comprises SEQ ID NO: 408, and optionally a linker | |
| (X)ASGAGGSEGGGSEGGTSGATGAGTSTSGGGASTGGGTGQAKHKQRKRLKSSCKR | 610 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 408 | |
| (X)QAKHKQRKRLKSSCKRHPLYVDFSDVGWNDWIVAPPGYHAFYCHGECPFPLADH | 611 |
| LNSTNHAIVQTLVNSVNSKIPKACCVPTELSAISMLYLDENEKVVLKNYQDMVVEGC | |
| GCR | |
| Wherein X comprises SEQ ID NO: 409, and optionally a linker | |
| (X)ASGAGGSEGGGSEGGTSGATGAGTSTSGGGASTGGGTGQAKHKQRKRLKSSCKR | 612 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 409 | |
| (X)QAKHKQRKRLKSSCKRHPLYVDFSDVGWNDWIVAPPGYHAFYCHGECPFPLADH | 613 |
| LNSTNHAIVQTLVNSVNSKIPKACCVPTELSAISMLYLDENEKVVLKNYQDMVVEGC | |
| GCR | |
| Wherein X comprises SEQ ID NO: 410, and optionally a linker | |
| (X)ASGAGGSEGGGSEGGTSGATGAGTSTSGGGASTGGGTGQAKHKQRKRLKSSCKR | 614 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 410 | |
| (X)QAKHKQRKRLKSSCKRHPLYVDFSDVGWNDWIVAPPGYHAFYCHGECPFPLADH | 615 |
| LNSTNHAIVQTLVNSVNSKIPKACCVPTELSAISMLYLDENEKVVLKNYQDMVVEGC | |
| GCR | |
| Wherein X comprises SEQ ID NO: 411, and optionally a linker | |
| (X)ASGAGGSEGGGSEGGTSGATGAGTSTSGGGASTGGGTGQAKHKQRKRLKSSCKR | 616 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 411 | |
| (X)QAKHKQRKRLKSSCKRHPLYVDFSDVGWNDWIVAPPGYHAFYCHGECPFPLADH | 617 |
| LNSTNHAIVQTLVNSVNSKIPKACCVPTELSAISMLYLDENEKVVLKNYQDMVVEGC | |
| GCR | |
| Wherein X comprises SEQ ID NO: 412, and optionally a linker | |
| (X)ASGAGGSEGGGSEGGTSGATGAGTSTSGGGASTGGGTGQAKHKQRKRLKSSCKR | 618 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 412 | |
| (X)QAKHKQRKRLKSSCKRHPLYVDFSDVGWNDWIVAPPGYHAFYCHGECPFPLADH | 619 |
| LNSTNHAIVQTLVNSVNSKIPKACCVPTELSAISMLYLDENEKVVLKNYQDMVVEGC | |
| GCR | |
| Wherein X comprises SEQ ID NO: 413, and optionally a linker | |
| (X)ASGAGGSEGGGSEGGTSGATGAGTSTSGGGASTGGGTGQAKHKQRKRLKSSCKR | 620 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 413 | |
| (X)QAKHKQRKRLKSSCKRHPLYVDFSDVGWNDWIVAPPGYHAFYCHGECPFPLADH | 621 |
| LNSTNHAIVQTLVNSVNSKIPKACCVPTELSAISMLYLDENEKVVLKNYQDMVVEGC | |
| GCR | |
| Wherein X comprises SEQ ID NO: 414, and optionally a linker | |
| (X)ASGAGGSEGGGSEGGTSGATGAGTSTSGGGASTGGGTGQAKHKQRKRLKSSCKR | 622 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 414 | |
| (X)QAKHKQRKRLKSSCKRHPLYVDFSDVGWNDWIVAPPGYHAFYCHGECPFPLADH | 623 |
| LNSTNHAIVQTLVNSVNSKIPKACCVPTELSAISMLYLDENEKVVLKNYQDMVVEGC | |
| GCR | |
| Wherein X comprises SEQ ID NO: 415, and optionally a linker | |
| (X)ASGAGGSEGGGSEGGTSGATGAGTSTSGGGASTGGGTGQAKHKQRKRLKSSCKR | 624 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 415 | |
| (X)QAKHKQRKRLKSSCKRHPLYVDFSDVGWNDWIVAPPGYHAFYCHGECPFPLADH | 625 |
| LNSTNHAIVQTLVNSVNSKIPKACCVPTELSAISMLYLDENEKVVLKNYQDMVVEGC | |
| GCR | |
| Wherein X comprises SEQ ID NO: 416, and optionally a linker | |
| (X)ASGAGGSEGGGSEGGTSGATGAGTSTSGGGASTGGGTGQAKHKQRKRLKSSCKR | 626 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 416 | |
| (X)QAKHKQRKRLKSSCKRHPLYVDFSDVGWNDWIVAPPGYHAFYCHGECPFPLADH | 627 |
| LNSTNHAIVQTLVNSVNSKIPKACCVPTELSAISMLYLDENEKVVLKNYQDMVVEGC | |
| GCR | |
| Wherein X comprises SEQ ID NO: 417, and optionally a linker | |
| (X)ASGAGGSEGGGSEGGTSGATGAGTSTSGGGASTGGGTGQAKHKQRKRLKSSCKR | 628 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 417 | |
| (X)QAKHKQRKRLKSSCKRHPLYVDFSDVGWNDWIVAPPGYHAFYCHGECPFPLADH | 629 |
| LNSTNHAIVQTLVNSVNSKIPKACCVPTELSAISMLYLDENEKVVLKNYQDMVVEGC | |
| GCR | |
| Wherein X comprises SEQ ID NO: 418, and optionally a linker | |
| (X)ASGAGGSEGGGSEGGTSGATGAGTSTSGGGASTGGGTGQAKHKQRKRLKSSCKR | 630 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 418 | |
| (X)QAKHKQRKRLKSSCKRHPLYVDFSDVGWNDWIVAPPGYHAFYCHGECPFPLADH | 631 |
| LNSTNHAIVQTLVNSVNSKIPKACCVPTELSAISMLYLDENEKVVLKNYQDMVVEGC | |
| GCR | |
| Wherein X comprises SEQ ID NO: 419, and optionally a linker | |
| (X)ASGAGGSEGGGSEGGTSGATGAGTSTSGGGASTGGGTGQAKHKQRKRLKSSCKR | 632 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 419 | |
| (X)QAKHKQRKRLKSSCKRHPLYVDFSDVGWNDWIVAPPGYHAFYCHGECPFPLADH | 633 |
| LNSTNHAIVQTLVNSVNSKIPKACCVPTELSAISMLYLDENEKVVLKNYQDMVVEGC | |
| GCR | |
| Wherein X comprises SEQ ID NO: 420, and optionally a linker | |
| (X)ASGAGGSEGGGSEGGTSGATGAGTSTSGGGASTGGGTGQAKHKQRKRLKSSCKR | 634 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 420 | |
| (X)QAKHKQRKRLKSSCKRHPLYVDFSDVGWNDWIVAPPGYHAFYCHGECPFPLADH | 635 |
| LNSTNHAIVQTLVNSVNSKIPKACCVPTELSAISMLYLDENEKVVLKNYQDMVVEGC | |
| GCR | |
| Wherein X comprises SEQ ID NO: 421, and optionally a linker | |
| (X)ASGAGGSEGGGSEGGTSGATGAGTSTSGGGASTGGGTGQAKHKQRKRLKSSCKR | 636 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 421 | |
| (X)QAKHKQRKRLKSSCKRHPLYVDFSDVGWNDWIVAPPGYHAFYCHGECPFPLADH | 637 |
| LNSTNHAIVQTLVNSVNSKIPKACCVPTELSAISMLYLDENEKVVLKNYQDMVVEGC | |
| GCR | |
| Wherein X comprises SEQ ID NO: 422, and optionally a linker | |
| (X)ASGAGGSEGGGSEGGTSGATGAGTSTSGGGASTGGGTGQAKHKQRKRLKSSCKR | 638 |
| HPLYVDFSDVGWNDWIVAPPGYHAFYCHGECPFPLADHLNSTNHAIVQTLVNSVNSK | |
| IPKACCVPTELSAISMLYLDENEKVVLKNYQDMVVEGCGCR | |
| Wherein X comprises SEQ ID NO: 422 | |

In some embodiments, any of the chimeric polypeptides described herein comprise one or more targeting polypeptides. In some embodiments, the chimeric polypeptide comprises one to about ten targeting polypeptides. For instance, the chimeric polypeptide comprises about 1, 2, 3, 4, 5, 6, 7, 8, 9, or 10 targeting polypeptides. In some embodiments, the targeting polypeptide comprises one or more polypeptides that bind to a carrier material. In some embodiments, the one or more polypeptides is about 1, 2, 3, 4, 5, 6, 7, 8, 9, or 10 polypeptides. In some embodiments, the targeting polypeptide comprises from about 10 to about 100, from about 10 to about 90, from about 10 to about 80, from about 10 to about 70, from about 10 to about 60, from about 10 to about 50, from about 10 to about 40, from about 10 to about 30, from about 20 to about 100, from about 20 to about 90, from about 20 to about 80, from about 20 to about 70, from about 20 to about 60, from about 20 to about 50, from about 20 to about 40, or from about 30 to about 80 amino acids.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 1. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 2. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 3. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 4. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 5. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 6. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 7. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 8. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 9. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 10. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 11. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 12. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 13. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 14. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 15. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 16. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 17. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 18. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises an amino acid sequence that has at least 70% (e.g., at least 75%, at least 80%, at least 85%, at least 90%, at least 95%, at least 96%, at least 97%, at least 98%, at least 99%, or 100%) sequence identity to SEQ ID NO: 19. In some embodiments, the chimeric polypeptide comprises one or more therapeutic agents of Table B, or a sequence at least about 70%, 75%, 80%, 85%, 90%, 95%, or 100% identical to a therapeutic agent of Table B. As a non-limiting example, the therapeutic agent comprises a sequence at least about 70%, 75%, 80%, 85%, 90%, 95%, or 100% identical to SEQ ID NO: 32. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 501. In some embodiments, the chimeric polypeptide comprises SEQ ID NO: 501. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 502. In some embodiments, the chimeric polypeptide comprises SEQ ID NO: 502. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, NO: 503. In some embodiments, the chimeric polypeptide comprises SEQ ID NO: 503. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 504. In some embodiments, the chimeric polypeptide comprises SEQ ID NO: 504. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 505. In some embodiments, the chimeric polypeptide comprises SEQ ID NO: 505. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 506. In some embodiments, the chimeric polypeptide comprises SEQ ID NO: 506. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, NO: 507. In some embodiments, the chimeric polypeptide comprises SEQ ID NO: 507. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 508, wherein the chimeric polypeptide comprises X. In some embodiments, the chimeric polypeptide comprises SEQ ID NO: 508. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 509, wherein the chimeric polypeptide comprises X. In some embodiments, the chimeric polypeptide comprises SEQ ID NO: 509. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, NO: 510, wherein the chimeric polypeptide comprises X. In some embodiments, the chimeric polypeptide comprises SEQ ID NO: 510. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 511, wherein the chimeric polypeptide comprises X. In some embodiments, the chimeric polypeptide comprises SEQ ID NO: 511. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 512, wherein the chimeric polypeptide comprises X. In some embodiments, the chimeric polypeptide comprises SEQ ID NO: 512. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 513, wherein the chimeric polypeptide comprises X. In some embodiments, the chimeric polypeptide comprises SEQ ID NO: 513. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 514, wherein the chimeric polypeptide comprises X. In some embodiments, the chimeric polypeptide comprises SEQ ID NO: 514. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 515, wherein the chimeric polypeptide comprises X. In some embodiments, the chimeric polypeptide comprises SEQ ID NO: 515. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 516, wherein the chimeric polypeptide comprises X. In some embodiments, the chimeric polypeptide comprises SEQ ID NO: 516. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, NO: 517, wherein the chimeric polypeptide comprises X. In some embodiments, the chimeric polypeptide comprises SEQ ID NO: 517. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 518, wherein the chimeric polypeptide comprises X. In some embodiments, the chimeric polypeptide comprises SEQ ID NO: 518. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 519, wherein the chimeric polypeptide comprises X. In some embodiments, the chimeric polypeptide comprises SEQ ID NO: 519. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 520, wherein the chimeric polypeptide comprises X. In some embodiments, the chimeric polypeptide comprises SEQ ID NO: 520. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 521, wherein the chimeric polypeptide comprises X. In some embodiments, the chimeric polypeptide comprises SEQ ID NO: 521. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 522, wherein the chimeric polypeptide comprises X. In some embodiments, the chimeric polypeptide comprises SEQ ID NO: 522. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 523, wherein the chimeric polypeptide comprises X. In some embodiments, the chimeric polypeptide comprises SEQ ID NO: 523. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 524, wherein the chimeric polypeptide comprises X. In some embodiments, the chimeric polypeptide comprises SEQ ID NO: 524. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 525, wherein the chimeric polypeptide comprises X. In some embodiments, the chimeric polypeptide comprises SEQ ID NO: 525. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 526, wherein the chimeric polypeptide comprises X. In some embodiments, the chimeric polypeptide comprises SEQ ID NO: 526. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, NO: 527, wherein the chimeric polypeptide comprises X. In some embodiments, the chimeric polypeptide comprises SEQ ID NO: 527. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 528, wherein the chimeric polypeptide comprises X. In some embodiments, the chimeric polypeptide comprises SEQ ID NO: 528. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 529, wherein the chimeric polypeptide comprises X. In some embodiments, the chimeric polypeptide comprises SEQ ID NO: 529. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 530, wherein the chimeric polypeptide comprises X. In some embodiments, the chimeric polypeptide comprises SEQ ID NO: 530. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 531, wherein the chimeric polypeptide comprises X. In some embodiments, the chimeric polypeptide comprises SEQ ID NO: 531. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 532, wherein the chimeric polypeptide comprises X. In some embodiments, the chimeric polypeptide comprises SEQ ID NO: 532. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 533, wherein the chimeric polypeptide comprises X. In some embodiments, the chimeric polypeptide comprises SEQ ID NO: 533. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 534, wherein the chimeric polypeptide comprises X. In some embodiments, the chimeric polypeptide comprises SEQ ID NO: 534. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 535, wherein the chimeric polypeptide comprises X. In some embodiments, the chimeric polypeptide comprises SEQ ID NO: 535. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 536, wherein the chimeric polypeptide comprises X. In some embodiments, the chimeric polypeptide comprises SEQ ID NO: 536. In some aspects, further provided are compositions comprising the chimeric polypeptide and a carrier material, e.g., a carrier material provided herein.

Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 537, wherein the chimeric polypeptide comprises X. In some embodiments, the chimeric polypeptide comprises SEQ ID NO: 537. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 538, wherein the chimeric polypeptide comprises X. In some embodiments, the chimeric polypeptide comprises SEQ ID NO: 538. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 539, wherein the chimeric polypeptide comprises X. In some embodiments, the chimeric polypeptide comprises SEQ ID NO: 539. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 540, wherein the chimeric polypeptide comprises X. In some embodiments, the chimeric polypeptide comprises SEQ ID NO: 540. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 541, wherein the chimeric polypeptide comprises X. In some embodiments, the chimeric polypeptide comprises SEQ ID NO: 541. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 542, wherein the chimeric polypeptide comprises X. In some embodiments, the chimeric polypeptide comprises SEQ ID NO: 542. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 543, wherein the chimeric polypeptide comprises X. In some embodiments, the chimeric polypeptide comprises SEQ ID NO: 543. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 544, wherein the chimeric polypeptide comprises X. In some embodiments, the chimeric polypeptide comprises SEQ ID NO: 544. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 545, wherein the chimeric polypeptide comprises X. In some embodiments, the chimeric polypeptide comprises SEQ ID NO: 545. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 546, wherein the chimeric polypeptide comprises X. In some embodiments, the chimeric polypeptide comprises SEQ ID NO: 546. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 547, wherein the chimeric polypeptide comprises X. In some embodiments, the chimeric polypeptide comprises SEQ ID NO: 547. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 548, wherein the chimeric polypeptide comprises X. In some embodiments, the chimeric polypeptide comprises SEQ ID NO: 548. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 549, wherein the chimeric polypeptide comprises X. In some embodiments, the chimeric polypeptide comprises SEQ ID NO: 549. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 550, wherein the chimeric polypeptide comprises X. In some embodiments, the chimeric polypeptide comprises SEQ ID NO: 550. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 551, wherein the chimeric polypeptide comprises X. In some embodiments, the chimeric polypeptide comprises SEQ ID NO: 551. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 552, wherein the chimeric polypeptide comprises X. In some embodiments, the chimeric polypeptide comprises SEQ ID NO: 552. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 553, wherein the chimeric polypeptide comprises X. In some embodiments, the chimeric polypeptide comprises SEQ ID NO: 553. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 554, wherein the chimeric polypeptide comprises X. In some embodiments, the chimeric polypeptide comprises SEQ ID NO: 554. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 555, wherein the chimeric polypeptide comprises X. In some embodiments, the chimeric polypeptide comprises SEQ ID NO: 555. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 556, wherein the chimeric polypeptide comprises X. In some embodiments, the chimeric polypeptide comprises SEQ ID NO: 556. In some aspects, further provided are compositions comprising the chimeric polypeptide and a carrier material, e.g., a carrier material provided herein.

Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 557, wherein the chimeric polypeptide comprises X. In some embodiments, the chimeric polypeptide comprises SEQ ID NO: 557. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 558, wherein the chimeric polypeptide comprises X. In some embodiments, the chimeric polypeptide comprises SEQ ID NO: 558. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 559, wherein the chimeric polypeptide comprises X. In some embodiments, the chimeric polypeptide comprises SEQ ID NO: 559. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 560, wherein the chimeric polypeptide comprises X. In some embodiments, the chimeric polypeptide comprises SEQ ID NO: 560. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 561, wherein the chimeric polypeptide comprises X. In some embodiments, the chimeric polypeptide comprises SEQ ID NO: 561. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 562, wherein the chimeric polypeptide comprises X. In some embodiments, the chimeric polypeptide comprises SEQ ID NO: 562. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 563, wherein the chimeric polypeptide comprises X. In some embodiments, the chimeric polypeptide comprises SEQ ID NO: 563. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 564, wherein the chimeric polypeptide comprises X. In some embodiments, the chimeric polypeptide comprises SEQ ID NO: 564. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 565, wherein the chimeric polypeptide comprises X. In some embodiments, the chimeric polypeptide comprises SEQ ID NO: 565. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 566, wherein the chimeric polypeptide comprises X. In some embodiments, the chimeric polypeptide comprises SEQ ID NO: 566. In some aspects, further provided are compositions comprising the chimeric polypeptide and a carrier material, e.g., a carrier material provided herein.

Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 567, wherein the chimeric polypeptide comprises X. In some embodiments, the chimeric polypeptide comprises SEQ ID NO: 567. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 568, wherein the chimeric polypeptide comprises X. In some embodiments, the chimeric polypeptide comprises SEQ ID NO: 568. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 569, wherein the chimeric polypeptide comprises X. In some embodiments, the chimeric polypeptide comprises SEQ ID NO: 569. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 570, wherein the chimeric polypeptide comprises X. In some embodiments, the chimeric polypeptide comprises SEQ ID NO: 570. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 571, wherein the chimeric polypeptide comprises X. In some embodiments, the chimeric polypeptide comprises SEQ ID NO: 571. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 572, wherein the chimeric polypeptide comprises X. In some embodiments, the chimeric polypeptide comprises SEQ ID NO: 572. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 573, wherein the chimeric polypeptide comprises X. In some embodiments, the chimeric polypeptide comprises SEQ ID NO: 573. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 574, wherein the chimeric polypeptide comprises X. In some embodiments, the chimeric polypeptide comprises SEQ ID NO: 574. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 575, wherein the chimeric polypeptide comprises X. In some embodiments, the chimeric polypeptide comprises SEQ ID NO: 575. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 576, wherein the chimeric polypeptide comprises X. In some embodiments, the chimeric polypeptide comprises SEQ ID NO: 576. In some aspects, further provided are compositions comprising the chimeric polypeptide and a carrier material, e.g., a carrier material provided herein.

Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 577, wherein the chimeric polypeptide comprises X. In some embodiments, the chimeric polypeptide comprises SEQ ID NO: 577. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 578, wherein the chimeric polypeptide comprises X. In some embodiments, the chimeric polypeptide comprises SEQ ID NO: 578. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 579, wherein the chimeric polypeptide comprises X. In some embodiments, the chimeric polypeptide comprises SEQ ID NO: 579. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 580, wherein the chimeric polypeptide comprises X. In some embodiments, the chimeric polypeptide comprises SEQ ID NO: 580. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 581, wherein the chimeric polypeptide comprises X. In some embodiments, the chimeric polypeptide comprises SEQ ID NO: 581. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 582, wherein the chimeric polypeptide comprises X. In some embodiments, the chimeric polypeptide comprises SEQ ID NO: 582. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 583, wherein the chimeric polypeptide comprises X. In some embodiments, the chimeric polypeptide comprises SEQ ID NO: 583. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 584, wherein the chimeric polypeptide comprises X. In some embodiments, the chimeric polypeptide comprises SEQ ID NO: 584. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 585, wherein the chimeric polypeptide comprises X. In some embodiments, the chimeric polypeptide comprises SEQ ID NO: 585. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 586, wherein the chimeric polypeptide comprises X. In some embodiments, the chimeric polypeptide comprises SEQ ID NO: 586. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 587, wherein the chimeric polypeptide comprises X. In some embodiments, the chimeric polypeptide comprises SEQ ID NO: 587. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 588, wherein the chimeric polypeptide comprises X. In some embodiments, the chimeric polypeptide comprises SEQ ID NO: 588. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 589, wherein the chimeric polypeptide comprises X. In some embodiments, the chimeric polypeptide comprises SEQ ID NO: 589. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 590, wherein the chimeric polypeptide comprises X. In some embodiments, the chimeric polypeptide comprises SEQ ID NO: 590. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 591, wherein the chimeric polypeptide comprises X. In some embodiments, the chimeric polypeptide comprises SEQ ID NO: 591. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 592, wherein the chimeric polypeptide comprises X. In some embodiments, the chimeric polypeptide comprises SEQ ID NO: 592. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 593, wherein the chimeric polypeptide comprises X. In some embodiments, the chimeric polypeptide comprises SEQ ID NO: 593. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 594, wherein the chimeric polypeptide comprises X. In some embodiments, the chimeric polypeptide comprises SEQ ID NO: 594. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 595, wherein the chimeric polypeptide comprises X. In some embodiments, the chimeric polypeptide comprises SEQ ID NO: 595. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 596, wherein the chimeric polypeptide comprises X. In some embodiments, the chimeric polypeptide comprises SEQ ID NO: 596. In some aspects, further provided are compositions comprising the chimeric polypeptide and a carrier material, e.g., a carrier material provided herein.

Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 597, wherein the chimeric polypeptide comprises X. In some embodiments, the chimeric polypeptide comprises SEQ ID NO: 597. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 598, wherein the chimeric polypeptide comprises X. In some embodiments, the chimeric polypeptide comprises SEQ ID NO: 598. In some aspects, further provided are compositions comprising the chimeric polypeptide and a carrier material, e.g., a carrier material provided herein.

Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 599, wherein the chimeric polypeptide comprises X. In some embodiments, the chimeric polypeptide comprises SEQ ID NO: 599. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 600, wherein the chimeric polypeptide comprises X. In some embodiments, the chimeric polypeptide comprises SEQ ID NO: 600. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 601, wherein the chimeric polypeptide comprises X. In some embodiments, the chimeric polypeptide comprises SEQ ID NO: 601. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 602, wherein the chimeric polypeptide comprises X. In some embodiments, the chimeric polypeptide comprises SEQ ID NO: 602. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 603, wherein the chimeric polypeptide comprises X. In some embodiments, the chimeric polypeptide comprises SEQ ID NO: 603. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 604, wherein the chimeric polypeptide comprises X. In some embodiments, the chimeric polypeptide comprises SEQ ID NO: 604. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 605, wherein the chimeric polypeptide comprises X. In some embodiments, the chimeric polypeptide comprises SEQ ID NO: 605. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 606, wherein the chimeric polypeptide comprises X. In some embodiments, the chimeric polypeptide comprises SEQ ID NO: 606. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 607, wherein the chimeric polypeptide comprises X. In some embodiments, the chimeric polypeptide comprises SEQ ID NO: 607. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 608, wherein the chimeric polypeptide comprises X. In some embodiments, the chimeric polypeptide comprises SEQ ID NO: 608. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 609, wherein the chimeric polypeptide comprises X. In some embodiments, the chimeric polypeptide comprises SEQ ID NO: 609. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 610, wherein the chimeric polypeptide comprises X. In some embodiments, the chimeric polypeptide comprises SEQ ID NO: 610. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 611, wherein the chimeric polypeptide comprises X. In some embodiments, the chimeric polypeptide comprises SEQ ID NO: 611. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 612, wherein the chimeric polypeptide comprises X. In some embodiments, the chimeric polypeptide comprises SEQ ID NO: 612. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 613, wherein the chimeric polypeptide comprises X. In some embodiments, the chimeric polypeptide comprises SEQ ID NO: 613. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 614, wherein the chimeric polypeptide comprises X. In some embodiments, the chimeric polypeptide comprises SEQ ID NO: 614. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 615, wherein the chimeric polypeptide comprises X. In some embodiments, the chimeric polypeptide comprises SEQ ID NO: 615. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 616, wherein the chimeric polypeptide comprises X. In some embodiments, the chimeric polypeptide comprises SEQ ID NO: 616. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 617, wherein the chimeric polypeptide comprises X. In some embodiments, the chimeric polypeptide comprises SEQ ID NO: 617. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 618, wherein the chimeric polypeptide comprises X. In some embodiments, the chimeric polypeptide comprises SEQ ID NO: 618. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 619, wherein the chimeric polypeptide comprises X. In some embodiments, the chimeric polypeptide comprises SEQ ID NO: 619. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 620, wherein the chimeric polypeptide comprises X. In some embodiments, the chimeric polypeptide comprises SEQ ID NO: 620. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 621, wherein the chimeric polypeptide comprises X. In some embodiments, the chimeric polypeptide comprises SEQ ID NO: 621. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 622, wherein the chimeric polypeptide comprises X. In some embodiments, the chimeric polypeptide comprises SEQ ID NO: 622. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 623, wherein the chimeric polypeptide comprises X. In some embodiments, the chimeric polypeptide comprises SEQ ID NO: 623. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 624, wherein the chimeric polypeptide comprises X. In some embodiments, the chimeric polypeptide comprises SEQ ID NO: 624. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 625, wherein the chimeric polypeptide comprises X. In some embodiments, the chimeric polypeptide comprises SEQ ID NO: 625. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 626, wherein the chimeric polypeptide comprises X. In some embodiments, the chimeric polypeptide comprises SEQ ID NO: 626. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 627, wherein the chimeric polypeptide comprises X. In some embodiments, the chimeric polypeptide comprises SEQ ID NO: 627. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 628, wherein the chimeric polypeptide comprises X. In some embodiments, the chimeric polypeptide comprises SEQ ID NO: 628. In some aspects, further provided are compositions comprising the chimeric polypeptide and a carrier material, e.g., a carrier material provided herein.

Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 629, wherein the chimeric polypeptide comprises X. In some embodiments, the chimeric polypeptide comprises SEQ ID NO: 629. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 630, wherein the chimeric polypeptide comprises X. In some embodiments, the chimeric polypeptide comprises SEQ ID NO: 630. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 631, wherein the chimeric polypeptide comprises X. In some embodiments, the chimeric polypeptide comprises SEQ ID NO: 631. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 632, wherein the chimeric polypeptide comprises X. In some embodiments, the chimeric polypeptide comprises SEQ ID NO: 632. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 633, wherein the chimeric polypeptide comprises X. In some embodiments, the chimeric polypeptide comprises SEQ ID NO: 633. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 634, wherein the chimeric polypeptide comprises X. In some embodiments, the chimeric polypeptide comprises SEQ ID NO: 634. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 635, wherein the chimeric polypeptide comprises X. In some embodiments, the chimeric polypeptide comprises SEQ ID NO: 635. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 636, wherein the chimeric polypeptide comprises X. In some embodiments, the chimeric polypeptide comprises SEQ ID NO: 636. In some aspects, further provided are compositions comprising the chimeric polypeptide and a carrier material, e.g., a carrier material provided herein.

Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 637, wherein the chimeric polypeptide comprises X. In some embodiments, the chimeric polypeptide comprises SEQ ID NO: 637. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 638, wherein the chimeric polypeptide comprises X. In some embodiments, the chimeric polypeptide comprises SEQ ID NO: 638. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide comprises a sequence at least about 75%, 80%, 85%, 90%, 91%, 92%, 93%, 94%, 95%, 96%, 97%, 98%, 99% or 100% identical to SEQ ID NO: 639, wherein the chimeric polypeptide comprises X. In some embodiments, the chimeric polypeptide comprises SEQ ID NO: 639. In some aspects, further provided are compositions comprising the chimeric polypeptide and a carrier material, e.g., a carrier material provided herein. Non-limiting exemplary carrier materials may comprise one or more of the following: tricalcium phosphate, beta tricalcium phosphate, alpha tricalcium phosphate, fluorapatite, bone (e.g., demineralized bone), glasses (bioglasses) such as silicates, vanadates, and related ceramic minerals, and chelated divalent metal ions. In some aspects, further provided are methods of treating a defect in an organ and/or tissue comprising administering to a subject in need thereof the chimeric polypeptide and/or composition.

In some embodiments, the chimeric polypeptide further comprises a linker sequence between two targeting polypeptides. In some embodiments, the chimeric polypeptide further comprises a linker sequence between the targeting polypeptide and therapeutic agent.

In some embodiments, the chimeric polypeptide comprises a signal sequence at its N-terminus. In some embodiments, the chimeric polypeptide comprises a tag sequence (e.g., a poly-His tag, chitin-binding protein (CBP), maltose-binding protein (MBP), strep-tag, glutathione-S-transferase (GST), thioredoxin, or Fc region). Additional examples of tags are known in the art.

In some embodiments, the chimeric polypeptide comprises a lead sequence at its N-terminus that may assist with expression of the polypeptide. As a non-limiting example, the lead sequence comprises MPIGS (SEQ ID NO: 704).

Non-limiting exemplary embodiments of a chimeric polypeptide are provided herein: (1) In a first embodiment, the chimeric polypeptide comprises a targeting polypeptide. (2) The chimeric polypeptide of embodiment 1, wherein the targeting polypeptide comprises a sequence at least about 70%, 75%, 80%, 85%, 90%, 95%, or 100% identical to SEQ ID NO: 1. (3) The chimeric polypeptide of embodiment 1, wherein the targeting polypeptide comprises SEQ ID NO: 1. (4) The chimeric polypeptide of any previous embodiment, wherein the targeting polypeptide comprises a sequence at least about 70%, 75%, 80%, 85%, 90%, 95%, or 100% identical to SEQ ID NO: 2. (5) The chimeric polypeptide of any previous embodiment, wherein the targeting polypeptide comprises SEQ ID NO: 2. (6) The chimeric polypeptide of any previous embodiment, wherein the targeting polypeptide comprises a sequence at least about 70%, 75%, 80%, 85%, 90%, 95%, or 100% identical to SEQ ID NO: 3. (7) The chimeric polypeptide of any previous embodiment, wherein the targeting polypeptide comprises SEQ ID NO: 3. (8) The chimeric polypeptide of any previous embodiment, wherein the targeting polypeptide comprises a sequence at least about 70%, 75%, 80%, 85%, 90%, 95%, or 100% identical to SEQ ID NO: 4. (9) The chimeric polypeptide of any previous embodiment, wherein the targeting polypeptide comprises SEQ ID NO: 4. (10) The chimeric polypeptide of any previous embodiment, wherein the targeting polypeptide comprises a sequence at least about 70%, 75%, 80%, 85%, 90%, 95%, or 100% identical to SEQ ID NO: 5. (11) The chimeric polypeptide of any previous embodiment, wherein the targeting polypeptide comprises SEQ ID NO: 5. (12) The chimeric polypeptide of any previous embodiment, wherein the targeting polypeptide comprises a sequence at least about 70%, 75%, 80%, 85%, 90%, 95%, or 100% identical to SEQ ID NO: 6. (13) The chimeric polypeptide of any previous embodiment, wherein the targeting polypeptide comprises SEQ ID NO: 6. (14) The chimeric polypeptide of any previous embodiment, wherein the targeting polypeptide comprises a sequence at least about 70%, 75%, 80%, 85%, 90%, 95%, or 100% identical to SEQ ID NO: 7. (15) The chimeric polypeptide of any previous embodiment, wherein the targeting polypeptide comprises SEQ ID NO: 7. (16) The chimeric polypeptide of any previous embodiment, wherein the targeting polypeptide comprises a sequence at least about 70%, 75%, 80%, 85%, 90%, 95%, or 100% identical to SEQ ID NO: 8. (17) The chimeric polypeptide of any previous embodiment, wherein the targeting polypeptide comprises SEQ ID NO: 8. (18) The chimeric polypeptide of any previous embodiment, wherein the targeting polypeptide comprises a sequence at least about 70%, 75%, 80%, 85%, 90%, 95%, or 100% identical to SEQ ID NO: 9. (19) The chimeric polypeptide of any previous embodiment, wherein the targeting polypeptide comprises SEQ ID NO: 9. (20) The chimeric polypeptide of any previous embodiment, wherein the targeting polypeptide comprises a sequence at least about 70%, 75%, 80%, 85%, 90%, 95%, or 100% identical to SEQ ID NO: 10. (21) The chimeric polypeptide of any previous embodiment, wherein the targeting polypeptide comprises SEQ ID NO: 10. (22) The chimeric polypeptide of any previous embodiment, wherein the targeting polypeptide comprises a sequence at least about 70%, 75%, 80%, 85%, 90%, 95%, or 100% identical to SEQ ID NO: 11. (23) The chimeric polypeptide of any previous embodiment, wherein the targeting polypeptide comprises SEQ ID NO: 11. (24) The chimeric polypeptide of any previous embodiment, wherein the targeting polypeptide comprises a sequence at least about 70%, 75%, 80%, 85%, 90%, 95%, or 100% identical to SEQ ID NO: 12. (25) The chimeric polypeptide of any previous embodiment, wherein the targeting polypeptide comprises SEQ ID NO: 12. (26) The chimeric polypeptide of any previous embodiment, wherein the targeting polypeptide comprises a sequence at least about 70%, 75%, 80%, 85%, 90%, 95%, or 100% identical to SEQ ID NO: 13. (27) The chimeric polypeptide of any previous embodiment, wherein the targeting polypeptide comprises SEQ ID NO: 13. (28) The chimeric polypeptide of any previous embodiment, wherein the targeting polypeptide comprises a sequence at least about 70%, 75%, 80%, 85%, 90%, 95%, or 100% identical to SEQ ID NO: 14. (29) The chimeric polypeptide of any previous embodiment, wherein the targeting polypeptide comprises SEQ ID NO: 14. (30) The chimeric polypeptide of any previous embodiment, wherein the targeting polypeptide comprises a sequence at least about 70%, 75%, 80%, 85%, 90%, 95%, or 100% identical to SEQ ID NO: 15. (31) The chimeric polypeptide of any previous embodiment, wherein the targeting polypeptide comprises SEQ ID NO: 15. (32) The chimeric polypeptide of any previous embodiment, wherein the targeting polypeptide comprises a sequence at least about 70%, 75%, 80%, 85%, 90%, 95%, or 100% identical to SEQ ID NO: 16. (33) The chimeric polypeptide of any previous embodiment, wherein the targeting polypeptide comprises SEQ ID NO: 16. (34) The chimeric polypeptide of any previous embodiment, wherein the targeting polypeptide comprises a sequence at least about 70%, 75%, 80%, 85%, 90%, 95%, or 100% identical to SEQ ID NO: 17. (35) The chimeric polypeptide of any previous embodiment, wherein the targeting polypeptide comprises SEQ ID NO: 17. (36) The chimeric polypeptide of any previous embodiment, wherein the targeting polypeptide comprises a sequence at least about 70%, 75%, 80%, 85%, 90%, 95%, or 100% identical to SEQ ID NO: 18. (37) The chimeric polypeptide of any previous embodiment, wherein the targeting polypeptide comprises SEQ ID NO: 18. (38) The chimeric polypeptide of any previous embodiment, wherein the targeting polypeptide comprises a sequence at least about 70%, 75%, 80%, 85%, 90%, 95%, or 100% identical to SEQ ID NO: 19. (39) The chimeric polypeptide of any previous embodiment, wherein the targeting polypeptide comprises SEQ ID NO: 19. (40) The chimeric polypeptide of any previous embodiment, wherein the targeting polypeptide comprises a sequence at least about 70%, 75%, 80%, 85%, 90%, 95%, or 100% identical to SEQ ID NO: 20. (41) The chimeric polypeptide of any previous embodiment, wherein the targeting polypeptide comprises SEQ ID NO: 20. (42) The chimeric polypeptide of any previous embodiment, wherein the targeting polypeptide comprises a sequence at least about 70%, 75%, 80%, 85%, 90%, 95%, or 100% identical to SEQ ID NO: 21. (43) The chimeric polypeptide of any previous embodiment, wherein the targeting polypeptide comprises SEQ ID NO: 21. (44) The chimeric polypeptide of any previous embodiment, wherein the targeting polypeptide comprises a sequence at least about 70%, 75%, 80%, 85%, 90%, 95%, or 100% identical to SEQ ID NO: 22. (45) The chimeric polypeptide of any previous embodiment, wherein the targeting polypeptide comprises SEQ ID NO: 22. (46) The chimeric polypeptide of any previous embodiment, wherein the targeting polypeptide comprises a sequence at least about 70%, 75%, 80%, 85%, 90%, 95%, or 100% identical to SEQ ID NO: 23. (47) The chimeric polypeptide of any previous embodiment, wherein the targeting polypeptide comprises SEQ ID NO: 23. (48) The chimeric polypeptide of any previous embodiment, wherein the targeting polypeptide comprises a sequence at least about 70%, 75%, 80%, 85%, 90%, 95%, or 100% identical to SEQ ID NO: 24. (49) The chimeric polypeptide of any previous embodiment, wherein the targeting polypeptide comprises SEQ ID NO: 24. (50) The chimeric polypeptide of any previous embodiment, wherein the targeting polypeptide comprises a sequence at least about 70%, 75%, 80%, 85%, 90%, 95%, or 100% identical to SEQ ID NO: 25. (51) The chimeric polypeptide of any previous embodiment, wherein the targeting polypeptide comprises SEQ ID NO: 25. (52) The chimeric polypeptide of any previous embodiment, wherein the targeting polypeptide comprises a sequence at least about 70%, 75%, 80%, 85%, 90%, 95%, or 100% identical to SEQ ID NO: 26. (53) The chimeric polypeptide of any previous embodiment, wherein the targeting polypeptide comprises SEQ ID NO: 26. (54) The chimeric polypeptide of any previous embodiment, wherein the targeting polypeptide comprises a sequence at least about 70%, 75%, 80%, 85%, 90%, 95%, or 100% identical to SEQ ID NO: 27. (55) The chimeric polypeptide of any previous embodiment, wherein the targeting polypeptide comprises SEQ ID NO: 27. (56) The chimeric polypeptide of any previous embodiment, wherein the targeting polypeptide comprises a sequence at least about 70%, 75%, 80%, 85%, 90%, 95%, or 100% identical to SEQ ID NO: 28. (57) The chimeric polypeptide of any previous embodiment, wherein the targeting polypeptide comprises SEQ ID NO: 28. (58) The chimeric polypeptide of any previous embodiment, wherein the targeting polypeptide comprises a sequence at least about 70%, 75%, 80%, 85%, 90%, 95%, or 100% identical to SEQ ID NO: 29. (59) The chimeric polypeptide of any previous embodiment, wherein the targeting polypeptide comprises SEQ ID NO: 29. (60) The chimeric polypeptide of any previous embodiment, wherein the targeting polypeptide comprises a sequence at least about 70%, 75%, 80%, 85%, 90%, 95%, or 100% identical to SEQ ID NO: 30. (61) The chimeric polypeptide of any previous embodiment, wherein the targeting polypeptide comprises SEQ ID NO: 30. (62) The chimeric polypeptide of any previous embodiment, wherein the targeting polypeptide comprises a sequence at least about 70%, 75%, 80%, 85%, 90%, 95%, or 100% identical to SEQ ID NO: 31. (63) The chimeric polypeptide of any previous embodiment, wherein the targeting polypeptide comprises SEQ ID NO: 31. (64) The chimeric polypeptide of any previous embodiment, wherein the targeting polypeptide comprises a sequence at least about 70%, 75%, 80%, 85%, 90%, 95%, or 100% identical to SEQ ID NO: 32. (65) The chimeric polypeptide of any previous embodiment, wherein the targeting polypeptide comprises SEQ ID NO: 32. (66) The chimeric polypeptide of any previous embodiment, wherein the targeting polypeptide comprises a sequence at least about 70%, 75%, 80%, 85%, 90%, 95%, or 100% identical to SEQ ID NO: 33. (67) The chimeric polypeptide of any previous embodiment, wherein the targeting polypeptide comprises SEQ ID NO: 33. (68) The chimeric polypeptide of any previous embodiment, wherein the targeting polypeptide comprises a sequence at least about 70%, 75%, 80%, 85%, 90%, 95%, or 100% identical to SEQ ID NO: 34. (69) The chimeric polypeptide of any previous embodiment, wherein the targeting polypeptide comprises SEQ ID NO: 34. (70) The chimeric polypeptide of any previous embodiment, wherein the targeting polypeptide comprises a sequence at least about 70%, 75%, 80%, 85%, 90%, 95%, or 100% identical to SEQ ID NO: 35. (71) The chimeric polypeptide of any previous embodiment, wherein the targeting polypeptide comprises SEQ ID NO: 35. (72) The chimeric polypeptide of any previous embodiment, wherein the targeting polypeptide comprises a sequence at least about 70%, 75%, 80%, 85%, 90%, 95%, or 100% identical to SEQ ID NO: 36. (73) The chimeric polypeptide of any previous embodiment, wherein the targeting polypeptide comprises SEQ ID NO: 36. (74) The chimeric polypeptide of any previous embodiment, wherein the targeting polypeptide comprises a sequence at least about 70%, 75%, 80%, 85%, 90%, 95%, or 100% identical to SEQ ID NO: 37. (75) The chimeric polypeptide of any previous embodiment, wherein the targeting polypeptide comprises SEQ ID NO: 37. (76) The chimeric polypeptide of any previous embodiment, wherein the targeting polypeptide comprises a sequence at least about 70%, 75%, 80%, 85%, 90%, 95%, or 100% identical to SEQ ID NO: 38. (77) The chimeric polypeptide of any previous embodiment, wherein the targeting polypeptide comprises SEQ ID NO: 38. (78) The chimeric polypeptide of any previous embodiment, wherein the targeting polypeptide comprises a sequence at least about 70%, 75%, 80%, 85%, 90%, 95%, or 100% identical to SEQ ID NO: 39. (79) The chimeric polypeptide of any previous embodiment, wherein the targeting polypeptide comprises SEQ ID NO: 39. (80) The chimeric polypeptide of any previous embodiment, wherein the targeting polypeptide comprises a sequence at least about 70%, 75%, 80%, 85%, 90%, 95%, or 100% identical to SEQ ID NO: 40. (81) The chimeric polypeptide of any previous embodiment, wherein the targeting polypeptide comprises SEQ ID NO: 40. (82) The chimeric polypeptide of any previous embodiment, wherein the targeting polypeptide comprises a sequence at least about 70%, 75%, 80%, 85%, 90%, 95%, or 100% identical to SEQ ID NO: 41. (83) The chimeric polypeptide of any previous embodiment, wherein the targeting polypeptide comprises SEQ ID NO: 41. (84) The chimeric polypeptide of any previous embodiment, wherein the targeting polypeptide comprises a sequence at least about 70%, 75%, 80%, 85%, 90%, 95%, or 100% identical to SEQ ID NO: 42. (85) The chimeric polypeptide of any previous embodiment, wherein the targeting polypeptide comprises SEQ ID NO: 42. (86) The chimeric polypeptide of any previous embodiment, wherein the targeting polypeptide comprises a sequence at least about 70%, 75%, 80%, 85%, 90%, 95%, or 100% identical to SEQ ID NO: 43. (87) The chimeric polypeptide of any previous embodiment, wherein the targeting polypeptide comprises SEQ ID NO: 43. (88) The chimeric polypeptide of any previous embodiment, wherein the targeting polypeptide comprises a sequence at least about 70%, 75%, 80%, 85%, 90%, 95%, or 100% identical to SEQ ID NO: 401. (89) The chimeric polypeptide of any previous embodiment, wherein the targeting polypeptide comprises SEQ ID NO: 401. (90) The chimeric polypeptide of any previous embodiment, wherein the targeting polypeptide comprises a sequence at least about 70%, 75%, 80%, 85%, 90%, 95%, or 100% identical to SEQ ID NO: 402. (91) The chimeric polypeptide of any previous embodiment, wherein the targeting polypeptide comprises SEQ ID NO: 402. (92) The chimeric polypeptide of any previous embodiment, wherein the targeting polypeptide comprises a sequence at least about 70%, 75%, 80%, 85%, 90%, 95%, or 100% identical to SEQ ID NO: 403. (93) The chimeric polypeptide of any previous embodiment, wherein the targeting polypeptide comprises SEQ ID NO: 403. (94) The chimeric polypeptide of any previous embodiment, wherein the targeting polypeptide comprises a sequence at least about 70%, 75%, 80%, 85%, 90%, 95%, or 100% identical to SEQ ID NO: 404. (95) The chimeric polypeptide of any previous embodiment, wherein the targeting polypeptide comprises SEQ ID NO: 404. (96) The chimeric polypeptide of any previous embodiment, wherein the targeting polypeptide comprises a sequence at least about 70%, 75%, 80%, 85%, 90%, 95%, or 100% identical to SEQ ID NO: 405. (97) The chimeric polypeptide of any previous embodiment, wherein the targeting polypeptide comprises SEQ ID NO: 405. (98) The chimeric polypeptide of any previous embodiment, wherein the targeting polypeptide comprises a sequence at least about 70%, 75%, 80%, 85%, 90%, 95%, or 100% identical to SEQ ID NO: 406. (99) The chimeric polypeptide of any previous embodiment, wherein the targeting polypeptide comprises SEQ ID NO: 406. (100) The chimeric polypeptide of any previous embodiment, wherein the targeting polypeptide comprises a sequence at least about 70%, 75%, 80%, 85%, 90%, 95%, or 100% identical to SEQ ID NO: 407. (101) The chimeric polypeptide of any previous embodiment, wherein the targeting polypeptide comprises SEQ ID NO: 407. (102) The chimeric polypeptide of any previous embodiment, wherein the targeting polypeptide comprises a sequence at least about 70%, 75%, 80%, 85%, 90%, 95%, or 100% identical to SEQ ID NO: 408. (103) The chimeric polypeptide of any previous embodiment, wherein the targeting polypeptide comprises SEQ ID NO: 408. (104) The chimeric polypeptide of any previous embodiment, wherein the targeting polypeptide comprises a sequence at least about 70%, 75%, 80%, 85%, 90%, 95%, or 100% identical to SEQ ID NO: 409. (105) The chimeric polypeptide of any previous embodiment, wherein the targeting polypeptide comprises SEQ ID NO: 409. (106) The chimeric polypeptide of any previous embodiment, wherein the targeting polypeptide comprises a sequence at least about 70%, 75%, 80%, 85%, 90%, 95%, or 100% identical to SEQ ID NO: 410. (107) The chimeric polypeptide of any previous embodiment, wherein the targeting polypeptide comprises SEQ ID NO: 410. (108) The chimeric polypeptide of any previous embodiment, wherein the targeting polypeptide comprises a sequence at least about 70%, 75%, 80%, 85%, 90%, 95%, or 100% identical to SEQ ID NO: 411. (109) The chimeric polypeptide of any previous embodiment, wherein the targeting polypeptide comprises SEQ ID NO: 411. (110) The chimeric polypeptide of any previous embodiment, wherein the targeting polypeptide comprises a sequence at least about 70%, 75%, 80%, 85%, 90%, 95%, or 100% identical to SEQ ID NO: 412. (111) The chimeric polypeptide of any previous embodiment, wherein the targeting polypeptide comprises SEQ ID NO: 412. (112) The chimeric polypeptide of any previous embodiment, wherein the targeting polypeptide comprises a sequence at least about 70%, 75%, 80%, 85%, 90%, 95%, or 100% identical to SEQ ID NO: 413. (113) The chimeric polypeptide of any previous embodiment, wherein the targeting polypeptide comprises SEQ ID NO: 413. (114) The chimeric polypeptide of any previous embodiment, wherein the targeting polypeptide comprises a sequence at least about 70%, 75%, 80%, 85%, 90%, 95%, or 100% identical to SEQ ID NO: 414. (115) The chimeric polypeptide of any previous embodiment, wherein the targeting polypeptide comprises SEQ ID NO: 414. (116) The chimeric polypeptide of any previous embodiment, wherein the targeting polypeptide comprises a sequence at least about 70%, 75%, 80%, 85%, 90%, 95%, or 100% identical to SEQ ID NO: 415. (117) The chimeric polypeptide of any previous embodiment, wherein the targeting polypeptide comprises SEQ ID NO: 415. (118) The chimeric polypeptide of any previous embodiment, wherein the targeting polypeptide comprises a sequence at least about 70%, 75%, 80%, 85%, 90%, 95%, or 100% identical to SEQ ID NO: 416. (119) The chimeric polypeptide of any previous embodiment, wherein the targeting polypeptide comprises SEQ ID NO: 416. (120) The chimeric polypeptide of any previous embodiment, wherein the targeting polypeptide comprises a sequence at least about 70%, 75%, 80%, 85%, 90%, 95%, or 100% identical to SEQ ID NO: 417. (121) The chimeric polypeptide of any previous embodiment, wherein the targeting polypeptide comprises SEQ ID NO: 417. (122) The chimeric polypeptide of any previous embodiment, wherein the targeting polypeptide comprises a sequence at least about 70%, 75%, 80%, 85%, 90%, 95%, or 100% identical to SEQ ID NO: 418. (123) The chimeric polypeptide of any previous embodiment, wherein the targeting polypeptide comprises SEQ ID NO: 418. (124) The chimeric polypeptide of any previous embodiment, wherein the targeting polypeptide comprises a sequence at least about 70%, 75%, 80%, 85%, 90%, 95%, or 100% identical to SEQ ID NO: 419. (125) The chimeric polypeptide of any previous embodiment, wherein the targeting polypeptide comprises SEQ ID NO: 419. (126) The chimeric polypeptide of any previous embodiment, wherein the targeting polypeptide comprises a sequence at least about 70%, 75%, 80%, 85%, 90%, 95%, or 100% identical to SEQ ID NO: 420. (127) The chimeric polypeptide of any previous embodiment, wherein the targeting polypeptide comprises SEQ ID NO: 420. (128) The chimeric polypeptide of any previous embodiment, wherein the targeting polypeptide comprises a sequence at least about 70%, 75%, 80%, 85%, 90%, 95%, or 100% identical to SEQ ID NO: 421. (129) The chimeric polypeptide of any previous embodiment, wherein the targeting polypeptide comprises SEQ ID NO: 421. (130) The chimeric polypeptide of any previous embodiment, wherein the targeting polypeptide comprises a sequence at least about 70%, 75%, 80%, 85%, 90%, 95%, or 100% identical to SEQ ID NO: 422. (131) The chimeric polypeptide of any previous embodiment, wherein the targeting polypeptide comprises SEQ ID NO: 422. (132) The chimeric polypeptide of any previous embodiments, wherein the targeting polypeptide comprises Formula I. (133) The chimeric polypeptide of any previous embodiment, comprising a therapeutic agent. (134) The chimeric polypeptide of embodiment 133, wherein the therapeutic agent comprises a mammalian growth factor. (135) The chimeric polypeptide of embodiment 133 or embodiment 134, wherein the therapeutic agent comprises a bone morphogenetic protein (BMP). (136) The chimeric polypeptide of embodiment 135, wherein the BMP comprises BMP-2. (137) The chimeric polypeptide of embodiment 135, wherein the BMP comprises a sequence at least about 70%, 75%, 80%, 85%, 90%, 95%, or 100% identical to SEQ ID NO: 32. (138) The chimeric polypeptide of embodiment 135, wherein the BMP comprises SEQ ID NO: 32. (139) The chimeric polypeptide of embodiment 133 or embodiment 134, wherein the therapeutic agent comprises a sequence at least about 70%, 75%, 80%, 85%, 90%, 95%, or 100% identical to a sequence set forth in Table B. (140) The chimeric polypeptide of embodiment 133 or embodiment 134, wherein the therapeutic agent comprises two or more therapeutic sequences, each therapeutic sequence at least about 70%, 75%, 80%, 85%, 90%, 95%, or 100% identical to a sequence set forth in Table B. (141) The chimeric polypeptide of embodiment 140, wherein the two or more is 2, 3, 4, or 5 therapeutic sequences. (142) The chimeric polypeptide of embodiment 140, wherein the two or more is from two to about ten. (143) The chimeric polypeptide of any previous embodiment, comprising a linker. (144) The chimeric polypeptide of embodiment 143, wherein the linker comprises GSEG (SEQ ID NO: 702). (145) The chimeric polypeptide of embodiment 143 or embodiment 144, wherein the linker comprises SEGG (SEQ ID NO: 703). (146) The chimeric polypeptide of any one of embodiments 143-145, wherein the linker comprises a sequence at least about 70%, 75%, 80%, 85%, 90%, 95%, 96%, 97%, 98%, or 99% identical to SEQ ID NO: 701, or wherein the linker comprises SEQ ID NO: 701. (147) The chimeric polypeptide of any previous embodiment, comprising a sequence at least about 70%, 75%, 80%, 85%, 90%, 95%, 96%, 97%, 98%, or 99% identical to SEQ ID NO: 501, or wherein the chimeric polypeptide comprises SEQ ID NO: 501. (148) The chimeric polypeptide of any previous embodiment, comprising a sequence at least about 70%, 75%, 80%, 85%, 90%, 95%, 96%, 97%, 98%, or 99% identical to SEQ ID NO: 502, or wherein the chimeric polypeptide comprises SEQ ID NO: 502. (149) The chimeric polypeptide of any previous embodiment, comprising a sequence at least about 70%, 75%, 80%, 85%, 90%, 95%, 96%, 97%, 98%, or 99% identical to SEQ ID NO: 503, or wherein the chimeric polypeptide comprises SEQ ID NO: 503. (150) The chimeric polypeptide of any previous embodiment, comprising a sequence at least about 70%, 75%, 80%, 85%, 90%, 95%, 96%, 97%, 98%, or 99% identical to SEQ ID NO: 504, or wherein the chimeric polypeptide comprises SEQ ID NO: 504. (151) The chimeric polypeptide of any previous embodiment, comprising a sequence at least about 70%, 75%, 80%, 85%, 90%, 95%, 96%, 97%, 98%, or 99% identical to SEQ ID NO: 505, or wherein the chimeric polypeptide comprises SEQ ID NO: 505. (152) The chimeric polypeptide of any previous embodiment, comprising a sequence at least about 70%, 75%, 80%, 85%, 90%, 95%, 96%, 97%, 98%, or 99% identical to SEQ ID NO: 506, or wherein the chimeric polypeptide comprises SEQ ID NO: 506. (153) The chimeric polypeptide of any previous embodiment, comprising a sequence at least about 70%, 75%, 80%, 85%, 90%, 95%, 96%, 97%, 98%, or 99% identical to SEQ ID NO: 507, or wherein the chimeric polypeptide comprises SEQ ID NO: 507. (154) The chimeric polypeptide of any previous embodiment, comprising a sequence at least about 70%, 75%, 80%, 85%, 90%, 95%, 96%, 97%, 98%, or 99% identical to SEQ ID NO: 508 and the sequence comprises X; or wherein the chimeric polypeptide comprises SEQ ID NO: 508. (155) The chimeric polypeptide of any previous embodiment, comprising a sequence at least about 70%, 75%, 80%, 85%, 90%, 95%, 96%, 97%, 98%, or 99% identical to SEQ ID NO: 509 and the sequence comprises X; or wherein the chimeric polypeptide comprises SEQ ID NO: 509. (156) The chimeric polypeptide of any previous embodiment, comprising a sequence at least about 70%, 75%, 80%, 85%, 90%, 95%, 96%, 97%, 98%, or 99% identical to SEQ ID NO: 510 and the sequence comprises X; or wherein the chimeric polypeptide comprises SEQ ID NO: 510. (157) The chimeric polypeptide of any previous embodiment, comprising a sequence at least about 70%, 75%, 80%, 85%, 90%, 95%, 96%, 97%, 98%, or 99% identical to SEQ ID NO: 511 and the sequence comprises X; or wherein the chimeric polypeptide comprises SEQ ID NO: 511. (158) The chimeric polypeptide of any previous embodiment, comprising a sequence at least about 70%, 75%, 80%, 85%, 90%, 95%, 96%, 97%, 98%, or 99% identical to SEQ ID NO: 512 and the sequence comprises X; or wherein the chimeric polypeptide comprises SEQ ID NO: 512. (159) The chimeric polypeptide of any previous embodiment, comprising a sequence at least about 70%, 75%, 80%, 85%, 90%, 95%, 96%, 97%, 98%, or 99% identical to SEQ ID NO: 513 and the sequence comprises X; or wherein the chimeric polypeptide comprises SEQ ID NO: 513. (160) The chimeric polypeptide of any previous embodiment, comprising a sequence at least about 70%, 75%, 80%, 85%, 90%, 95%, 96%, 97%, 98%, or 99% identical to SEQ ID NO: 514 and the sequence comprises X; or wherein the chimeric polypeptide comprises SEQ ID NO: 514. (161) The chimeric polypeptide of any previous embodiment, comprising a sequence at least about 70%, 75%, 80%, 85%, 90%, 95%, 96%, 97%, 98%, or 99% identical to SEQ ID NO: 515 and the sequence comprises X; or wherein the chimeric polypeptide comprises SEQ ID NO: 515. (162) The chimeric polypeptide of any previous embodiment, comprising a sequence at least about 70%, 75%, 80%, 85%, 90%, 95%, 96%, 97%, 98%, or 99% identical to SEQ ID NO: 516 and the sequence comprises X; or wherein the chimeric polypeptide comprises SEQ ID NO: 516. (163) The chimeric polypeptide of any previous embodiment, comprising a sequence at least about 70%, 75%, 80%, 85%, 90%, 95%, 96%, 97%, 98%, or 99% identical to SEQ ID NO: 517 and the sequence comprises X; or wherein the chimeric polypeptide comprises SEQ ID NO: 517. (164) The chimeric polypeptide of any previous embodiment, comprising a sequence at least about 70%, 75%, 80%, 85%, 90%, 95%, 96%, 97%, 98%, or 99% identical to SEQ ID NO: 518 and the sequence comprises X; or wherein the chimeric polypeptide comprises SEQ ID NO: 518. (165) The chimeric polypeptide of any previous embodiment, comprising a sequence at least about 70%, 75%, 80%, 85%, 90%, 95%, 96%, 97%, 98%, or 99% identical to SEQ ID NO: 519 and the sequence comprises X; or wherein the chimeric polypeptide comprises SEQ ID NO: 519. (166) The chimeric polypeptide of any previous embodiment, comprising a sequence at least about 70%, 75%, 80%, 85%, 90%, 95%, 96%, 97%, 98%, or 99% identical to SEQ ID NO: 520 and the sequence comprises X; or wherein the chimeric polypeptide comprises SEQ ID NO: 520. (167) The chimeric polypeptide of any previous embodiment, comprising a sequence at least about 70%, 75%, 80%, 85%, 90%, 95%, 96%, 97%, 98%, or 99% identical to SEQ ID NO: 521 and the sequence comprises X; or wherein the chimeric polypeptide comprises SEQ ID NO: 521. (168) The chimeric polypeptide of any previous embodiment, comprising a sequence at least about 70%, 75%, 80%, 85%, 90%, 95%, 96%, 97%, 98%, or 99% identical to SEQ ID NO: 522 and the sequence comprises X; or wherein the chimeric polypeptide comprises SEQ ID NO: 522. (169) The chimeric polypeptide of any previous embodiment, comprising a sequence at least about 70%, 75%, 80%, 85%, 90%, 95%, 96%, 97%, 98%, or 99% identical to SEQ ID NO: 523 and the sequence comprises X; or wherein the chimeric polypeptide comprises SEQ ID NO: 523. (170) The chimeric polypeptide of any previous embodiment, comprising a sequence at least about 70%, 75%, 80%, 85%, 90%, 95%, 96%, 97%, 98%, or 99% identical to SEQ ID NO: 524 and the sequence comprises X; or wherein the chimeric polypeptide comprises SEQ ID NO: 524. (171) The chimeric polypeptide of any previous embodiment, comprising a sequence at least about 70%, 75%, 80%, 85%, 90%, 95%, 96%, 97%, 98%, or 99% identical to SEQ ID NO: 525 and the sequence comprises X; or wherein the chimeric polypeptide comprises SEQ ID NO: 525. (172) The chimeric polypeptide of any previous embodiment, comprising a sequence at least about 70%, 75%, 80%, 85%, 90%, 95%, 96%, 97%, 98%, or 99% identical to SEQ ID NO: 526 and the sequence comprises X; or wherein the chimeric polypeptide comprises SEQ ID NO: 526. (173) The chimeric polypeptide of any previous embodiment, comprising a sequence at least about 70%, 75%, 80%, 85%, 90%, 95%, 96%, 97%, 98%, or 99% identical to SEQ ID NO: 527 and the sequence comprises X; or wherein the chimeric polypeptide comprises SEQ ID NO: 527. (174) The chimeric polypeptide of any previous embodiment, comprising a sequence at least about 70%, 75%, 80%, 85%, 90%, 95%, 96%, 97%, 98%, or 99% identical to SEQ ID NO: 528 and the sequence comprises X; or wherein the chimeric polypeptide comprises SEQ ID NO: 528. (175) The chimeric polypeptide of any previous embodiment, comprising a sequence at least about 70%, 75%, 80%, 85%, 90%, 95%, 96%, 97%, 98%, or 99% identical to SEQ ID NO: 529 and the sequence comprises X; or wherein the chimeric polypeptide comprises SEQ ID NO: 529. (176) The chimeric polypeptide of any previous embodiment, comprising a sequence at least about 70%, 75%, 80%, 85%, 90%, 95%, 96%, 97%, 98%, or 99% identical to SEQ ID NO: 530 and the sequence comprises X; or wherein the chimeric polypeptide comprises SEQ ID NO: 530. (177) The chimeric polypeptide of any previous embodiment, comprising a sequence at least about 70%, 75%, 80%, 85%, 90%, 95%, 96%, 97%, 98%, or 99% identical to SEQ ID NO: 531 and the sequence comprises X; or wherein the chimeric polypeptide comprises SEQ ID NO: 531. (178) The chimeric polypeptide of any previous embodiment, comprising a sequence at least about 70%, 75%, 80%, 85%, 90%, 95%, 96%, 97%, 98%, or 99% identical to SEQ ID NO: 532 and the sequence comprises X; or wherein the chimeric polypeptide comprises SEQ ID NO: 532. (179) The chimeric polypeptide of any previous embodiment, comprising a sequence at least about 70%, 75%, 80%, 85%, 90%, 95%, 96%, 97%, 98%, or 99% identical to SEQ ID NO: 533 and the sequence comprises X; or wherein the chimeric polypeptide comprises SEQ ID NO: 533. (180) The chimeric polypeptide of any previous embodiment, comprising a sequence at least about 70%, 75%, 80%, 85%, 90%, 95%, 96%, 97%, 98%, or 99% identical to SEQ ID NO: 534 and the sequence comprises X; or wherein the chimeric polypeptide comprises SEQ ID NO: 534. (181) The chimeric polypeptide of any previous embodiment, comprising a sequence at least about 70%, 75%, 80%, 85%, 90%, 95%, 96%, 97%, 98%, or 99% identical to SEQ ID NO: 535 and the sequence comprises X; or wherein the chimeric polypeptide comprises SEQ ID NO: 535. (182) The chimeric polypeptide of any previous embodiment, comprising a sequence at least about 70%, 75%, 80%, 85%, 90%, 95%, 96%, 97%, 98%, or 99% identical to SEQ ID NO: 536 and the sequence comprises X; or wherein the chimeric polypeptide comprises SEQ ID NO: 536. (183) The chimeric polypeptide of any previous embodiment, comprising a sequence at least about 70%, 75%, 80%, 85%, 90%, 95%, 96%, 97%, 98%, or 99% identical to SEQ ID NO: 537 and the sequence comprises X; or wherein the chimeric polypeptide comprises SEQ ID NO: 537. (184) The chimeric polypeptide of any previous embodiment, comprising a sequence at least about 70%, 75%, 80%, 85%, 90%, 95%, 96%, 97%, 98%, or 99% identical to SEQ ID NO: 538 and the sequence comprises X; or wherein the chimeric polypeptide comprises SEQ ID NO: 538. (185) The chimeric polypeptide of any previous embodiment, comprising a sequence at least about 70%, 75%, 80%, 85%, 90%, 95%, 96%, 97%, 98%, or 99% identical to SEQ ID NO: 539 and the sequence comprises X; or wherein the chimeric polypeptide comprises SEQ ID NO: 539. (186) The chimeric polypeptide of any previous embodiment, comprising a sequence at least about 70%, 75%, 80%, 85%, 90%, 95%, 96%, 97%, 98%, or 99% identical to SEQ ID NO: 540 and the sequence comprises X; or wherein the chimeric polypeptide comprises SEQ ID NO: 540. (187) The chimeric polypeptide of any previous embodiment, comprising a sequence at least about 70%, 75%, 80%, 85%, 90%, 95%, 96%, 97%, 98%, or 99% identical to SEQ ID NO: 541 and the sequence comprises X; or wherein the chimeric polypeptide comprises SEQ ID NO: 541. (188) The chimeric polypeptide of any previous embodiment, comprising a sequence at least about 70%, 75%, 80%, 85%, 90%, 95%, 96%, 97%, 98%, or 99% identical to SEQ ID NO: 542 and the sequence comprises X; or wherein the chimeric polypeptide comprises SEQ ID NO: 542. (189) The chimeric polypeptide of any previous embodiment, comprising a sequence at least about 70%, 75%, 80%, 85%, 90%, 95%, 96%, 97%, 98%, or 99% identical to SEQ ID NO: 543 and the sequence comprises X; or wherein the chimeric polypeptide comprises SEQ ID NO: 543. (190) The chimeric polypeptide of any previous embodiment, comprising a sequence at least about 70%, 75%, 80%, 85%, 90%, 95%, 96%, 97%, 98%, or 99% identical to SEQ ID NO: 544 and the sequence comprises X; or wherein the chimeric polypeptide comprises SEQ ID NO: 544. (191) The chimeric polypeptide of any previous embodiment, comprising a sequence at least about 70%, 75%, 80%, 85%, 90%, 95%, 96%, 97%, 98%, or 99% identical to SEQ ID NO: 545 and the sequence comprises X; or wherein the chimeric polypeptide comprises SEQ ID NO: 545. (192) The chimeric polypeptide of any previous embodiment, comprising a sequence at least about 70%, 75%, 80%, 85%, 90%, 95%, 96%, 97%, 98%, or 99% identical to SEQ ID NO: 546 and the sequence comprises X; or wherein the chimeric polypeptide comprises SEQ ID NO: 546. (193) The chimeric polypeptide of any previous embodiment, comprising a sequence at least about 70%, 75%, 80%, 85%, 90%, 95%, 96%, 97%, 98%, or 99% identical to SEQ ID NO: 547 and the sequence comprises X; or wherein the chimeric polypeptide comprises SEQ ID NO: 547. (194) The chimeric polypeptide of any previous embodiment, comprising a sequence at least about 70%, 75%, 80%, 85%, 90%, 95%, 96%, 97%, 98%, or 99% identical to SEQ ID NO: 548 and the sequence comprises X; or wherein the chimeric polypeptide comprises SEQ ID NO: 548. (195) The chimeric polypeptide of any previous embodiment, comprising a sequence at least about 70%, 75%, 80%, 85%, 90%, 95%, 96%, 97%, 98%, or 99% identical to SEQ ID NO: 549 and the sequence comprises X; or wherein the chimeric polypeptide comprises SEQ ID NO: 549. (196) The chimeric polypeptide of any previous embodiment, comprising a sequence at least about 70%, 75%, 80%, 85%, 90%, 95%, 96%, 97%, 98%, or 99% identical to SEQ ID NO: 550 and the sequence comprises X; or wherein the chimeric polypeptide comprises SEQ ID NO: 550. (197) The chimeric polypeptide of any previous embodiment, comprising a sequence at least about 70%, 75%, 80%, 85%, 90%, 95%, 96%, 97%, 98%, or 99% identical to SEQ ID NO: 551 and the sequence comprises X; or wherein the chimeric polypeptide comprises SEQ ID NO: 551. (198) The chimeric polypeptide of any previous embodiment, comprising a sequence at least about 70%, 75%, 80%, 85%, 90%, 95%, 96%, 97%, 98%, or 99% identical to SEQ ID NO: 552 and the sequence comprises X; or wherein the chimeric polypeptide comprises SEQ ID NO: 552. (199) The chimeric polypeptide of any previous embodiment, comprising a sequence at least about 70%, 75%, 80%, 85%, 90%, 95%, 96%, 97%, 98%, or 99% identical to SEQ ID NO: 553 and the sequence comprises X; or wherein the chimeric polypeptide comprises SEQ ID NO: 553. (200) The chimeric polypeptide of any previous embodiment, comprising a sequence at least about 70%, 75%, 80%, 85%, 90%, 95%, 96%, 97%, 98%, or 99% identical to SEQ ID NO: 554 and the sequence comprises X; or wherein the chimeric polypeptide comprises SEQ ID NO: 554. (201) The chimeric polypeptide of any previous embodiment, comprising a sequence at least about 70%, 75%, 80%, 85%, 90%, 95%, 96%, 97%, 98%, or 99% identical to SEQ ID NO: 555 and the sequence comprises X; or wherein the chimeric polypeptide comprises SEQ ID NO: 555. (202) The chimeric polypeptide of any previous embodiment, comprising a sequence at least about 70%, 75%, 80%, 85%, 90%, 95%, 96%, 97%, 98%, or 99% identical to SEQ ID NO: 556 and the sequence comprises X; or wherein the chimeric polypeptide comprises SEQ ID NO: 556. (203) The chimeric polypeptide of any previous embodiment, comprising a sequence at least about 70%, 75%, 80%, 85%, 90%, 95%, 96%, 97%, 98%, or 99% identical to SEQ ID NO: 557 and the sequence comprises X; or wherein the chimeric polypeptide comprises SEQ ID NO: 557. (204) The chimeric polypeptide of any previous embodiment, comprising a sequence at least about 70%, 75%, 80%, 85%, 90%, 95%, 96%, 97%, 98%, or 99% identical to SEQ ID NO: 558 and the sequence comprises X; or wherein the chimeric polypeptide comprises SEQ ID NO: 558. (205) The chimeric polypeptide of any previous embodiment, comprising a sequence at least about 70%, 75%, 80%, 85%, 90%, 95%, 96%, 97%, 98%, or 99% identical to SEQ ID NO: 559 and the sequence comprises X; or wherein the chimeric polypeptide comprises SEQ ID NO: 559. (206) The chimeric polypeptide of any previous embodiment, comprising a sequence at least about 70%, 75%, 80%, 85%, 90%, 95%, 96%, 97%, 98%, or 99% identical to SEQ ID NO: 560 and the sequence comprises X; or wherein the chimeric polypeptide comprises SEQ ID NO: 560. (207) The chimeric polypeptide of any previous embodiment, comprising a sequence at least about 70%, 75%, 80%, 85%, 90%, 95%, 96%, 97%, 98%, or 99% identical to SEQ ID NO: 561 and the sequence comprises X; or wherein the chimeric polypeptide comprises SEQ ID NO: 561. (208) The chimeric polypeptide of any previous embodiment, comprising a sequence at least about 70%, 75%, 80%, 85%, 90%, 95%, 96%, 97%, 98%, or 99% identical to SEQ ID NO: 562 and the sequence comprises X; or wherein the chimeric polypeptide comprises SEQ ID NO: 562. (209) The chimeric polypeptide of any previous embodiment, comprising a sequence at least about 70%, 75%, 80%, 85%, 90%, 95%, 96%, 97%, 98%, or 99% identical to SEQ ID NO: 563 and the sequence comprises X; or wherein the chimeric polypeptide comprises SEQ ID NO: 563. (210) The chimeric polypeptide of any previous embodiment, comprising a sequence at least about 70%, 75%, 80%, 85%, 90%, 95%, 96%, 97%, 98%, or 99% identical to SEQ ID NO: 564 and the sequence comprises X; or wherein the chimeric polypeptide comprises SEQ ID NO: 564. (211) The chimeric polypeptide of any previous embodiment, comprising a sequence at least about 70%, 75%, 80%, 85%, 90%, 95%, 96%, 97%, 98%, or 99% identical to SEQ ID NO: 565 and the sequence comprises X; or wherein the chimeric polypeptide comprises SEQ ID NO: 565. (212) The chimeric polypeptide of any previous embodiment, comprising a sequence at least about 70%, 75%, 80%, 85%, 90%, 95%, 96%, 97%, 98%, or 99% identical to SEQ ID NO: 566 and the sequence comprises X; or wherein the chimeric polypeptide comprises SEQ ID NO: 566. (213) The chimeric polypeptide of any previous embodiment, comprising a sequence at least about 70%, 75%, 80%, 85%, 90%, 95%, 96%, 97%, 98%, or 99% identical to SEQ ID NO: 567 and the sequence comprises X; or wherein the chimeric polypeptide comprises SEQ ID NO: 567. (214) The chimeric polypeptide of any previous embodiment, comprising a sequence at least about 70%, 75%, 80%, 85%, 90%, 95%, 96%, 97%, 98%, or 99% identical to SEQ ID NO: 568 and the sequence comprises X; or wherein the chimeric polypeptide comprises SEQ ID NO: 568. (215) The chimeric polypeptide of any previous embodiment, comprising a sequence at least about 70%, 75%, 80%, 85%, 90%, 95%, 96%, 97%, 98%, or 99% identical to SEQ ID NO: 569 and the sequence comprises X; or wherein the chimeric polypeptide comprises SEQ ID NO: 569. (216) The chimeric polypeptide of any previous embodiment, comprising a sequence at least about 70%, 75%, 80%, 85%, 90%, 95%, 96%, 97%, 98%, or 99% identical to SEQ ID NO: 570 and the sequence comprises X; or wherein the chimeric polypeptide comprises SEQ ID NO: 570. (217) The chimeric polypeptide of any previous embodiment, comprising a sequence at least about 70%, 75%, 80%, 85%, 90%, 95%, 96%, 97%, 98%, or 99% identical to SEQ ID NO: 571 and the sequence comprises X; or wherein the chimeric polypeptide comprises SEQ ID NO: 571. (218) The chimeric polypeptide of any previous embodiment, comprising a sequence at least about 70%, 75%, 80%, 85%, 90%, 95%, 96%, 97%, 98%, or 99% identical to SEQ ID NO: 572 and the sequence comprises X; or wherein the chimeric polypeptide comprises SEQ ID NO: 572. (219) The chimeric polypeptide of any previous embodiment, comprising a sequence at least about 70%, 75%, 80%, 85%, 90%, 95%, 96%, 97%, 98%, or 99% identical to SEQ ID NO: 573 and the sequence comprises X; or wherein the chimeric polypeptide comprises SEQ ID NO: 573. (220) The chimeric polypeptide of any previous embodiment, comprising a sequence at least about 70%, 75%, 80%, 85%, 90%, 95%, 96%, 97%, 98%, or 99% identical to SEQ ID NO: 574 and the sequence comprises X; or wherein the chimeric polypeptide comprises SEQ ID NO: 574. (221) The chimeric polypeptide of any previous embodiment, comprising a sequence at least about 70%, 75%, 80%, 85%, 90%, 95%, 96%, 97%, 98%, or 99% identical to SEQ ID NO: 575 and the sequence comprises X; or wherein the chimeric polypeptide comprises SEQ ID NO: 575. (222) The chimeric polypeptide of any previous embodiment, comprising a sequence at least about 70%, 75%, 80%, 85%, 90%, 95%, 96%, 97%, 98%, or 99% identical to SEQ ID NO: 576 and the sequence comprises X; or wherein the chimeric polypeptide comprises SEQ ID NO: 577. (223) The chimeric polypeptide of any previous embodiment, comprising a sequence at least about 70%, 75%, 80%, 85%, 90%, 95%, 96%, 97%, 98%, or 99% identical to SEQ ID NO: 578 and the sequence comprises X; or wherein the chimeric polypeptide comprises SEQ ID NO: 579. (224) The chimeric polypeptide of any previous embodiment, comprising a sequence at least about 70%, 75%, 80%, 85%, 90%, 95%, 96%, 97%, 98%, or 99% identical to SEQ ID NO: 580 and the sequence comprises X; or wherein the chimeric polypeptide comprises SEQ ID NO: 580. (225) The chimeric polypeptide of any previous embodiment, comprising a sequence at least about 70%, 75%, 80%, 85%, 90%, 95%, 96%, 97%, 98%, or 99% identical to SEQ ID NO: 581 and the sequence comprises X; or wherein the chimeric polypeptide comprises SEQ ID NO: 581. (226) The chimeric polypeptide of any previous embodiment, comprising a sequence at least about 70%, 75%, 80%, 85%, 90%, 95%, 96%, 97%, 98%, or 99% identical to SEQ ID NO: 582 and the sequence comprises X; or wherein the chimeric polypeptide comprises SEQ ID NO: 582. (227) The chimeric polypeptide of any previous embodiment, comprising a sequence at least about 70%, 75%, 80%, 85%, 90%, 95%, 96%, 97%, 98%, or 99% identical to SEQ ID NO: 583 and the sequence comprises X; or wherein the chimeric polypeptide comprises SEQ ID NO: 583. (228) The chimeric polypeptide of any previous embodiment, comprising a sequence at least about 70%, 75%, 80%, 85%, 90%, 95%, 96%, 97%, 98%, or 99% identical to SEQ ID NO: 584 and the sequence comprises X; or wherein the chimeric polypeptide comprises SEQ ID NO: 584. (229) The chimeric polypeptide of any previous embodiment, comprising a sequence at least about 70%, 75%, 80%, 85%, 90%, 95%, 96%, 97%, 98%, or 99% identical to SEQ ID NO: 585 and the sequence comprises X; or wherein the chimeric polypeptide comprises SEQ ID NO: 585. (230) The chimeric polypeptide of any previous embodiment, comprising a sequence at least about 70%, 75%, 80%, 85%, 90%, 95%, 96%, 97%, 98%, or 99% identical to SEQ ID NO: 586 and the sequence comprises X; or wherein the chimeric polypeptide comprises SEQ ID NO: 586. (231) The chimeric polypeptide of any previous embodiment, comprising a sequence at least about 70%, 75%, 80%, 85%, 90%, 95%, 96%, 97%, 98%, or 99% identical to SEQ ID NO: 587 and the sequence comprises X; or wherein the chimeric polypeptide comprises SEQ ID NO: 587. (232) The chimeric polypeptide of any previous embodiment, comprising a sequence at least about 70%, 75%, 80%, 85%, 90%, 95%, 96%, 97%, 98%, or 99% identical to SEQ ID NO: 588 and the sequence comprises X; or wherein the chimeric polypeptide comprises SEQ ID NO: 588. (233) The chimeric polypeptide of any previous embodiment, comprising a sequence at least about 70%, 75%, 80%, 85%, 90%, 95%, 96%, 97%, 98%, or 99% identical to SEQ ID NO: 589 and the sequence comprises X; or wherein the chimeric polypeptide comprises SEQ ID NO: 589. (234) The chimeric polypeptide of any previous embodiment, comprising a sequence at least about 70%, 75%, 80%, 85%, 90%, 95%, 96%, 97%, 98%, or 99% identical to SEQ ID NO: 590 and the sequence comprises X; or wherein the chimeric polypeptide comprises SEQ ID NO: 590. (235) The chimeric polypeptide of any previous embodiment, comprising a sequence at least about 70%, 75%, 80%, 85%, 90%, 95%, 96%, 97%, 98%, or 99% identical to SEQ ID NO: 591 and the sequence comprises X; or wherein the chimeric polypeptide comprises SEQ ID NO: 591. (236) The chimeric polypeptide of any previous embodiment, comprising a sequence at least about 70%, 75%, 80%, 85%, 90%, 95%, 96%, 97%, 98%, or 99% identical to SEQ ID NO: 592 and the sequence comprises X; or wherein the chimeric polypeptide comprises SEQ ID NO: 592. (237) The chimeric polypeptide of any previous embodiment, comprising a sequence at least about 70%, 75%, 80%, 85%, 90%, 95%, 96%, 97%, 98%, or 99% identical to SEQ ID NO: 593 and the sequence comprises X; or wherein the chimeric polypeptide comprises SEQ ID NO: 593. (238) The chimeric polypeptide of any previous embodiment, comprising a sequence at least about 70%, 75%, 80%, 85%, 90%, 95%, 96%, 97%, 98%, or 99% identical to SEQ ID NO: 594 and the sequence comprises X; or wherein the chimeric polypeptide comprises SEQ ID NO: 594. (239) The chimeric polypeptide of any previous embodiment, comprising a sequence at least about 70%, 75%, 80%, 85%, 90%, 95%, 96%, 97%, 98%, or 99% identical to SEQ ID NO: 595 and the sequence comprises X; or wherein the chimeric polypeptide comprises SEQ ID NO: 595. (240) The chimeric polypeptide of any previous embodiment, comprising a sequence at least about 70%, 75%, 80%, 85%, 90%, 95%, 96%, 97%, 98%, or 99% identical to SEQ ID NO: 596 and the sequence comprises X; or wherein the chimeric polypeptide comprises SEQ ID NO: 596. (241) The chimeric polypeptide of any previous embodiment, comprising a sequence at least about 70%, 75%, 80%, 85%, 90%, 95%, 96%, 97%, 98%, or 99% identical to SEQ ID NO: 597 and the sequence comprises X; or wherein the chimeric polypeptide comprises SEQ ID NO: 597. (242) The chimeric polypeptide of any previous embodiment, comprising a sequence at least about 70%, 75%, 80%, 85%, 90%, 95%, 96%, 97%, 98%, or 99% identical to SEQ ID NO: 598 and the sequence comprises X; or wherein the chimeric polypeptide comprises SEQ ID NO: 598. (243) The chimeric polypeptide of any previous embodiment, comprising a sequence at least about 70%, 75%, 80%, 85%, 90%, 95%, 96%, 97%, 98%, or 99% identical to SEQ ID NO: 599 and the sequence comprises X; or wherein the chimeric polypeptide comprises SEQ ID NO: 599. (244) The chimeric polypeptide of any previous embodiment, comprising a sequence at least about 70%, 75%, 80%, 85%, 90%, 95%, 96%, 97%, 98%, or 99% identical to SEQ ID NO: 600 and the sequence comprises X; or wherein the chimeric polypeptide comprises SEQ ID NO: 600. (245) The chimeric polypeptide of any previous embodiment, comprising a sequence at least about 70%, 75%, 80%, 85%, 90%, 95%, 96%, 97%, 98%, or 99% identical to SEQ ID NO: 601 and the sequence comprises X; or wherein the chimeric polypeptide comprises SEQ ID NO: 601. (246) The chimeric polypeptide of any previous embodiment, comprising a sequence at least about 70%, 75%, 80%, 85%, 90%, 95%, 96%, 97%, 98%, or 99% identical to SEQ ID NO: 602 and the sequence comprises X; or wherein the chimeric polypeptide comprises SEQ ID NO: 602. (247) The chimeric polypeptide of any previous embodiment, comprising a sequence at least about 70%, 75%, 80%, 85%, 90%, 95%, 96%, 97%, 98%, or 99% identical to SEQ ID NO: 603 and the sequence comprises X; or wherein the chimeric polypeptide comprises SEQ ID NO: 603. (248) The chimeric polypeptide of any previous embodiment, comprising a sequence at least about 70%, 75%, 80%, 85%, 90%, 95%, 96%, 97%, 98%, or 99% identical to SEQ ID NO: 604 and the sequence comprises X; or wherein the chimeric polypeptide comprises SEQ ID NO: 604. (249) The chimeric polypeptide of any previous embodiment, comprising a sequence at least about 70%, 75%, 80%, 85%, 90%, 95%, 96%, 97%, 98%, or 99% identical to SEQ ID NO: 605 and the sequence comprises X; or wherein the chimeric polypeptide comprises SEQ ID NO: 605. (250) The chimeric polypeptide of any previous embodiment, comprising a sequence at least about 70%, 75%, 80%, 85%, 90%, 95%, 96%, 97%, 98%, or 99% identical to SEQ ID NO: 606 and the sequence comprises X; or wherein the chimeric polypeptide comprises SEQ ID NO: 606. (251) The chimeric polypeptide of any previous embodiment, comprising a sequence at least about 70%, 75%, 80%, 85%, 90%, 95%, 96%, 97%, 98%, or 99% identical to SEQ ID NO: 607 and the sequence comprises X; or wherein the chimeric polypeptide comprises SEQ ID NO: 607. (252) The chimeric polypeptide of any previous embodiment, comprising a sequence at least about 70%, 75%, 80%, 85%, 90%, 95%, 96%, 97%, 98%, or 99% identical to SEQ ID NO: 608 and the sequence comprises X; or wherein the chimeric polypeptide comprises SEQ ID NO: 608. (253) The chimeric polypeptide of any previous embodiment, comprising a sequence at least about 70%, 75%, 80%, 85%, 90%, 95%, 96%, 97%, 98%, or 99% identical to SEQ ID NO: 609 and the sequence comprises X; or wherein the chimeric polypeptide comprises SEQ ID NO: 609. (254) The chimeric polypeptide of any previous embodiment, comprising a sequence at least about 70%, 75%, 80%, 85%, 90%, 95%, 96%, 97%, 98%, or 99% identical to SEQ ID NO: 610 and the sequence comprises X; or wherein the chimeric polypeptide comprises SEQ ID NO: 610. (255) The chimeric polypeptide of any previous embodiment, comprising a sequence at least about 70%, 75%, 80%, 85%, 90%, 95%, 96%, 97%, 98%, or 99% identical to SEQ ID NO: 611 and the sequence comprises X; or wherein the chimeric polypeptide comprises SEQ ID NO: 611. (256) The chimeric polypeptide of any previous embodiment, comprising a sequence at least about 70%, 75%, 80%, 85%, 90%, 95%, 96%, 97%, 98%, or 99% identical to SEQ ID NO: 612 and the sequence comprises X; or wherein the chimeric polypeptide comprises SEQ ID NO: 612. (257) The chimeric polypeptide of any previous embodiment, comprising a sequence at least about 70%, 75%, 80%, 85%, 90%, 95%, 96%, 97%, 98%, or 99% identical to SEQ ID NO: 613 and the sequence comprises X; or wherein the chimeric polypeptide comprises SEQ ID NO: 613. (258) The chimeric polypeptide of any previous embodiment, comprising a sequence at least about 70%, 75%, 80%, 85%, 90%, 95%, 96%, 97%, 98%, or 99% identical to SEQ ID NO: 614 and the sequence comprises X; or wherein the chimeric polypeptide comprises SEQ ID NO: 614. (259) The chimeric polypeptide of any previous embodiment, comprising a sequence at least about 70%, 75%, 80%, 85%, 90%, 95%, 96%, 97%, 98%, or 99% identical to SEQ ID NO: 615 and the sequence comprises X; or wherein the chimeric polypeptide comprises SEQ ID NO: 615. (260) The chimeric polypeptide of any previous embodiment, comprising a sequence at least about 70%, 75%, 80%, 85%, 90%, 95%, 96%, 97%, 98%, or 99% identical to SEQ ID NO: 616 and the sequence comprises X; or wherein the chimeric polypeptide comprises SEQ ID NO: 616. (261) The chimeric polypeptide of any previous embodiment, comprising a sequence at least about 70%, 75%, 80%, 85%, 90%, 95%, 96%, 97%, 98%, or 99% identical to SEQ ID NO: 617 and the sequence comprises X; or wherein the chimeric polypeptide comprises SEQ ID NO: 617. (262) The chimeric polypeptide of any previous embodiment, comprising a sequence at least about 70%, 75%, 80%, 85%, 90%, 95%, 96%, 97%, 98%, or 99% identical to SEQ ID NO: 618 and the sequence comprises X; or wherein the chimeric polypeptide comprises SEQ ID NO: 618. (263) The chimeric polypeptide of any previous embodiment, comprising a sequence at least about 70%, 75%, 80%, 85%, 90%, 95%, 96%, 97%, 98%, or 99% identical to SEQ ID NO: 619 and the sequence comprises X; or wherein the chimeric polypeptide comprises SEQ ID NO: 619. (264) The chimeric polypeptide of any previous embodiment, comprising a sequence at least about 70%, 75%, 80%, 85%, 90%, 95%, 96%, 97%, 98%, or 99% identical to SEQ ID NO: 620 and the sequence comprises X; or wherein the chimeric polypeptide comprises SEQ ID NO: 620. (265) The chimeric polypeptide of any previous embodiment, comprising a sequence at least about 70%, 75%, 80%, 85%, 90%, 95%, 96%, 97%, 98%, or 99% identical to SEQ ID NO: 621 and the sequence comprises X; or wherein the chimeric polypeptide comprises SEQ ID NO: 621. (266) The chimeric polypeptide of any previous embodiment, comprising a sequence at least about 70%, 75%, 80%, 85%, 90%, 95%, 96%, 97%, 98%, or 99% identical to SEQ ID NO: 622 and the sequence comprises X; or wherein the chimeric polypeptide comprises SEQ ID NO: 622. (267) The chimeric polypeptide of any previous embodiment, comprising a sequence at least about 70%, 75%, 80%, 85%, 90%, 95%, 96%, 97%, 98%, or 99% identical to SEQ ID NO: 623 and the sequence comprises X; or wherein the chimeric polypeptide comprises SEQ ID NO: 623. (268) The chimeric polypeptide of any previous embodiment, comprising a sequence at least about 70%, 75%, 80%, 85%, 90%, 95%, 96%, 97%, 98%, or 99% identical to SEQ ID NO: 624 and the sequence comprises X; or wherein the chimeric polypeptide comprises SEQ ID NO: 624. (269) The chimeric polypeptide of any previous embodiment, comprising a sequence at least about 70%, 75%, 80%, 85%, 90%, 95%, 96%, 97%, 98%, or 99% identical to SEQ ID NO: 625 and the sequence comprises X; or wherein the chimeric polypeptide comprises SEQ ID NO: 625. (270) The chimeric polypeptide of any previous embodiment, comprising a sequence at least about 70%, 75%, 80%, 85%, 90%, 95%, 96%, 97%, 98%, or 99% identical to SEQ ID NO: 626 and the sequence comprises X; or wherein the chimeric polypeptide comprises SEQ ID NO: 626. (271) The chimeric polypeptide of any previous embodiment, comprising a sequence at least about 70%, 75%, 80%, 85%, 90%, 95%, 96%, 97%, 98%, or 99% identical to SEQ ID NO: 627 and the sequence comprises X; or wherein the chimeric polypeptide comprises SEQ ID NO: 627. (272) The chimeric polypeptide of any previous embodiment, comprising a sequence at least about 70%, 75%, 80%, 85%, 90%, 95%, 96%, 97%, 98%, or 99% identical to SEQ ID NO: 628 and the sequence comprises X; or wherein the chimeric polypeptide comprises SEQ ID NO: 628. (273) The chimeric polypeptide of any previous embodiment, comprising a sequence at least about 70%, 75%, 80%, 85%, 90%, 95%, 96%, 97%, 98%, or 99% identical to SEQ ID NO: 629 and the sequence comprises X; or wherein the chimeric polypeptide comprises SEQ ID NO: 629. (274) The chimeric polypeptide of any previous embodiment, comprising a sequence at least about 70%, 75%, 80%, 85%, 90%, 95%, 96%, 97%, 98%, or 99% identical to SEQ ID NO: 630 and the sequence comprises X; or wherein the chimeric polypeptide comprises SEQ ID NO: 630. (275) The chimeric polypeptide of any previous embodiment, comprising a sequence at least about 70%, 75%, 80%, 85%, 90%, 95%, 96%, 97%, 98%, or 99% identical to SEQ ID NO: 631 and the sequence comprises X; or wherein the chimeric polypeptide comprises SEQ ID NO: 631. (276) The chimeric polypeptide of any previous embodiment, comprising a sequence at least about 70%, 75%, 80%, 85%, 90%, 95%, 96%, 97%, 98%, or 99% identical to SEQ ID NO: 632 and the sequence comprises X; or wherein the chimeric polypeptide comprises SEQ ID NO: 632. (277) The chimeric polypeptide of any previous embodiment, comprising a sequence at least about 70%, 75%, 80%, 85%, 90%, 95%, 96%, 97%, 98%, or 99% identical to SEQ ID NO: 633 and the sequence comprises X; or wherein the chimeric polypeptide comprises SEQ ID NO: 633. (278) The chimeric polypeptide of any previous embodiment, comprising a sequence at least about 70%, 75%, 80%, 85%, 90%, 95%, 96%, 97%, 98%, or 99% identical to SEQ ID NO: 634 and the sequence comprises X; or wherein the chimeric polypeptide comprises SEQ ID NO: 634. (279) The chimeric polypeptide of any previous embodiment, comprising a sequence at least about 70%, 75%, 80%, 85%, 90%, 95%, 96%, 97%, 98%, or 99% identical to SEQ ID NO: 635 and the sequence comprises X; or wherein the chimeric polypeptide comprises SEQ ID NO: 635. (280) The chimeric polypeptide of any previous embodiment, comprising a sequence at least about 70%, 75%, 80%, 85%, 90%, 95%, 96%, 97%, 98%, or 99% identical to SEQ ID NO: 636 and the sequence comprises X; or wherein the chimeric polypeptide comprises SEQ ID NO: 636. (281) The chimeric polypeptide of any previous embodiment, comprising a sequence at least about 70%, 75%, 80%, 85%, 90%, 95%, 96%, 97%, 98%, or 99% identical to SEQ ID NO: 637 and the sequence comprises X; or wherein the chimeric polypeptide comprises SEQ ID NO: 637. (282) The chimeric polypeptide of any previous embodiment, comprising a sequence at least about 70%, 75%, 80%, 85%, 90%, 95%, 96%, 97%, 98%, or 99% identical to SEQ ID NO: 638 and the sequence comprises X; or wherein the chimeric polypeptide comprises SEQ ID NO: 638. (283) A composition comprising the chimeric polypeptide of any previous embodiment. (284) The composition of embodiment 283, further comprising a carrier material. (285) The composition of embodiment 284, wherein the targeting polypeptide binds to the carrier material. (286) The composition of embodiment 284 or embodiment 285, wherein the carrier material comprises calcium phosphate. (287) The composition of embodiment 286, wherein the calcium phosphate comprises tri-calcium phosphate. (288) The composition of embodiment 287, wherein the tri-calcium phosphate comprises β-tricalcium phosphate. (289) The composition of any one of embodiments 284-288, wherein the carrier material comprises hydroxyapatite. (290) The composition of any one of embodiments 284-289, wherein the carrier material comprises alpha tricalcium phosphate, fluorapatite, bone (e.g., demineralized bone), glasses (bioglasses) such as silicates, vanadates, and related ceramic minerals, or chelated divalent metal ions, or any combination thereof. (291) The composition of any one of embodiments 284-290, wherein the carrier material comprises a fiber, powder, putty, paste, mesh, sponge, or scaffold, or a combination thereof. (292) A method of treating a subject in need thereof, the method comprising delivering to the subject the chimeric polypeptide of any one of embodiments 1-282. (293) A method of treating a subject in need thereof, the method comprising delivering to the subject the composition of any one of embodiments 283-291. (294) The method of embodiment 292 or embodiment 293, wherein the chimeric polypeptide is used to treat a bone defect in the subject. (295) The method of embodiment 292 or embodiment 293, wherein the chimeric polypeptide is used to treat a cartilage defect in the subject. (296) The method of embodiment 292 or embodiment 293, wherein the chimeric polypeptide is used to treat a soft tissue defect in the subject. (297) The method of embodiment 292 or embodiment 293, wherein the chimeric polypeptide is used to treat a tendon defect in the subject. (298) The method of embodiment 292 or embodiment 293, wherein the chimeric polypeptide is used to treat a fascia defect in the subject. (299) The method of embodiment 292 or embodiment 293, wherein the chimeric polypeptide is used to treat a ligament defect in the subject. (300) The method of embodiment 292 or embodiment 293, wherein the chimeric polypeptide is used to treat an organ defect in the subject. (301) The method of embodiment 292 or embodiment 293, wherein the chimeric polypeptide is used to treat a osteotendinous tissue defect in the subject. (302) The method of embodiment 292 or embodiment 293, wherein the chimeric polypeptide is used to treat a dermal defect in the subject. (303) The method of embodiment 292 or embodiment 293, wherein the chimeric polypeptide is used to treat a osteochondral defect in the subject. (304) The method of embodiment 292 or embodiment 293, wherein the method is performed for spinal fusion in the subject. (305) The method of embodiment 304, wherein the spinal fusion comprises posterior lumbar fusion (PLF). (306) The method of embodiment 304, wherein the spinal fusion comprises interbody fusion. (307) The method of embodiment 292 or embodiment 293, wherein the method is performed for trauma repair of bone. (308) The method of embodiment 292 or embodiment 293, wherein the method is performed for dental repair. (309) The method of embodiment 292 or embodiment 293, wherein the method is performed for craniomaxillofacial repair. (310) The method of embodiment 292 or embodiment 293, wherein the method is performed for ankle fusion. (311) The method of embodiment 292 or embodiment 293, wherein the method is performed for kyphoplasty. (312) The method of embodiment 292 or embodiment 293, wherein the method is performed for balloon osteoplasty. (313) The method of embodiment 292 or embodiment 293, wherein the method is performed for scaphoid facture repair. (314) The method of embodiment 292 or embodiment 293, wherein the method is performed for tendeno-osseous repair. (315) The method of embodiment 292 or embodiment 293, wherein the method is performed to treat osteoporosis. (316) The method of embodiment 292 or embodiment 293, wherein the method is performed to treat avascular necrosis. (317) The method of embodiment 292 or embodiment 293, wherein the method is performed to treat congenital skeletal malformations. (318) The method of embodiment 292 or embodiment 293, wherein the method is performed for costal reconstruction. (319) The method of embodiment 292 or embodiment 293, wherein the method is performed for subchondral bone repair. (320) The method of embodiment 292 or embodiment 293, wherein the method is performed for cartilage repair. (321) The method of embodiment 292 or embodiment 293, wherein the method is performed on a hair follicle (BMP-2 is involved in hair follicle development). (322) The method of any one of embodiments 292-321, wherein the subject is a mammal. (323) The method of any one of embodiments 292-321, wherein the subject is a human. (324) The method of any one of embodiments 292-321, wherein the subject is a non-human mammal. (325) The method of embodiment 324, wherein the method is used in veterinary applications. (326) The method of any one of embodiments 292-325, wherein the delivery comprises surgical implantation into the subject.

A variety of different methods known in the art can be used to determine the K_{D} values of any of the chimeric polypeptides described herein for binding of a target polypeptide to a carrier material, e.g., β-TCP. As non-limiting examples, an electrophoretic mobility shift assay, a filter binding assay, surface plasmon resonance, and a biomolecular binding kinetics assay, etc.

In some embodiments, the chimeric polypeptides provided herein are useful orthopedic materials, and can be used as bone void fillers and for bone reconstructions. In some embodiments, the chimeric polypeptides described herein are osteo-conductive and easily resorbed.

### Linkers

In some instances, a neighboring pair of two targeting polypeptides directly abut each other. In some instances, a neighboring pair of two targeting polypeptides are separated by a linker sequence.

In some embodiments of a chimeric polypeptide provided herein comprising a targeting polypeptide and a therapeutic agent, the targeting polypeptide and therapeutic agent are separated by a linker sequence. In some embodiments, a linker sequence is present between two targeting polypeptides and/or between a targeting polypeptide and a therapeutic agent. In some instances, the therapeutic agent comprises a mammalian growth factor. For instance, BMP-2 comprising SEQ ID NO: 32. In some instances, the targeting polypeptide comprises one or more sequences of Table B. In some embodiments of a chimeric polypeptide comprising a linker positioned between the targeting polypeptide and a therapeutic agent, the presence of the linker increases solubility of the chimeric polypeptide as compared to a chimeric polypeptide without the linker. In some embodiments, the linker is optimized in sequence and/or length. In some embodiments, a chimeric polypeptide comprising the linker is more soluble than the chimeric polypeptide without the linker. In some embodiments, a therapeutic agent in a chimeric polypeptide comprising the linker is more bioavailable than without the linker.

In some embodiments, the linker comprises a sequence of about 5 to about 50 amino acids. In some embodiments, the linker is a flexible linker, where at least about five of the amino acids have no regular secondary structure. In some embodiments, regular secondary structure comprises any helical structure like an alpha helix or 3₁₀ helix or π helix, a beta turn, omega loop, and/or a beta sheet. In some embodiments, the linker comprises at least about 50%, 55%, 60%, 65%, 70%, 75%, 80%, 85% or 90% glycine, alanine, serine, glycine and alanine, glycine and serine, alanine and serine, or glycine, alanine, and serine. In some embodiments, the linker comprises at least about 50%, 55%, 60%, 65%, 70%, 75%, 80%, 85% or 90% a combination of alanine, serine, glycine, and threonine. In some embodiments, the linker comprises 1, 2, 3, 4, or 5 sequences having GSEG (SEQ ID NO: 702). In some embodiments, the linker comprises 1, 2, 3, 4, or 5 sequences having SEGG (SEQ ID NO: 703). In some embodiments, a linker comprising a majority of A, S, G, and T provide improved solubility of the chimeric polypeptide. In some embodiments, a linker comprising a length of about 30 to about 45 amino acids provides improved solubility of the chimeric polypeptide.

In some embodiments, the linker comprises a sequence at least about 70%, 75%, 80%, 85%, 90%, 95%, or 100% identical to ASGAGGSEGGGSEGGTSGATGAGTSTSGGGASTGGGTG (SEQ ID NO: 701). In some embodiments, the linker comprises at least about 10, 15, 20, 25, or 30 contiguous amino acids of SEQ ID NO: 701. In some embodiments, the linker comprises a sequence at least about 90% identical to SEQ ID NO: 701. In some embodiments, the linker comprises SEQ ID NO: 701. In some embodiments, the linker sequence is positioned between a targeting polypeptide and a therapeutic agent. In some instances, the therapeutic agent comprises a mammalian growth factor. For instance, BMP-2 comprising SEQ ID NO: 32. In some instances, the targeting polypeptide comprises one or more sequences of Table A.

In some embodiments, a linker sequence comprises or consists of TGGSGEGGTGASTGGSAGTGGSGGTTSGEAGGSSGAG (SEQ ID NO: 33) or GSGATG (SEQ ID NO: 45). In some instances the chimeric polypeptide comprises a linker that has an amino acid sequence that has at least 70% (e.g., at least 75%, at least 80%, at least 85%, at least 90%, at least 95%, at least 96%, at least 97%, at least 98%, at least 99%) sequence identity to SEQ ID NO: 33.

In some embodiments, a linker sequence comprises GSGS (SEQ ID NO: 705), GSGSGS (SEQ ID NO: 706), SGSG (SEQ ID NO: 707), SGSGSG (SEQ ID NO: 708), GSSG (SEQ ID NO: 709), SS, or GGGGS (SEQ ID NO: 710), or a combination thereof.

In some embodiments, a linker sequence can be 1 amino acid to about 100 amino acids, 1 amino acid to about 95 amino acids, 1 amino acid to about 90 amino acids, 1 amino acid to about 85 amino acids, 1 amino acid to about 80 amino acids, 1 amino acid to about 75 amino acids, 1 amino acid to about 70 amino acids, 1 amino acid to about 65 amino acids, 1 amino acid to about 60 amino acids, 1 amino acid to about 55 amino acids, 1 amino acid to about 50 amino acids, 1 amino acid to about 45 amino acids, 1 amino acid to about 40 amino acids, 1 amino acid to about 35 amino acids, 1 amino acid to about 30 amino acids, 1 amino acid to about 25 amino acids, 1 amino acid to about 20 amino acids, 1 amino acid to about 15 amino acids, 1 amino acid to about 10 amino acids, 1 amino acid to about 5 amino acids, about 5 amino acids to about 100 amino acids, about 5 amino acids to about 95 amino acids, about 5 amino acids to about 90 amino acids, about 5 amino acids to about 85 amino acids, about 5 amino acids to about 80 amino acids, about 5 amino acids to about 75 amino acids, about 5 amino acids to about 70 amino acids, about 5 amino acids to about 65 amino acids, about 5 amino acids to about 60 amino acids, about 5 amino acids to about 55 amino acids, about 5 amino acids to about 50 amino acids, about 5 amino acids to about 45 amino acids, about 5 amino acids to about 40 amino acids, about 5 amino acids to about 35 amino acids, about 5 amino acids to about 30 amino acids, about 5 amino acids to about 25 amino acids, about 5 amino acids to about 20 amino acids, about 5 amino acids to about 15 amino acids, about 5 amino acids to about 10 amino acids, about 10 amino acids to about 100 amino acids, about 10 amino acids to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids.

### Growth Factors

A targeting polypeptide as described herein can be tethered to a mammalian growth factor. In some instances, a linker sequence (e.g., any of the linker sequences described herein or known in the art) is disposed between the targeting polypeptide and the mammalian growth factor.

Mammalian growth factors can be osteoinductive molecules that are capable of initiating and enhancing the bone repair process. Bone morphogenetic proteins (BMP) represent a distinct subset of the transforming growth factor-β (TGFbeta) family. A number of these BMP (BMP-2, BMP-7, and BMP-14) enhance the speed of bone healing in defects and non-unions.

Non-limiting examples of mammalian growth factors are described herein. In some instances, the mammalian growth factor comprises: epidermal growth factor (EGF), platelet derived growth factor (PDGF), insulin like growth factor (IGF-1), fibroblast growth factor (FGF), fibroblast growth factor 2 (FGF2), fibroblast growth factor 18 (FGF18), transforming growth factor alpha (TGF-α), transforming growth factor beta (TGF-β), transforming growth factor beta 1 (TGF-β1), transforming growth factor beta 3 (TGF-β3), osteogenic protein 1 (OP-1), osteogenic protein 2 (OP-2), osteogenic protein 3 (OP-3), bone morphogenetic protein 2 (BMP-2), bone morphogenetic protein 3 (BMP-3), bone morphogenetic protein 4 (BMP-4), bone morphogenetic protein 5 (BMP-5), bone morphogenetic protein 6 (BMP-6), bone morphogenetic protein 7 (BMP-7), bone morphogenetic protein (BMP-9), bone morphogenetic protein 10 (BMP-10), bone morphogenetic protein 11 (BMP-11), bone morphogenetic protein 12 (BMP-12), bone morphogenetic protein 13 (BMP-13), bone morphogenetic protein 15 (BMP-15), dentin phosphoprotein (DPP), vegetal related growth factor (Vgr), growth differentiation factor 1 (GDF-1), growth differentiation factor 3 (GDF-3), growth differentiation factor 5 (GDF-5), growth differentiation factor 6 (GDF-6), growth differentiation factor 7 (GDF-7), growth differentiation factor 8 (GDF8), growth differentiation factor 11 (GDF11), growth differentiation factor 15 (GDF15), vascular endothelial growth factor (VEGF), hyaluronic acid binding protein (HABP), and collagen binding protein (CBP), fibroblast growth factor 18 (FGF-18), keratinocyte growth factor (KGF), tumor necrosis factor alpha (TNFα), tumor necrosis factor (TNF)-related apoptosis inducing ligand (TRAIL), wnt family member 1 (WNT1), wnt family member 2 (WNT2), wnt family member 2B (WNT2B), wnt family member 3 (WNT3), wnt family member 3A (WNT3A), wnt family member 4 (WNT4), wnt family member 5A (WNT5A), wnt family member 5B (WNT5B), wnt family member 6 (WNT6), wnt family member 7A (WNT7A), wnt family member 7B (WNT7B), wnt family member 8A (WNT8A), wnt family member 8B (WNT8B), wnt family member 9A (WNT9A), wnt family member 9B (WNT9B), wnt family member 10A (WNT10A), wnt family member 10B (WNT10B), wnt family member 11 (WNT11), neuregulin 1 (NRG1), or wnt family member 16 (WNT16), or a mature peptide or functional portion thereof. In some embodiments, a chimeric polypeptide provided herein comprises one or more mammalian growth factors.

For example, the mammalian growth factor can be a human growth factor. Non-limiting examples of mammalian growth factors and mature peptides and/or functional portions thereof are provided in Table B. As used herein, reference to a mammalian growth factor includes a non-human mammalian growth factor. For instance, a mammalian growth factor includes a non-human mammalian growth factor homologous to a human growth factor, such as one or more of the human growth factors of Table B.

In some embodiments, a non-human mammalian growth factor is homologous to a human growth factor if the non-human mammalian growth factor is at least about 80% identical to the human mammalian growth factor as determined using the NCBI Blast alignment algorithm as of the date of this filing. In some cases, the coverage is at least about 90%.

In some embodiments, a non-human mammalian growth factor is homologous to a human growth factor if the non-human mammalian growth factor has at least about 80% positives as compared to the human mammalian growth factor as determined using the NCBI Blast alignment algorithm as of the date of this filing. In some cases, the coverage is at least about 90%.

In some embodiments, a non-human mammalian growth factor is homologous to a human growth factor if the non-human mammalian growth factor aligned with the human growth factor using the NCBI Blast as of the date of this filing has an E value of less than about 1E-40, at least about 1E-50, 1E-60, 1E-70, or 1E-10, with a query cover of at least about 90%.

TABLE B
Exemplary human growth factors

| | | SEQ ID |
|---|---|---|
| Name | Exemplary Human Protein Sequence | NO: |
| EGF | NSDSECPLSHDGYCLHDGVCMYIEALDKYACNCVVGYIGERCQY | 46 |
| | RDLKWWELR | |
| PDGF | EEAEIPREVIERLARSQIHSIRDLQRLLEIDSVGSEDSLDTSLRAHGV | 47 |
| | HATKHVPEKRPLPIRRKR | |
| IGF-1 | GPETLCGAELVDALQFVCGDRGFYFNKPTGYGSSSRRAPQTGIVDE | 48 |
| | CCFRSCDLRRLEMYCAPLKPAKSA | |
| FGF | FNLPPGNYKKPKLLYCSNGGHFLRILPDGTVDGTRDRSDQHIQLQL | 49 |
| | SAESVGEVYIKSTETGQYLAMDTDGLLYGSQTPNEECLFLERLEEN | |
| | HYNTYISKKHAEKNWFVGLKKNGSCKRGPRTHYGQKAILFLPLPV | |
| | SSD | |
| FGF2 | PALPEDGGSGAFPPGHFKDPKRLYCKNGGFFLRIHPDGRVDGVRE | 50 |
| | KSDPHIKLQLQAEERGVVSIKGVCANRYLAMKEDGRLLASKCVTD | |
| | ECFFFERLESNNYNTYRSRKYTSWYVALKRTGQYKLGSKTGPGQK | |
| | AILFLPMSAKS | |
| FGF18 | EENVDFRIHVENQTRARDDVSRKQLRLYQLYSRTSGKHIQVLGRRI | 51 |
| | SARGEDGDKYAQLLVETDTFGSQVRIKGKETEFYLCMNRKGKLV | |
| | GKPDGTSKECVFIEKVLENNYTALMSAKYSGWYVGFTKKGRPRK | |
| | GPKTRENQQDVHFMKRYPKGQPELQKPFKYTTVTKRSRRIRPTHP | |
| | A | |
| TGF-α | ENSTSPLSADPPVAAAVVSHFNDCPDSHTQFCFHGTCRFLVQEDKP | 52 |
| | ACVCHSGYVGARCEHADLLAVVAASQKKQAITALVVVSIVALAV | |
| | LIITCVLIHCCQVRKHCEWCRALICRHEKPSALLKGRTACCHSETV | |
| | V | |
| TGF-α | VVSHFNDCPDSHTQFCFHGTCRFLVQEDKPACVCHSGYVGARCEH | 152 |
| | ADLLA | |
| TGF-β1 | ALDTNYCFSSTEKNCCVRQLYIDFRKDLGWKWIHEPKGYHANFCL | 53 |
| | GPCPYIWSLDTQYSKVLALYNQHNPGASAAPCCVPQALEPLPIVYY | |
| | VGRKPKVEQLSNMIVRSCKCS | |
| TGF-β3 | ALDTNYCFRNLEENCCVRPLYIDFRQDLGWKWVHEPKGYYANFC | 54 |
| | SGPCPYLRSADTTHSTVLGLYNTLNPEASASPCCVPQDLEPLTILYY | |
| | VGRTPKVEQLSNMVVKSCKCS | |
| OP-2 | AVRPLRRRQPKKSNELPQANRLPGIFDDVHGSHGRQVCRRHELYV | 55 |
| (BMP-8) | SFQDLGWLDWVIAPQGYSAYYCEGECSFPLDSCMNATNHAILQSL | |
| | VHLMMPDAVPKACCAPTKLSATSVLYYDSSNNVILRKHRNMVVK | |
| | ACGCH | |
| BMP8A | AVRPLRRRQPKKSNELPQANRLPGIFDDVRGSHGRQVCRRHELYV | 56 |
| | SFQDLGWLDWVIAPQGYSAYYCEGECSFPLDSCMNATNHAILQSL | |
| | VHLMKPNAVPKACCAPTKLSATSVLYYDSSNNVILRKHRNMVVK | |
| | ACGCH | |
| BMP-2 | QAKHKQRKRLKSSCKRHPLYVDFSDVGWNDWIVAPPGYHAFYCH | 32 |
| | GECPFPLADHLNSTNHAIVQTLVNSVNSKIPKACCVPTELSAISMLY | |
| | LDENEKVVLKNYQDMVVEGCGCR | |
| BMP-3 | QWIEPRNCARRYLKVDFADIGWSEWIISPKSFDAYYCSGACQFPMP | 57 |
| | KSLKPSNHATIQSIVRAVGVVPGIPEPCCVPEKMSSLSILFFDENKN | |
| | VVLKVYPNMTVESCACR | |
| BMP-4 | SPKHHSQRARKKNKNCRRHSLYVDFSDVGWNDWIVAPPGYQAFY | 58 |
| | CHGDCPFPLADHLNSTNHAIVQTLVNSVNSSIPKACCVPTELSAISM | |
| | LYLDEYDKVVLKNYQEMVVEGCGCR | |
| BMP-5 | AANKRKNQNRNKSSSHQDSSRMSSVGDYNTSEQKQACKKHELYV | 59 |
| | SFRDLGWQDWIIAPEGYAAFYCDGECSFPLNAHMNATNHAIVQTL | |
| | VHLMFPDHVPKPCCAPTKLNAISVLYFDDSSNVILKKYRNMVVRS | |
| | CGCH | |
| BMP- | SASSRRRQQSRNRSTQSQDVARVSSASDYNSSELKTACRKHELYV | 60 |
| 6/VGR | SFQDLGWQDWIIAPKGYAANYCDGECSFPLNAHMNATNHAIVQT | |
| | LVHLMNPEYVPKPCCAPTKLNAISVLYFDDNSNVILKKYRNMVVR | |
| | ACGCH | |
| BMP- | STGSKQRSQNRSKTPKNQEALRMANVAENSSSDQRQACKKHELY | 61 |
| 7/OP-l | VSFRDLGWQDWIIAPEGYAAYYCEGECAFPLNSYMNATNHAIVQT | |
| | LVHFINPETVPKPCCAPTQLNAISVLYFDDSSNVILKKYRNMVVRA | |
| | CGCH | |
| BMP-9 | SAGAGSHCQKTSLRVNFEDIGWDSWIIAPKEYEAYECKGGCFFPLA | 62 |
| | DDVTPTKHAIVQTLVHLKFPTKVGKACCVPTKLSPISVLYKDDMG | |
| | VPTLKYHYEGMSVAECGCR | |
| BMP-10 | NAKGNYCKRTPLYIDFKEIGWDSWIIAPPGYEAYECRGVCNYPLAE | 63 |
| | HLTPTKHAIIQALVHLKNSQKASKACCVPTKLEPISILYLDKGVVTY | |
| | KFKYEGMAVSECGCR | |
| BMP- | NLGLDCDEHSSESRCCRYPLTVDFEAFGWDWIIAPKRYKANYCSG | 64 |
| 11/GDF-11 | QCEYMFMQKYPHTHLVQQANPRGSAGPCCTPTKMSPINMLYFND | |
| | KQQIIYGKIPGMVVDRCGCS | |
| BMP-12 | TALAGTRTAQGSGGGAGRGHGRRGRSRCSRKPLHVDFKELGWDD | 65 |
| | WIIAPLDYEAYHCEGLCDFPLRSHLEPTNHAIIQTLLNSMAPDAAPA | |
| | SCCVPARLSPISILYIDAANNVVYKQYEDMVVEACGCR | |
| BMP- | TAFASRHGKRHGKKSRLRCSKKPLHVNFKELGWDDWIIAPLEYEA | 66 |
| 13/GDF-6 | YHCEGVCDFPLRSHLEPTNHAIIQTLMNSMDPGSTPPSCCVPTKLTP | |
| | ISILYIDAGNNVVYKQYEDMVVESCGCR | |
| BMP-15 | QADGISAEVTASSSKHSGPENNQCSLHPFQISFRQLGWDHWIIAPPF | 67 |
| | YTPNYCKGTCLRVLRDGLNSPNHAIIQNLINQLVDQSVPRPSCVPY | |
| | KYVPISVLMIEANGSILYKEYEGMIAESCTCR | |
| DPP | IPVPQSKPLERHVEKSMNLHLLARSNVSVQDELNASGTIKESGVLV | 68 |
| isoform 1 | HEGDRGRQENTQDGHKGEGNGSKWAEVGGKSFSTYSTLANEEGN | |
| | IEGWNGDTGKAETYGHDGIHGKEENITANGIQGQVSIIDNAGATNR | |
| | SNTNGNTDKNTQNGDVGDAGHNEDVAVVQEDGPQVAGSNNSTD | |
| | NEDEIIENSCRNEGNTSEITPQINSKRNGTKEAEVTPGTGEDAGLDN | |
| | SDGSPSGNGADEDEDEGSGDDEDEEAGNGKDSSNNSKGQEGQDH | |
| | GKEDDHDSSIGQNSDSKEYYDPEGKEDPHNEVDGDKTSKSEENSA | |
| | GIPEDNGSQRIEDTQKLNHRESKRVENRITKESETHAVGKSQDKGI | |
| | EIKGPSSGNRNITKEVGKGNEGKEDKGQHGMILGKGNVKTQGEVV | |
| | NIEGPGQKSEPGNKVGHSNTGSDSNSDGYDSYDFDDKSMQG | |
| DPP | IPVPQSKPLERHVEKSMNLHLLARSNVSVQDELNASGTIKESGVLV | 168 |
| isoform 2 | HEGDRGRQENTQDGHKGEGNGSKWAEVGGKSFSTYSTLANEEGN | |
| | IEGWNGDTGKAETYGHDGIHGKEENITANGIQGQVSIIDNAGATNR | |
| | SNTNGNTDKNTQNGDVGDAGHNEDVAVVQEDGPQVAGSNNSTD | |
| | NEDEIIENSCRNEGNTSEITPQINSKRNGTKEAEVTPGTGEDAGLDN | |
| | SDGSPSGNGADEDEDEGSGDDEDEEAGNGKDSSNNSKGQEGQDH | |
| | GKEDDHDSSIGQNSDSKEYYDPEGKEDPHNEVDGDKTSKSEENSA | |
| | GIPEDNGSQRIEDTQKLNHRESKRVENRITKESETHAVGKSQDKGI | |
| | EIKGPSSGNRNITKEVGKGNEGKEDKGQHGMILGKGNVKTQGEVV | |
| | NIEGPGQKSEPGNKVGHSNTGSDSNSDGYDSYDFDDKSMQGDDP | |
| | NSSDESNGNDDANSESDNNSSSRGDASYNSDESKDNGNGSDSKGA | |
| | EDDDSDSTSDTNNSDSNGNGNNGNDDNDKSDSGKGKSDSSDSDSS | |
| | DSSNSSDSSDSSDSDSSDSNSSSDSDSSDSDSSDSSDSDSSDSSNSSD | |
| | SSDSSDSSDSSDSSDSSDSKSDSSKSESDSSDSDSKSDSSDSNSSDSS | |
| | DNSDSSDSSNSSNSSDSSDSSDSSDSSSSSDSSNSSDSSDSSDSSNSSE | |
| | SSDSSDSSDSDSSDSSDSSNSNSSDSDSSNSSDSSDSSNSSDSSDSSDS | |
| | SNSSDSSDSSDSSNSSDSSDSSDSSDSSDSSNSSDSNDSSNSSDSSDSS | |
| | NSSDSSNSSDSSDSSDSSDSDSSNSSDSSNSSDSSDSSNSSDSSDSSDS | |
| | SDGSDSDSSNRSDSSNSSDSSDSSDSSNSSDSSDSSDSNESSNSSDSS | |
| | DSSNSSDSDSSDSSNSSDSSDSSNSSDSSESSNSSDNSNSSDSSNSSD | |
| | SSDSSDSSNSSDSSNSSDSSNSSDSSDSNSSDSSDSSNSSDSSDSSDSS | |
| | DSSDSSDSSNSSDSSDSSDSSDSSNSSDSSNSSDSSNSSDSSDSSDSSD | |
| | SSDSSDSSDSSDSSNSSDSSDSSDSSDSSDSSDSSDSSDSSESSDSSDS | |
| | SNSSDSSDSSDSSDSSDSSDSSDSSDSSDSSNSSDSSDSSDSSDSSDSS | |
| | NSSDSSDSSESSDSSDSSDSSDSSDSSDSSDSSDSSDSSNSSDSSDSSD | |
| | SSDSSDSSDSSDSSDSSDSSDSSDSSDSSDSSDSSDSSDSSDSNESSDS | |
| | SDSSDSSDSSNSSDSSDSSDSSDSTSDSNDESDSQSKSGNGNNNGSD | |
| | SDSDSEGSDSNHSTSDD | |
| DPP | DDPNSSDESNGNDDANSESDNNSSSRGDASYNSDESKDNGNGSDS | 268 |
| isoform 3 | KGAEDDDSDSTSDTNNSDSNGNGNNGNDDNDKSDSGKGKSDSSD | |
| | SDSSDSSNSSDSSDSSDSDSSDSNSSSDSDSSDSDSSDSSDSDSSDSS | |
| | NSSDSSDSSDSSDSSDSSDSSDSKSDSSKSESDSSDSDSKSDSSDSNS | |
| | SDSSDNSDSSDSSNSSNSSDSSDSSDSSDSSSSSDSSNSSDSSDSSDSS | |
| | NSSESSDSSDSSDSDSSDSSDSSNSNSSDSDSSNSSDSSDSSNSSDSS | |
| | DSSDSSNSSDSSDSSDSSNSSDSSDSSDSSDSSDSSNSSDSNDSSNSS | |
| | DSSDSSNSSDSSNSSDSSDSSDSSDSDSSNSSDSSNSSDSSDSSNSSDS | |
| | SDSSDSSDGSDSDSSNRSDSSNSSDSSDSSDSSNSSDSSDSSDSNESS | |
| | NSSDSSDSSNSSDSDSSDSSNSSDSSDSSNSSDSSESSNSSDNSNSSD | |
| | SSNSSDSSDSSDSSNSSDSSNSSDSSNSSDSSDSNSSDSSDSSNSSDSS | |
| | DSSDSSDSSDSSDSSNSSDSSDSSDSSDSSNSSDSSNSSDSSNSSDSSD | |
| | SSDSSDSSDSSDSSDSSDSSNSSDSSDSSDSSDSSDSSDSSDSSDSSES | |
| | SDSSDSSNSSDSSDSSDSSDSSDSSDSSDSSDSSDSSNSSDSSDSSDSS | |
| | DSSDSSNSSDSSDSSESSDSSDSSDSSDSSDSSDSSDSSDSSDSSNSSD | |
| | SSDSSDSSDSSDSSDSSDSSDSSDSSDSSDSSDSSDSSDSSDSSDSSDS | |
| | NESSDSSDSSDSSDSSNSSDSSDSSDSSDSTSDSNDESDSQSKSGNG | |
| | NNNGSDSDSDSEGSDSNHSTSDD | |
| GDF-1 | DAEPVLGGGPGGACRARRLYVSFREVGWHRWVIAPRGFLANYCQ | 69 |
| | GQCALPVALSGSGGPPALNHAVLRALMHAAAPGAADLPCCVPAR | |
| | LSPISVLFFDNSDNVVLRQYEDMVVDECGCR | |
| GDF-3 | AAIPVPKLSCKNLCHRHQLFINFRDLGWHKWIIAPKGFMANYCHG | 70 |
| | ECPFSLTISLNSSNYAFMQALMHAVDPEIPQAVCIPTKLSPISMLYQ | |
| | DNNDNVILRHYEDMVVDECGCG | |
| GDF-5 | APLATRQGKRPSKNLKARCSRKALHVNFKDMGWDDWIIAPLEYE | 71 |
| | AFHCEGLCEFPLRSHLEPTNHAVIQTLMNSMDPESTPPTCCVPTRLS | |
| | PISILFIDSANNVVYKQYEDMVVESCGCR | |
| GDF8 | DFGLDCDEHSTESRCCRYPLTVDFEAFGWDWIIAPKRYKANYCSG | 73 |
| | ECEFVFLQKYPHTHLVHQANPRGSAGPCCTPTKMSPINMLYFNGK | |
| | EQIIYGKIPAMVVDRCGCS | |
| GDF15 | ARARNGDHCPLGPGRCCRLHTVRASLEDLGWADWVLSPREVQVT | 74 |
| | MCIGACPSQFRAANMHAQIKTSLHRLKPDTVPAPCCVPASYNPMV | |
| | LIQKTDTGVSLQTYDDLLAKDCHCI | |
| VEGF | APMAEGGGQNHHEVVKFMDVYQRSYCHPIETLVDIFQEYPDEIEYI | 75 |
| | FKPSCVPLMRCGGCCNDEGLECVPTEESNITMQIMRIKPHQGQHIG | |
| | EMSFLQHNKCECRPKKDRARQEKKSVRGKGKGQKRKRKKSRYKS | |
| | WSVYVGARCCLMPWSLPGPHPCGPCSERRKHLFVQDPQTCKCSC | |
| | KNTDSRCKARQLELNERTCRCDKPRR | |
| HABP | FSLMSLLESLDPDWTPDQYDYSYEDYNQEENTSSTLTHAENPDWY | 76 |
| Isoform 1 | YTEDQADPCQPNPCEHGGDCLVHGSTFTCSCLAPFSGNKCQKVQN | |
| | TCKDNPCGRGQCLITQSPPYYRCVCKHPYTGPSCSQVVPVCRPNPC | |
| | QNGATCSRHKRRSKFTCACPDQFKGKFCEIGSDDCYVGDGYSYRG | |
| | KMNRTVNQHACLYWNSHLLLQENYNMFMEDAETHGIGEHNFCR | |
| | NPDADEKPWCFIKVTNDKVKWEYCDVSACSAQDVAYPEESPTEPS | |
| | TKLPGFDSCGKTEIAERKIKR | |
| HABP | IYGGFKSTAGKHPWQASLQSSLPLTISMPQGHFCGGALIHPCWVLT | 176 |
| Isoform 2 | AAHCTDIKTRHLKVVLGDQDLKKEEFHEQSFRVEKIFKYSHYNER | |
| | DEIPHNDIALLKLKPVDGHCALESKYVKTVCLPDGSFPSGSECHISG | |
| | WGVTETGKGSRQLLDAKVKLIANTLCNSRQLYDHMIDDSMICAG | |
| | NLQKPGQDTCQGDSGGPLTCEKDGTYYVYGIVSWGLECGKRPGV | |
| | YTQVTKFLNWIKATIKSESGF | |
| CBP | AEVKKPAAAAAPGTAEKLSPKAATLAERSAGLAFSLYQAMAKDQ | 77 |
| | AVENILVSPVVVASSLGLVSLGGKATTASQAKAVLSAEQLRDEEV | |
| | HAGLGELLRSLSNSTARNVTWKLGSRLYGPSSVSFADDFVRSSKQ | |
| | HYNCEHSKINFRDKRSALQSINEWAAQTTDGKLPEVTKDVERTDG | |
| | ALLVNAMFFKPHWDEKFHHKMVDNRGFMVTRSYTVGVMMMHR | |
| | TGLYNYYDDEKEKLQIVEMPLAHKLSSLIILMPHHVEPLERLEKLL | |
| | TKEQLKIWMGKMQKKAVAISLPKGVVEVTHDLQKHLAGLGLTEA | |
| | IDKNKADLSRMSGKKDLYLASVFHATAFELDTDGNPFDQDIYGRE | |
| | ELRSPKLFYADHPFIFLVRDTQSGSLLFIGRLVRPKGDKMRDEL | |
| KGF | CNDMTPEQMATNVNCSSPERHTRSYDYMEGGDIRVRRLFCRTQW | 79 |
| | YLRIDKRGKVKGTQEMKNNYNIMEIRTVAVGIVAIKGVESEFYLA | |
| | MNKEGKLYAKKECNEDCNFKELILENHYNTYASAKWTHNGGEMF | |
| | VALNQKGIPVRGKKTKKEQKTAHFLPMAIT | |
| TNFα | GPQREEFPRDLSLISPLAQAVRSSSRTPSDKPVAHVVANPQAEGQL | 80 |
| | QWLNRRANALLANGVELRDNQLVVPSEGLYLIYSQVLFKGQGCPS | |
| | THVLLTHTISRIAVSYQTKVNLLSAIKSPCQRETPEGAEAKPWYEPI | |
| | YLGGVFQLEKGDRLSAEINRPDYLDFAESGQVYFGIIAL | |
| TRAIL | TNELKQMQDKYSKSGIACFLKEDDSYWDPNDEESMNSPCWQVK | 81 |
| | WQLRQLVRKMILRTSEETISTVQEKQQNISPLVRERGPQRVAAHIT | |
| | GTRGRSNTLSSPNSKNEKALGRKINSWESSRSGHSFLSNLHLRNGE | |
| | LVIHEKGFYYIYSQTYFRFQEEIKENTKNDKQMVQYIYKYTSYPDPI | |
| | LLMKSARNSCWSKDAEYGLYSIYQGGIFELKENDRIFVSVTNEHLI | |
| | DMDHEASFFGAFLVG | |
| WNT1 | ANSSGRWWGIVNVASSTNLLTDSKSLQLVLEPSLQLLSRKQRRLIR | 82 |
| | QNPGILHSVSGGLQSAVRECKWQFRNRRWNCPTAPGPHLFGKIVN | |
| | RGCRETAFIFAITSAGVTHSVARSCSEGSIESCTCDYRRRGPGGPDW | |
| | HWGGCSDNIDFGRLFGREFVDSGEKGRDLRFLMNLHNNEAGRTT | |
| | VFSEMRQECKCHGMSGSCTVRTCWMRLPTLRAVGDVLRDRFDGA | |
| | SRVLYGNRGSNRASRAELLRLEPEDPAHKPPSPHDLVYFEKSPNFC | |
| | TYSGRLGTAGTAGRACNSSSPALDGCELLCCGRGHRTRTQRVTER | |
| | CNCTFHWCCHVSCRNCTHTRVLHECL | |
| WNT2 | SWWYMRATGGSSRVMCDNVPGLVSSQRQLCHRHPDVMRAISQG | 83 |
| | VAEWTAECQHQFRQHRWNCNTLDRDHSLFGRVLLRSSRESAFVY | |
| | AISSAGVVFAITRACSQGEVKSCSCDPKKMGSAKDSKGIFDWGGC | |
| | SDNIDYGIKFARAFVDAKERKGKDARALMNLHNNRAGRKAVKRF | |
| | LKQECKCHGVSGSCTLRTCWLAMADFRKTGDYLWRKYNGAIQV | |
| | VMNQDGTGFTVANERFKKPTKNDLVYFENSPDYCIRDREAGSLGT | |
| | AGRVCNLTSRGMDSCEVMCCGRGYDTSHVTRMTKCGCKFHWCC | |
| | AVRCQDCLEALDVHTCKAPKNADWTTAT | |
| WNT2B | SWWYIGALGARVICDNIPGLVSRQRQLCQRYPDIMRSVGEGAREW | 84 |
| | IRECQHQFRHHRWNCTTLDRDHTVFGRVMLRSSREAAFVYAISSA | |
| | GVVHAITRACSQGELSVCSCDPYTRGRHHDQRGDFDWGGCSDNIH | |
| | YGVRFAKAFVDAKEKRLKDARALMNLHNNRCGRTAVRRFLKLEC | |
| | KCHGVSGSCTLRTCWRALSDFRRTGDYLRRRYDGAVQVMATQD | |
| | GANFTAARQGYRRATRTDLVYFDNSPDYCVLDKAAGSLGTAGRV | |
| | CSKTSKGTDGCEIMCCGRGYDTTRVTRVTQCECKFHWCCAVRCK | |
| | ECRNTVDVHTCKAPKKAEWLDQT | |
| WNT3 | GYPIWWSLALGQQYTSLGSQPLLCGSIPGLVPKQLRFCRNYIEIMPS | 85 |
| | VAEGVKLGIQECQHQFRGRRWNCTTIDDSLAIFGPVLDKATRESAF | |
| | VHAIASAGVAFAVTRSCAEGTSTICGCDSHHKGPPGEGWKWGGCS | |
| | EDADFGVLVSREFADARENRPDARSAMNKHNNEAGRTTILDHMH | |
| | LKCKCHGLSGSCEVKTCWWAQPDFRAIGDFLKDKYDSASEMVVE | |
| | KHRESRGWVETLRAKYSLFKPPTERDLVYYENSPNFCEPNPETGSF | |
| | GTRDRTCNVTSHGIDGCDLLCCGRGHNTRTEKRKEKCHCIFHWCC | |
| | YVSCQECIRIYDVHTCK | |
| WNT3A | SYPIWWSLAVGPQYSSLGSQPILCASIPGLVPKQLRFCRNYVEIMPS | 86 |
| | VAEGIKIGIQECQHQFRGRRWNCTTVHDSLAIFGPVLDKATRESAF | |
| | VHAIASAGVAFAVTRSCAEGTAAICGCSSRHQGSPGKGWKWGGC | |
| | SEDIEFGGMVSREFADARENRPDARSAMNRHNNEAGRQAIASHM | |
| | HLKCKCHGLSGSCEVKTCWWSQPDFRAIGDFLKDKYDSASEMVV | |
| | EKHRESRGWVETLRPRYTYFKVPTERDLVYYEASPNFCEPNPETGS | |
| | FGTRDRTCNVSSHGIDGCDLLCCGRGHNARAERRREKCRCVFHW | |
| | CCYVSCQECTRVYDVHTCK | |
| WNT4 | SNWLYLAKLSSVGSISEEETCEKLKGLIQRQVQMCKRNLEVMDSV | 87 |
| | RRGAQLAIEECQYQFRNRRWNCSTLDSLPVFGKVVTQGTREAAFV | |
| | YAISSAGVAFAVTRACSSGELEKCGCDRTVHGVSPQGFQWSGCSD | |
| | NIAYGVAFSQSFVDVRERSKGASSSRALMNLHNNEAGRKAILTHM | |
| | RVECKCHGVSGSCEVKTCWRAVPPFRQVGHALKEKFDGATEVEP | |
| | RRVGSSRALVPRNAQFKPHTDEDLVYLEPSPDFCEQDMRSGVLGT | |
| | RGRTCNKTSKAIDGCELLCCGRGFHTAQVELAERCSCKFHWCCFV | |
| | KCRQCQRLVELHTCR | |
| WNT5A | IIGAQPLCSQLAGLSQGQKKLCHLYQDHMQYIGEGAKTGIKECQY | 88 |
| | QFRHRRWNCSTVDNTSVFGRVMQIGSRETAFTYAVSAAGVVNAM | |
| | SRACREGELSTCGCSRAARPKDLPRDWLWGGCGDNIDYGYRFAK | |
| | EFVDARERERIHAKGSYESARILMNLHNNEAGRRTVYNLADVACK | |
| | CHGVSGSCSLKTCWLQLADFRKVGDALKEKYDSAAAMRLNSRGK | |
| | LVQVNSRFNSPTTQDLVYIDPSPDYCVRNESTGSLGTQGRLCNKTS | |
| | EGMDGCELMCCGRGYDQFKTVQTERCHCKFHWCCYVKCKKCTE | |
| | IVDQFVCK | |
| WNT5B | QLLTDANSWWSLALNPVQRPEMFIIGAQPVCSQLPGLSPGQRKLC | 89 |
| | QLYQEHMAYIGEGAKTGIKECQHQFRQRRWNCSTADNASVFGRV | |
| | MQIGSRETAFTHAVSAAGVVNAISRACREGELSTCGCSRTARPKDL | |
| | PRDWLWGGCGDNVEYGYRFAKEFVDAREREKNFAKGSEEQGRV | |
| | LMNLQNNEAGRRAVYKMADVACKCHGVSGSCSLKTCWLQLAEF | |
| | RKVGDRLKEKYDSAAAMRVTRKGRLELVNSRFTQPTPEDLVYVD | |
| | PSPDYCLRNESTGSLGTQGRLCNKTSEGMDGCELMCCGRGYNQF | |
| | KSVQVERCHCKFHWCCFVRCKKCTEIVDQYICK | |
| WNT6 | LWWAVGSPLVMDPTSICRKARRLAGRQAELCQAEPEVVAELARG | 90 |
| | ARLGVRECQFQFRFRRWNCSSHSKAFGRILQQDIRETAFVFAITAA | |
| | GASHAVTQACSMGELLQCGCQAPRGRAPPRPSGLPGTPGPPGPAG | |
| | SPEGSAAWEWGGCGDDVDFGDEKSRLFMDARHKRGRGDIRALV | |
| | QLHNNEAGRLAVRSHTRTECKCHGLSGSCALRTCWQKLPPFREVG | |
| | ARLLERFHGASRVMGTNDGKALLPAVRTLKPPGRADLLYAADSP | |
| | DFCAPNRRTGSPGTRGRACNSSAPDLSGCDLLCCGRGHRQESVQL | |
| | EENCLCRFHWCCVVQCHRCRVRKELSLCL | |
| WNT7A | LGASIICNKIPGLAPRQRAICQSRPDAIIVIGEGSQMGLDECQFQFRN | 91 |
| | GRWNCSALGERTVFGKELKVGSREAAFTYAIIAAGVAHAITAACT | |
| | QGNLSDCGCDKEKQGQYHRDEGWKWGGCSADIRYGIGFAKVFV | |
| | DAREIKQNARTLMNLHNNEAGRKILEENMKLECKCHGVSGSCTTK | |
| | TCWTTLPQFRELGYVLKDKYNEAVHVEPVRASRNKRPTFLKIKKP | |
| | LSYRKPMDTDLVYIEKSPNYCEEDPVTGSVGTQGRACNKTAPQAS | |
| | GCDLMCCGRGYNTHQYARVWQCNCKFHWCCYVKCNTCSERTE | |
| | MYTCK | |
| WNT7B | ALSSVVALGANIICNKIPGLAPRQRAICQSRPDAIIVIGEGAQMGINE | 92 |
| | CQYQFRFGRWNCSALGEKTVFGQELRVGSREAAFTYAITAAGVA | |
| | HAVTAACSQGNLSNCGCDREKQGYYNQAEGWKWGGCSADVRY | |
| | GIDFSRRFVDAREIKKNARRLMNLHNNEAGRKVLEDRMQLECKC | |
| | HGVSGSCTTKTCWTTLPKFREVGHLLKEKYNAAVQVEVVRASRL | |
| | RQPTFLRIKQLRSYQKPMETDLVYIEKSPNYCEEDAATGSVGTQGR | |
| | LCNRTSPGADGCDTMCCGRGYNTHQYTKVWQCNCKFHWCCFVK | |
| | CNTCSERTEVFTCK | |
| WNT8A | VNNFLITGPKAYLTYTTSVALGAQSGIEECKFQFAWERWNCPENA | 93 |
| | LQLSTHNRLRSATRETSFIHAISSAGVMYIITKNCSMGDFENCGCDG | |
| | SNNGKTGGHGWIWGGCSDNVEFGERISKLFVDSLEKGKDARALM | |
| | NLHNNRAGRLAVRATMKRTCKCHGISGSCSIQTCWLQLAEFREM | |
| | GDYLKAKYDQALKIEMDKRQLRAGNSAEGHWVPAEAFLPSAEAE | |
| | LIFLEESPDYCTCNSSLGIYGTEGRECLQNSHNTSRWERRSCGRLCT | |
| | ECGLQVEERKTEVISSCNCKFQWCCTVKCDQCRHVVSKYYCARSP | |
| | GSAQSLGKGSA | |
| WNT8B | WSVNNFLMTGPKAYLIYSSSVAAGAQSGIEECKYQFAWDRWNCP | 94 |
| | ERALQLSSHGGLRSANRETAFVHAISSAGVMYTLTRNCSLGDFDN | |
| | CGCDDSRNGQLGGQGWLWGGCSDNVGFGEAISKQFVDALETGQ | |
| | DARAAMNLHNNEAGRKAVKGTMKRTCKCHGVSGSCTTQTCWLQ | |
| | LPEFREVGAHLKEKYHAALKVDLLQGAGNSAAGRGAIADTFRSIS | |
| | TRELVHLEDSPDYCLENKTLGLLGTEGRECLRRGRALGRWERRSC | |
| | RRLCGDCGLAVEERRAETVSSCNCKFHWCCAVRCEQCRRRVTKY | |
| | FCSRAERPRGGAAHKPGRKP | |
| WNT9A | YFGLTGSEPLTILPLTLEPEAAAQAHYKACDRLKLERKQRRMCRR | 95 |
| | DPGVAETLVEAVSMSALECQFQFRFERWNCTLEGRYRASLLKRGF | |
| | KETAFLYAISSAGLTHALAKACSAGRMERCTCDEAPDLENREAWQ | |
| | WGGCGDNLKYSSKFVKEFLGRRSSKDLRARVDFHNNLVGVKVIK | |
| | AGVETTCKCHGVSGSCTVRTCWRQLAPFHEVGKHLKHKYETALK | |
| | VGSTTNEAAGEAGAISPPRGRASGAGGSDPLPRTPELVHLDDSPSF | |
| | CLAGRFSPGTAGRRCHREKNCESICCGRGHNTQSRVVTRPCQCQV | |
| | RWCCYVECRQCTQREEVYTCKG | |
| WNT9B | SYFGLTGREVLTPFPGLGTAAAPAQGGAHLKQCDLLKLSRRQKQL | 96 |
| | CRREPGLAETLRDAAHLGLLECQFQFRHERWNCSLEGRMGLLKR | |
| | GFKETAFLYAVSSAALTHTLARACSAGRMERCTCDDSPGLESRQA | |
| | WQWGVCGDNLKYSTKFLSNFLGSKRGNKDLRARADAHNTHVGI | |
| | KAVKSGLRTTCKCHGVSGSCAVRTCWKQLSPFRETGQVLKLRYD | |
| | SAVKVSSATNEALGRLELWAPARQGSLTKGLAPRSGDLVYMEDSP | |
| | SFCRPSKYSPGTAGRVCSREASCSSLCCGRGYDTQSRLVAFSCHCQ | |
| | VQWCCYVECQQCVQEELVYTCKH | |
| WNT10A | MPRSAPNDILDLRLPPEPVLNANTVCLTLPGLSRRQMEVCVRHPDV | 97 |
| | AASAIQGIQIAIHECQHQFRDQRWNCSSLETRNKIPYESPIFSRGFRE | |
| | SAFAYAIAAAGVVHAVSNACALGKLKACGCDASRRGDEEAFRRK | |
| | LHRLQLDALQRGKGLSHGVPEHPALPTASPGLQDSWEWGGCSPD | |
| | MGFGERFSKDFLDSREPHRDIHARMRLHNNRVGRQAVMENMRRK | |
| | CKCHGTSGSCQLKTCWQVTPEFRTVGALLRSRFHRATLIRPHNRN | |
| | GGQLEPGPAGAPSPAPGAPGPRRRASPADLVYFEKSPDFCEREPRL | |
| | DSAGTVGRLCNKSSAGSDGCGSMCCGRGHNILRQTRSERCHCRFH | |
| | WCCFVVCEECRITEWVSVCK | |
| WNT10B | NEILGLKLPGEPPLTANTVCLTLSGLSKRQLGLCLRNPDVTASALQ | 98 |
| | GLHIAVHECQHQLRDQRWNCSALEGGGRLPHHSAILKRGFRESAF | |
| | SFSMLAAGVMHAVATACSLGKLVSCGCGWKGSGEQDRLRAKLL | |
| | QLQALSRGKSFPHSLPSPGPGSSPSPGPQDTWEWGGCNHDMDFGE | |
| | KFSRDFLDSREAPRDIQARMRIHNNRVGRQVVTENLKRKCKCHGT | |
| | SGSCQFKTCWRAAPEFRAVGAALRERLGRAIFIDTHNRNSGAFQPR | |
| | LRPRRLSGELVYFEKSPDFCERDPTMGSPGTRGRACNKTSRLLDGC | |
| | GSLCCGRGHNVLRQTRVERCHCRFHWCCYVLCDECKVTEWVNV | |
| | CK | |
| WNT11 | IKWLALSKTPSALALNQTQHCKQLEGLVSAQVQLCRSNLELMHTV | 99 |
| | VHAAREVMKACRRAFADMRWNCSSIELAPNYLLDLERGTRESAF | |
| | VYALSAAAISHAIARACTSGDLPGCSCGPVPGEPPGPGNRWGGCA | |
| | DNLSYGLLMGAKFSDAPMKVKKTGSQANKLMRLHNSEVGRQAL | |
| | RASLEMKCKCHGVSGSCSIRTCWKGLQELQDVAADLKTRYLSAT | |
| | KVVHRPMGTRKHLVPKDLDIRPVKDSELVYLQSSPDFCMKNEKV | |
| | GSHGTQDRQCNKTSNGSDSCDLMCCGRGYNPYTDRVVERCHCKY | |
| | HWCCYVTCRRCERTVERYVCK | |
| WNT16 | NWMWLGIASFGVPEKLGCANLPLNSRQKELCKRKPYLLPSIREGA | 100 |
| | RLGIQECGSQFRHERWNCMITAAATTAPMGASPLFGYELSSGTKET | |
| | AFIYAVMAAGLVHSVTRSCSAGNMTECSCDTTLQNGGSASEGWH | |
| | WGGCSDDVQYGMWFSRKFLDFPIGNTTGKENKVLLAMNLHNNE | |
| | AGRQAVAKLMSVDCRCHGVSGSCAVKTCWKTMSSFEKIGHLLKD | |
| | KYENSIQISDKTKRKMRRREKDQRKIPIHKDDLLYVNKSPNYCVED | |
| | KKLGIPGTQGRECNRTSEGADGCNLLCCGRGYNTHVVRHVERCEC | |
| | KFIWCCYVRCRRCESMTDVHTCK | |
| NRG1 | SHLVKCAEKEKTFCVNGGECFMVKDLSNPSRYLCKCPNEFTGDRC | 101 |
| | QNYVMASFYKHLGIEFMEAE | |

As used herein, reference to a mammalian growth factor includes the mature peptide of any mammalian growth factor herein. In some embodiments, the mature peptide is a canonically accepted mature peptide. For instance, in some embodiments the mammalian growth factor does not comprise a signal sequence. In some embodiments, the mammalian growth factor does not comprise the full-length pro-protein. In some embodiments, the mammalian growth factor has the amino acid sequence of the secreted growth factor in vivo. Furthermore, as used herein, reference to a mammalian growth factor includes a functional portion of the mammalian growth factor. A function portion of the mammalian growth factor is a region that has a therapeutic effect. For instance, a functional portion of a mammalian growth factor is osteoinductive. As another example, a functional portion of a mammalian growth factor is capable of initiating and/or enhancing bone repair. A functional portion of a mammalian growth factor may have osteogenic activity.

Non-limiting in vitro assays to determine whether a growth factor or portion thereof, such as those described herein, has osteogenic activity are described in Kim et al., Amino Acids 42:1455-1465, 2012; Lee et al., ACS Med. Chem. Lett. 2(3):248-251, 2011; and Wang et al., Genetics Mol. Res. 13(2):4456-4465, 2014.

In some embodiments, the mammalian growth factor comprises a sequence that is at least 70% identical (e.g., at least 75% identical, at least 80% identical, at least 85% identical, at least 90% identical, at least 95% identical, or at least 99% identical) to any of the sequences in Table B or any secreted human growth factor, and has osteogenic activity. In some embodiments, the amino acids in a mammalian growth factor that are conserved between different species are likely important for osteogenic activity and may not be mutated, while amino acids in a mammalian growth factor that are not conserved between different species are not likely important for osteogenic activity and may be mutated.

In some embodiments, the mammalian growth factor comprises BMP-2. In some embodiments, the mammalian growth factor is a mature peptide of BMP-2 (e.g., does not comprise a signal sequence). In some embodiments, the mammalian growth factor comprises a functional portion of BMP-2. In some embodiments, the functional portion of BMP-2 comprises a sequence at least about 70%, 75%, 80%, 85%, 90%, 95%, or 100% identical to QAKHKQRKRLKSSCKRHPLYVDFSDVGWNDWIVAPPGYHAFYCHGECPFPLADHLNSTNH AIVQTLVNSVNSKIPKACCVPTELSAISMLYLDENEKVVLKNYQDMVVEGCGCR (SEQ ID NO: 32). In some embodiments, the mammalian growth factor comprises a sequence at least about 70%, 75%, 80%, 85%, 90%, 95%, or 100% identical to SEQ ID NO: 32. In some embodiments, the mammalian growth factor comprises a sequence at least about 90% identical to SEQ ID NO: 32. In some embodiments, the mammalian growth factor comprises SEQ ID NO: 32.

### Targeting Polypeptides

Also provided herein are targeting polypeptides. In some embodiments, the targeting polypeptide comprises two or more targeting polypeptides. In some embodiments, two or more targeting polypeptides is no more than about 50, 45, 40, 35, 30, 25, 20, 15, or 10 targeting polypeptides. In some embodiments, two or more targeting polypeptides is about 2, 3, 4, 5, 6, 7, 8, 9, 10, 11, 12, 13, 14, 15, 20, 25, or 30 targeting polypeptides. In some embodiments, two or more targeting polypeptides is about 2 to about 10 targeting polypeptides. In some embodiments, two or more targeting polypeptides is about 5 targeting polypeptides. In some embodiments, the targeting polypeptide binds to a carrier material, as noted elsewhere herein. As a non-limiting example, the targeting polypeptide binds to a calcium phosphate.

In some embodiments, the targeting polypeptide comprises a sequence at least about 70%, 75%, 80%, 85%, 90%, 95%, or 100% identical to SEQ ID NO: 1. In some embodiments, the targeting polypeptide comprises a sequence at least about 70%, 75%, 80%, 85%, 90%, 95%, or 100% identical to SEQ ID NO: 2. In some embodiments, the targeting polypeptide comprises a sequence at least about 70%, 75%, 80%, 85%, 90%, 95%, or 100% identical to SEQ ID NO: 3. In some embodiments, the targeting polypeptide comprises a sequence at least about 70%, 75%, 80%, 85%, 90%, 95%, or 100% identical to SEQ ID NO: 4. In some embodiments, the targeting polypeptide comprises a sequence at least about 70%, 75%, 80%, 85%, 90%, 95%, or 100% identical to SEQ ID NO: 5. In some embodiments, the targeting polypeptide comprises a sequence at least about 70%, 75%, 80%, 85%, 90%, 95%, or 100% identical to SEQ ID NO: 6. In some embodiments, the targeting polypeptide comprises a sequence at least about 70%, 75%, 80%, 85%, 90%, 95%, or 100% identical to SEQ ID NO: 7. In some embodiments, the targeting polypeptide comprises a sequence at least about 70%, 75%, 80%, 85%, 90%, 95%, or 100% identical to SEQ ID NO: 8. In some embodiments, the targeting polypeptide comprises a sequence at least about 70%, 75%, 80%, 85%, 90%, 95%, or 100% identical to SEQ ID NO: 9. In some embodiments, the targeting polypeptide comprises a sequence at least about 70%, 75%, 80%, 85%, 90%, 95%, or 100% identical to SEQ ID NO: 10. In some embodiments, the targeting polypeptide comprises a sequence at least about 70%, 75%, 80%, 85%, 90%, 95%, or 100% identical to SEQ ID NO: 11. In some embodiments, the targeting polypeptide comprises a sequence at least about 70%, 75%, 80%, 85%, 90%, 95%, or 100% identical to SEQ ID NO: 12. In some embodiments, the targeting polypeptide comprises a sequence at least about 70%, 75%, 80%, 85%, 90%, 95%, or 100% identical to SEQ ID NO: 13. In some embodiments, the targeting polypeptide comprises a sequence at least about 70%, 75%, 80%, 85%, 90%, 95%, or 100% identical to SEQ ID NO: 14. In some embodiments, the targeting polypeptide comprises a sequence at least about 70%, 75%, 80%, 85%, 90%, 95%, or 100% identical to SEQ ID NO: 15. In some embodiments, the targeting polypeptide comprises a sequence at least about 70%, 75%, 80%, 85%, 90%, 95%, or 100% identical to SEQ ID NO: 16. In some embodiments, the targeting polypeptide comprises a sequence at least about 70%, 75%, 80%, 85%, 90%, 95%, or 100% identical to SEQ ID NO: 17. In some embodiments, the targeting polypeptide comprises a sequence at least about 70%, 75%, 80%, 85%, 90%, 95%, or 100% identical to SEQ ID NO: 18. In some embodiments, the targeting polypeptide comprises a sequence at least about 70%, 75%, 80%, 85%, 90%, 95%, or 100% identical to SEQ ID NO: 19. In some embodiments, the targeting polypeptide comprises a sequence at least about 70%, 75%, 80%, 85%, 90%, 95%, or 100% identical to SEQ ID NO: 20. In some embodiments, the targeting polypeptide comprises a sequence at least about 70%, 75%, 80%, 85%, 90%, 95%, or 100% identical to SEQ ID NO: 21. In some embodiments, the targeting polypeptide comprises a sequence at least about 70%, 75%, 80%, 85%, 90%, 95%, or 100% identical to SEQ ID NO: 22. In some embodiments, the targeting polypeptide comprises a sequence at least about 70%, 75%, 80%, 85%, 90%, 95%, or 100% identical to SEQ ID NO: 23. In some embodiments, the targeting polypeptide comprises a sequence at least about 70%, 75%, 80%, 85%, 90%, 95%, or 100% identical to SEQ ID NO: 24. In some embodiments, the targeting polypeptide comprises a sequence at least about 70%, 75%, 80%, 85%, 90%, 95%, or 100% identical to SEQ ID NO: 25. In some embodiments, the targeting polypeptide comprises a sequence at least about 70%, 75%, 80%, 85%, 90%, 95%, or 100% identical to SEQ ID NO: 26. In some embodiments, the targeting polypeptide comprises a sequence at least about 70%, 75%, 80%, 85%, 90%, 95%, or 100% identical to SEQ ID NO: 27. In some embodiments, the targeting polypeptide comprises a sequence at least about 70%, 75%, 80%, 85%, 90%, 95%, or 100% identical to SEQ ID NO: 28. In some embodiments, the targeting polypeptide comprises a sequence at least about 70%, 75%, 80%, 85%, 90%, 95%, or 100% identical to SEQ ID NO: 29. In some embodiments, the targeting polypeptide comprises a sequence at least about 70%, 75%, 80%, 85%, 90%, 95%, or 100% identical to SEQ ID NO: 30. In some embodiments, the targeting polypeptide comprises a sequence at least about 70%, 75%, 80%, 85%, 90%, 95%, or 100% identical to SEQ ID NO: 31. In some embodiments, the targeting polypeptide comprises a sequence at least about 70%, 75%, 80%, 85%, 90%, 95%, or 100% identical to SEQ ID NO: 36. In some embodiments, the targeting polypeptide comprises a sequence at least about 70%, 75%, 80%, 85%, 90%, 95%, or 100% identical to SEQ ID NO: 37. In some embodiments, the targeting polypeptide comprises a sequence at least about 70%, 75%, 80%, 85%, 90%, 95%, or 100% identical to SEQ ID NO: 38. In some embodiments, the targeting polypeptide comprises a sequence at least about 70%, 75%, 80%, 85%, 90%, 95%, or 100% identical to SEQ ID NO: 39. In some embodiments, the targeting polypeptide comprises a sequence at least about 70%, 75%, 80%, 85%, 90%, 95%, or 100% identical to SEQ ID NO: 40. In some embodiments, the targeting polypeptide comprises a sequence at least about 70%, 75%, 80%, 85%, 90%, 95%, or 100% identical to SEQ ID NO: 41. In some embodiments, the targeting polypeptide comprises a sequence at least about 70%, 75%, 80%, 85%, 90%, 95%, or 100% identical to SEQ ID NO: 42. In some embodiments, the targeting polypeptide comprises a sequence at least about 70%, 75%, 80%, 85%, 90%, 95%, or 100% identical to SEQ ID NO: 43. In some embodiments, the targeting polypeptide comprises a sequence at least about 70%, 75%, 80%, 85%, 90%, 95%, or 100% identical to SEQ ID NO: 401. In some embodiments, the targeting polypeptide comprises a sequence at least about 70%, 75%, 80%, 85%, 90%, 95%, or 100% identical to SEQ ID NO: 402. In some embodiments, the targeting polypeptide comprises a sequence at least about 70%, 75%, 80%, 85%, 90%, 95%, or 100% identical to SEQ ID NO: 403. In some embodiments, the targeting polypeptide comprises a sequence at least about 70%, 75%, 80%, 85%, 90%, 95%, or 100% identical to SEQ ID NO: 404. In some embodiments, the targeting polypeptide comprises a sequence at least about 70%, 75%, 80%, 85%, 90%, 95%, or 100% identical to SEQ ID NO: 405. In some embodiments, the targeting polypeptide comprises a sequence at least about 70%, 75%, 80%, 85%, 90%, 95%, or 100% identical to SEQ ID NO: 406. In some embodiments, the targeting polypeptide comprises a sequence at least about 70%, 75%, 80%, 85%, 90%, 95%, or 100% identical to SEQ ID NO: 407. In some embodiments, the targeting polypeptide comprises a sequence at least about 70%, 75%, 80%, 85%, 90%, 95%, or 100% identical to SEQ ID NO: 408. In some embodiments, the targeting polypeptide comprises a sequence at least about 70%, 75%, 80%, 85%, 90%, 95%, or 100% identical to SEQ ID NO: 409. In some embodiments, the targeting polypeptide comprises a sequence at least about 70%, 75%, 80%, 85%, 90%, 95%, or 100% identical to SEQ ID NO: 410. In some embodiments, the targeting polypeptide comprises a sequence at least about 70%, 75%, 80%, 85%, 90%, 95%, or 100% identical to SEQ ID NO: 411. In some embodiments, the targeting polypeptide comprises a sequence at least about 70%, 75%, 80%, 85%, 90%, 95%, or 100% identical to SEQ ID NO: 412. In some embodiments, the targeting polypeptide comprises a sequence at least about 70%, 75%, 80%, 85%, 90%, 95%, or 100% identical to SEQ ID NO: 413. In some embodiments, the targeting polypeptide comprises a sequence at least about 70%, 75%, 80%, 85%, 90%, 95%, or 100% identical to SEQ ID NO: 414. In some embodiments, the targeting polypeptide comprises a sequence at least about 70%, 75%, 80%, 85%, 90%, 95%, or 100% identical to SEQ ID NO: 415. In some embodiments, the targeting polypeptide comprises a sequence at least about 70%, 75%, 80%, 85%, 90%, 95%, or 100% identical to SEQ ID NO: 416. In some embodiments, the targeting polypeptide comprises a sequence at least about 70%, 75%, 80%, 85%, 90%, 95%, or 100% identical to SEQ ID NO: 417. In some embodiments, the targeting polypeptide comprises a sequence at least about 70%, 75%, 80%, 85%, 90%, 95%, or 100% identical to SEQ ID NO: 418. In some embodiments, the targeting polypeptide comprises a sequence at least about 70%, 75%, 80%, 85%, 90%, 95%, or 100% identical to SEQ ID NO: 419. In some embodiments, the targeting polypeptide comprises a sequence at least about 70%, 75%, 80%, 85%, 90%, 95%, or 100% identical to SEQ ID NO: 420. In some embodiments, the targeting polypeptide comprises a sequence at least about 70%, 75%, 80%, 85%, 90%, 95%, or 100% identical to SEQ ID NO: 421. In some embodiments, the targeting polypeptide comprises a sequence at least about 70%, 75%, 80%, 85%, 90%, 95%, or 100% identical to SEQ ID NO: 422.

In some embodiments, the targeting polypeptide comprises Formula I: A₀B₀C₀D₀E₀F₀G₀H₀I₀J₀K₀L₀ (Formula I) (SEQ ID NO: 35), where: A₀ is V, L, I, G, S, T, or A; B₀ is I, L, V, Q, T, S, G, or A; C₀ is G, A, V, or S; D₀ is E, D, L, or G; E₀ is S, T, P T, E, or D; F₀ is T or S; G₀ is H, T, or S; H₀ is H or T; I₀ is R, S, K, P, or H; J₀ is P, S, R, or K; K₀ is W, F, S, P, V, A, or G; and L₀ is absent or is S, T, G, (or A). In some embodiments, Formula I does not include LLADTTHHRPWT (SEQ ID NO: 1).

(1) Provided herein is a first embodiment of a targeting polypeptide comprising Formula I. (2) The targeting polypeptide of embodiment 1, wherein A0 is V. (3) The targeting polypeptide of embodiment 1, wherein A0 is L. (4) The targeting polypeptide of embodiment 1, wherein A0 is I. (5) The targeting polypeptide of embodiment 1, wherein A0 is G. (6) The targeting polypeptide of embodiment 1, wherein A0 is S. (7) The targeting polypeptide of embodiment 1, wherein A0 is T. (8) The targeting polypeptide of embodiment 1, wherein A0 is A. (9) The targeting polypeptide of any one of embodiments 1-8, wherein B0 is I. (10) The targeting polypeptide of any one of embodiments 1-8, wherein B0 is L. (11) The targeting polypeptide of any one of embodiments 1-8, wherein B0 is V. (12) The targeting polypeptide of any one of embodiments 1-8, wherein B0 is Q. (13) The targeting polypeptide of any one of embodiments 1-8, wherein B0 is T. (14) The targeting polypeptide of any one of embodiments 1-8, wherein B0 is S. (15) The targeting polypeptide of any one of embodiments 1-8, wherein B0 is G. (16) The targeting polypeptide of any one of embodiments 1-8, wherein B0 is A. (17) The targeting polypeptide of any one of embodiments 1-16, wherein C0 is G. (18) The targeting polypeptide of any one of embodiments 1-16, wherein C0 is A. (19) The targeting polypeptide of any one of embodiments 1-16, wherein C0 is V. (20) The targeting polypeptide of any one of embodiments 1-16, wherein C0 is S. (21) The targeting polypeptide of any one of embodiments 1-20, wherein D0 is E. (22) The targeting polypeptide of any one of embodiments 1-20, wherein D0 is D. (23) The targeting polypeptide of any one of embodiments 1-20, wherein D0 is L. (24) The targeting polypeptide of any one of embodiments 1-20, wherein D0 is G. (25) The targeting polypeptide of any one of embodiments 1-24, wherein E0 is S. (26) The targeting polypeptide of any one of embodiments 1-24, wherein E0 is T. (27) The targeting polypeptide of any one of embodiments 1-24, wherein E0 is P. (28) The targeting polypeptide of any one of embodiments 1-24, wherein E0 is E. (29) The targeting polypeptide of any one of embodiments 1-24, wherein E0 is D. (30) The targeting polypeptide of any one of embodiments 1-29, wherein F0 is T. (31) The targeting polypeptide of any one of embodiments 1-29, wherein F0 is S. (32) The targeting polypeptide of any one of embodiments 1-31, wherein G0 is H. (33) The targeting polypeptide of any one of embodiments 1-31, wherein G0 is T. (34) The targeting polypeptide of any one of embodiments 1-31, wherein G0 is S. (35) The targeting polypeptide of any one of embodiments 1-34, wherein H0 is H. (36) The targeting polypeptide of any one of embodiments 1-34, wherein H0 is T. (37) The targeting polypeptide of any one of embodiments 1-36, wherein 10 is R. (38) The targeting polypeptide of any one of embodiments 1-36, wherein 10 is S. (39) The targeting polypeptide of any one of embodiments 1-36, wherein I0 is K. (40) The targeting polypeptide of any one of embodiments 1-36, wherein I0 is P. (41) The targeting polypeptide of any one of embodiments 1-36, wherein I0 is H. (42) The targeting polypeptide of any one of embodiments 1-41, wherein J0 is P. (43) The targeting polypeptide of any one of embodiments 1-41, wherein J0 is S. (44) The targeting polypeptide of any one of embodiments 1-41, wherein J0 is R. (45) The targeting polypeptide of any one of embodiments 1-41, wherein J0 is K. (46) The targeting polypeptide of any one of embodiments 1-45, wherein K0 is W. (47) The targeting polypeptide of any one of embodiments 1-45, wherein K0 is F. (48) The targeting polypeptide of any one of embodiments 1-45, wherein K0 is S. (49) The targeting polypeptide of any one of embodiments 1-45, wherein K0 is P. (50) The targeting polypeptide of any one of embodiments 1-45, wherein K0 is V. (51) The targeting polypeptide of any one of embodiments 1-45, wherein K0 is A. (52) The targeting polypeptide of any one of embodiments 1-45, wherein K0 is G. (53) The targeting polypeptide of any one of embodiments 1-52, wherein L0 is absent. (54) The targeting polypeptide of any one of embodiments 1-52, wherein L0 is S. (55) The targeting polypeptide of any one of embodiments 1-52, wherein L0 is T. (56) The targeting polypeptide of any one of embodiments 1-52, wherein L0 is G. (57) The targeting polypeptide of any one of embodiments 1-52, wherein L0 is A. (58) A chimeric polypeptide comprising the targeting polypeptide of any one of embodiments 1-57. (59) The chimeric polypeptide of embodiment 58, comprising a therapeutic agent. (60) The chimeric polypeptide of embodiment 59, wherein the therapeutic agent comprises a sequence at least about 70%, 75%, 80%, 85%, 90%, 91%, 92%, 93%, 94%, 95%, 96%, 97%, 98%, 99%, or 100% identical one or more sequences of Table B.

In some embodiments, the targeting polypeptides described herein can have a total length of about 5 amino acids to about 200 amino acids, about 5 amino acids to about 100 amino acids, about 5 amino acids to about 80 amino acids, about 5 amino acids to about 60 amino acids, about 10 amino acids to about 100 amino acids, about 10 amino acids to about 80 amino acids, about 10 amino acids to about 80 amino acids, about 20 amino acids to about 100 amino acids, about 20 amino acids to about 80 amino acids, about 20 amino acids to about 60 amino acids, about 30 amino acids to about 100 amino acids, about 30 amino acids to about 80 amino acids, about 30 amino acids to about 60 amino acids, about 40 amino acids to about 100 amino acids, about 40 amino acids to about 80 amino acids, about 40 amino acids to about 60 amino acids, or about 60 amino acids.

### Nucleic Acids/Vectors

Also provided herein are nucleic acids that encode any of the chimeric polypeptides or targeting polypeptides described herein.

Also provided herein are vectors that include any of the nucleic acids provided herein. A “vector” according to the present disclosure is a polynucleotide capable of inducing the expression of a recombinant protein (e.g., any of the chimeric polypeptides or targeting polypeptides described) in a host cell. A vector provided herein can be, e.g., in circular or linearized form. Non-limiting examples of vectors include plasmids, SV40 vectors, adenoviral viral vectors, and adeno-associated virus (AAV) vectors. Non-limiting examples of vectors include lentiviral vectors or retroviral vectors, e.g., gamma-retroviral vectors. See, e.g., Carlens et al., Exp. Hematol. 28(10:1137-1146, 2000; Park et al., Trends Biotechnol. 29(11):550-557, 2011; and Alonso-Camino et al., Mol. Ther. Nucleic Acids 2:e93, 2013. Non-limiting examples of retroviral vectors include those derived from Moloney murine leukemia virus, myeloproliferative sarcoma virus, murine embryonic stem cell virus, murine stem cell virus, spleen focus forming virus, or adeno-associated virus. Non-limiting examples of retroviral vectors are described in, e.g., U.S. Pat. Nos. 5,219,740 and 6,207,453; Miller et al., BioTechniques 7:980-990, 1989; Miller, Human Gene Therapy 1:5-14, 1990; Scarpa et al., Virology 180:849-852, 1991; Burns et al., Proc. Natl. Acad. Sci. U.S.A. 90:8033-8037, 1993; and Boris-Lawrie et al., Cur. Opin. Genet. Develop. 3:102-109, 1993. Exemplary lentiviral vectors are described in, e.g., Wang et al., J. Immunother. 35(9):689-701, 2003; Cooper et al., Blood 101:1637-1644, 2003; Verhoeyen et al., Methods Mol. Biol. 506:97-114, 2009; and Cavalieri et al., Blood 102(2):497-505, 2003.

Exemplary vectors, in which any of the nucleic acids provided herein can be inserted, are described in, e.g., Ausubel et al., Eds. “Current Protocols in Molecular Biology” Current Protocols, 1993; and Sambrook et al., Eds. “Molecular Cloning: A Laboratory Manual,” 2nd ed., Cold Spring Harbor Press, 1989.

In some embodiments, the vectors further include a promoter and/or enhancer operably linked to any of the nucleic acids described herein. Non-limiting examples of promoters include promoters from human cytomegalovirus (CMV), mouse phosphoglycerate kinase 1, polyoma adenovirus, thyroid stimulating hormone a, vimentin, simian virus 40 (SV40), tumor necrosis factor, β-globin, α-fetoprotein, γ-globin, β-interferon, γ-glutamyl transferase, human ubiquitin C (UBC), mouse mammary tumor virus (MMTV), Rous sarcoma virus, glyceraldehyde-3-phosphate dehydrogenase, β-actin, metallothionein II (MT II), amylase, human EF1α, cathepsin, MI muscarinic receptor, retroviral LTR (e.g. human T-cell leukemia virus HTLV), AAV ITR, interleukin-2, collagenase, platelet-derived growth factor, adenovirus E2, stromelysin, murine MX, rat insulin, glucose regulated protein 78, human immunodeficiency virus, glucose regulated protein 94, α-2-macroglobulin, MHC class I, HSP70, proliferin, immunoglobulin light chain, T-cell receptor, HLA DQα, HLA DQβ, interleukin-2 receptor, MHC class II, prealbumin (transthyretin), elastase I, albumin, c-fos, neural cell adhesion molecule (NCAM), H2B histone, rat growth hormone, human serum amyloid (SAA), muscle creatinine kinase, troponin I (TN I), and Gibbon Ape Leukemia Virus (GALV). In some embodiments, the promoter may be an inducible promoter or a constitutive promoter. Additional examples of promoters are known in the art.

In some examples, the vectors provided herein further include a poly(A) sequence, which is operably linked and positioned 3′ to the sequence encoding the chimeric polypeptide or targeting polypeptide. Non-limiting examples of a poly(A) sequence include those derived from bovine growth hormone (Woychik et al., Proc. Natl. Acad. Sci. U.S.A. 81(13):3944-3948, 1984, and U.S. Pat. No. 5,122,458), mouse-β-globin, mouse-α-globin (Orkin et al., EMBO J. 4(2):453-456, 1985), human collagen, polyoma virus (Batt et al., Mol. Cell Biol. 15(9):4783-4790, 1995), the Herpes simplex virus thymidine kinase gene (HSV TK), IgG heavy chain gene polyadenylation signal (U.S. Patent Application Publication No. 2006/0040354), human growth hormone (hGH) (Szymanski et al., Mol. Therapy 15(7):1340-1347, 2007), SV40 poly(A) site, e.g., SV40 late and early poly(A) site (Schek et al., Mol. Cell Biol. 12(12):5386-5393, 1992). In some embodiments, the poly(A) sequence includes a highly conserved upstream element (AATAAA). The this AATAAA sequence can, e.g., be substituted with other hexanucleotide sequences with homology to AATAAA which are capable of signaling polyadenylation, including, e.g., ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, and AATAAG. See, e.g., WO 06012414 A2).

A poly(A) sequence can, e.g., be a synthetic polyadenylation site. See, e.g., Levitt el al, Genes Dev. 3(7):1019-1025, 1989). In some examples, a poly(A) sequence can be the polyadenylation signal of soluble neuropilin-1: AAATAAAATACGAAATG (SEQ ID NO: 711). Additional examples of poly(A) sequences are known in the art. Additional examples and aspects of vectors are also known in the art.

### Methods of Making a Chimeric Polypeptide

Also provided herein are methods of making a chimeric polypeptide (e.g., any of the chimeric polypeptides described herein) or a targeting polypeptide (e.g., any of the targeting polypeptides described herein) that include: introducing into a cell a nucleic acid sequence encoding the chimeric polypeptide or the targeting polypeptide to produce a recombinant cell; and culturing the recombinant cell under conditions sufficient for the expression of the chimeric polypeptide or targeting polypeptide. In some embodiments, the introducing step includes introducing into a cell an expression vector including a nucleic acid sequence encoding the chimeric polypeptide or the targeting polypeptide to produce a recombinant cell. In some embodiments, the expression vector includes chaperones (e.g., GroES, GroEL) and glutathione to aid with in vitro folding.

A chimeric polypeptide or targeting polypeptide described herein can be produced by any cell, e.g., a eukaryotic cell or a prokaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. In some embodiments, the eukaryotic cell is a mammalian cell (e.g., a Chinese Hamster Ovary (CHO) cell). As used herein, the term “prokaryotic cell” refers to a cell that does not have a distinct, membrane-bound nucleus. In some embodiments, the prokaryotic cell is a bacterial cell. In some embodiments, the bacterial cell is a chemically competent E. coli K12 cell (e.g., Shuffle® T7; New England BioLabs) or a BL21 (DE3) pLysS chemically competent E. coli cell.

Methods of culturing cells are well known in the art. Cells can be maintained in vitro under conditions that favor proliferation, differentiation, and growth. Briefly, cells can be cultured by contacting a cell (e.g., any cell) with a cell culture medium that includes the necessary growth factors and supplements to support cell viability and growth.

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalection, hydrodynamic delivery, magnetofection, viral transduction (e.g., adenoviral and lentiviral transduction), and nanoparticle transfection.

Provided herein are methods that further include isolation of the chimeric polypeptide or the targeting polypeptide from a cell (e.g., a eukaryotic cell) using techniques well-known in the art (e.g., ammonium sulfate precipitation, polyethylene glycol precipitation, cobalt column, heparin column, ion-exchange chromatography (anion or cation), chromatography based on hydrophobic interaction, metal-affinity chromatography, ligand-affinity chromatography, and size exclusion chromatography).

### Carrier Materials

In one aspect, provided herein are carrier materials that may be combined with a targeting polypeptide, therapeutic agent, and/or chimeric polypeptide herein. In some embodiments, the targeting polypeptide binds to the carrier material. In some embodiments, a carrier material is a material for which a targeting polypeptide herein is capable of binding. In some embodiments, the targeting polypeptide is coated on the surface of the carrier material. Non-limiting examples of carrier materials include calcium phosphate (e.g., tricalcium phosphate), hydroxyapatite, fluorapatite, bone (e.g., demineralized bone), glasses (bioglasses) such as silicates, and vanadates. In an example embodiment, the carrier material comprises a ceramic material.

In some embodiments, provided are devices comprising a carrier material and an agent. In some cases, the agent comprises a therapeutic agent. In some cases, the agent comprises a targeting polypeptide. In some cases, the agent comprises a chimeric polypeptide comprising a targeting polypeptide and a therapeutic agent.

In some embodiments, the targeting polypeptide has an affinity for the carrier material, or a component of the carrier material. In some embodiments, the dissociation constant (KD) for binding between the targeting polypeptide and the carrier material or component thereof is: (i) at least about 1 fM, at least about 10 fM, at least about 100 fM, or at least about 1 pM; and (ii) less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 1 μM, or less than about 100 pM.

In some embodiments, the carrier material is a material in a subject, e.g., a mammalian subject. In some embodiments, the carrier material comprises bone. In some embodiments, the targeting polypeptide binds to bone with a KD of: (i) at least about 1 fM, at least about 10 fM, at least about 100 fM, or at least about 1 pM; and (ii) less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 1 μM, or less than about 100 pM.

In some embodiments, the carrier material comprises demineralized bone. In some embodiments, the targeting polypeptide binds to bone with a KD of: (i) at least about 1 fM, at least about 10 fM, at least about 100 fM, or at least about 1 pM; and (ii) less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 1 μM, or less than about 100 pM.

In some embodiments, the carrier material comprises cartilage. In some embodiments, the targeting polypeptide binds to cartilage with a KD of: (i) at least about 1 fM, at least about 10 fM, at least about 100 fM, or at least about 1 pM; and (ii) less than about 100 μM, less than about 90 μM, less than about 80 μM, less than about 70 μM, less than about 60 μM, less than about 50 M, less than about 40 μM, less than about 30 μM, less than about 20 μM, less than about 10 μM, less than about 5 μM, less than about 1 μM, or less than about 100 pM.

In some embodiments, the carrier material comprises calcium phosphate. In some embodiments, the carrier material comprises tricalcium phosphate. In some embodiments, the targeting polypeptide binds to tricalcium phosphate with a KD of: (i) at least about 1 fM, at least about 10 fM, at least about 100 fM, or at least about 1 pM; and (ii) less than about 100 μM, less than about 90 μM, less than about 80 μM, less than about 70 μM, less than about 60 M, less than about 50 μM, less than about 40 μM, less than about 30 μM, less than about 20 μM, less than about 10 μM, less than about 5 μM, less than about 1 μM, or less than about 100 pM.

In some embodiments, the carrier material comprises β-TCP. In some embodiments, the targeting polypeptide binds to β-TCP with a KD of: (i) at least about 1 fM, at least about 10 fM, at least about 100 fM, or at least about 1 pM; and (ii) less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 1 μM, or less than about 100 pM.

In some embodiments, the carrier material comprises alpha tricalcium phosphate. In some embodiments, the targeting polypeptide binds to alpha tricalcium phosphate with a KD of: (i) at least about 1 fM, at least about 10 fM, at least about 100 fM, or at least about 1 pM; and (ii) less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 1 μM, or less than about 100 pM.

In some embodiments, the carrier material comprises hydroxyapatite. In some embodiments, the targeting polypeptide binds to hydroxyapatite with a KD of: (i) at least about 1 fM, at least about 10 fM, at least about 100 fM, or at least about 1 pM; and (ii) less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 1 μM, or less than about 100 pM.

In some embodiments, the carrier material comprises fluorapatite. In some embodiments, the targeting polypeptide binds to β-TCP with a KD of: (i) at least about 1 fM, at least about 10 fM, at least about 100 fM, or at least about 1 pM; and (ii) less than about 100 μM, less than about 90 M, less than about 80 μM, less than about 70 μM, less than about 60 μM, less than about 50 μM, less than about 40 M, less than about 30 μM, less than about 20 μM, less than about 10 μM, less than about 5 μM, less than about 1 μM, or less than about 100 pM.

In some embodiments, the carrier material comprises glass. In some embodiments, the glass comprises a bioglass. In some embodiments, the glass comprises a silicate. In some embodiments, the targeting polypeptide binds to a glass with a KD of: (i) at least about 1 fM, at least about 10 fM, at least about 100 fM, or at least about 1 pM; and (ii) less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 1 μM, or less than about 100 pM.

In some embodiments, the carrier material comprises a ceramic mineral. In some embodiments, the targeting polypeptide binds to the ceramic mineral with a KD of: (i) at least about 1 fM, at least about 10 fM, at least about 100 fM, or at least about 1 pM; and (ii) less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 5 μM, less than about 1 μM, or less than about 100 pM.

In some embodiments, the carrier material comprises a vanadate.

In some embodiments, the carrier material comprises a chelated divalent metal ion.

In some embodiments, a dissociation constant is measured using any method known in the art and/or mentioned herein. In some embodiments, the dissociation constant is measured using a release assay, wherein sample protein released into solution is quantified using ELISA. In some embodiments, the dissociation constant is measured using released fluorescence, e.g., using a carrier material bound to green fluorescent protein (GFP). In some embodiments, the dissociation constant is measured using the targeting polypeptide. In some embodiments, the dissociation constant is measured using a chimeric polypeptide comprising the targeting polypeptide. In some embodiments, the dissociation constant is measured using the carrier material. In some embodiments, the dissociation constant is measured using the component of the carrier material (e.g., a tricalcium phosphate or a hydroxyapatite).

In some embodiments, the targeting polypeptide is capable of remaining bound to the carrier polypeptide in a physiological buffer (e.g., phosphate buffered saline) at 37° C. for at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, the binding is measured using the targeting polypeptide. In some embodiments, the binding is measured using a chimeric polypeptide comprising the targeting polypeptide. In some embodiments, the binding is measured using the carrier material. In some embodiments, the binding is measured using the component of the carrier material (e.g., a tricalcium phosphate or a hydroxyapatite).

### β-TCP

Sintering of tricalcium phosphate, Ca₃(PO₄)₂, causes its structure to convert to β-TCP (CAS No. 7758-87-4). β-TCP is an osteoconductive material that supports bone mineralization by easily dissolving at low pH and serves as a rigid substrate for cell attachment.

In some embodiments, reference to β-TCP includes reference to a carrier material comprising β-TCP. For instance, compositions described herein comprising a chimeric polypeptide and β-TCP include compositions comprising the chimeric polypeptide and a carrier material comprising β-TCP. In some embodiments, the β-TCP carrier material comprises at least about 10%, 15%, 20%, 25%, 30%, 35%, 40%, 45%, 50%, 55%, 60%, 65%, 70%, 75%, 80%, 85%, 90%, or 95% by weight β-TCP. In a non-limiting example, the β-TCP carrier material comprises about 30% to about 50%, or about 40%, β-TCP. β-TCP as described herein can be in a variety of different forms. Examples of such forms include a granular form, a porous form, a powder, a putty (e.g., a moldable putty), a paste, a scaffold, fiber form, a coating on a solid surface (e.g., a coating on a medical device), or any combination thereof. In addition, the β-TCP can be used in a variety of different shapes (e.g., a cross, a ladder, a sphere, an ellipsoid, a square, a triangular pyramid, a rod, a cone, a torus, or a wedge, or any combination thereof) and sizes (e.g., largest average diameter of about 1 mm, about 2 mm, about 3 mm, about 4 mm, about 5 mm, about 6 mm, about 7 mm, about 8 mm, about 9 mm, about 1 cm, about 2 cm, about 3 cm, about 4 cm, about 5 cm, about 6 cm, about 7 cm, about 8 cm, about 9 cm, or about 10 cm). In some embodiments, a β-TCP carrier material is porous (e.g., about 90%, about 91%, about 92%, about 93%, about 94%, about 95%, about 96%, about 97%, about 98%, or about 99% porous when dry (no hydration)). In some embodiments, a β-TCP carrier material is about 90% to about 99%, or about 95% to about 99% porous when dry. In some embodiments, a β-TCP carrier material is about 98% porous when dry.

In some embodiments, a β-TCP carrier material comprises one or more additional materials. In some embodiments, the one or more additional materials comprises siloxane-containing vaterite (SiV). In some embodiments, the one or more additional materials comprises poly(L-lactide-co-glycolide) (PLGa, CAS No. 30846-39-0). In some embodiments, the carrier material comprises about 40% by weight β-TCP, about 30% by weight SiV, and about 30% by weight PLGa.

In some embodiments, the β-TCP is in the form of fibers. In some embodiments, the fiber is formed by electrospinning. In some embodiments, the fiber is resorbable. In some embodiments, the fiber diameter is from about 1 μM to about 500 μM, or from about 1 μM to about 300 μM, from about 3 μM to about 250 μM, or from about 3 μM to about 150 μM. In some embodiments, the maximum fiber diameter is about 500 μM, about 400 μM, about 300 μM, or about 250 μM. In some embodiments, the true density (pycnometry) is from about 1 g/cm³ to about 10 g/cm³, from about 1 g/cm³ to about 5 g/cm³, or about 1, 2, 3, 4, or 5 g/cm³. For example, the true density is about 2.5 g/cm³.

In some embodiments, the β-TCP fiber comprises β-TCP and a bioabsorbable polymer. In some embodiments, the β-TCP fiber comprises β-TCP and calcium carbonate. In some cases, the calcium carbonate comprises silicone. In some embodiments, the β-TCP fiber comprises β-TCP, a bioabsorbable polymer or resin, and calcium carbonate. In some embodiments, the β-TCP fiber is biodegradable. In some embodiments, the bioabsorbable polymer comprises polylactic acid (PLA) and/or polylactic acid-glycolic acid copolymer (PLGA). A non-limiting example for preparing a β-TCP fiber herein comprises electrospinning. In some embodiments, the β-TCP fiber comprises a PLGA resin comprising calcium phosphate particles, where the fiber is produced using electrospinning. In some embodiments, the calcium phosphate particles comprise silicon.

In some embodiments, the β-TCP fiber comprises PLA and β-TCP silicon-comprising vaterite. In some embodiments, the β-TCP fiber comprises a biodegradable fiber produced by electrospinning, wherein the biodegradable fiber comprises calcium phosphate particles in an amount of 40% to 60% by weight, silicon-releasing calcium carbonate particles in an amount of 10% by weight or more, and a poly-L-lactic acid polymer in an amount of 30% by weight or more as the remainder, and wherein an amount of amorphous phase of the poly-L-lactic acid polymer is from 75% to 98% by weight.

As a non-limiting example, the β-TCP fiber comprises ReBOSSIS® (ORTHOReBIRTH). As another non-limiting example, the β-TCP fiber comprises ReBOSSIS85 Bone Void Filler (ORTHOReBIRTH).

In some embodiments, a targeting polypeptide herein is combined with a β-TCP fiber. In some embodiments, the targeting polypeptide comprises a sequence at least about 70%, 75%, 80%, 85%, 90%, 95%, or 100% identical to one or more of SEQ ID NO: 1-31, 36-43, or 401-422. In some embodiments, the targeting polypeptide is part of a chimeric polypeptide further comprising a therapeutic agent. In some embodiments, the therapeutic agent comprises a mammalian growth factor. In some embodiments, the mammalian growth factor comprises the mature peptide of the mammalian growth factor. As a non-limiting example, the mammalian growth factor comprises the mature peptide of BMP-2, comprising a sequence at least about 70%, 75%, 80%, 85%, 90%, 95%, or 100% identical to SEQ ID NO: 32. In some embodiments, the β-TCP fiber is ReBOSSIS85 Bone Void Filler.

In an exemplary embodiment, a chimeric polypeptide comprises a sequence at least about 70%, 75%, 80%, 85%, 90%, 95%, or 100% identical to SEQ ID NO: 502.

### Compositions and Kits

Also provided herein are compositions (e.g., pharmaceutical compositions) that include any of the chimeric polypeptides or targeting polypeptides described herein. In some examples, the compositions can further include β-TCP (e.g., any of the types of β-TCP described herein). In some examples, the β-TCP is formulated as a powder, a putty (e.g., a moldable putty), a paste, a scaffold (e.g., a porous scaffold), a sponge, and/or a coating on a solid surface (e.g., a coating on a medical device). In some examples, the β-TCP can be disposed on or in a scaffold, a mesh, or a sponge (e.g., a resorbable sponge).

In some instances, the compositions (e.g., pharmaceutical compositions) are disposed in a sterile vial or a pre-loaded syringe.

In some instances, the compositions (e.g., pharmaceutical compositions) are formulated for different routes of administration (e.g., intraarticular, injection into a joint, or injection proximal to a bone fissure or fracture). Single or multiple administrations of any of the pharmaceutical compositions described herein can be given to a subject depend on, for example: the dosage and frequency as required and tolerated by the subject. A dosage of the pharmaceutical composition should provide a sufficient quantity of the chimeric polypeptide to effective treat or ameliorate conditions (e.g., bone defects, bone fractures, cartilage defects, or cartilage loss), or symptoms.

Also provided herein are kits that include any of the chimeric polypeptides or any of the targeting polypeptides described herein, or any of the compositions (e.g., pharmaceutical compositions) described herein. In some embodiments, the kits can include instructions for performing any of the methods described herein. In some instances, the kits can include at least one dose of any of the compositions (e.g., pharmaceutical compositions) described herein. In some embodiments, the kits can include a syringe for administering any of the pharmaceutical compositions described herein. The kits described herein are not so limited; other variations will be apparent to one of ordinary skill in the art.

### Methods of Making a Composition

Also provided herein are methods of producing any of the compositions described herein. Any of the compositions provided herein can be produced using the methods described herein or methods known in the art. For example, to create a β-TCP scaffold, granulated β-TCP powder can be sintered, sieved, and fabricated into a desired shape (e.g., any of the shapes described herein). In some examples, the purity of the β-TCP present in any of the compositions described herein can be greater than about 75%, greater than 80%, greater than 85%, greater than 90%, greater than 95%, or greater than 99% pure. In some examples, the purity of β-TCP present in any of the compositions described herein can be greater than 1%, greater than 2%, greater than 3%, greater than 4%, greater than 5%, greater than 6%, greater than 7%, greater than 8%, greater than 9%, greater than 10%, greater than 12%, greater than 14%, greater than 15%, greater than 16%, greater than 18%, greater than 20%, greater than 22%, greater than 24%, greater than 25%, greater than 26%, greater than 28%, greater than 30%, greater than 32%, greater than 34%, greater than 35%, greater than 36%, greater 38%, greater 40%, greater than 42%, greater than 44%, greater than 45%, greater than 46%, greater than 48%, greater than 50%, greater than 52%, greater than 54%, greater than 55%, greater than 56%, greater than 58%, greater than 60%, greater than 62%, greater than 64%, greater than 65%, greater than 66%, greater than 68%, greater than 70%, greater than 72%, greater than 74%. In some examples, the β-TCP is made using a similar method but as a composite with other agents, such as a biocompatible polymer, e.g., polylactide-co-glycolide.

As will be apparent to those of skill in the art, the composition can further include one or more pore forming agents. Examples of pore forming agents include, e.g., inorganic salts, such as sodium chloride, saccharides (e.g., sucrose or glucose), gelatin (e.g., gelatin spheres), or paraffin (e.g., paraffin spheres).

The compositions described herein can be generated by contacting any of the chimeric polypeptides or any of the targeting polypeptides to any of the types of β-TCP described herein. In some embodiments, the β-TCP can be in the form of a granular/powder form, a porous form, a putty (e.g., a moldable putty), a paste, a scaffold, and/or a coating on a solid surface (e.g., a coating on a medical device).

### Methods of Treatment

In one aspect, provided herein are methods of treating a subject with a polypeptide or composition described herein. For example, the subject is treated with a chimeric polypeptide described herein. As another example, the subject is treated with a composition described herein. For instance, a chimeric polypeptide comprising BMP-2 and a carrier comprising calcium phosphate. In some embodiments, the subject is suffering from a defect in bone, cartilage, soft tissue, tendon, fascia, ligament, organ, osteotendinous tissue, dermal, or osteochondral, or a combination of one or more of the aforementioned defects. In some embodiments, a defect is a lack of bone, cartilage, soft tissue, tendon, fascia, ligament, organ, osteotendinous tissue, dermal, or osteochondral, or a combination of one or more of the aforementioned defects. In some embodiments, a defect in the subject arises from trauma. In some embodiments, a defect in the subject arises due to a congenital condition. In some embodiments, a defect in the subject arises due to an acquired condition. Non-limiting examples of conditions suitable for treatment with a polypeptide or composition described herein include osteoarthritis, disc degeneration, congenital defect, spinal stenosis, spondylolisthesis, spondylosis, bone fracture, scoliosis, kyphosis, spinal fusion (PLF, and interbody fusions), trauma repair of bone, dental repair, craniomaxillofacial repair, ankle fusion, kyphoplasty, balloon osteoplasty, scaphoid facture repair, tendeno-osseous repair, osteoporosis, avascular necrosis, congenital skeletal malformations, costal reconstruction, subchondral bone repair, cartilage repair (e.g., at low doses), or trauma, or a combination thereof. BMP-2 is also involved in hair follicle development, therefore the methods may comprise treatment to hair follicles. The trauma may be to the bone, cartilage, soft tissue, tendon, fascia, ligament, organ, osteotendinous tissue, or dermal tissue, or osteochondral tissue. In some embodiments, the method is to treat an osteochondral injury.

In some embodiments, a defect refers to the absence, loss, and/or break in a tissue and/or organ of the body.

In some embodiments, a “bone defect” refers to the absence or loss (e.g., partial loss) of bone at an anatomical location in a subject where it would otherwise be present in a control healthy subject. A bone defect may be the result of, e.g., an infection (e.g., osteomyelitis), a tumor, a trauma, or an adverse event of a treatment. A bone defect may also affect the muscles, soft tissue, tendons, or joints surrounding the bone defect and cause injury. In some embodiments, a bone defect includes damage to a soft tissue.

In some embodiments, a “cartilage defect” refers to the absence or loss (e.g., partial loss) of cartilage at an anatomical location in a subject where it would otherwise be present in a control healthy subject. A cartilage defect may be the result of, e.g., disease, osteochondritis, osteonecrosis, or trauma. For example, a cartilage defect may affect the knee joint.

The method may comprise spinal fusion. In some embodiments, spinal fusion is a surgical technique to join two or more vertebrae. In some embodiments, the spinal fusion comprises PLF. In some embodiments, the spinal fusion comprises interbody fusions

Provided herein are methods of promoting bone or cartilage formation in a subject in need thereof that include: administering to the subject a therapeutically effective amount of any of the compositions described herein. Some embodiments of these methods can further include first selecting a subject in need of bone or cartilage formation. In some embodiments, the composition is administered to the subject proximal to the desired site of bone or cartilage formation in the subject.

Also provided herein are methods of replacing and/or repairing bone or cartilage in a subject in need thereof that include: administering to the subject a therapeutically effective amount of any of the compositions described herein. Some embodiments of these methods can further include first selecting a subject in need of bone replacement, bone repair, cartilage replacement, or cartilage repair. In some embodiments, the composition is administered to the subject proximal to the desired site of bone or cartilage replacement or repair in the subject.

Also provided herein are methods of treating a bone fracture or bone loss in a subject in need thereof, the method comprising administering to the subject a therapeutically effective amount of any of the compositions described herein. Some embodiments of these methods can further include first selecting a subject having a bone fracture or bone loss. In some embodiments, the composition is administered to the subject proximal to the bone fracture or the site of bone loss in the subject.

Also provided herein are methods of repairing soft tissue in a subject in need thereof that include: administering to the subject a therapeutically effective amount of any of the compositions described herein. Some embodiments of these methods can further include first selecting a subject having a bone fracture or bone loss. In some embodiments, the composition is administered to the subject proximal to the bone fracture or the site of bone loss in the subject.

Also provided herein are methods of localized delivery of a therapeutic to a subject in need thereof that include: administering to the subject a therapeutically effective amount of any of the compositions described herein. Some embodiments of these methods can further include first selecting a subject having a bone fracture or bone loss. In some embodiments, the composition is administered to the subject proximal to the bone fracture or the site of bone loss in the subject.

In some instances, the subject has a bone fracture or a bone defect.

In some instances, the subject requires a vertebral fusion of the spine.

In some instances, the subject has a cartilage tear or cartilage defect.

In other instances, the subject has cartilage loss.

Methods of determining the efficacy of treatment of a bone fracture or bone loss in a subject are known in the art and include, e.g., imaging techniques (e.g., magnetic resonance imaging, X-ray, or computed tomography).

Methods of detecting bone or cartilage formation, or replacement or repair of bone or cartilage in a subject are also known in the art and include, e.g., imaging techniques (e.g., magnetic resonance imaging, X-ray, or computed tomography).

Suitable animal models for treatment of a bone fraction or bone loss, bone or cartilage formation, or bone or cartilage replacement or repair are known in the art. Non-limiting examples of such animal models are described in the Examples and in, e.g., Drosse et al., Tissue Engineering Part C 14(1):79-88, 2008; Histing et al., Bone 49:591-599, 2011; and Poser et al., Hindawi Publishing Corporation, BioMed Research International; Article ID 348635, 2014.

As used herein, a method of treatment comprises administering to the subject a polypeptide or composition herein. In some embodiments, the subject is administered a chimeric peptide described herein, (e.g., comprising a targeting polypeptide and optionally a growth factor). In some embodiments, the subject is administered a composition comprising a chimeric peptide described herein and a carrier material. As a non-limiting example, the carrier material is a ceramic material. For instance, the ceramic material comprises calcium phosphate and/or hydroxyapatite.

In some embodiments, administration comprises implanting a polypeptide or composition herein.

In some embodiments, a polypeptide and/or composition herein comprising BMP-2 is administered to the subject. In some embodiments, the BMP-2 comprises a sequence at least about 70%, 75%, 80%, 85%, 90%, 95%, or 100% identical to SEQ ID NO: 32. In some embodiments, the BMP-2 is administered to induce formation of bone in the subject. In some embodiments, the BMP-2 is administered to induce formation of cartilage. In some embodiments, the BMP-2 is administered in a spinal fusion.

In some embodiments, about 0.5 mg to about 10 mg of a polypeptide is administered for every cubic centimeter (cc) of defect volume. For instance, about 0.5-10 mg, about 0.5-9 mg, about 0.5-8 mg, about 0.5-7 mg, about 0.5-6 mg, about 0.5-5 mg, about 0.5-4 mg, about 0.5-3 mg, about 0.5-2 mg, about 1-10 mg, about 1-9 mg, about 1-8 mg, about 1-7 mg, about 1-6 mg, about 1-5 mg, about 1-4 mg, about 1-3 mg, about 1-2 mg, about 2-10 mg, about 2-9 mg, about 2-8 mg, about 2-7 mg, about 2-6 mg, about 2-5 mg, about 2-4 mg, about 2-3 mg, about 3-10 mg, about 3-9 mg, about 3-8 mg, about 3-7 mg, about 3-6 mg, about 3-5 mg, about 3-4 mg, about 4-10 mg, about 4-9 mg, about 4-8 mg, about 4-7 mg, about 4-6 mg, or about 4-5 mg polypeptide is administered for every cc of the defect volume. In some embodiments, at least about 0.5, 1, 1.5, 2, 2.5 or 3 mg of the polypeptide is administered for every cc of the defect volume. In some cases, the polypeptide comprises a chimeric peptide described herein. In some cases, the polypeptide comprises one or more targeting polypeptide, e.g., as described herein. In some cases, the polypeptide comprises a growth factor, e.g., as described herein. In some cases, the defect volume is calculated or estimated by multiplying the length of the defect by the area of the defect (pi multiplied by defect radius squared).

Additional non-limiting embodiments of methods provided herein:

(1) In a first embodiment, provided is a method of delivering a therapeutic agent to an organ or tissue of a subject, the method comprising delivering to the organ or tissue a carrier material comprising the therapeutic agent. (2) The method of embodiment 1, wherein delivery comprises surgically introducing the carrier material to the organ or tissue. (3) The method of embodiment 1 or embodiment 2, wherein the tissue comprises cartilage. (4) The method of anyone of embodiments 1-3, wherein the organ comprises bone. (5) The method of any one of embodiments 1-4, wherein the therapeutic agent is bound to the carrier material. (6) The method of any one of embodiments 1-5, wherein the therapeutic agent is non-covalently bound to the carrier material. (7) The method of any one of embodiments 1-6, wherein the therapeutic agent binds to the carrier material or component thereof with a dissociation constant from about 1 fM to about 100 μM. (8) The method of embodiment 7, wherein the dissociation constant is from about 1 pM to about 100 μM. (9) The method of embodiment 7, wherein the dissociation constant is from about 1 nM to about 100 μM. (10) The method of embodiment 7, wherein the dissociation constant is from about 10 nM to about 100 μM. (11) The method of embodiment 7, wherein the dissociation constant is from about 10 nM to about 10 μM. (12) The method of any one of embodiments 1-11, wherein the carrier material comprises calcium phosphate. (13) The method of any one of embodiments 1-12, wherein the carrier material comprises tricalcium phosphate. (14) The method of any one of embodiments 1-13, wherein the carrier material comprises beta tricalcium phosphate. (15) The method of any one of embodiments 1-14, wherein the carrier material comprises about 20% to about 60% beta tricalcium phosphate by weight. (16) The method of any one of embodiments 1-15, wherein the carrier material comprises about 30% to about 50% beta tricalcium phosphate by weight. (17) The method of any one of embodiments 1-16, wherein the carrier material comprises about 35% to about 45% beta tricalcium phosphate by weight. (18) The method of any one of embodiments 1-17, wherein the carrier material comprises about 35%, 36%, 37%, 38%, 39%, 40%, 41%, 42%, 43%, 44%, or 45% beta tricalcium phosphate by weight. (19) The method of any one of embodiments 1-18, wherein the carrier material comprises about 40% beta tricalcium phosphate by weight. (20) The method of any one of embodiments 1-19, wherein the carrier material comprises alpha tricalcium phosphate. (21) The method of any one of embodiments 1-20, wherein the carrier material comprises hydroxyapatite. (22) The method of any one of embodiments 1-21, wherein the carrier material comprises fluorapatite. (23) The method of any one of embodiments 1-22, wherein the carrier material comprises bone. (24) The method of any one of embodiments 1-23, wherein the carrier material comprises demineralized bone. (25) The method of any one of embodiments 1-24, wherein the carrier material comprises a glass. (26) The method of embodiment 25, wherein the glass comprises a silicate. (27) The method of any one of embodiments 1-26, wherein the carrier material comprises a vanadate. (28) The method of any one of embodiments 1-27, wherein the carrier material comprises a ceramic mineral. (29) The method of any one of embodiments 1-28, wherein the carrier material comprises a chelated divalent metal ion. (30) The method of any one of embodiments 1-28, wherein the carrier material or a component thereof is in the form of fibers. (31) The method of any one of embodiments 1-28, wherein the carrier material or a component thereof is in a granular form. (32) The method of any one of embodiments 1-28, wherein the carrier material or a component thereof is in a porous form. (33) The method of any one of embodiments 1-28, wherein the carrier material or a component thereof is a powder. (34) The method of any one of embodiments 1-28, wherein the carrier material or a component thereof is a putty. (35) The method of any one of embodiments 1-28, wherein the carrier material or a component thereof is a paste. (36) The method of any one of embodiments 30-35, wherein the component thereof comprises calcium phosphate (e.g., tricalcium phosphate such as beta-TCP or alpha-TCP). (37) The method of any one of embodiments 30-35, wherein the component thereof comprises hydroxyapatite, demineralized bone, fluorapatite, a glass, vanadate, ceramic material, or chelated divalent metal ion, or any combination thereof. (38) The method of any one of embodiments 1-37, wherein the subject comprises a defect in organ or tissue, and the therapeutic agent is delivered to reduce or eliminate the defect in the organ or tissue. (39) The method of embodiment 38, wherein the defect in the organ or tissue comprises a bone defect. (40) The method of embodiment 38 or embodiment 39, wherein the defect in the organ or tissue comprises a cartilage defect. (41) The method of any one of embodiments 38-40, wherein the defect in the organ or tissue comprises a soft tissue defect. (42) The method of any one of embodiments 38-41, wherein the defect in the organ or tissue comprises a tendon defect. (43) The method of any one of embodiments 38-42, wherein the defect in the organ or tissue comprises a fascia defect. (44) The method of any one of embodiments 38-43, wherein the defect in the organ or tissue comprises a ligament defect. (45) The method of any one of embodiments 38-44, wherein the defect in the organ or tissue comprises an osteotendinous tissue defect. (46) The method of any one of embodiments 38-45, wherein the defect in the organ or tissue comprises a dermal defect. (47) The method of any one of embodiments 38-46, wherein the defect in the organ or tissue comprises an osteochondral defect. (48) The method of any one of embodiments 1-47, wherein the method is performed for spinal fusion in the subject. (49) The method of embodiment 48, wherein the spinal fusion comprises posterior lumbar fusion (PLF). (50) The method of embodiment 48, wherein the spinal fusion comprises interbody fusion. (51) The method of any one of embodiments 1-47, wherein the method is performed for trauma repair of bone. (52) The method of any one of embodiments 1-47, wherein the method is performed for dental repair. (53) The method of any one of embodiments 1-47, wherein the method is performed for craniomaxillofacial repair. (54) The method of any one of embodiments 1-47, wherein the method is performed for ankle fusion. (55) The method of any one of embodiments 1-47, wherein the method is performed for kyphoplasty. (56) The method of any one of embodiments 1-47, wherein the method is performed for balloon osteoplasty. (57) The method of any one of embodiments 1-47, wherein the method is performed for scaphoid facture repair. (58) The method of any one of embodiments 1-47, wherein the method is performed for tendeno-osseous repair. (59) The method of any one of embodiments 1-47, wherein the method is performed to treat osteoporosis. (60) The method of any one of embodiments 1-47, wherein the method is performed to treat avascular necrosis. (61) The method of any one of embodiments 1-47, wherein the method is performed to treat congenital skeletal malformations. (62) The method of any one of embodiments 1-47, wherein the method is performed for costal reconstruction. (63) The method of any one of embodiments 1-47, wherein the method is performed for subchondral bone repair. (64) The method of any one of embodiments 1-47, wherein the method is performed for cartilage repair. (65) The method of any one of embodiments 1-47, wherein the method is performed on a hair follicle (BMP-2 is involved in hair follicle development). (66) The method of any one of embodiments 1-65, wherein the subject is a mammal. (67) The method of any one of embodiments 1-66, wherein the subject is a human. (68) The method of any one of embodiments 1-67, wherein the subject is a non-human mammal. (69) The method of embodiment 68, wherein the method is used in veterinary applications. (70) The method of any one of embodiments 1-69, wherein the therapeutic agent is bound to the carrier material via a targeting polypeptide. (71) The method of embodiment 70, wherein the targeting polypeptide is connected to the therapeutic agent. (72) The method of embodiment 71, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 1. (73) The method of embodiment 71 or embodiment 72, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 2. (74) The method of any one of embodiments 71-73, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 3. (75) The method of any one of embodiments 71-74, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 4. (76) The method of any one of embodiments 71-75, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 5. (77) The method of any one of embodiments 71-76, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 6. (78) The method of any one of embodiments 71-77, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 7. (79) The method of any one of embodiments 71-78, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 8. (80) The method of any one of embodiments 71-79, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 9. (81) The method of any one of embodiments 71-80, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 10. (82) The method of any one of embodiments 71-81, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 11. (83) The method of any one of embodiments 71-82, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 12. (84) The method of any one of embodiments 71-83, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 13. (85) The method of any one of embodiments 71-84, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 14. (86) The method of any one of embodiments 71-85, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 15. (87) The method of any one of embodiments 71-86, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 16. (88) The method of any one of embodiments 71-87, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 17. (89) The method of any one of embodiments 71-88, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 18. (90) The method of any one of embodiments 71-89, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 19. (91) The method of any one of embodiments 71-90, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 20. (92) The method of any one of embodiments 71-91, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 21. (93) The method of any one of embodiments 71-92, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 22. (94) The method of any one of embodiments 71-93, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 23. (95) The method of any one of embodiments 71-94, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 24. (96) The method of any one of embodiments 71-95, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 25. (97) The method of any one of embodiments 71-96, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 26. (98) The method of any one of embodiments 71-97, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 27. (99) The method of any one of embodiments 71-98, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 28. (100) The method of any one of embodiments 71-99, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 29. (101) The method of any one of embodiments 71-100, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 30. (102) The method of any one of embodiments 71-101, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 31. (103) The method of any one of embodiments 71-102, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 35. (104) The method of any one of embodiments 71-103, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 36. (105) The method of any one of embodiments 71-104, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 37. (106) The method of any one of embodiments 71-105, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 38. (107) The method of any one of embodiments 71-106, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 39. (108) The method of any one of embodiments 71-107, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 40. (109) The method of any one of embodiments 71-108, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 41. (110) The method of any one of embodiments 71-109, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 42. (111) The method of any one of embodiments 71-110, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 43. (112) The method of any one of embodiments 71-111, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 401. (113) The method of any one of embodiments 71-112, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 402. (114) The method of any one of embodiments 71-113, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 403. (115) The method of any one of embodiments 71-114, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 404. (116) The method of any one of embodiments 71-115, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 405. (117) The method of any one of embodiments 71-116, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 406. (118) The method of any one of embodiments 71-117, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 407. (119) The method of any one of embodiments 71-118, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 408. (120) The method of any one of embodiments 71-119, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 409. (121) The method of any one of embodiments 71-120, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 410. (122) The method of any one of embodiments 71-121, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 411. (123) The method of any one of embodiments 71-122, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 412. (124) The method of any one of embodiments 71-123, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 413. (125) The method of any one of embodiments 71-124, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 414. (126) The method of any one of embodiments 71-125, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 415. (127) The method of any one of embodiments 71-126, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 416. (128) The method of any one of embodiments 71-127, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 417. (129) The method of any one of embodiments 71-128, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 418. (130) The method of any one of embodiments 71-129, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 419. (131) The method of any one of embodiments 71-130, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 420. (132) The method of any one of embodiments 71-131, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 421. (133) The method of any one of embodiments 71-132, wherein the targeting polypeptide comprises a sequence at least about 70%, 75%, 80%, 85%, 90%, 91%, 92%, 93%, 94%, 95%, 96%, 97%, 98%, 99%, or 100% identical to SEQ ID NO: 422. (134) The method of any one of embodiments 70-133, wherein the therapeutic agent and targeting polypeptide are connected via a linker. (135) The method of embodiment 134, wherein the linker comprises a peptide. (136) The method of embodiment 135, wherein the linker comprises a sequence at least about 70%, 75%, 80%, 85%, 90%, 91%, 92%, 93%, 94%, 95%, 96%, 97%, 98%, 99%, or 100% identical to SEQ ID NO: 701. (137) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 32. (138) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 46. (139) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 47. (140) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 152. (141) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 168. (142) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 268. (143) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 176. (144) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 48. (145) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 49. (146) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 50. (147) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 51. (148) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 52. (149) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 53. (150) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 54. (151) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 55. (152) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 56. (153) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 57. (154) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 58. (155) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 59. (156) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 60. (157) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 61. (158) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 62. (159) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 63. (160) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 64. (161) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 65. (162) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 66. (163) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 67. (164) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 68. (165) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 69. (166) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 70. (167) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 71. (168) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 72. (169) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 73. (170) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 74. (171) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 75. (172) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 76. (173) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 77. (174) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 78. (175) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 79. (176) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 80. (177) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 81. (178) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 82. (179) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 83. (180) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 84. (181) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 85. (182) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 86. (183) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 87. (184) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 88. (185) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 89. (186) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 90. (187) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 91. (188) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 92. (189) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 93. (190) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 94. (191) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 95. (192) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 96. (193) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 97. (194) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 98. (195) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 99. (196) The method of any previous embodiment, wherein the therapeutic agent comprises a sequence at least about 70%, 75%, 80%, 85%, 90%, 91%, 92%, 93%, 94%, 95%, 96%, 97%, 98%, 99%, or 100% identical to SEQ ID NO: 100. (197) The method of any previous embodiment, wherein the therapeutic agent is part of a composition comprising any one of SEQ ID NOS: 501-648.

Also provided herein are devices comprising a biological material. In some cases, the biological material comprises a growth factor as listed in Table B, or a polypeptide comprising a sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to a sequence of Table B.

In some embodiments, provided is a method of coating a device with a biologic material, the method comprising coating the device with any targeting polypeptide herein. In some embodiments, provided is a method of coating a device with a biologic material, the method comprising coating the device with any chimeric polypeptide herein. In some embodiments, provided is a method of coating a device with a biologic material, the method comprising coating the device with any polypeptide comprising a polypeptide of Table A. In some embodiments, provided is a method of coating a device with a biologic material, the method comprising coating the device with any polypeptide comprising a polypeptide of Table B. In some embodiments, provided is a method of coating a device with a biologic material, the method comprising coating the device with any polypeptide comprising a polypeptide of Table C. In any of the previous embodiments, the composition and/or biologic material is non-covalently bound to the device. In any of the previous embodiments, the device comprises one or more carrier materials described herein.

The following examples do not limit the scope of the claims.

### EXAMPLES

### Example 1. A Preclinical Study of tBMP-2 in a β-TCP Carrier in a Rat Critical Size Femoral Defect Model

The aim of this study was to examine the safety and effect of modified recombinant human bone morphogenetic protein (rhBMP) known as tBMP-2 in a beta-tricalcium phosphate (β-TCP) putty matrix in the replacement and repair of bone in a rat critical size defect model. The tBMP-2 used in this study comprises MPIGSLLADTTHHRPWTVIGESTHHRPWSIIGESSHHKPFTGLGDTTHHRPWGILAESTHHKP WTASGAGGSEGGGSEGGTSGATGAGTSTSGGGASTGGGTGQAKHKQRKRLKSSCKRHPLY VDFSDVGWNDWIVAPPGYHAFYCHGECPFPLADHLNSTNHAIVQTLVNSVNSKIPKACCVPT ELSAISMLYLDENEKVVLKNYQDMVVEGCGCR (SEQ ID NO: 502). The tBMP-2 variant exhibits very tight binding to calcium phosphate ceramics and thus allows for targeted delivery of tBMP-2 to implant sites. This was achieved by surgically creating the defect using the RatFix internal fixator system, then filling that defect with tBMP-2+β-TCP putty, or β-TCP putty alone as a control. The positive control was a dose-adjusted commercially available (Medtronic) preparation for human use of rhBMP with an absorbable collagen sponge (ACS). The animals were monitored clinically and had bi-weekly radiographs until their scheduled end-points at four or eight weeks post-administration of treatment. Ex vivo histology was conducted on organs and tissues, and the treated leg was subjected to μCT, histomorphometry, and/or mechanical strength testing.

This report covers animal arrival, assignment to groups, surgical creation of defect and administration of test, reference or control item. In addition, this report covers the post-operative period up to and including Week 8, and end-point in vivo and ex vivo measurements. Included are the clinical summaries, radiographs, end-point clinical pathology and organ histopathology, histomorphometry of treated legs (odd-numbered animals), and mechanical strength testing of treated legs (even-numbered animals).

On arrival at the Test Facility, all animals underwent a Veterinary health check and were weighed. Based on the weights, the animals were assigned to groups A) tBMP-2+β-TCP putty; B) rhBMP+ACS; or C) β-TCP putty alone. The animals then had at least five days to acclimatize to their new surroundings and diet.

On the day of surgery, the animals were anaesthetized, the defect was created and the bone supported with the internal fixator following the method supplied by the manufacturer with some refinements to the surgical procedure. The pre-prepared test, reference or control item was then inserted into the defect. The surgical site was closed and the animal went immediately for X-ray. Following completion of the baseline radiograph, post-operative pain relief was administered and the animal was returned to its cage for recovery. Post-operative analgesia and antibiotic therapy were continued for at least two days, and longer when required.

A number of the original animals were removed from the study and replaced prior to surgery as they were getting too large for the fixator system specifications. The second, replacement group also had a thorough Veterinary health check and were weighed prior to being assigned to groups. The allocation of animals to either four or eight-week end-point groups was adjusted to include animals from both weight ranges at both end-points.

A total of 61 animals were successfully administered assigned treatment and recovered from surgery. Of these, one animal was euthanized five days post-operatively due to poor recovery and inappetence. The animal was replaced. Two animals which were found to have dislodged bone plates during their 2-week post-operative radiographs were humanely killed and not replaced. Two animals did not successfully recover from surgery, one died immediately post-surgery, and the other had an unstable bone plate which could not be fixed, and so was humanely killed. Both of these animals were replaced immediately.

The animals were monitored at least once daily, and their clinical signs scored. Any animal with unusual findings or considered to be not eating and gaining weight normally was monitored more frequently, and veterinary advice was sought. When unusual findings were noted, this resulted in possible treatment with additional antibiotic or analgesic therapy, fluid supplementation), and in four cases euthanasia (three broken bones and one with continued weight loss).

All relevant animals received bone-labelling dyes at 10 days (calcein green) and 3 days (xylenol orange) prior to their scheduled end-point date. All animals had end-point blood samples collected. There were no clinically significant differences found between any of the groups for any biochemical or haematological parameter at either end-point time. Three haematology samples were clotted and could not be analyzed.

In all specimens examined, there was evidence of extensive mineralization akin to mature trabecular bone in samples from animals in the tBMP-2+β-TCP putty group (Group A). This mineralization appears to be lamella (mature) bone rather than the woven bone that occurs in early callus formation (presence of osteocytes). Group A animals also had osteoclasts present (suggesting active remodeling), and red blood cells and adipocytes, indicating angiogenesis and infiltration of cells to most regions of the callus. Whilst there is mineralized bone in the specimens from the rhBMP+ACS group animals, there are extensive regions of un-mineralized fibrous and cartilaginous tissue within the callus region. The callus development in these samples appears to be at an earlier stage than that observed in the Group A animals. The Group C (β-TCP putty) samples show no evidence of mineralization, bone formation or bone remodeling even at eight weeks post-application of treatment.

The mechanical testing analyses showed the tBMP-2+β-TCP group animals (Group A) had significantly stronger bones at the four and eight week end-point times than either of the other treatment groups.

In conclusion, the bone specimens form the tBMP-2+β-TCP putty group clearly demonstrated vastly improved bone healing at 4 and 8 weeks post-surgery when compared to samples from animals in the rhBMP+ACS or the β-TCP putty alone treatment groups. The aim of this study was to evaluate the performance of a modified variant of recombinant human BMP-2 (rhBMP-2) called tBMP-2. The tBMP-2 variant exhibits very tight binding to calcium phosphate ceramics and thus allows for targeted delivery of tBMP-2 to implant sites. The ability to tether BMP-2 in this manner can allow for longer persistence times, lower doses, and, it is expected, superior outcomes as compared to the treatment controls studied herein.

### Animals

The rat is a validated animal model for assessing the effect of treatments on critical-sized defects in the femur (4-6). Sixty male rats (plus spares) were required: ten animals in each group per time point. One animal was replaced after successful administration of the test item at day 5 post-surgery as he was losing weight and not eating. Animals that were euthanized or humanely killed after successful administration of the test item were necropsied and had tissue collected for histopathology. The number of animals used in this study was considered sufficient for evaluation of results.

The animals were housed in conventional conditions (targeted temperature 22±3° C., 12/12-hour light/dark cycle) in standard open top cages that satisfy the size requirements specified in the Animal Welfare Regulations (Animal Welfare Act 1985, v 15.10.2015. Attorney General's Department, Gouverment of South Australia). Animals were housed individually for the entirety of the experiment. The animals had access to standard laboratory rat chow (Specialty Feeds, Glen Forest, Australia) 25-30 g/day. Immediately post-operatively, and when rats were not thriving, the food was soaked in water for easier palatability. Chlorinated tap water was provided to the animals ad libitum. Food and water were not withheld at any time.

Each animal was uniquely identified by a subcutaneously implanted microchip which was scanned using a barcode reader. For the purpose of the study, each animal was also given a number from 1-70 (to include replacement and spared required). As the initial batch of animals gained weight too quickly, some animals were replaced prior to surgery. The replacement animals were given the number of the animal they replaced and the suffix (a) was added. Numbers went up to 68a with a couple of gaps.

Animals were assigned to groups by a weight-ordered distribution. The heavier animals were used first. The animals gained weight rapidly, even on the restricted food allowance. Two weeks into the surgeries, it was deemed necessary to remove some animals from the study (n=16) as these animals were already too heavy (>425 g). These animals were replaced with animals in the 250-300 g range. The replacement animals were also assigned to treatment groups on a weight-ordered distribution. As the first surgical group had originally been assigned to the 8-week end-point group, animals were reassigned to either 4- or 8-week end-points to ensure the lighter animals were evenly distributed amongst the treatment and end-point times.

### Analgesia/Antibiotic Therapy

All interventional procedures were performed under isoflurane in O₂ anaesthesia. Induction and maintenance of surgical depth anesthesia was at 2-4% isoflurane (Baxter International, Sydney, Australia) in a flow of 1-2 L/minute O₂. Post-operatively, after closure of the wound, a topical application of local anesthetic in the form of Marcaine (Bupivacaine, 0.5%, AstraZeneca, Frewville, SA, Australia) up to 2.5 mg/Kg was applied to the area around the wound. The animals received 0.1 mg/kg buprenorphine (Temgesic, Reckitt-Benckiser, Melrose Park, Australia) subcutaneously post-operatively after X-ray. They also received 0.1 mg/kg subcutaneously twice daily for two days post-operatively. Additionally, they were given a non-steroidal anti-inflammatory treatment in the form of Carprieve (Carprofen, 50 mg/mL, Norbrook Laboratories, Tullamarine, Australia) at a dose of 5 mg/kg subcutaneously post-operatively and once daily for at least two days post-surgery.

All animals received cephalosporin (Cefazolin, Hospira Inc, Lake Forest IL, USA) 20 mg/kg subcutaneously intra-operatively and twice daily for two days post-operatively. Any animals that needed re-suturing of their wounds received antibiotic treatment for up to fourteen days post-repair. Antibiotic therapy was ceased if suspected to be causing diarrhea and/or weight loss.

### Microchip Implantation

The microchip was inserted into the scruff of the neck (after clipping hair) using a microchip implanter. The microchip was inserted until completely covered by skin. The wounds were closed with one or two wound clips if required. The area was swabbed with betadine. This was performed under isoflurane in O₂ anaesthesia at the same time as the defect surgery and test- or reference-item placement.

### Critical-Size Defect Creation

The defects were created in the right femur of all rats as described for the RatFix RISystem. Briefly, all anaesthetized rats were placed on a warming pad in lateral recumbency with the right leg facing upwards. The surgical site was shaved and aseptically prepared with iodine or chlorhexidine scrub and solution. A skin incision between the greater trochanter and the knee joint was made and the superficial fascia incised. The intermuscular plane between the vastus lateralis and the biceps femoris was separated and the periosteum of the femur incised. The PEEK plate was fitter into the jug and secured with suture material. The jig-plate assembly was fixed to the craniolateral surface of the femur by pulling the sutures through under the femur, allowing the assembly to be tightened to the femur.

After predrilling the holes in the PEEL plate using the supplied drill bit, the plate was attached to the femur by six bicortical titanium screws. Standardised 6-mm defects were created (marked on the plate) using the Gigli wire saw guided by the sawing device of the jig. After defect sawing, the jig and bone piece were removed. The fresh defect was flushed with sterile saline and dried with gauze in preparation of test item or reference item, or vehicle administration into the defect size. In the event that the plate itself was damaged with the cutting wire, reducing stability, that animal would be replaced. No replacements due to plate damage were required.

### Baseline Radiographs

Baseline radiographs were taken immediately after creation of the defect and administration of the test or reference item to show position of treatment articles (where visible), and placement of the plate, whilst the animal was still under anaesthesia.

### Radiographic Assessments

X-rays were taken on the anaesthetized animals (isoflurane) immediately after surgery, and at 2, 4 (end-point for half of the animals), 6 and 8 weeks (end-point for the remaining animals) post-operatively. The radiographs were taken in lateromedial and craiocaudal projections for assessment of bone healing and to exclude implant loosening or failure. All in-life radiographs were taken using a Villa Visitor Mobile X-ray Unit (Villa Sistemi Medicali, Buccinasco, Italy), using a dental image capture device (Soredex, Digora Optime, Tuusula, Finland). The images were transferred to the PIRL picture archiving and communication system (PACS, Carestream Vue Motion, Rochester, USA) by the radiographer for storage and access.

### Bone Labelling

At 10 days prior to their prescribed end-point, all animals received an intraperitoneal injection of calcein green (25 mg/kg in saline). At 3 days prior to their prescribed end-point all animals received an injection of xylenol orange (10 mg/mL, 25 mg/kg, intraperitoneal) to double-label the bone for histomorphometric analyses.

### Blood Collection/Haematology

All blood collections were performed on anaesthetized animals at their end-point radiograph. Blood was collected via cardiac puncture.

Blood samples were evaluated for the parameters specified in Tables 1 and 2. For Table 1, samples of approximately 2 mL were collected into tubes containing K2EDTA anticoagulant for haemotological analyses. Analyses were performed on an Abbott Cell Dyn. 3700 (Abbott Laboratories, North Ryde, Australia). For Table 2, samples of approximately 5 mL were collected into tubes containing a clot activator for biochemical analysis. Analyses were performed on a Siemens Advia 1800 (Siemens Healthcare Diagnostics Inc., Flanders, NJ, USA).

TABLE 1
Haematology Parameters to be reported

| | |
|---|---|
| Haemoglobin (Hb) | White Cell Count (WCC) |
| Erythrocyte count (RBC) | Mean Corpuscular Volume (MCV) |
| Packed Cell Volume (PCV)/Haematocrit | Mean Corpuscular Haemoglobin (MCH)* |
| (HCT) | |
| Mean Corpuscular Haemoglobin Concentration | Red Cell Distribution Width (RDW) |
| (MCHC)* | |
| Platelets (Pit) | |

White Blood Cell Differential: Neutrophils, Lymphocytes, Monocytes, Eosinophils, Basophils
*Calculated values

TABLE 2
Serum Chemistry Parameters to be reported

| | | |
|---|---|---|
| Electrolytes: | Anion Gap* (AG) | Albumin (Alb) |
| Sodium (Sod) | Glucose (Glue) | Globulin* (Glob) |
| Potassium (Pot) | Urea | Protein (Tot Prot) |
| Chloride (Chi) | Creatinine (Creat) | Total bilirubin (Tot Bili) |
| Bicarbonate (Bicarb) | Cholesterol (Chol) | Lactate Dehydrogenase (LD) |
| Lipid Studies: | Urate (Uric Acid, UA) | Alkaline phosphatase (ALP) |
| Triglycerides (Trig) | Phosphate (Phos) | Total Calcium (Tot. Ca) |
| High Density Lipoproteins | Gamma glutamyltransferase (GGT) | Alanine aminotransferase (ALT) |
| (HDL) | Asparatate aminotransferase (AST) | |
| Low Density Lipoproteins | | |
| (LDL) | | |
| Chol/HDL* | | |

*Calculated values

### Mechanical Strength Testing

This was performed on half of the animals at each time point. All tissue was placed in 0.9% NaCl-soaked gauzed in 50-mL sterile urine pots to avoid freezer burn and stored frozen at −20° C. until testing. Tissue was prepared immediately and frozen within one hour of collection to avoid autolysis. Immediately before testing, tissues were thawed, and the internal fixator device carefully cut in the middle section. Tissue was test at room temperature (approximately 23° C.). The ultimate breaking strength was measured by using a load frame (model 5542, Instron, Canton, MA) and a 3-point bend fixture (model 2810-400, Instron) at a crosshead speed of 10 mm/min. The load cell for this testing (Instron 2530-416) had a maximum capacity of 500 N. The data (force in kg and extension in mm) was collected and analyzed with a vendor-provided commercial mathematical software package (Bluehill2, Instron).

### Histological Analyses

Specimen were fixed in 10% formalin for 7 days prior to processing. Formalin-fixed bones were cut using a slow-speed saw (Buehler Ltd, IL, USA) along the sagittal plane using a diamond-tipped cutting blade before being submerged in 70% ethanol. Subsequently, bones were processed for resin embedding via several dehydration steps. Briefly, bones were submerged in 2×90% ethanol and 1×100% ethanol steps over 48 hours. Bones were then transferred into a solution containing methymethacrylate (MMA) and 10% v/v polyethylene glycol (PEG) and stored at room temperature for 10-14 days. Resin embedding then occurs by preparing a solution of MMA, 10% PEG, and 0.4% peroxydicarbonate (Perkadox) and incubation at 37° C. for 24 hours to allow the resin to harden. The exposed cut surface was placed facing down in the tube. The resin-set bones were then removed from their tubes and fixed to stubs for sectioning. For each bone, 5-μm thick sections were cut using a tungsten-carbide blade (Leica RM2255, Wetzlar, GER). Sections were placed on to gel-coated slides and dipped 2 times in a spreading solution (70% ethanol/30% 2-ethoxyethanol) heated to 70° C. To ensure adherence to the slide, sections were flattened, covered by strips of polyethylene and clamped together separated by blotting paper, before being placed into a 37° C. oven. Prior to commencing staining procedures, sections were placed in acetone for 15 minutes, unless otherwise stated. All sections were digitally scanned at 100× magnification (3D Histech scanner, TMA-MASTER01).

### Von Koss+Haemaotoxylin and Eosin (H&E) Staining

Acetone treated sections were rinsed in demineralised water for 2 minutes. Sections were then placed in a 1% silver nitrate solution and placed in front of a UV lamp for 60 minutes. Washed slides were then treated with 2.5% sodium thiosulphate solution for 5 minutes. Washed slides were then counterstained with H&E. To stain for H&E, sections were placed in haematoxylin for 8 minutes to stain cell nuclei before being rinsed in demineralised water for 2 minutes and dipped in acid alcohol (%), typically, 4-5 minutes. After rinsing in demineralised water, sections were dipped in lithium carbonate 4-5 times and placed in eosin for 4 minutes. Once removed, excess eosin was removed with a squeeze bottle of absolute alcohol, sections were hydrated, placed in xylene and mounted in xylene-based mounting medium.

### Tartrate-Resistant Acid Phosphatase (TRAP) Stain for Analysis of Osteoclasts

Acetone treated sections underwent a 60-minute incubation in Tris-HCL buffer (pH 9.4) at 37° C. for 60 minutes in an acid phosphatase (AcP) stain prepared by adding 0.0355 g of tartaric acid dissolved in 35 mL of sodium acetate (pH 5.2) to 100 μL basic fucshin in a 100 μL solution containing 0.4 mg of sodium nitrite. This solution was then added to a solution containing 0.04 g Napthol ASBI phosphate (Sigma-Aldrich, Missouri, USA) in 2 mL dimethylformamide. Subsequently, two washed were performed before the sections were counterstained in haematoxylin for 8 minutes, then rinsed in demineralised water, then dipped in acid alcohol 4 times, rinsed again, dipped in lithium carbonate 4 times and then dehydrated, placed in xylene and mounted in DPX. Quantification of osteoclast number per bone perimeter mm was performed by identifying cells (stained pink-red) on the surface of bone (stained blue-purple) and calculated by the OsteoMeasure histomorphometry system.

### Double Fluorochrome Labelling of Bone Sections for Measures of Bone Formation

Acetone sections were immediately placed in xylene and mounted in DPX. Slides were viewed under a fluorescent microscope (Olympus BX53; Olympus, Tokyo, JP).

### Histomorphometry

Those animals which did not have mechanical testing of femurs will undergo histological analysis of the defect and treatments. Following μCT of the femurs, they were opened at the wound site, and the internal fixator device carefully removed. They were placed in 70% ethanol. Femurs were placed in PMMA, polymethyl methacrylate (10% polyethylene glycol [PEG] in methylmethacrylate) for 10-14 days, then polymerized in PMMA containing 10% Perkadox 16 (di[4-tert-butylcyclohexyl] peroxydicarbonate) at 37° C. for 24 hours. Sections were stained with H&E, and tartrate resistant acid phosphatase (TRAP) for analysis.

### Humane Killing

Animals were humanely killed on day 28±2 days (n=30) or day 56±3 (n=30) immediately following their end-point radiograph and blood collection, and whilst still under anaesthesia, with an intracardiac injection of a lethal dose of sodium pentobarbitone of 60 mg/mL formulation (Lethabarb®) at 200 mg/kg. Death was confirmed by lack of respiration and palpable heartbeat, loss of corneal reflex and loss of colour of mucous membranes.

### Necropsy

All animals were subjected to a comprehensive necropsy. A comprehensive necropsy is defined as examination of the external surface of the body, all orifices, and the cranial, thoracic, and abdominal cavities, and their contents. If abnormalities were found in tissues or organs other than those listed below, they were also collected for histology.

### Organ and Tissue Collection

Whole organs, sections of the issues listed in Table 3 below were dissected free and fixed in 10% neutral buffered formalin. This was done for twelve animals from each groups (six animals from each group at each time-point). The animals were selected such that at least one from each group was selected at each end-point (staggered as surgeries were staggered).

TABLE 3
Organ and Tissue Samples Collected From Each Animal

| | | |
|---|---|---|
| Adrenal gland | Lesions | Skin (containing implant[s]) |
| Brain | Liver | Spinal cord (cervical, |
| | | thoracic, and lumbar) |
| Cecum | Lymph node | Spleen |
| | (mesenteric) | |
| Duodenum | Pancreas | Sternum (+ bone marrow) |
| Eye | Pituitary Gland | Stomach |
| Heart | Rectum | Thymus |
| Ileum | Salivary gland | Tongue |
| | (mandibular) | |
| Jejunum | Sciatic nerve | Epididymis |
| Kidney | Skeletal muscle | |
| | (thigh) | |
| Femur with bone | Lung (with mainstem | Prostate/seminal vesicles, |
| marrow (articular | bronchi) | bladder, testes |
| surface of the | | |
| distal end) | | |

### Treated Limb Collection and Storage

Immediately following confirmation of death, the treated leg was removed and placed in either 10% neutral buffered formalin (n=5 animals/group/time point), or wrapped in saline-soaked gauze and placed at −20° C. for at least 48 hours (n=10 animals/group/time point).

### μCT

All legs had μCT measurements (Bruker Skyscan 1076, Brussels, Belgium) of the defect performed to quantify newly mineralized bone volume. This was done on all animals at each end-point time. The bones were either formalin-fixed (105 neutral buffered formalin) or frozen at −20° C. for 3-5 days prior to testing. The plate was adjusted to the longitudinal axis of the device. Scanning parameters and intensity were recorded and were in the vicinity of a source voltage of 70 kV with an intensity of 114 μA. The end-point μCT was manually and subjectively scored as described by Chhabra et al. (2005) using the grading system described in Table 4.

TABLE 4
Radiographic Grading Scale of Fracture Callus Formation

| Grade | Amount of Callus Formation |
|---|---|
| 0 | No callus |
| 1 | Little-to-moderate callus |
| 2 | Profuse callus tissue |
| 3 | Bridging periosteal callus |
| 4 | Mature callus with interfragmentary |
| | bridging |
| 5 | Callus resorption after solid union |

### Study Design

The study was performed as outlined in Table 5. There were three groups with 20 animals/group. All animals underwent surgery to create a critical-size defect (6-mm) in the mid diaphysis of the right femur (RatFix RISystem), and had inserted into the defect test item, reference item or vehicle. The animals were monitored at least once daily and were weighed at least twice weekly. They underwent radiographic evaluation immediately post-operatively, and at 2, 4, 6, and 8 weeks post-surgery. Ten animals from each group (n=30 animals) were scheduled to have end-point data (μCT, mechanical testing, histomorphometry, clinical pathology) collected 4 weeks post-operatively and the remaining animals (n=30 animals) were scheduled for end-point data collection 8-weeks post operatively. Of these, six from each group (chosen such that there was a representative from each group from the majority of surgical days) at each end-point had tissues and organs collected for histology.

TABLE 5
Study design

| | | | End-Point (n) | |
|---|---|---|---|---|
| Group | Treatment | Nomenclature | Week 4 | Week 8 |
| A | tBMP-2 + β-TCP putty | tBMP-2 + | 10 | 10 |
| | | β-TCP putty | | |
| B | rhBMP-2 + absorbable | rhBMP2 + ACS | 10 | 10 |
| | collagen sponge (ACS) | | | |
| C | β-TCP putty | β-TCP putty | 10 | 10 |

Group A animals received tBMP-2+β-TCP (0.615-0.620 mg tBMP-2 in β-TCP putty to fill defect); Group B animals received InFUSE™ Bone Graft of rhBMP-2+ACS (2 μg rhBMP-2 on ACS to fill defect); Group C animals received β-TCP to fill the defect. All test and control items were mixed on the day of surgery (tBMP-coated β-TCP to putty or rhBMP to ACS). Following administration of the treatment article, the wounds were closed in two muscle layers with subcutis and intracutaneous vicryl sutures. The wounds were closed exteriorly with intradermal sutures to prevent chewing by the rats. The wounds were washed with liberal amounts chlorhexidine solution. A small amount of tea tree oil was applied to the area surrounding the actual wound to prevent the animals from worrying the suture side.

### Route of Administration

The anticipated route of human administration is by surgical implantation into a bone defect. Therefore, that route was used in this study. The bone defects were surgically created, the area flushed with saline, then the test or control items were placed in the defect immediately.

Preparation of Test Item (tBMP-2+-TCP Putty)

For each rat, 195 μL of tBMP-2 (stock 3.53 mg/mL) were added to a pre-weighed aliquot of 47 mg β-TCP granules and mixed gently for 2-3 hours. All liquid was removed, and retained for analysis. The pellet was washed with 1 mL of sterile phosphate-buffered saline (PBS pH 7.4) and mixed gently to wash away any excess unbound protein. As much as possible of the liquid was removed with a pipette, and sterile pre-weighed putty was added to the protein-coated β-TCP. β-TCP and putty were mixed 1:1. The entire tBMP-2+β-TCP putty formulation was placed in the surgically-created defect. Assuming approximately 90% binding, the final dose of tBMP which was administered to the rat in the defect was 615-620 μg. Sterility was maintained at all steps. β-TCP binding to tBMP-2 was done no more than 48 hours prior to surgery. If β-TCP was bound/washed the day before surgery, it was stored at 4° C. β-TCP and putty were not mixed more than one hour prior to implantation, as it will dehydrate with time and become less malleable.

### Reference Item

Recombinant human bone morphogenetic protein+absorbable collagen sponge (InFUSE™ Bone Graft; size XX small; Medtronic Sofamor Danek, Inc) was used as per packet insert with modifications as described below. BMP-2 vial contents were reconstituted with the provided sterile water to give 1.5 mg/mL rhBMP-2. This concentration is intended for human use. To bring this concentration to the range typically used in rats, it was diluted 1 in 60 with PBS (25 μg/mL). A defect volume of approximately 75.5 mm³ was assumed, therefore the ACS was trimmed to form a 6-mm×4-mm block for insertion into the defect. Diluted rhBMP solution (80 μL) were added to the ACS in a dropwise fashion at least 15 minutes prior to insertion into the defect. This resulted in 2 μg tBMP-2 in the defect. Sterility was maintained.

### β-TCP Putty

β-TCP granules (46-48 mg aliquots), carboxymethyl cellulose putty (48-50 mg aliquots). Under sterile conditions, the β-TCP granules were washed with 1 mL of sterile PBS and mixed gently. As much as possible of the liquid was removed using a pipette. Pre-weighed sterile carboxymethyl cellulose (approximately 2 mg more than the β-TCP putty formulation was inserted into the defect.

### Dose Formulation

The rhBMP-2+absorbable collagen sponge and the tBMP-2+β-TCP putty were prepared as described above. The final dose each rat received of the BMP-2 formulations was 2 μg rhBMP-2 for the reference item and approximately 615-620 μg tBMP of the test item.

### Animal Assignments and Body Weights

On arrival, the initial animals were numbered 1-63, of which 60 were assigned to the study. The animals were fed ad lib on arrival, but this was reduced to 35-30 g when it was found at their third weighing that they were gaining weight too quickly. The initial surgeries were delayed by one week, and as the animal's weights continued to increase, the decision was made to remove the heaviest sixteen animals from the study and get replacement animals. The replacement animals were assigned numbers.

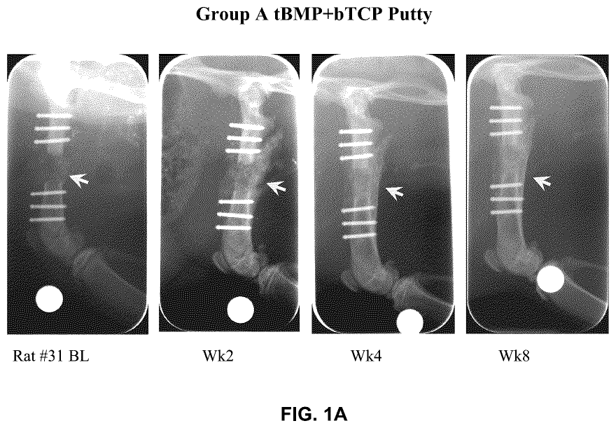

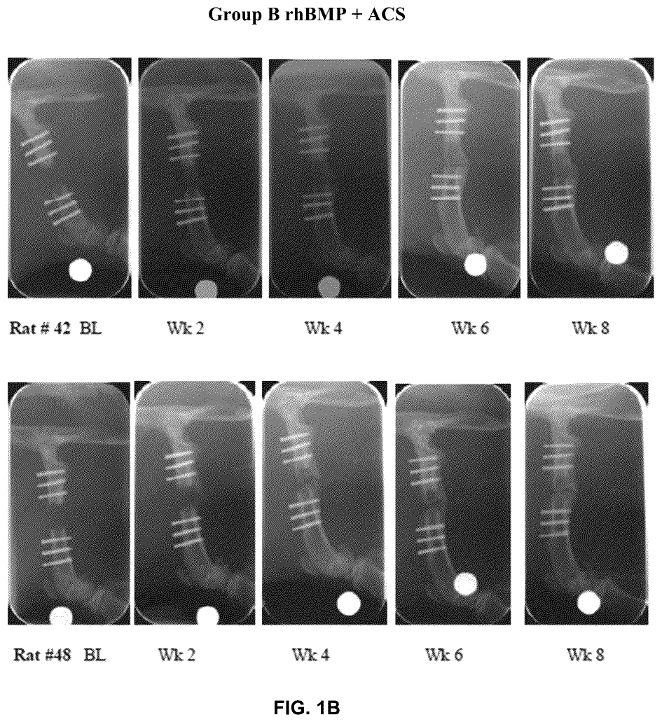

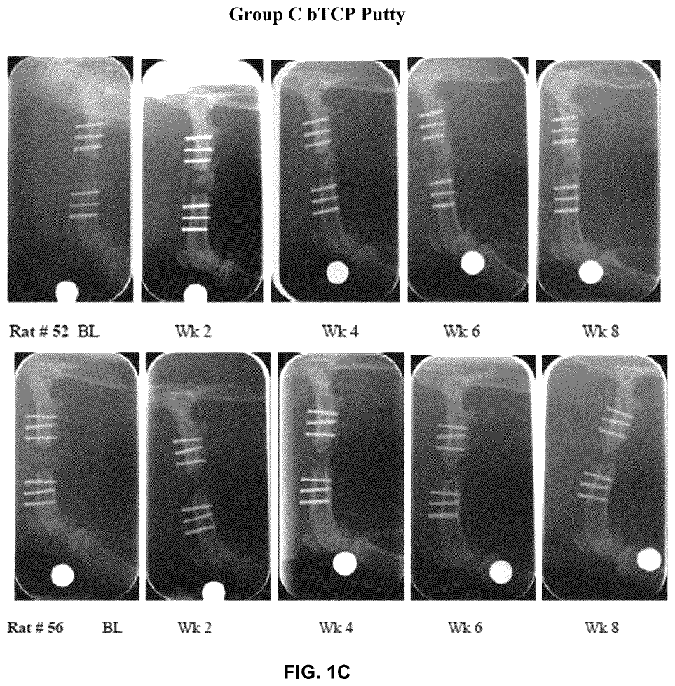

All animals were weighed on arrival and assigned to one of three groups on a weight-ordered distribution. All allocations to group and end-point times were done to ensure there were no significant differences in weight prior to the surgical intervention between any of the groups in terms of treatment to be administered, or time to end-point (either 4- or 8-weeks post-surgery). There were no significant differences in weights at assignment to study between any of the groups for either the initial group of animals or the replacement group, or at any time for the duration of the study (FIG. 1A, FIG. 1B and FIG. 1C).

### Example 2. Radiographic, Mechanical, Histomorphometric and Histological Analyses of 4 and 8 Week Post-Fracture Healing in Three Experimental Rat Fracture Groups (a, B and C)

To assess the extent of mineralized bone material within the callus region of fractures in three experimental rat groups (A, B and C), radiographic scoring of callus formation, mechanical testing of the callus strength, high resolution micro computed tomography (μCT), and quantitative histological analyses of bone formation and resorption were conducted.

### Radiographic Assessment

Sagittal radiographic images were generated using a Faxitron X-ray with fixed exposure settings. Radiographic scoring specifically on callus formation healing was based on Chhabra et al. (2005) J Orthop Trauma 19(9): 629-634, and modified from Marino et al. (1979) Clin Orthop Relat Res (145): 239-244 and Makley et al. (1967) J Bone Joint Surg Am 49 (5): 903-914 (Table 4). Scoring was performed in one session on de-identified, randomised radiographs. Both the posterior and anterior aspect of the cortex at the site of fracture was scored. The average score for each specimen was recorded.

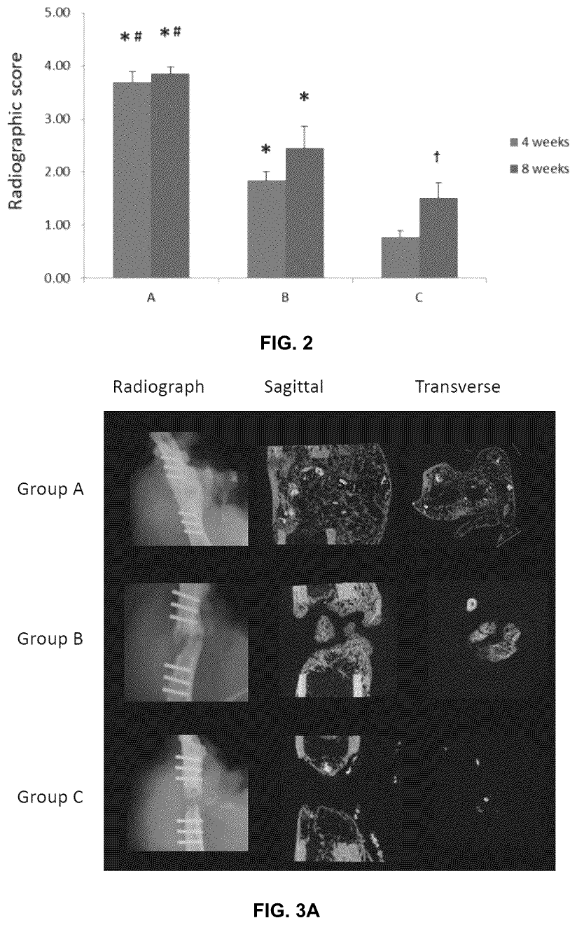

Baseline radiographs were taken on all animals as soon as possible after surgery whilst still under anaesthetic, at two weeks, four weeks (end-point for half of the animals), six weeks and eight weeks (end-point for remaining animals) post-surgery. They were taken in lateromedial and craniocaudal views. FIG. 2 shows representative radiographs of animals from all groups at 0, 2, 4, 6, and 8 weeks post-surgery. The defect was only visible in Group B animals that received the rhBMP+ACS. The putty (with or without protein) in animals from Groups A and C appear to be outside the confines of the defect (more obvious in mediolateral view). This was the case in all animals, even when the surgeon reported that the putty appeared to be completely contained within the defect, and touching the muscle tissue had been completely avoided on insertion of the test or control items.

Ex vivo radiographs, as with the in vivo radiographs showed that the putty and/or bone growth was not confined to the defect area in Groups A and C, as it was in Group B images (FIG. 2).

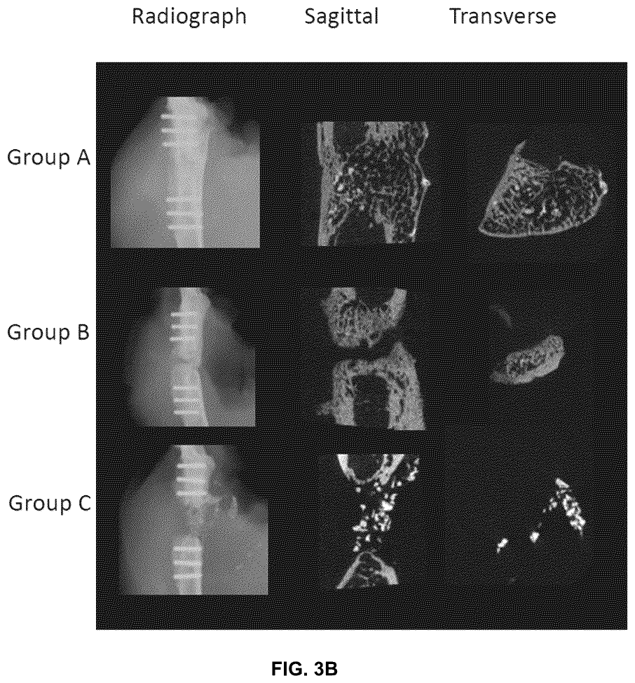

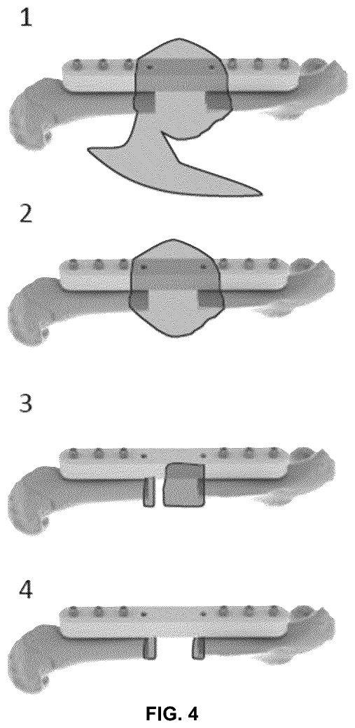

At 4-weeks post-surgery, Group A routinely demonstrated bridging of the periosteal callus and frequently demonstrated mature callus with interfragmentary bridging (FIG. 3A). This as in clear contrast to Groups B and C where only callus tissue and little or no callus scores were observed respectively. At 8-weeks post-surgery, while there appears to be no significant radiographic change in the scoring of the mature callus, Group A remained advanced in the healing stage when compared to Group B and C, both of which only marginally improved their scores in the intervening 4 weeks (FIG. 3B). It is worth noting that in Group A, frequent larger boney callus volumes were observed intact but distal to the fracture site suggesting that the treatment in this group was not contained to the fracture region. Representative images from each group at 4- and 8-weeks post-surgery are located in FIGS. 3A and 3B. A schematic of the varied callus formations is represented in FIG. 4.

### Micro-Computed Tomography

To quantify the total callus and bone mineral volumes, each specimen was scanned by high resolution μCT (Skyscan, Model 1174, Bruker microCT). Specimens were wrapped in PBS-soaked gauze and secured within a humidified tube prior to scanning. All scans were performed at 6.4 μm voxel resolution and X-ray tube potential of 50 kV and 800 μA. Images were acquired over 180 degrees with intervals of 0.8 degrees and frames were averaged from 2 images taken at each step. For each scan, the images were then reconstructed into a z-stack of images to represent the transverse plane of the femur (N-Recon software, Bruker microCT). All reconstructions used a modified Feldkamp cone-beam algorithm with a smoothing setting of 1, a ring artefact reduction level set to 12, a beam hardening level of 20% and a post-alignment value of no greater than +1.0. Reconstructions of the callus region excluded the adjacent screws which secured the rat-fix plate, in order to remove the interfering signal of the titanium screws from subsequent analyses. Uniform realignment of datasets were performed using Dataviewer v.1.5.1 (Bruker, BEL). Reconstructed z-stack images for each specimen were then analysed using CTan software (Bruker microCT). Analyses of bone volume in the fracture region were performed by three manually defined volumes of interest (VOI's). Prior to 3D analyses, all volumes of interest binarised using an adaptive thresholding technique using a specimen-specific hydroxyapatite standard control. 3D analyses were performed to establish total volume and bone volume fractions in each volume of interest.

During necropsy, all treated legs were removed, and placed in either 10% neutral-buffered formalin and kept at room temperature, or gauze-soaked saline and frozen at −20° C., for μCT imaging and mechanical strength testing. All legs were also X-rayed (Faxitron X-ray Imager) and the images graded as per Table 3 above. FIG. 4 shows a graphical representation of bridging of periosteal callus. Moderate callus was observed in Group B samples, whereas Group C samples showed little or no callus.

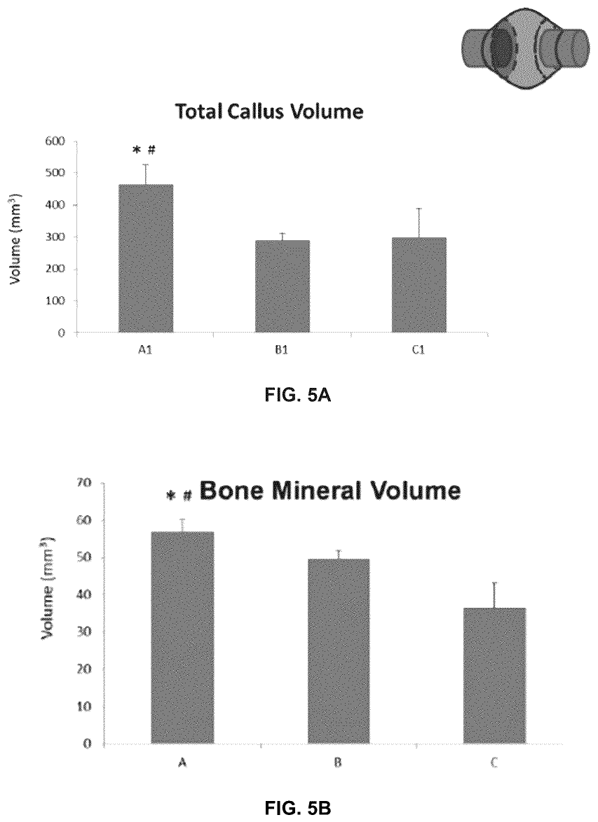

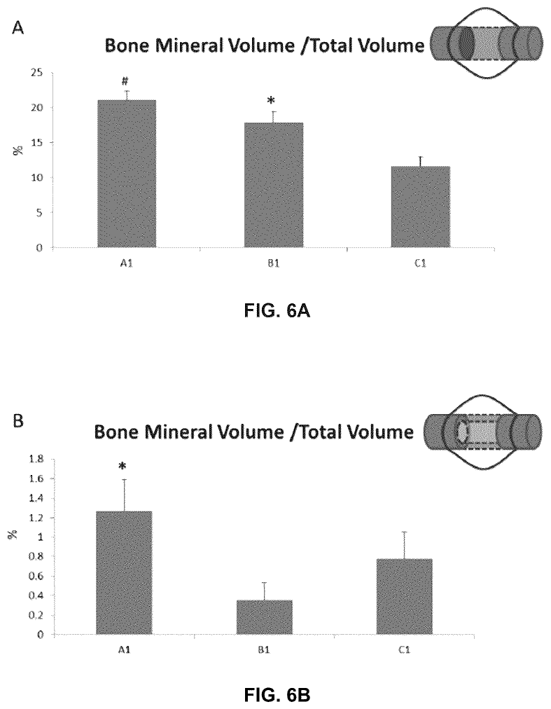

Analyses of the μCT images for bone volume in the fracture region showed that the total callus volume and the bone mineral volume were significantly higher in Group A (tBMP+β-TCP putty-treated) animals than in either the β-TCP putty (Group C) or the rhBMP+ACS-treated animals (p<0.01 in both cases). There were no significant differences between the Group B and Group C animals for either total callus volume or bone mineral volume. These are shown in FIG. 5A and FIG. 5B. When restricting the region to the interpolated shaft region, in both Group A and Group B, the bone mineral volume as a percentage of the tissue volume (BV/TV) was approximately 2-fold greater than Group C (P<0.05). The BV/TV for Group A was trending to be increased by 15% when compared to Group B (P=0.059) (FIG. 6A). When restricting the region to the interpolated cortical bone region only, in Group A, the cortical bone mineral volume, in BV/TV terms, was 4-fold greater than levels in Group B (P<0.05) but not significantly greater than in Group C (FIG. 6B).

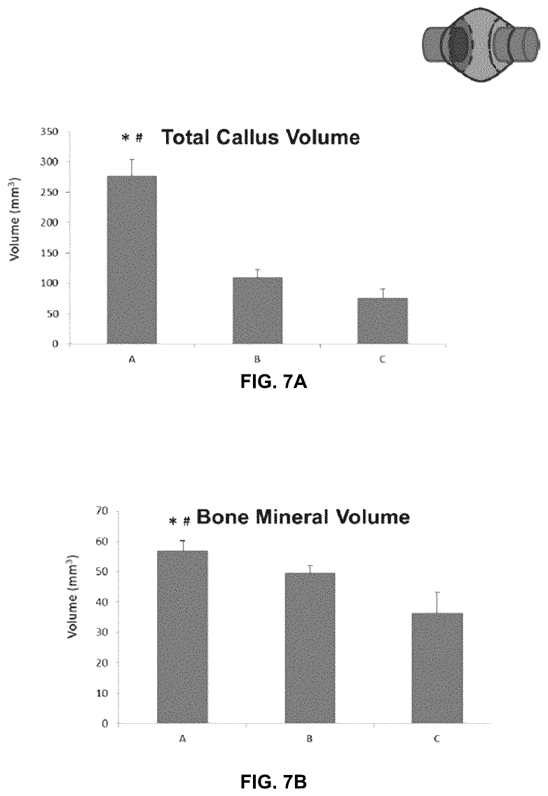

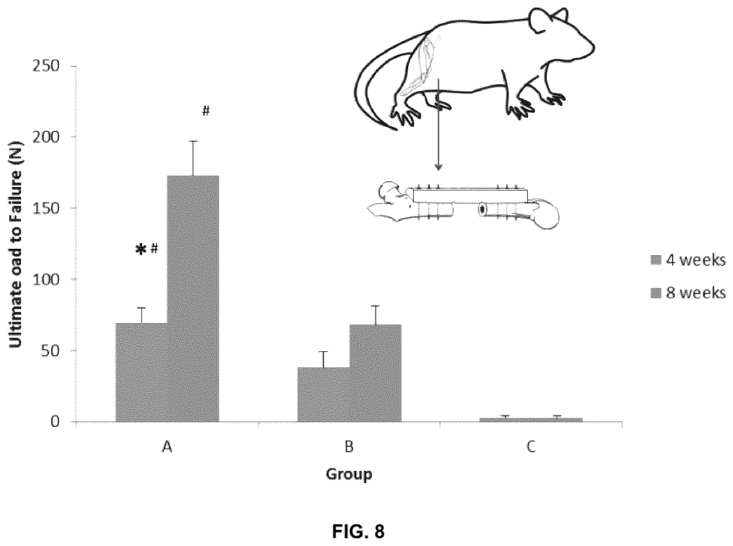

At 8-weeks post-surgery, TCB was markedly increased in Group A when compared to Groups B and C (FIG. 7A and FIG. 7B). However, BMV in Group A is not significantly increased when compared to Group B. These data suggested that while a larger bony callus existed in Group A, the density of bone mineral was more comparable to Group B.

### Mechanical Strength Testing of Treated Femur

The mechanical strength testing of femoral callus was performed using 3 point bending method (5940 and BlueHill 3 software, v3.25, Instron, MA, USA). Each specimen was stored in PBS-soaked gauze at −80° C. until ready for testing. Prior to testing, specimens were gradually thawed to 4° C. over three days to maintain tissue integrity. On the day of testing, samples were scanned by micro-CT in a humidified chamber and kept cool prior to allowing specimens to reach room temperature for mechanical testing. For each specimen, the rat-fix plate was cut immediately prior to testing using a slow speed rotary diamond tipped saw lubricated with cooled phosphate buffered saline. The mid-point of the rat-fix plate was cut to remove the support of the plate in the fracture stabilization. This cut was done so as to cause minimal disturbance to the surrounding callus. After cutting the plate, each bone was then loaded into the 3-point jig with the rat-fix plate in contact with the low anvils with a 10 mm span such that the upper anvil was positioned directly above the cut-point on the plate. The downward motion of the upper anvil tests the resistance, and thus the strength, of the callus. As the bone deforms during testing, the plate separates at the mid-point cut, minimizing the contribution of the plate to the measure of Ultimate Load to Failure (ULF). T Compression testing was performed by gradually increasing the force applied, at a rate of 0.01 mm per second. ULF was determined by the peak force immediately prior to the failure of callus as determined by the force-displacement curve.

The mechanical testing of femoral strength in each fracture specimen required that the contribution of the rat-fix plate be removed without disturbing the callus. Typically this would involve removing the rat-fix plate altogether. However, in numerous samples, in particular in Group A, the extent of callus enveloped the plate making removal impractical. Thus, the compromise was to cut the plate exactly at the mid-point such that it no longer contributed to femoral support. The cut was done in such a way as to only cause minimal damage to the callus. The downward motion of the upper anvil thus tests the resistance, and thus the strength, of the callus. Group A exhibited markedly increased ULF at 4 and 8 week post-surgery when compared to Groups B and C (FIG. 8). At 8 weeks post-surgery, Group A exhibited 2.5-fold increase in ULF when compared to Group B at the same time point (P<0.001). Group B demonstrated increased ULF when compared to Group C and both time points. At 8 weeks post-surgery, in Group B, a trend for increased ULF was observed when compared to 4 weeks post-surgery (P=0.059).

### Histomorphometry of Treated Femur

At ten days prior to end-point, all animals were administered calcein green (25 mg/kg of 10 mg/mL), with the exception of Rat #6, 8, 9, 10, 18, 20, 23, 30 and 50, which were inadvertently administered 12.5 mg/Kg of 10 mg/mL solution. All rats received 100 mg/Kg of xylenol orange (100 mg/mL solution) at three days prior to end-point. All administrations were intraperitoneal.

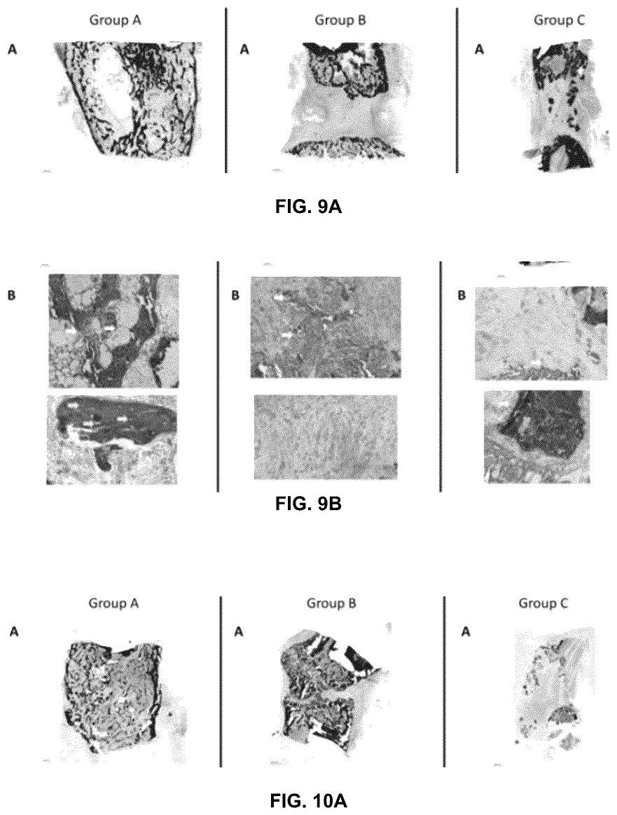

At 4 weeks post-surgery, Group A exhibits extensive evidence of mineralization akin to mature trabecular bone (FIG. 9A). Significant remodelling of bone was observed by the presence of osteoclasts (FIG. 9B, white arrows), and the presence of osteocytes in Group A specimens (FIG. 9B, yellow arrows) which is indicative of mature mineralized bone. Osteoblastic activity was observed in the form of double-fluorochrome labelled mineral accretion. The osteoblastic bone formation was frequently observed as ‘lines’ colored green and red which are in association with each other (FIG. 9B, blue arrows). This represented the formation of lamella (mature) bone, rather than the ad-hoc mineralization of woven bone that occurs in early callus formation, a common observation in Group B. Furthermore, the presence of red-blood cells, and adipocytes indicated that an angiogenesis has occurred and suggested good infiltration of cells to most regions of the callus in Group A specimens.

In Group B, while bone mineral was observed, there were extensive regions of fibrous and cartilage tissue which was unmineralized within the callus region (FIG. 9A). Where new bone mineral occurs, double labels were observed indicating bone formation. However, these double labels were frequently disorganized and often occurred as single labels suggesting that woven bone formation (i.e. initial bone callus formation) was continuing to be formed at this time point (FIG. 9B). While not quantified, it appeared that osteoclasts predominantly existed in regions between the unmineralized and mineralized portions of the callus which suggested an earlier stage of callus development than what was observed for Group A animals.

In Group C, there was no evidence of mineralization, bone formation or bone remodelling. Indeed, there was no evidence of cartilage formation. A fibrous-like material with foreign mineralized spicules existed within the fracture site. There were some instances where osteoclasts as present on the surface of the foreign material, suggesting that this material could be resorbed (FIG. 9B).

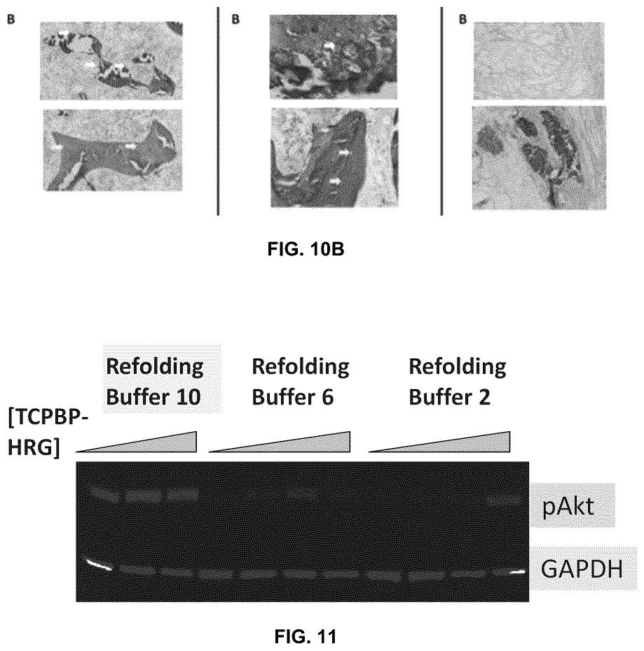

At 8 weeks post-surgery, the same pattern of activity between the groups was continued (FIG. 10A and FIG. 10B). Of note, however, in Group A, there appeared to be greater distribution of mature bone across all sections, consistent with μCT analyses. Also, a degree of intra-trabecular, or intra-cortical labelling of mineral (FIG. 10B, orange arrows) suggested a further maturing of bone consistent with late-stage bone healing. Also of note, Group B specimens appeared to exhibit all stages of callus formation and remodelling. That is, there was evidence of lack of periosteal bridging at times, the presence of cartilage, woven bone, lamellae bone, and evidence of remodelling as well. In Group C, the persistent lack of healing suggested that this group contained a treatment which was most comparable to a critical-sized defect model without any scaffold of healing agents.

In summary, sixty-one animals were successfully treated with one of three test items following surgical creation of a critical size defect in the right femur. The post-operative recovery was unremarkable in the majority of animals. The test items were all placed within the defects, but on X-ray, it could be seen that the compounds containing the β-TCP putty were spread out of the defect area and into the surrounding tissue. As the collagen sponge was not visible on X-ray, it was not possible to judge if the sponge had moved out of the defect area.

Most animals recovered from surgery well and in most cases were moving as per usual within 24 hours of surgery. A number of the animals were larger than the recommended weight for the plates, but the femurs were not so large that the screws of the internal fixator system were not going through the whole bone, so there should not have been loss of stability while new bone was forming at the site. Two animals had evidence of displaced bones on the two week radiographs, these animals were not heavier than others. One animal was in the rhBMP+ACS group, the other was in the β-TCP putty group. Another animal (Group C) had a broken screw and displaced bone at the six-week post-operative time point. There was no obvious reason why this had occurred.

There were no differences between any of the groups in any clinical pathology parameters or histological analyses of organs and tissues.

Radiographic and histological examination of the treated femurs showed that the Group A animals (tBMP-2+β-TCP putty) had the most evidence of formation of mature bone in the defect site compared to the other two groups. This was shown by bridging of the periosteal callus, increased mineralization, presence of osteocytes, active remodelling of the bone (as shown by the presence of osteoclasts) and evidence of angiogenesis (shown by the presence of red blood cells and adipocytes). Whilst some evidence of mineralization of callus is present in the Group B (rhBMP+ACS) samples, this appeared to be less developed than for Group A. Group C (β-TCP putty) showed no evidence of mineralization, suggesting that having a scaffold alone could not induce new bone growth within eight weeks of application into a critical-sized defect.

Group A animals also had significantly stronger bones than either Group B or Group C animals as evidenced by ultimate load to failure tests. This was evident at both four and eight weeks post-treatment.

The differences in callus formation in the process of healing are distinct between all three groups, clearly indicating that there were unambiguous differences in the treatments of fracture in each group.

In Group C, a frank lack of periosteal bridging and minimal bony callus formation at both 4 and 8 weeks was associated with a lack of cellular activity within the fracture. These observations were consistent with the near complete absence of mechanical integrity at either time point.

In Group B, the mineralized callus was more extensive than the levels in Group C both 4 and 8 weeks post-surgery. This was evident in the radiographic and histomorphometric analyses. Furthermore, while periosteal bridging was observed only in some Group B specimens at 8 week post-surgery, there was a clear and overt difference in the histological assessment at both time points with regards to bone formation and cellular activity. Unlike in Group C, the presence of bone formation, frequently in the form of woven bone and occasionally as lamellae bone, was associated with increase in mechanical strength when compared to Group C. Interestingly, neither radiographic nor mechanical strength measures were significantly improved at 8 weeks when compared to 4 weeks post-surgery. This suggested that the majority of activity in callus formation and structural integrity occurred in the first 4 weeks.

Group A exhibited the most advanced healing when compared to the other groups. At 4 weeks post-surgery radiographic evidence of periosteal bridging was a measure that no samples achieved in Group C and often was not observed in Group B, even at 8 weeks post-surgery. Interestingly, while radiographic scores at 8 weeks post-surgery were comparable to 4 week scores, the mechanical strength of the callus was more than double the levels at 4 weeks post-surgery and at least 3-fold greater than levels in Group B, suggesting that considerable bone mineralization occurred within the callus region in the second 4 week period. This observation was consistent with high levels of lamellae bone formation in Group A at 4 and 8 weeks post-surgery, which was distinct from the weaker woven bone formation that occurred in Group B, even at 8 weeks post-surgery. It is important to note that callus volume at 8 weeks of age was lower than levels at 4 weeks post-surgery, suggesting that callus volume was being remodelled into a more compact callus at the site of fracture. In Group A, this was consistent with fewer observations of extraneous mineralized callus distal to the fracture site at 8 weeks post-surgery. With regards to the histological assessment of Group A specimens, observations of osteoclastic none resorption adjacent to regions of bone undergoing bone formation is entirely typical of bone undergoing remodelling which is known to be done to provide a stronger and more efficient structure for skeletal integrity. The additional observations of the presence of osteocytes as well as other cellular structures such as vascular structures and adipocytes clearly makes the bone often indistinguishable from endogenous trabecular micro-anatomy.

In conclusion, the bone specimens form the tBMP-2+β-TCP putty group clearly demonstrated vastly improved bone healing at 4- and 8-weeks post-surgery when compared to samples from animals in the rhBMP+ACS or the β-TCP putty alone treatment groups.

### Example 3. Overview of Chimeric β-TCP Polypeptides

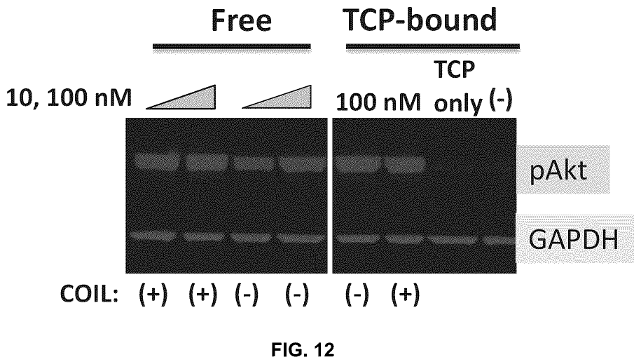

The present inventors found that chimeric polypeptides comprising a targeting polypeptide that binds to β-TCP maintained activity of the tethered protein (in this example, HRG). The chimeric polypeptide (10×β-TCP-histidine rich glycoprotein HRG) refolded at 22° C. in buffer 10, and following incubation of the chimeric polypeptide with MCF-7 breast cancer cells, Akt activity was detected (FIG. 11). This result indicated that the chimeric polypeptide stimulated signalling activity in the target cell and was able to cause Akt phosphorylation. Next, the Akt activity was determined in 10×β-TCP-HRG bound to a β-TCP peptide (FIG. 12). The β-TCP peptide on its own was unable to cause Akt phosphorylation in MCF-7 cells, however, incubation of MCF-7 cells with β-TCP peptide bound to 10×β-TCP-HRG led to Akt phosphorylation. Interestingly, the extent of Akt phosphorylation was not affected whether the chimeric polypeptide was bound to a β-TCP peptide or not.

### Example 4. Binding Affinity of tBMP-2 to Various Substrate Materials

Binding assays were performed to quantify the affinity of tBMP-2 protein to various substrates, including commercially available bone graft materials, ceramic powders, 3D printable scaffold materials, and a plasma-sprayed hydroxyapatite coating. The tBMP-2 used in this study comprises MPIGSLLADTTHHRPWTVIGESTHHRPWSIIGESSHHKPFTGLGDTTHHRPWGILAESTHHKP WTASGAGGSEGGGSEGGTSGATGAGTSTSGGGASTGGGTGQAKHKQRKRLKSSCKRHPLY VDFSDVGWNDWIVAPPGYHAFYCHGECPFPLADHLNSTNHAIVQTLVNSVNSKIPKACCVPT ELSAISMLYLDENEKVVLKNYQDMVVEGCGCR (SEQ ID NO: 502). First, 10 mg of each material was washed with deionized water followed by acetate buffer. Then either a 150 μg or 250 μg load of tBMP-2 protein was applied to each material and allowed to bind under light agitation for 90-120 minutes. The flow through was aspirated (removing the tBMP-2 which did not bind to the substrate material), and a wash was conducted with acetate buffer. A subsequent overnight elution was done by immersing the substrate material in a sodium phosphate buffer (100 mM NaPhos, 4 M Urea, 1 M NaCl, 10% 1.6 Hexanediol, pH=8) to remove the bound protein from the substrate for measurement.

The bound tBMP-2 was quantified by either 1) running non-reducing SDS Page gel electrophoresis and comparing the eluted tBMP-2 band to the tBMP-2 load band with Image J Gel Analysis tool, or 2) conducting a Bradford assay (with a BSA standard curve) on the eluted tBMP-2 and tBMP-2 load. Dividing the eluted tBMP-2 quantity by the original tBMP-2 load quantity and multiplying by 100 gave the % of tBMP-2 load that was strongly tethered to the substrate after the binding incubation step.

Table 6 summarizes the materials that were evaluated as well as the % of tBMP-2 load which was effectively tethered to the substrate (% Bound).

TABLE 6
Summary of Binding Activity

| | | | Load tBMP2 | | % of tBMP2 |
|---|---|---|---|---|---|
| | | | Applied to | Method of | load bound |
| Product | Description | Manufacturer | Material | measurement | to material |
| Mastergraft | Biphasic TCP | Medtronic | 15 ug tBMP2/mg | Bradford Assay | 69% |
| Strip | (85% β- | | material | | |
| | Tricalcium | | | | |
| | Phosphate: 15% | | | | |
| | Hydroxyapatite)/ | | | | |
| | Type I bovine | | | | |
| | collagen | | | | |
| | composite | | | | |
| Vitoss Foam | β-Tricalcium | Stryker | 15 ug tBMP2/mg | Bradford Assay | 82% |
| Pack | Phosphate/Type | | material | | |
| | I bovine | | | | |
| | collagen | | | | |
| | composite | | | | |
| chronOS | β-Tricalcium | DePuy Synthes | 15 ug tBMP2/mg | Image J Gel | 75% |
| Strip | Phosphate/poly | | material | Analysis Tool | |
| | (lactide co-ε- | | | | |
| | caprolactone) | | | | |
| | composite | | | | |
| Vitoss | β-Tricalcium | Stryker | 15 ug tBMP2/mg | Image J Gel | 74% |
| Micromorsels | Phosphate | | material | Analysis Tool | |
| | granules | | | | |
| | (1 mm-2 mm) | | | | |
| Lifeink 500 | Calcium | Advanced | 15 ug tBMP2/mg | Image J Gel | 87% |
| | Phosphate | Biomatrix | material | Analysis Tool | |
| | Cement 3D | | | | |
| | printable ink | | | | |
| Hyperelastic | Hydroxapatite/ | Dimension Inx | 15 ug tBMP2/mg | Image J Gel | 58% |
| Bone | Poly(lactic-co- | | material | Analysis Tool | |
| | glycolic acid) | | | | |
| | 3D printable ink | | | | |
| Bioactive | Combeite 45S5 | Stryker | 25 ng tBMP2/mg | Image J Gel | 49% |
| Glass | Bioactive Glass | | material | Analysis Tool | |
| | powder | | | | |
| Silicon | Si₃N₄ powder, | Chemsavers | 25 ng tBMP2/mg | Image J Gel | 54% |
| Nitride | 99% purity | | material | Analysis Tool | |
| Powder | | | | | |
| β-TCP | β-Tricalcium | CaP Biomaterials | 25 ug tBMP2/mg | Image J Gel | 79% |
| Powder | Phosphate | | material | Analysis Tool | |
| | powder, 3-5 μm | | | | |
| | particle size | | | | |
| β-TCP Spray | β-Tricalcium | CaP Biomaterials | 25 ug tBMP2/mg | Image J Gel | 78% |
| Dried | Phosphate spray | | material | Analysis Tool | |
| Powder | dried powder, | | | | |
| | <38 μm particle | | | | |
| | size | | | | |
| Hydroxyapatite | Hydroxyapatite | CaP Biomaterials | 25 ug tBMP2/mg | Image J Gel | 57% |
| Powder | powder, 3-5 μm | | material | Analysis Tool | |
| | particle size | | | | |
| HA-coated | Hydroxyapatite | Citieffe | 25 ug tBMP2/mg | Bradford Assay | 44% |
| Bone Screw | plasma spray- | | material | | |
| | coated stainless | | | | |
| | steel bone screw | | | | |
| | (6 mm dia × 5 | | | | |
| | mm long piece | | | | |
| | cut from screw) | | | | |
| β-TCP | β-Tricalcium | CaP Biomaterials | 15 ug tBMP2/mg | Bradford Assay | 70% |
| Granules | Phosphate | | material | | |
| | granules, | | | | |
| | 250-1000 μm | | | | |
| | particle size | | | | |
| Hydroxyapatite | Hydroxyapatite | CaP Biomaterials | 15 ug tBMP2/mg | Bradford Assay | 52% |
| Granules | granules, | | material | | |
| | 250-1000 μm | | | | |
| | particle size | | | | |
| ReBOSSIS | Cotton-like bone | Orthorebirth | 15 ug tBMP2/mg | Bradford Assay | 83% |
| | void filler. | | material | | |
| | Main | | | | |
| | ingredients are | | | | |
| | β-Tricalcium | | | | |
| | Phosphate, | | | | |
| | bioabsorbable | | | | |
| | polymer, and | | | | |
| | SiV (silicon- | | | | |
| | containing | | | | |
| | calcium | | | | |
| | carbonate) | | | | |

### Example 5. Generation of Ceramic-Binding Protein Variants

Ceramic-binding protein variants were generated by fusing targeting polypeptides that bind to ceramics such as calcium phosphate (Ca-PO4) and hydroxyapatite to protein biologics, including BMP2, BMP7, IGF1, FGF18, TGFβ3, EGF, and NRG1. The resulting protein variants (tBMP2, tBMP7, tIGF1, tFGF18, tTGFβ3, tEGF, and tNRG1, respectively) and the modification details are provided in Table 7. Dimeric binding motif refers to the presence of two binding domains due to the dimeric nature of the ligand.

TABLE 7
Modified Proteins

| | Total | | Dimeric | | |
|---|---|---|---|---|---|
| Modified | MW | Terminal | Binding | Ca-PO4 | Hydroxyapatite |
| Protein | (kDa) | Modification | Motif | binding | binding |
| tBMP2 | 22.8 | N-only | Y | Y | Y |
| tBMP7 | 23 | N-only | Y | Y | Y |
| tIGF1 | 17.6 | N-and/or C- | N | Y | Y |
| tFGF18 | 30.1 | N-and/or C- | N | Y | Y |
| tTGFβ3 | 22.6 | N-only | Y | Y | Y |
| tEGF | 16.1 | N-and/or C- | N | Y | Y |
| tNRG1-β1 | 17.4 | N-and/or C- | N | Y | Y |

### Example 6. Safety and Efficacy of tBMP-2 on a Ceramic Carrier in a Challenging Caprine Critical Defect Model

This example demonstrates that tBMP-2 accelerates bone repair in a rigorous and challenging segmental bone defect animal model (CCTDM).

Reconstruction of severe segmental bone loss remains a major challenge for treating wounded warriors and is frequently complicated by other conditions and concomitant injuries. In particular, there is an unmet need for precise tissue regeneration following trauma. Bone morphogenetic protein-2 (BMP-2) is the most effective osteoinductive agent identified to date and is used in clinical orthopaedic practice to actively promote bone formation. However, BMP-2 use is limited by cost and the need to control release and delivery.

To address this, a method for controlled, local delivery of BMP-2 activity has been engineered using a fusion protein that enables specific binding and retention of bioactive BMP-2 (tBMP-2) on beta-tricalcium phosphate ceramic substrates (CS). This example demonstrates the ability of CS-bound tBMP-2 to accelerate bone repair in a dose-responsive manner in the most rigorous bone defect model yet reported. tBMP-2 (SEQ ID NO: 502) was generated having a binding site that binds ceramic with high affinity and remains tightly bound under physiological conditions, thus reducing off-target effects. tBMP-2 behaves like a paint when applied to orthopaedic ceramics, thus reducing off-target effects. Targeted delivery of tBMP-2 can potentially improve the safety and efficacy of the current standard of care for bone repair.

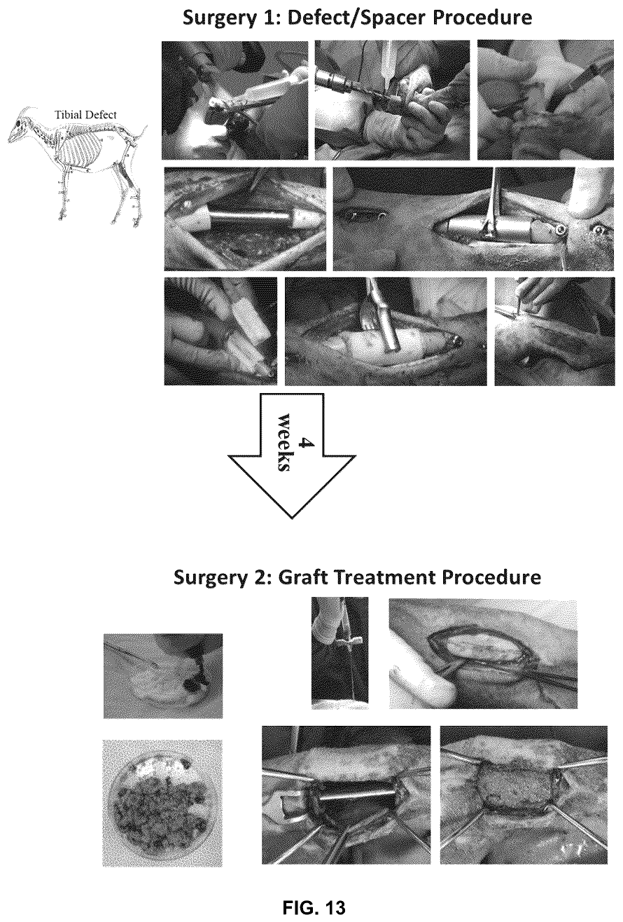

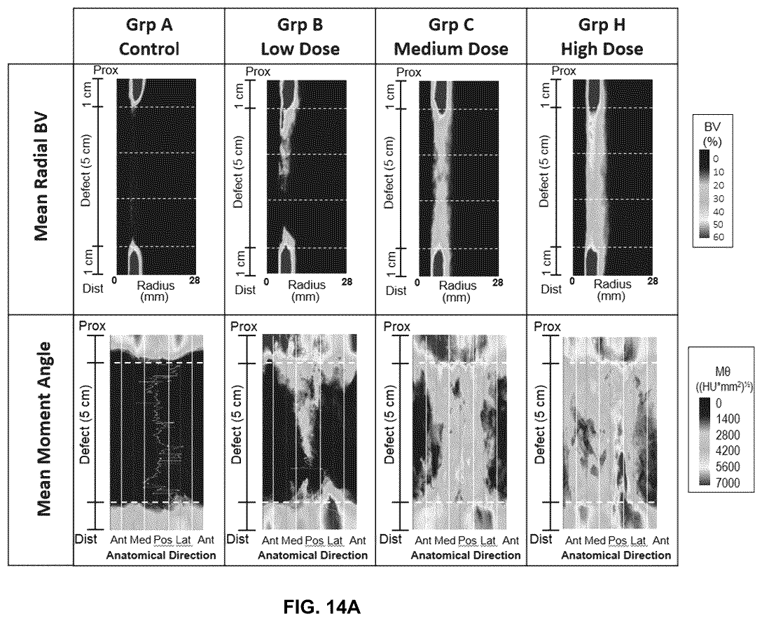

Briefly, twenty-four goats (female, 4-6 years; Body weight=43±9 kg) underwent the Caprine Chronic Tibial Defect Model (CCTDM) protocol after IACUC approval. As used in this example, tBMP-2=Ceramic scaffold composed of β-TCP granules (CaP Biomaterials, East Troy, WI) and fibers (ORTHOReBIRTH, Georgetown, Texas). Treatment groups included group A=Ceramic substrate only (control) (n=4), group B=Low dose tBMP-2 added to the scaffold (0.214 mg/cc defect) (n=4), group C=Medium dose tBMP-2 added to the scaffold (2.15 mg/cc defect) (n=8), group H=High dose tBMP-2 (8 mg/cc defect) (n=8). The treatment procedures are outlined in FIG. 13. The Surgery 1 procedure included creating a 5-cm bone/periosteal defect in tibial diaphysis, stripping 2-cm periosteum from each segment, removing 10 cm³ of cranial tibialis/gastrocnemius, placing 5-cm custom smooth or textured PMMA spacer, and stabilizing with interlocking intramedullary nail. Surgery 2, performed 4 weeks after Surgery 1, included aspirating 6 cc of sternal raw bone marrow and adding it to each graft, removal of the spacer while preserving the Induced Membrane (IM) using a bomb bay door opening, and placing the graft into the defect. The implant at each graft site was supplemented with 6 cc of bone marrow aspirated from the sternum to provide a source of osteogenic connective tissue progenitors in a marrow-derived clot. Follow up procedures were performed during the 12 weeks after Surgery 2, including anterior-posterior (AP) and mediolateral (ML) Radiographs every 4 weeks and physical examination (lameness). Twelve weeks after Surgery 2, tibia were harvested and fixed in Formalin. Outcomes measured included Micro CT (primary outcome), Radial % Bone Volume (BV) and Moment Angle plots, Total Bone Volume (mm3) in 2.5 cm central region, and 12-weeks radiographs (AP and ML views).

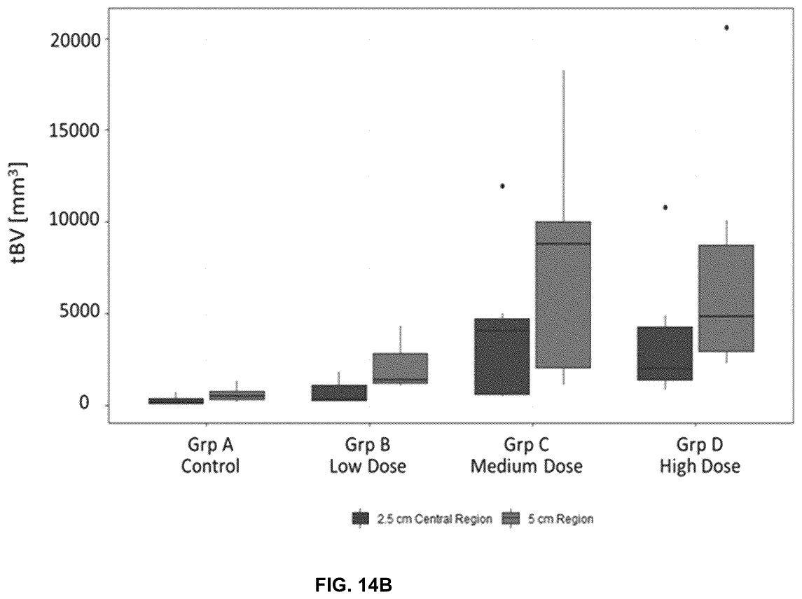

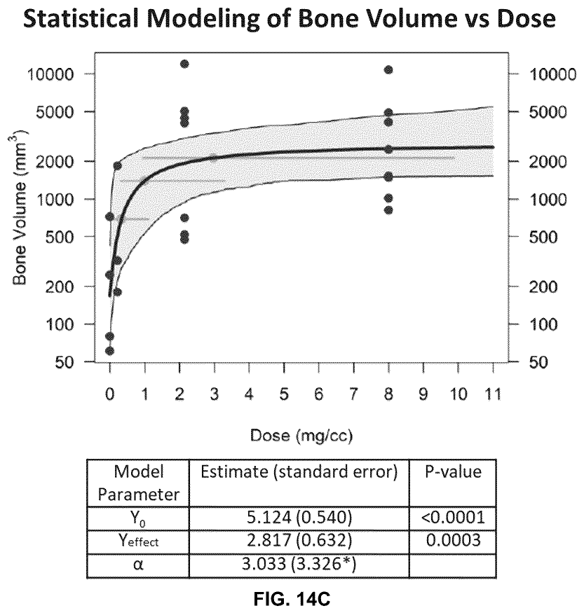

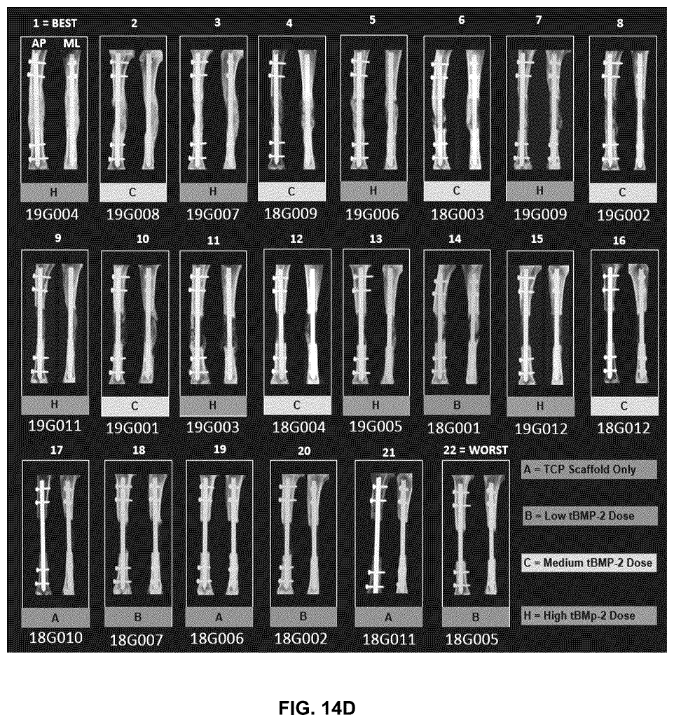

The radiographic data (FIG. 14D) show that tBMP-2 demonstrates superior bone formation in a greater number of animals than the low dose and control conditions and the amount of new bone formed in both tBMP-2 groups was significantly higher than in the substrate-only group. Mean radial percent BV analysis illustrate a tendency for bone to form most readily in the medial-posterior aspect of the defect (FIG. 14A).

The results show a statistically significant dose-response (FIG. 14C), with medium dose and high dose tBMP-2 demonstrating comparable bone formation that was superior to the carrier only group (FIG. 14B). Even the lowest dose elicited bone formation. No adverse effects were noted in any animals, including the highest dosed animals.

This study demonstrates that tBMP-2 delivered on a ceramic carrier can maintain local BMP-2 concentrations necessary to induce bone formation with high efficacy in a severely compromised tissue bed with a good safety margin, even when combined with autogenous bone marrow-derived cells. By binding tBMP-2 to resorbable substrates, the local concentration can be maintained over the time scales required to induce complete osteoinduction. In addition, the tBMP-2 technology tested in this study has demonstrated safety and efficacy in the most challenging large animal model that is available. It was notable that bioactivity remained only where the device was implanted. These data enable the selection of tBMP-2 dosing and formulation for subsequent preclinical model testing and future clinical trials. Improved materials and strategies for bone regeneration in compromised tissue beds will enable better outcomes for military and civilian patients suffering from large traumatic lower extremity injuries and reduce the need for amputation.

### Example 7. Safety and Efficacy of tBMP-2 on a Ceramic Carrier in a Sheep Critical Defect Model

This example demonstrates the effectiveness of tBMP-2 (SEQ ID NO: 502) in a challenging ovine critical tibial defect model (OCTDM) as compared to autograft, the current standard of care.

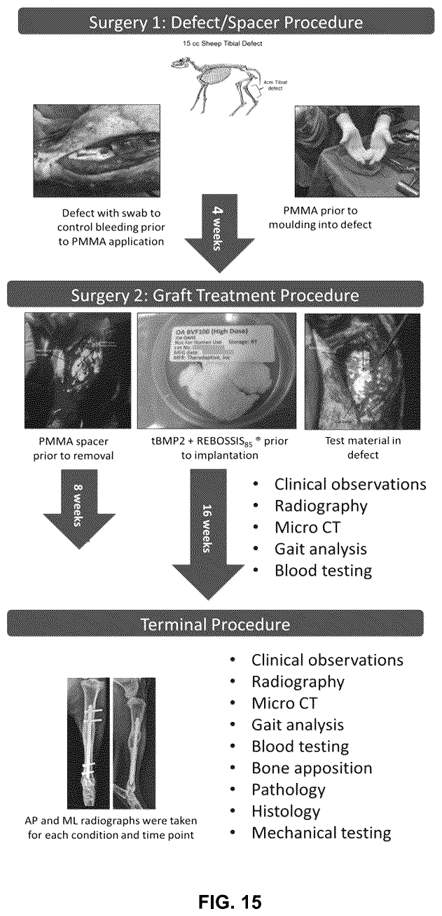

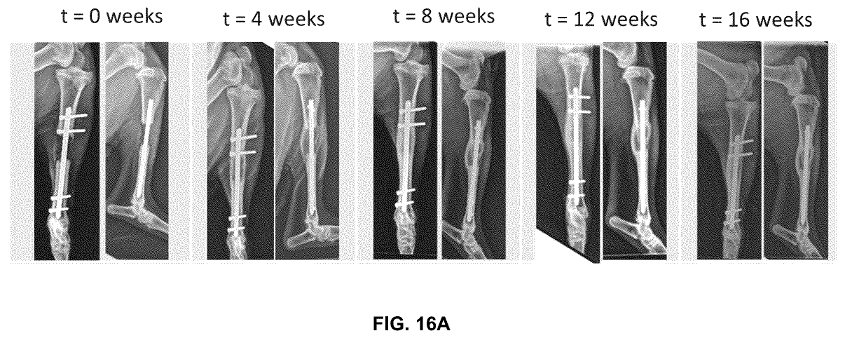

Briefly, twenty-four male skeletally mature wethers (castrated sheep) underwent the two-surgical OCTDM procedures (using the Masquelet technique) under approved SAHMRI IACUC and ACURO protocols. FIG. 15 provides an outline of the first and second surgeries, Surgery 1 and Surgery 2, respectively. Surgery 1 included excision of 4 cm of the tibial diaphysis, reaming of the tibia if necessary, stabilization of the tibia with a stainless-steel intramedullary nail and two proximal and two distal cross-locking bolts, placing a polymethyl methacrylate (PMMA) spacer and molding such that it replicated the dimensions of the tibia and overlapped the ends of the osteotomy site by a few millimetres. Surgery 2 was performed 4 weeks after Surgery 1, where the spacer was removed and the defect (contained within an induced fibrous membrane) was filled with the test materials or control item. Treatment group 1 animals (n=8) received 2 mg tBMP-2 per cc of defect (CS, ReBOSSIS₈₅®), treatment group 2 animals (n=8) received 5 mg tBMP-2 per cc defect (CS), and treatment group 3 animals received autograft harvested from the iliac crest as a control.

Radiographs were taken immediately after each surgery and every two weeks following one month of post-graft recovery to evaluate bone formation, union and remodelling of the defect. The groups were divided between 8- and 16-week endpoints and the following methods were used for analysis: hematology, biochemistry, pathology, mechanical testing, micro computed tomography and bone histology. Two sheep were excluded from data analyses due to nail failure and two sheep presented with infection (not related to surgery or treatment) that may have impacted healing.

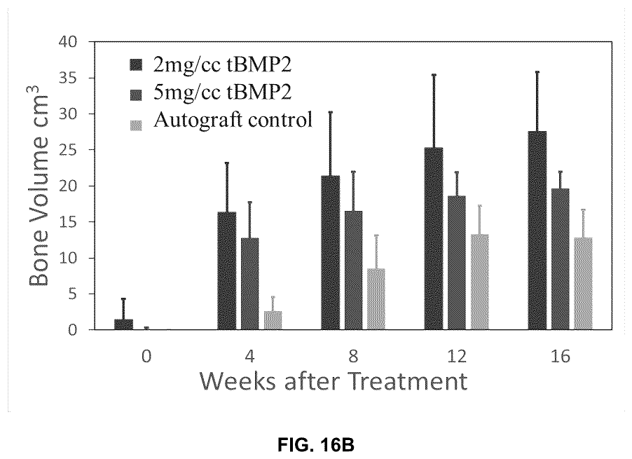

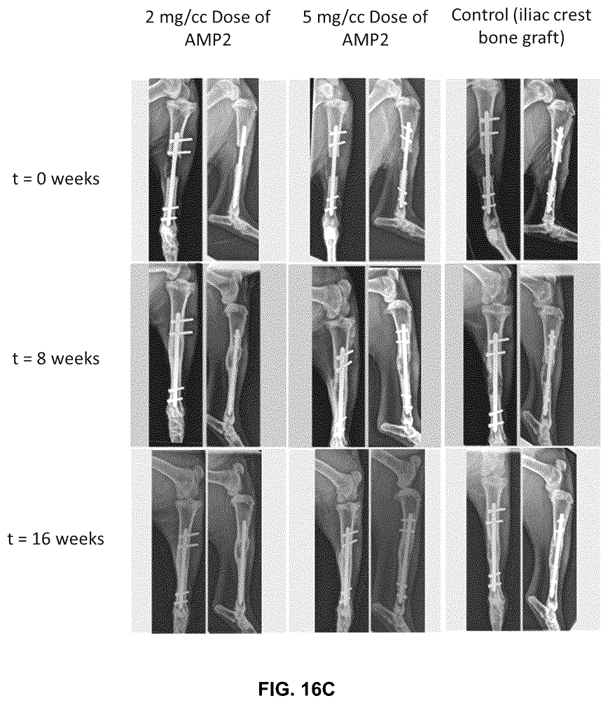

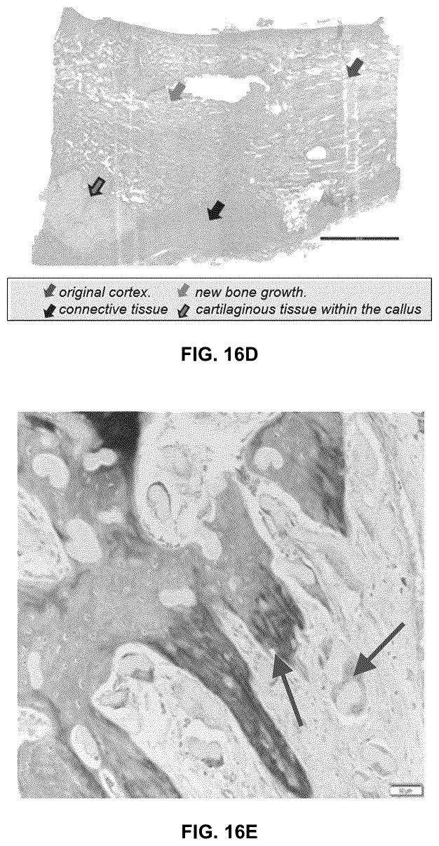

All three groups showed significant bone formation throughout the time course of the study and the two tBMP-2 doses showed similar increases in new bone formation (FIGS. 16A-16E). tBMP-2 delivered by a cotton-like ceramic bone void filler provides an adequate osteoinductive signal to induce new bone formation comparable to autograft as demonstrated by radiographic (FIG. 16A) and CT (FIG. 16B) data, and it can maintain local BMP-2 concentrations necessary to induce bone formation with high efficacy in a severely compromised tissue bed with a good safety margin as shown in FIGS. 16D-16E.

No adverse clinical effects related to test or control item administration including heterotopic bone formation were seen in any group at either end-point time. No animals exhibited signs of illness in-life and all continued to eat and gain weight over the course of the study.

The tBMP-2 technology tested in this study demonstrated safety and efficacy in this challenging large animal model, enabling the selection of tBMP-2 formulation for future preclinical and clinical trials. Improved materials and strategies for bone regeneration in compromised tissue beds will enable better outcomes for military and civilian patients suffering from large traumatic lower extremity injuries and reduce the need for amputation.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A polypeptide composition comprising: a targeting polypeptide comprising a sequence at least 80% identical to SEQ ID NO: 22 (LLADTTHHRPWT VIGESTHHRPWS IIGESSHHKPFT GLGDTTHHRPWG ILAESTHHKPWT), and a therapeutic polypeptide comprising a sequence at least 80% identical to a sequence selected from SEQ ID NOS: 32, 46-71, 73-77, 79-101, 152, 168, 176, 268.

2. The polypeptide composition of claim 1, wherein the therapeutic polypeptide comprises a sequence at least 90% identical to a sequence selected from SEQ ID NOS: 32, 46-71, 73-77, 79-101, 152, 168, 176, 268.

3. The polypeptide composition of claim 1, wherein the therapeutic polypeptide comprises a sequence selected from SEQ ID NOS: 32, 46-71, 73-77, 79-101, 152, 168, 176, 268.

4. The polypeptide composition of claim 1, wherein the therapeutic polypeptide comprises a sequence at least 80%, 85%, 90%, or 95% identical to SEQ ID NO: 32.

5. The polypeptide composition of claim 1, wherein the therapeutic polypeptide comprises SEQ ID NO: 32.

6. The polypeptide composition of claim 1, wherein the targeting polypeptide comprises a sequence at least 90% or 95% identical to SEQ ID NO: 22.

7. The polypeptide composition of claim 1, wherein the targeting polypeptide comprises SEQ ID NO: 22.

8. The polypeptide composition of claim 2, wherein the targeting polypeptide comprises a sequence at least 90% or 95% identical to SEQ ID NO: 22.

9. The polypeptide composition of claim 2, wherein the targeting polypeptide comprises SEQ ID NO: 22.

10. The polypeptide composition of claim 3, wherein the targeting polypeptide comprises a sequence at least 90% or 95% identical to SEQ ID NO: 22.

11. The polypeptide composition of claim 3, wherein the targeting polypeptide comprises SEQ ID NO: 22.

12. The polypeptide composition of claim 4, wherein the targeting polypeptide comprises a sequence at least 90% or 95% identical to SEQ ID NO: 22.

13. The polypeptide composition of claim 4, wherein the targeting polypeptide comprises SEQ ID NO: 22.

14. The polypeptide composition of claim 5, wherein the targeting polypeptide comprises a sequence at least 90% or 95% identical to SEQ ID NO: 22.

15. The polypeptide composition of claim 5, wherein the targeting polypeptide comprises SEQ ID NO: 22.

16. A polypeptide composition comprising a sequence at least about 80% identical to SEQ ID NO: 551 ((X)QAKHKQRKRLKSSCKRHPLYVDFSDVGWNDWIVAPPGYHAFYCHGECPFPLADHL NSTNHAIVQTLVNSVNSKIPKACCVPTELSAISMLYLDENEKVVLKNYQDMVVEGCGCR), wherein the polypeptide composition comprises X, and X comprises SEQ ID NO: 22
(LLADTTHHRPWTVIGESTHHRPWSIIGESSHHKPFTGLGDTTHHRPWGI
LAESTHHKPWT).

17. The polypeptide composition of claim 16, comprising a sequence at least about 90% or 95% identical to SEQ ID NO: 551.

18. A polypeptide composition comprising a sequence at least about 80% identical to SEQ ID NO: 639 ((X)QAKHKQRKRLKSSCKRHPLYVDFSDVGWNDWIVAPPGYHAFYCHGECPFPLADHL NSTNHAIVQTLVNSVNSKIPKACCVPTELSAISMLYLDENEKVVLKNYQDMVVEGCGCR), wherein the polypeptide composition comprises X, and X comprises SEQ ID NO: 2 (VIGESTHHRPWS).

19. The polypeptide composition of claim 18, comprising a sequence at least about 90% or 95% identical to SEQ ID NO: 639.

20. A polypeptide composition comprising a sequence at least about 80% identical to SEQ ID NO: 519 ((X)QAKHKQRKRLKSSCKRHPLYVDFSDVGWNDWIVAPPGYHAFYCHGECPFPLADHL NSTNHAIVQTLVNSVNSKIPKACCVPTELSAISMLYLDENEKVVLKNYQDMVVEGCGCR), wherein the polypeptide composition comprises X, and X comprises SEQ ID NO: 6 (IIGESSHHKPFT).

21. The polypeptide composition of claim 20, comprising a sequence at least about 90% or 95% identical to SEQ ID NO: 519.

22. A polypeptide composition comprising a sequence at least about 80% identical to SEQ ID NO: 521 ((X)QAKHKQRKRLKSSCKRHPLYVDFSDVGWNDWIVAPPGYHAFYCHGECPFPLADHL NSTNHAIVQTLVNSVNSKIPKACCVPTELSAISMLYLDENEKVVLKNYQDMVVEGCGCR), wherein the polypeptide composition comprises X, and X comprises SEQ ID NO: 7 (GLGDTTHHRPWG).

23. The polypeptide composition of claim 22, comprising a sequence at least about 90% or 95% identical to SEQ ID NO: 521.

24. A polypeptide composition comprising a sequence at least about 80% identical to SEQ ID NO: 515 ((X)QAKHKQRKRLKSSCKRHPLYVDFSDVGWNDWIVAPPGYHAFYCHGECPFPLADHL NSTNHAIVQTLVNSVNSKIPKACCVPTELSAISMLYLDENEKVVLKNYQDMVVEGCGCR), wherein the polypeptide composition comprises X, and X comprises SEQ ID NO: 4
(ILAESTHHKPWT).

25. The polypeptide composition of claim 24, comprising a sequence at least about 90% or 95% identical to SEQ ID NO: 515.

26. A polypeptide composition comprising a sequence at least about 80% identical to SEQ ID NO: 501.

27. The polypeptide composition of claim 26, wherein the sequence is at least about 90% or 95% identical to SEQ ID NO: 501.

28. The polypeptide composition of claim 26, wherein the sequence comprises SEQ ID NO: 501.

29. A polypeptide composition comprising a sequence at least about 80% identical to SEQ ID NO: 507.

30. The polypeptide composition of claim 29, wherein the sequence is at least about 90% or 95% identical to SEQ ID NO: 507.

31. The polypeptide composition of claim 29, wherein the sequence comprises SEQ ID NO: 507.

32. A polypeptide composition comprising a sequence at least about 80% identical to SEQ ID NO: 506.

33. The polypeptide composition of claim 32, wherein the sequence is at least about 90% or 95% identical to SEQ ID NO: 506.

34. The polypeptide composition of claim 32, wherein the sequence comprises SEQ ID NO: 506.

35. A polypeptide composition comprising a sequence at least about 80% identical to SEQ ID NO: 505.

36. The polypeptide composition of claim 35, wherein the sequence is at least about 90% or 95% identical to SEQ ID NO: 505.

37. The polypeptide composition of claim 35, wherein the sequence comprises SEQ ID NO: 505.

38. A polypeptide composition comprising a sequence at least about 80% identical to SEQ ID NO: 504.

39. The polypeptide composition of claim 38, wherein the sequence is at least about 90% or 95% identical to SEQ ID NO: 504.

40. The polypeptide composition of claim 38, wherein the sequence comprises SEQ ID NO: 504.

41. A polypeptide composition comprising a sequence at least about 80% identical to SEQ ID NO: 503.

42. The polypeptide composition of claim 41, wherein the sequence is at least about 90% or 95% identical to SEQ ID NO: 503.

43. The polypeptide composition of claim 41, wherein the sequence comprises SEQ ID NO: 503.

44. A polypeptide composition comprising a sequence at least about 80% identical to SEQ ID NO: 502.

45. The polypeptide composition of claim 44, wherein the sequence is at least about 90% or 95% identical to SEQ ID NO: 502.

46. The polypeptide composition of claim 44, wherein the sequence comprises SEQ ID NO: 502.

47. A polypeptide composition comprising: a targeting polypeptide comprising a sequence at least 80%, identical to SEQ ID NO: 22 (LLADTTHHRPWTVIGESTHHRPWSIIGESSHHKPFTGLGDTTHHRPWGILAESTHHKPWT), and a therapeutic polypeptide comprising a bone morphogenetic protein (BMP).

48. The polypeptide composition of claim 47, wherein the BMP is BMP-2.

49. A nucleic acid encoding the polypeptide composition of any one of claims 1-48.

50. A vector comprising the nucleic acid of claim 49.

51. A device comprising the polypeptide composition of any one of claims 1-48, and a carrier material.

52. The device of claim 51, wherein the polypeptide composition is bound to the carrier material.

53. The device of claim 51 or claim 52, wherein the carrier material comprises calcium phosphate.

54. The device of claim 51 or claim 52, wherein the carrier material comprises hydroxyapatite.

55. The device of claim 51 or claim 52, wherein the carrier material comprises alpha tricalcium phosphate, fluorapatite, bone (e.g., demineralized bone), glasses (bioglasses) such as silicates, vanadates, and related ceramic minerals, or chelated divalent metal ions, or any combination thereof.

56. A method of treating a subject in need thereof, the method comprising delivering to the subject the polypeptide composition of any one of claims 1-48, or the device of any one of claims 51-55.

57. The method of claim 56, wherein the polypeptide composition or the device is delivered to treat a bone defect in the subject.

58. The method of claim 57, wherein at least about 0.5 mg of polypeptide composition per cc of defect volume is delivered to the subject.

59. The method of claim 57 or claim 58, wherein about 0.5 mg to about 10 mg of polypeptide composition per cc of defect volume is delivered to the subject.

60. A method of delivering a therapeutic agent to an organ or tissue of a subject, the method comprising delivering to the organ or tissue a carrier material and the therapeutic agent, wherein the therapeutic agent is bound to the carrier material via a targeting polypeptide comprising:
(a) a sequence at least 80% identical to SEQ ID NO: 22,
(b) a sequence at least 80% identical to SEQ ID NO: 402,
(c) a sequence at least 80% identical to SEQ ID NO: 401,
(d) a sequence at least 80% identical to SEQ ID NO: 413 ((X1)(X2)), wherein X1 comprises SEQ ID NO: 1, and X2 comprises SEQ ID NO: 2 and SEQ ID NO: 6,
(e) a sequence at least 80% identical to SEQ ID NO: 21,
(f) a sequence at least 80% identical to SEQ ID NO: 414 ((X1)(X2)), wherein X1 comprises SEQ ID NO: 2, and X2 comprises SEQ ID NO: 6 and SEQ ID NO: 7,
(g) a sequence at least 80% identical to SEQ ID NO: 416 ((X1)(X2)), wherein X1 comprises SEQ ID NO: 6, and X2 comprises SEQ ID NO: 7 and SEQ ID NO: 4,
(h) a sequence at least 80% identical to SEQ ID NO: 408 ((X1)(X2)), wherein X1 comprises SEQ ID NO: 1, and X2 comprises SEQ ID NO: 2,
(i) a sequence at least 80% identical to SEQ ID NO: 20,
(j) a sequence at least 80% identical to SEQ ID NO: 409 ((X1)(X2)), wherein X1 comprises SEQ ID NO: 2, and X2 comprises SEQ ID NO: 6,
(k) a sequence at least 80% identical to SEQ ID NO: 411 ((X1)(X2)), wherein X1 comprises SEQ ID NO: 6, and X2 comprises SEQ ID NO: 7,
(l) a sequence at least 80% identical to SEQ ID NO: 412 ((X1)(X2)), wherein X1 comprises SEQ ID NO: 7, and X2 comprises SEQ ID NO: 4,
(m) a sequence at least 80% identical to SEQ ID NO: 2 (VIGESTHHRPWS),
(n) a sequence at least 80% identical to SEQ ID NO: 4 (ILAESTHHKPWT),
(o) a sequence at least 80% identical to SEQ ID NO: 6 (IIGESSHHKPFT),
(p) a sequence at least 80% identical to SEQ ID NO: 7 (GLGDTTHHRPWG), or
(q) any combination of two or more of (a) to (p).

61. The method of claim 60, wherein the carrier material comprises calcium phosphate.

62. The method of claim 60 or claim 61, wherein the targeting polypeptide is connected to the therapeutic agent.

63. The method of any one of claims 60-62, wherein the therapeutic agent comprises a growth factor.

64. The method of any one of claims 60-63, wherein the therapeutic agent comprises BMP.

65. The method of claim 64, wherein the BMP comprises BMP-2.

66. The method of any one of claims 60-63, wherein the therapeutic agent comprises a sequence at least about 80% identical to SEQ ID NO: 32.

67. The method of any one of claims 60-63, wherein the therapeutic agent comprises a sequence at least about 80% identical to any one of SEQ ID NOS: 46-71, 73-77, 79-101, 152, 168, 176, 268.

68. The method of any one of claims 60-67, wherein the therapeutic agent is delivered to treat a bone defect in the subject.

69. The method of claim 68, wherein at least about 0.5 mg of the therapeutic agent is delivered per cc of defect volume.

70. The method of claim 68 or claim 69, wherein about 0.5 mg to about 10 mg of the therapeutic agent is delivered per cc of defect volume.
